(19) United States
(12) Patent Application Publication
(10) Pub. No.: US 2019/0359937 A1
(43) Pub. Date: Nov. 28, 2019

(54) CHIMERIC RECEPTORS, BIOSENSOR HOST CELLS AND METHODS/USES THEREOF

(71) Applicant: Innovative Targeting Solutions Inc., Vancouver (CA)

(72) Inventors: Craig Robin Pigott, Vancouver (CA); Michael Lajos Gallo, North Vancouver (CA); Jaspal Singh Kang, Surrey (CA)

(73) Assignee: Innovative Targeting Solutions Inc., Vancouver, BC (CA)

(21) Appl. No.: 16/461,772

(22) PCT Filed: Nov. 17, 2017
(86) PCT No.: PCT/CA2017/051376

§ 371 Date: May 16, 2019

Related U.S. Application Data
(60) Provisional application No. 62/423,700, filed on Nov. 17, 2016.
(60) Provisional application No. 62/423,715, filed on Nov. 17, 2016.

(51) Int. Cl. C12N 5/0783; C07K 14/715; C07K 16/28; C07K 16/42; C40B 30/04; C40B 30/06; G01N 33/50; G01N 33/68; G01N 33/569; C40B 40/02; C12N 15/10

(52) U.S. Cl. CPC C12N 5/0636; G01N 2510/00; C07K 16/2809; C07K 16/4283; C40B 30/04; C40B 30/06; G01N 33/5023; G01N 33/5041; G01N 33/6863; G01N 33/56972; C40B 40/02; C12N 15/1037; C07K 2317/32; C07K 2317/35; C07K 2317/52; C07K 2317/55; C07K 2317/622; C07K 2317/75; C07K 2317/92; C07K 2318/20; C07K 2319/03; C07K 2319/30; G01N 2333/7051; G01N 2333/7151; C07K 14/7151

(57) ABSTRACT

A receptor is provided having a heterologous binding site that activates, when bound, a signaling domain related to the TNF receptor superfamily. Methods/uses of the foregoing in whole cell biosensors are also provided. There is also provided a library comprising a plurality of unique biosensor cells for binding unknown binding substrates. Each unique biosensor is a host cell having a receptor which signals production of a positive selectable marker and/or a negative selectable marker in response to the receptor being bound. Also provided is a method of identifying biosensor cells from a library that is specifically activated by a target, involving (a) contacting the library with the target substrate under positive selection conditions; (b) contacting the library with a control substrate under negative selection conditions; and (c) identifying biosensor cells which survive (a) and (b) as biosensor cells which are specifically activated by the target.

### FIELD OF INVENTION

The present invention relates to chimeric receptors and biosensors, libraries thereof and methods/uses thereof.

### BACKGROUND OF THE INVENTION

Whole cell biosensors respond to the presence of a specific analyte by producing an intracellular signal that causes the production of a specific protein product that allows for the rapid and sensitive detection of a target analyte in a complex sample. In classic biosensor formats the intracellular signal results in a product (e.g. a fluorescent reporter) that is then read out by a detector system.

High throughput functional assays are examples of biosensors used in research; functional screens look for analytes that activate the biosensor or inhibitors that prevent the activation. These systems all use physical detectors to read receptor activation and are designed for a single target readout. In general, biosensors are leveraged for their specificity and are not suitable for large libraries of specificities as each biosensor would need to be linked to a different readout.

As a platform for antibody or target discovery combined with sufficient sensitivity to detect extremely rare binding events it would also be necessary to identify the specifically bound biosensor from a large library of biosensors. The use of conventional reporters which require detection devices limits the ability to screen billions of variants and isolate the rare biosensor that was activated. An additional limitation of current reporter systems is that although they provide a sensitive method for detecting binding, these systems are suboptimal for discriminating target-specific interactions in the context of a large library of binders with diverse or unspecified target specificities, making the use of biosensors for drug and target discovery impractical. For example, in the case where a purified sample is utilized all the contaminants in the sample are potential targets for activating a biosensor library containing a diverse repertoire. Unlike traditional biosensors where the specificity is an essential element and allows for detection of a target in a complex mixture, to utilitize a diverse set of biosensors any and all contaminants would activate biosensors that cannot be distinguished from biosensors activated by the desired “target”. The same issue occurs when the purified material has a “tag” (e.g. a His tag, FLAG tag) or is expressed as an Fc fusion. In addition to the contaminants in the sample, these tags will also activate biosensors to generate a complex pool of biosensors. The problem is greatly compounded in the case where the target is a protein expressed on the cell surface.

Furthermore, finding an antibody (or other binding moiety) which specifically binds a cell surface receptor (e.g. GPCR or ion channel) is a significant challenge using a traditional biosensor. In order to identify relevant binders, it is preferred that the receptor be in its native context on the cell surface to be utilized as the antigen/binding substrate. In this case, all the other membrane proteins on the surface of the cell will activate a large set of biosensors in a complex and undefined biosensor library. The presence of all these activated biosensors makes the identification of the biosensors that are specific to the “target” a significant challenge.

A challenging class of targets for generating useful biologics are complex membrane targets such as ion channels, GPCRs and transporters. Part of the challenge is that many of these targets are expressed at low levels and the immunogenicity of all the other membrane proteins universally dominates the immune response. For example, finding antibodies that specifically bind to ion channels using hybridoma technologies is a significant challenge for the field. In phage systems, the binding to a majority of the surface expressed proteins needs to be differentiated from the rare phage that is binding to the target protein of interest and cell based panning has proven to also be a technical challenge. For yeast display systems, using whole cells is not technically feasible and as a result substitutes such as membrane preps, stabilized membranes, nanoparticles, or other methods to mimic the membrane environment are employed as suboptimal substitutes for the native membrane. The field is currently not able to routinely generate drug candidates to these important classes of membrane targets and improved methods to find rare binders (e.g. antibodies) specific to complex integral membrane proteins like ion channels are needed.

No admission is necessarily intended, nor should it be construed, that any of the preceding information constitutes prior art against the present invention.

### SUMMARY OF THE INVENTION

Various embodiments of the present invention relate to a host cell comprising: a receptor with unknown binding specificity, the receptor being natural or artificial, which signals production of a positive selectable marker and a negative selectable marker in response to the receptor being bound by a specific binding substrate, wherein the production of the positive selectable marker and/or the negative selectable marker is encoded by at least one selection cassette that is heterologous to the host cell.

The host cell may be a eukaryotic cell, a yeast cell, a vertebrate cell, a mammalian cell, or a human cell line.

The receptor may comprise an antibody, or an antigen binding fragment of the antibody, which specifically binds the specific binding substrate. The receptor may be a chimeric receptor in which the antibody or the antigen binding fragment is fused to a tumor necrosis factor receptor superfamily (TNFRSF) member or a deletion construct thereof which retains the intracellular signaling domain of the TNFRSF member.

The positive selectable marker may mediate survival of the host cell and/or the negative selectable marker may mediate death of the host cell. The positive selectable marker may be an antibiotic resistance protein. The negative selectable marker may cause apoptosis of the host cell. The negative selectable marker may be a death receptor that activates apoptosis of the host cell in response to a death receptor ligand.

Various embodiments of the present invention relate to a library of biosensor cells comprising a plurality of unique biosensor cells which collectively bind a plurality of unknown binding substrates, each unique biosensor cell being a host cell as defined herein, wherein the plurality of unique biosensor cells comprises at least 1000, at least 10,000, at least 100,000, at least 1 million, at least 10 million, at least 100, million, at least 1 billion, or at least 10 billion unique biosensor cells.

Various embodiments of the present invention relate to an in vitro method of identifying a biosensor cell from the library that is specifically activated by a target substrate, comprising: (a) contacting the library with the target substrate under positive selection conditions; (b) contacting the library with a control substrate under negative selection conditions; and (c) identifying biosensor cells which survive (a) and (b) as biosensor cells which are specifically activated by the target substrate. Step (a) may precede step (b). Step (b) may precede step (a). Steps (a) and (b) may be iterative.

Various embodiments of the present invention relate to a chimeric receptor comprising: a signaling portion comprising a tumor necrosis factor receptor superfamily (TNFRSF) member or a fragment of the TNFRSF member which retains an intracellular signaling domain of the TNFRSF member; a transmembrane domain; and a binding portion comprising a binding site which specifically binds a binding substrate, wherein the binding site is not native to the TNFRSF family member; wherein the binding portion and the intracellular signaling domain and the of signaling portion are oriented such that the binding portion is extracellular and the intracellular signaling domain is intracellular when the chimeric receptor is expressed in a vertebrate cell.

The transmembrane domain of the chimeric receptor may be comprised within the TNFRSF member or the fragment of the TNFRSF member.

The TNFRSF member may be a death receptor, wherein the death receptor has at least 80% sequence identity to TNFR1, FAS, TRAILR1, TRAILR2, TRAMP or CD358 and retains TNFRSF membrane localization and TNFRSF intracellular signaling activity when expressed in the vertebrate cell. The TNFRSF member may be a death receptor, wherein the death receptor is TNFR1, FAS, TRAILR1, TRAILR2, TRAMP or CD358. The signaling portion may comprise the amino acid sequence of SEQ ID NO: 6, 7, 8, 9 or 10.

The signaling portion of the chimeric receptor may comprise the TNFRSF member in its full length. The binding portion and the signaling portion may be fused with a peptide linker.

The binding portion of the chimeric receptor may comprise a monobody, an affibody, an anticalin, a DARPin, a Kunitz domain, an avimer or a soluble T-cell receptor.

The binding portion of the chimeric receptor may comprise an antibody or an antigen-binding fragment of the antibody. The antibody or the antigen-binding fragment may bind to the binding substrate with a K_{D} of less than 200 nM. The binding portion may comprise an IgG antibody.

Various embodiments of the present invention relate to at least one nucleic acid comprising one or more coding sequences which collectively encode the chimeric receptor defined herein.

The at least one nucleic acid may further comprise at least one promoter operably linked to the one or more coding sequences. The at least one promoter may comprise one or both of weak promoter and an inducible promoter. The inducible promoter may be a tetracycline-regulated promoter.

The one or more coding sequences of the at least one nucleic acid may comprise or may be operably linked to one or more genetic elements which, when the chimeric receptor is expressed in the vertebrate cell, cause expression of the chimeric receptor at a level that is sufficiently low such that signaling caused by binding of the binding substrate to the chimeric receptor is distinguishable over background signaling. The one or more genetic elements may comprise: a Kozak sequence in the nucleic acid which causes inefficient translation of the chimeric receptor; codons in the at least one coding sequence which are not optimized for efficient translation in the vertebrate cell; one or more RNA destabilizing sequences in the nucleic acid for reducing the half-life of an RNA transcribed from the nucleic acid which encodes the chimeric receptor; intron and/or exon sequences in the one or more coding sequence which cause inefficient intron splicing; the chimeric receptor encoded by the nucleic acid further comprises one or more ubiquination sequences; or a combination thereof.

Various embodiments of the present invention relate to a vertebrate cell comprising the at least one nucleic defined herein. The at least one promoter of the at least one nucleic acid of the vertebrate cell may comprise an inducible promoter, wherein the vertebrate cell expresses a repressor which binds an operator of the inducible promoter. The vertebrate cell may or may not express TetR.

The vertebrate cell may further comprise at least one nucleic acid sequence for expressing antisense RNA or RNAi configured to reduce expression levels of the chimeric protein.

The vertebrate cell may further comprise a marker gene operably linked to a second promoter and a NFκB response element such that expression of the marker gene is activated by NFκB binding the NFκB response element and repressed in the absence of said NFκB binding. The marker gene may encode a surface antigen or expression of the marker gene may cause expression of the surface antigen. The marker gene may encode CD19 antigen fused to puromycin N-acetyl-transferase (Puro) and may be configured for intracellular display of Puro and extracellular display of the CD19 antigen. The marker gene may encode a resistance protein which confers resistance to a toxic compound or condition or causes expression of the resistance protein when the marker gene is expressed. The marker gene may encode a toxin or enzyme which converts a precursor compound to a toxic compound or expression of the marker gene causes the expression of the toxin or the enzyme. The marker gene may encode an apoptosis-inducing protein. The apoptosis-inducing protein may be a death receptor that is activated by a ligand that does not activate other death receptors expressed by the vertebrate cell.

The vertebrate cell may further comprise two or more genes of interest in a polycistronic operon that is operably linked to a second promoter and a NFκB response element such that expression of the two or more genes of interest is activated by NFκB binding the NFκB response element and repressed in the absence of said NFκB binding.

The at least one nucleic acid of the vertebrate cell may be integrated in a chromosome of the vertebrate cell.

The vertebrate cell may be a human cell or human-derived cell line.

Various embodiments of the present invention relate to a method of detecting binding between a biosensor and a multivalent binding substrate, the method comprising: contacting the biosensor with the multivalent binding substrate, the biosensor comprising a first vertebrate cell that expresses a chimeric protein, wherein the chimeric protein comprises: a signaling portion comprising a transmembrane tumor necrosis factor receptor superfamily (TNFRSF) member or a fragment of the TNFRSF member which retains an intracellular signaling domain of the TNFRSF member; a transmembrane domain; and an extracellular binding portion comprising a binding site which specifically binds a binding substrate, wherein the binding site is not native to the TNFRSF family member, wherein the binding portion and the intracellular signaling domain and the of signaling portion are oriented such that the binding portion is extracellular and the intracellular signaling domain is intracellular when the chimeric receptor is expressed in a vertebrate cell; wherein binding of the multivalent binding substrate to the binding site of the extracellular binding portion activates intracellular signaling activity of the signaling portion; and identifying binding between the biosensor and the multivalent binding substrate based on a level of the intracellular signaling activity compared with a background level.

The level of the intracellular signaling activity may positively correspond to a rate of cell death of the biosensor or positively corresponds to a rate of cell survival of the biosensor.

The method may further comprise contacting the biosensor with an exogenous mediator.

The level of the intracellular signaling activity positively corresponds to an expression level of a marker gene that is activated by NFκB, the marker gene being a one or more of a screenable marker gene, a selectable marker gene or a screenable-selectable marker gene.

The marker gene may be a death receptor that is activated by a ligand that does not activate other death receptors expressed by the first vertebrate cell, and the method may further comprise contacting the biosensor with the ligand.

TNFRSF member in the method may be a death receptor, and the method may further comprise contacting the biosensor with a caspase inhibitor prior to or during said contacting the biosensor with the multivalent binding substrate.

Said contacting the biosensor with the multivalent binding substrate may comprise co-culturing the biosensor with a second vertebrate cell, the second vertebrate cell comprising the multivalent binding substrate.

The method may further comprise preparing the multivalent binding substrate prior to said contacting the biosensor with the multivalent binding substrate by oligomerizing a binding substrate.

Various embodiments of the present invention relate to a chimeric receptor comprising: a signaling portion comprising a tumor necrosis factor receptor superfamily (TNFRSF) member or a fragment of the TNFRSF member which retains an intracellular signaling domain of the TNFRSF member; a transmembrane domain; and a binding portion comprising an extracellular binding site which specifically binds a binding substrate, wherein the binding portion comprises a monobody, an affibody, an anticalin, a DARPin, a Kunitz domain, an avimer, a soluble T-cell receptor (TCR), an antibody or an antigen-binding fragment of the antibody, or wherein the chimeric receptor comprises, and the binding portion is comprised within, a TCR or an antigen-binding fragment of the TCR. The transmembrane domain may be comprised within the TNFRSF member or the fragment of the TNFRSF member. The transmembrane domain may be a single-spanning transmembrane domain, with the proviso that it is not from the TNFRSF member. The transmembrane domain may be from PDGFR, glucagon-like peptide 1 receptor or CD20. The transmembrane domain may be a multi-spanning transmembrane receptor. The chimeric receptor may comprise a truncation of the TNFRSF member. The chimeric receptor may comprise the TNFRSF member in its full length. The TNFRSF member may have at least 80% sequence identity to TNFR1, FAS, TRAILR1, TRAILR2, TRAMP, CD358 or CD27 and retain functional membrane localization and TNFRSF intracellular signaling activity when expressed in the vertebrate cell. The signaling portion may comprise an amino acid sequence that is at least 80% identical to SEQ ID NO: 63 or 64 and retain intracellular signaling activity. The binding portion may comprise an antibody or an antigen-binding fragment of the antibody. The antibody or the antigen-binding fragment may bind the binding substrate with a K_{D} of less than 200 nM. The binding portion may comprise an IgG antibody. The binding portion may be fused to the transmembrane domain with a first peptide linker; and/or the signaling portion may be fused to the transmembrane domain with a second peptide linker.

Various embodiments of the present invention relate to at least one nucleic acid comprising one or more coding sequences which collectively encode the chimeric receptor defined herein. The at least one nucleic acid may further comprise at least one promoter operably linked to the one or more coding sequences. The least one promoter may comprise one or both of a weak promoter and an inducible promoter. The inducible promoter may be a tetracycline-regulated promoter. The one or more coding sequences may comprise or be operably linked to one or more genetic elements which, when the chimeric receptor is expressed in a eukaryotic cell that is NF-κB-competent, cause expression of the chimeric receptor at a level that is sufficiently low such that signaling caused by binding of the binding substrate to the chimeric receptor is distinguishable over background signaling in the absence of the binding substrate. The one or more genetic elements may comprise: a Kozak sequence in the nucleic acid which causes inefficient translation of the chimeric receptor; codons in the at least one coding sequence which are not optimized for efficient translation in the eukaryotic cell; one or more RNA destabilizing sequences in the nucleic acid for reducing the half-life of an RNA transcribed from the nucleic acid which encodes the chimeric receptor; intron and/or exon sequences in the one or more coding sequences which cause inefficient intron splicing; the chimeric receptor encoded by the at least one nucleic acid further comprises one or more ubiquination sequences; or a combination thereof.

Various embodiments of the present invention relate to a eukaryotic cell comprising the at least one nucleic acid defined herein, wherein the eukaryotic cell expresses TetR.

Various embodiments of the present invention relate to a eukaryotic cell comprising the at least one nucleic acid defined herein, wherein the at least one promoter comprises an inducible promoter, and wherein the eukaryotic cell expresses a repressor which binds an operator of the inducible promoter.

Various embodiments of the present invention relate to a eukaryotic cell comprising the at least one nucleic acid defined herein, further comprising at least one nucleic acid sequence for expressing antisense RNA or RNAi configured to reduce expression levels of the chimeric receptor.

Various embodiments of the present invention relate to a eukaryotic cell comprising the at least one nucleic acid defined herein, wherein the eukaryotic cell is NF-κB competent in response to activation of the TNFRSF member and wherein the eukaryotic cell further comprises a marker gene, heterologous to the eukaryotic cell, operably linked to a second promoter and a NF-κB response element such that expression of the marker gene is activated by NF-κB binding the NF-κB response element and inactive or repressed in the absence of said NF-κB binding. The marker gene may encode a surface antigen or expression of the marker gene may cause expression of the surface antigen. The marker gene may encode an integral membrane protein that displays an extracellular surface antigen and an intracellular resistance protein which confers resistance to a toxic compound or condition. The marker gene may encode a resistance protein which confers resistance to a toxic compound or condition or may cause expression of the resistance protein when the marker gene is expressed. The marker gene may encode a toxin or enzyme which converts a precursor compound to a toxic compound or expression of the marker gene may cause the expression of the toxin or the enzyme. The marker gene may encode an apoptosis-inducing protein. The apoptosis-inducing protein may be a death receptor.

Various embodiments of the present invention relate to a eukaryotic cell comprising the at least one nucleic acid defined herein, wherein the eukaryotic cell is a vertebrate cell which further comprises: two or more genes of interest in a polycistronic operon that is operably linked to a second promoter and a NF-κB response element; and/or two or more genes of interest in separate operons that are operably linked to two or more additional promoters and NF-κB response elements; such that expression of the two or more genes of interest is activated by NF-κB binding the NF-κB response element and inactive or repressed in the absence of said NF-κB binding. The eukaryotic cell may further comprise an expression cassette for expressing a cell surface protein comprising an extracellular domain comprising: a multivalent binding substrate; or a univalent binding substrate that forms the multivalent binding substrate through multimerization of the cell surface protein. The expression cassette for the cell surface protein may comprise an inducible promoter operably linked to a nucleic acid sequence or sequences encoding the cell surface protein.

Any of the eukaryotic cells above may be a vertebrate cell. Any of the eukaryotic cells above may be a human cell or a human-derived cell line.

For any of the eukaryotic cells above, the at least one nucleic acid may be integrated in a chromosome of the eukaryotic cell.

Various embodiments of the present invention relate to a method of detecting binding between a biosensor and a multivalent binding substrate, the method comprising: contacting the biosensor with the multivalent binding substrate, the biosensor comprising a first vertebrate cell that expresses a chimeric protein, wherein the chimeric protein comprises: a signaling portion comprising a transmembrane tumor necrosis factor receptor superfamily (TNFRSF) member or a fragment of the TNFRSF member which retains an intracellular signaling domain of the TNFRSF member; a transmembrane domain; and a binding portion comprising an extracellular binding site which specifically binds a binding substrate, wherein the extracellular binding site is not native to the TNFRSF member; wherein binding of the multivalent binding substrate to the extracellular binding site activates intracellular signaling activity of the signaling portion; and identifying binding between the biosensor and the multivalent binding substrate based on a level of the intracellular signaling activity compared with a background level. The level of the intracellular signaling activity may positively correspond to a measure of cell death of the biosensor or positively correspond to a measure of cell survival of the biosensor. The method may further comprise contacting the biosensor with an exogenous mediator. The level of the intracellular signaling activity may positively correspond to an expression level of a marker gene that is activated by NF-κB, the marker gene being a one or more of a screenable marker gene, a selectable marker gene or a screenable-selectable marker gene. The marker gene may be a death receptor that is activated by a ligand that does not activate other death receptors expressed by the first vertebrate cell if the other death receptors are present, and the method may further comprise contacting the biosensor with the ligand. The TNFRSF member may be a death receptor, and the method may further comprise contacting the biosensor with a caspase inhibitor prior to or during said contacting the biosensor with the multivalent binding substrate. The chimeric protein may be any chimeric receptor as defined herein, or the first vertebrate cell may comprise any at least one nucleic acid as defined herein, or the first vertebrate cell may be any eukaryotic cell as defined herein. The at least one nucleic acid may be integrated in a chromosome of the first vertebrate cell. Contacting the biosensor with the multivalent binding substrate may comprise co-culturing the biosensor with a second vertebrate cell, the second vertebrate cell comprising the multivalent binding substrate. The method may further comprise preparing the multivalent binding substrate prior to said contacting the biosensor with the multivalent binding substrate by oligomerizing a binding substrate. Contacting the biosensor with the multivalent binding substrate may comprise co-expressing a cell surface protein in the first vertebrate cell with the chimeric protein, the cell surface protein comprising an extracellular domain comprising: the multivalent binding substrate; or a univalent binding substrate that forms the multivalent binding substrate through multimerization of the cell surface protein. Co-expressing the cell surface protein may be inducible, the method may further comprise inducing expression of the cell surface protein.

Various embodiments of the present invention relate to a library of biosensor cells comprising a plurality of unique biosensor cells which collectively bind a plurality of unknown binding substrates, each unique biosensor cell being a host cell comprising: a receptor comprising a binding site having unique binding specificity compared to other receptors in the plurality of unique biosensor cells, wherein the receptor is artificial, wherein the receptor signals production of a positive selectable marker and/or a negative selectable marker in response to the binding site being bound by a specific binding substrate, and wherein the production of the positive selectable marker and/or the negative selectable marker is encoded by at least one selection cassette that is heterologous to the host cell; wherein the plurality of unique biosensor cells comprises at least 1000, at least 10,000, at least 100,000, at least 1 million, at least 10 million, at least 100, million, at least 1 billion, or at least 10 billion unique biosensor cells. The host cell may be a eukaryotic cell, a yeast cell, a vertebrate cell, a mammalian cell, a human cell or a human cell line. The receptor may comprise, and the unique binding specificity may be from, an antibody, an antigen binding fragment of the antibody which specifically binds the specific binding substrate, a T-cell receptor (TCR), a soluble TCR, an antigen binding fragment of the TCR or the soluble TCR which specifically binds the specific binding substrate, a monobody, an affibody, an anticalin, a DARPin, a Kunitz domain, an avimer or a peptide of at least 7 amino acid residues. The host cell may be NF-κB competent and the receptor may be a transmembrane receptor which further comprises: a signaling portion comprising a tumor necrosis factor receptor superfamily (TNFRSF) member or a fragment of the TNFRSF member which retains an intracellular signaling domain of the TNFRSF member; a transmembrane domain; and a binding portion comprising the binding site, wherein the binding portion is extracellular and the intracellular signaling domain of the signaling portion is intracellular. The host cell may be a vertebrate cell, mammalian cell, a human cell or a human cell line, and: the receptor may be any chimeric receptor as defined herein; or the host cell may comprise any at least one nucleic acid as defined herein, or the host cell may be any eukaryotic cell as defined herein which is a vertebrate cell. The at least one nucleic acid may be integrated in a chromosome of the host cell. The positive selectable marker may mediate survival of the host cell and/or the negative selectable marker may mediate death of the host cell. The positive selectable marker may be an antibiotic resistance protein. The negative selectable marker may cause apoptosis of the host cell. The negative selectable marker may be a death receptor that activates apoptosis of the host cell in response to presence of a death receptor ligand.

Various embodiments of the present invention relate to an in vitro method of identifying a biosensor cell from any library as defined herein that is specifically activated by a target substrate or target substrates, wherein the receptor of each unique biosensor cell signals production of both a positive selectable marker and a negative selectable marker in response to the binding site being bound by the specific binding substrate for that unique biosensor cell, the method comprising: (a) contacting the library with the target substrate or the target substrates under positive selection conditions; (b) contacting the library with a control substrate or control substrates under negative selection conditions; and (c) identifying biosensor cells which survive (a) and (b) as biosensor cells which are specifically activated by the target substrate or the target substrates. Step (a) may precede step (b) or step (b) may precede (a). Steps (a) and (b) may be iterative.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1. FIGS. 1A and 1B show two examples of dual selection biosensors in use.

FIG. 2. FIG. 2A shows a schematic representation of plasmid C112. FIG. 2B shows the nucleic acid sequence of plasmid C112.

FIG. 3. FIG. 3A shows a schematic map of plasmid C659. FIG. 3B shows the nucleic acid sequence of plasmid C659 with the NF-κB response element underlined.

FIG. 3C shows the amino acid sequence of CD19-Puro, with CD19 shown in bold, linker shown underlined and Puro shown in italics.

FIG. 4. FIG. 4A shows a schematic map of plasmid C601. FIG. 4B shows the nucleic acid sequence of plasmid C601. FIG. 4C shows the amino acid sequences of the C601-expressed IgG(heavy chain)-TNFR1(full length) with leader sequence for surface expression (IgG shown in bold, including leader sequence underlined and bold; peptide linker shown underlined; TNFR1 shown in italics) and the IgG(light chain) without leader sequence (LoxP at 412-379; FLAG tag at 415-783; Hygromycin resistance gene at 784-1806; BGH poly(A) at 1837-2061; TK promoter at 2069-2200; Tet operators (2) at 2219-2201, 2248-2230; TATA Box at 2222-2228; Anti-CD3 Heavy Chain 2354-3757; TNFR1 at 3764-5068; BGH poly(A) at 5075-5326; CMV promoter at 7691-8311; Anti-CD3 Light Chain at 8353-9060; BGH poly(A) at 9098-9322).

FIG. 5. FIG. 5A shows a schematic map of plasmid C638. FIG. 5B shows the nucleic acid sequence of plasmid C638. FIG. 5C shows the amino acid sequences of the C638-expressed IgG(heavy chain)-TNFR1(full length) with leader sequence for surface expression (IgG shown in bold, including leader sequence underlined and bold; peptide linker shown underlined; TNFR1 shown in italics) and the IgG(light chain) with leader sequence (shown underlined). The light chain sequence is the same as for the light chain expressed by plasmid C644.

FIG. 6. FIG. 6A shows a bar graph depicting expression of CD19 as a reporter in cell lines L1122 (chimeric receptor: IgG(anti-CD3)-TNFR1) and L1123 (chimeric receptor: IgG(unknown specificity)-TNFR1) when untreated, treated with anti-IgG Fc antibody, or treated by co-culturing with Jurkat cells (which express CD3 on their cell surfaces). FIG. 6B shows micrographs of L1122 and L1123 cells in the presence of puromycin when previously untreated, treated with anti-IgG Fc antibody, or treated by co-culturing with Jurkat cells (which express CD3 on their cell surfaces).

FIG. 7. FIG. 7A shows a schematic map of plasmid C644. FIG. 7B shows the nucleic acid sequence of plasmid C645 (sequence encoding anti-HER2 heavy chain shown underlined; sequence encoding anti-HER2 light chain shown-double-underlined; CMV promoter bolded). FIG. 7C shows the amino acid sequence of IgG(heavy chain)-TNFR1 (with leader sequence for surface expression) encoded by either plasmid C644 or plasmid C645 (IgG shown in bold, including leader sequence underlined and bold; peptide linker shown underlined; TNFR1 shown in italics). FIG. 7D shows the amino acid sequence of IgG(light chain) (with leader sequence shown underlined) encoded by plasmid C645. FIG. 7E shows nucleotide and amino acid sequences of HER2ECD-PDFR. FIG. 7F shows CD19-Puro expression in L1077 cells (C644-integrated) and L1078 cells (C645-integrated) co-cultured with L707.3 cells (control, no/low HER2 expression), co-cultured with L1101 cells (HER2 overexpression) and incubated with anti-IgG Fc antibody (multivalent binding substrate).

FIG. 8. FIG. 8A shows schematic representations of five IgG(heavy chain)-TNFR1 chimeric receptor constructs, including full-length TNFR1 (ITS017-V030) and four deletion constructs. FIG. 8B shows amino acid sequences for the five constructs, in which the heavy chain IgG is shown in bold (including leader sequence underlined and bold), the linker is shown underlined, and the TNFR1 portion is shown in italics. FIG. 8C shows the amino acid sequences of the TNFR1 portions for each deletion construct alone. FIG. 8D shows the nucleic acid sequence of a plasmid for expressing ITS017-V030. Plasmids for the other constructs are identical, except the nucleotides corresponding to the deleted amino acids are omitted for the deletion constructs. FIG. 8E shows the nucleic acid sequence of plasmid V707, which encodes the light chain for the IgG-TNFR1 constructs in FIG. 8, showing the sequence encoding the light chain (without leader) in bold (note the plasmid has an intron between leader and mature variable region). FIG. 8F shows the amino acids sequence of the IgG(light chain) (mature protein; no leader sequence) expressed by plasmid V707.

FIG. 9 shows (A) a schematic map and (B) a nucleotide sequence (SEQ ID NO: 48) for plasmid C487, a CD19 NF-κB reporter construct (NF-κB at 411-462, underlined; HindlIl at 482-487, bolded; CD19 ORF at 558-2228, underlined; XbaI at 2242-2247, bolded).

FIG. 10 shows (A) a schematic map and (B) a nucleotide sequence (SEQ ID NO: 49) for plasmid C639 (MluI at 3761-3766, bolded; LoxP at 412-379; MYC tag at 415-780; Irrelevant Heavy Chain at 2351-3760; Truncated TNFR1 at 3767-4546, underlined). Plasmid C639 is the parent vector for T96, T101 and T145.

FIG. 11 shows (A) a schematic and (B) a nucleotide sequence (SEQ ID NO: 50) for the portion of plasmid C884 encoding TRAILR1 linked to NF-κB responsive elements, constructed by cloning into the HindIII and XbaI restriction sites of plasmid C487. In the sequence, restriction sites are shown bolded and TRAILR1 (i.e. TNFRSF10A CDS) is shown underlined.

FIG. 12 shows flow cytometry analysis using a monoclonal anti-Myc tag antibody linked to Alexa 647 to visualize (A) Myc tag expression on L1262 and L1280 cells observed and (B) Myc tag expression on a mixed population of L1262 and L1280 cells cultured for 8 days in the presence or absence of a combination of 1 μg/ml anti-human IgG Fc and 20 ng/ml TRAIL.

FIG. 13 shows (A) a schematic and (B) a nucleotide sequence (SEQ ID NO: 51) for the portion of plasmid T99 encoding an IgG(anti-CD3)-TRAILR1 chimeric receptor, constructed by cloning the chimeric receptor into the BamHI and SalI restriction sites of plasmid C601. In the sequence, restriction sites are shown bolded, the heavy chain is shown underlined, and TRAILR1 (i.e. TNFRSF10A CDS) is shown double-underlined. Among other things, T99 also encodes a FLAG tag.

FIG. 14 shows (A) a schematic and (B) a nucleotide sequence (SEQ ID NO: 52) for the portion of plasmid T100 encoding an IgG(anti-CD3)-TRAILR2 chimeric receptor, constructed by cloning the chimeric receptor into the BamHI and SalI restriction sites of plasmid C601. In the sequence, restriction sites are shown bolded, the heavy chain is shown underlined, and TRAILR1 (i.e. TNFRSF10A CDS) is shown double-underlined. Among other things, T100 also encodes a FLAG tag.

FIG. 15A shows a schematic for the portion of plasmid ITS017-V057 encoding an IgG(anti-HLA-A*02:01-restrictedNY-ESO-1 (SLLMWITQC) antigenic peptide)-CD27 chimeric receptor, constructed by cloning the chimeric receptor into the BamHI and SalI restriction sites of plasmid C601. FIG. 15B shows a bar graph depicting expression of reporter in cell line ITS017-L021 when treated with anti-human IgG Fc, biotin-labeled NY-ESO-1/MHC/Steptavidin complex or biotin-labeled HIV gag/MHC/Streptavidin complex. Reporter expression from the untreated control was subtracted from the treatment conditions.

FIG. 16 shows (A) a schematic and (B) a nucleotide sequence (SEQ ID NO: 53) for the portion of plasmid T96 encoding a IL8-TNFR1 chimeric receptor, constructed by cloning the chimeric receptor into the BamHI and SalI restriction sites of plasmid C639. In the sequence, restriction sites are shown bolded, IL-8 is shown underlined, and TNFR1 is shown double-underlined. Among other things, T96 also encodes a Myc tag.

FIG. 17 shows (A) a schematic and (B) a nucleotide sequence (SEQ ID NO: 54) for the portion of plasmid T101 encoding a CD73-TNFR1 chimeric receptor, constructed by cloning the chimeric receptor into the BamHI and SalI restriction sites of plasmid C639. In the sequence, restriction sites are shown bolded, CD73 is shown underlined, and TNFR1 is shown double-underlined. Among other things, T101 also encodes a Myc tag.

FIG. 18 shows (A) a schematic map and (B) a nucleotide sequence (SEQ ID NO: 55) for plasmid C58 (LoxP at 412-379; V5 tag at 415-804; Hygromycin at 805-1827; BGH pA at 1858-2082; CMV promoter at 2090-2710; light chain at 2752-3462, underlined; EMCV IRES at 3476-4063; heavy chain at 4120-5679, underlined; PDGFR TM at 5530-5679, bolded; SV40 PA region at 5749-5879).

FIG. 19 shows (A) a schematic and (B) a nucleotide sequence (SEQ ID NO: 56) for the portion of plasmid T145 encoding a CD73-TNFR1(no ECD) chimeric receptor, constructed by cloning into the BamHI and SalI restriction sites of plasmid C639. In the sequence, restriction sites are shown bolded, CD73 is shown underlined, and TNFR1(no ECD) is shown double-underlined. Among other things, T145 also encodes a Myc tag.

FIG. 20 shows (A) a schematic and (B) a nucleotide sequence (SEQ ID NO: 57) for the portion of plasmid T110 encoding a IgG(anti-CD3)-TNFR1ECD-CD4TM-TNFR1ICD chimeric receptor, constructed by cloning into the BamHI and SalI restriction sites of plasmid C601. In the sequence, restriction sites are shown bolded, IgG heavy chain is shown underlined, and CD4(TM) is shown double-underlined. Among other things, T110 also encodes a FLAG tag.

FIG. 21 shows (A) a schematic and (B) a nucleotide sequence (SEQ ID NO: 58) for the portion of plasmid T111 encoding a IgG(anti-CD3)-TNFR1(ECD)-PDGFR(TM)-TNFR1(ICD) chimeric receptor, constructed by cloning into the BamHI and SalI restriction sites of plasmid C601. In the sequence, restriction sites are shown bolded, IgG heavy chain is shown underlined, and PDGFR(TM) is shown double-underlined. Among other things, T111 also encodes a FLAG tag.

FIG. 22 shows (A) a schematic and (B) a nucleotide sequence (SEQ ID NO: 59) for the portion of plasmid T146 encoding a CD73(no anchor)-PDGFR(TM)-TNFR1(ICD) chimeric receptor, constructed by cloning into the BamHI and SalI restriction sites of plasmid C601. In the sequence, restriction sites are shown bolded, CD73(no anchor) is shown underlined, PDGFR(TM) is shown bold underlined and TNFR1(ICD) is shown double-underlined. Among other things, T146 also encodes a FLAG tag. FIG. 22(C) shows an amino acid sequence that contains the TNFR1(ICD) (SEQ ID NO: 63).

FIG. 23 shows (A) a schematic and (B) a nucleotide sequence (SEQ ID NO: 60) for the portion of plasmid T147 encoding a CD73(no anchor)-PDGFR(TM)-TRAILR2(ICD) chimeric receptor, constructed by cloning into the BamHI and SalI restriction sites of plasmid C601. In the sequence, restriction sites are shown bolded, CD73(no anchor) is shown underlined, PDGFR(TM) is shown bold underlined and TRAILR2(ICD) is shown double-underlined. Among other things, T147 also encodes a FLAG tag. FIG. 23(C) shows an amino acid sequence that contains the TRAILR2(ICD) (SEQ ID NO: 64).

FIG. 24 shows (A) a schematic map and (B) a nucleotide sequence (SEQ ID NO: 61) for plasmid T173 (LoxP at 412-379; FLAG tag at 415-783; Hygromycin at 784-1806; BGH pA at 1837-2061; TK promoter at 2069-2200; Nod at 2330-2337; GLP1R(no ICD) at 2362-3573, bolded; TNFR1(ICD) at 3574-4239, underlined; XbaI at 4241-4246; BGH pA at 4278-4502).

FIG. 25 shows (A) a schematic map for plasmid T175, (B) a schematic of the NotI to XbaI region of the plasmid, and (C) the nucleotide sequence (SEQ ID NO: 62) of the NotI to XbaI region (CD20 (no C-terminal ICD) at 222-648, bolded; TNFR1(ICD) at 649-1314, underlined).

### DETAILED DESCRIPTION

### I. General Definitions

As used herein, the terms “comprising,” “having”, “including” and “containing,” and grammatical variations thereof, are inclusive or open-ended and do not exclude additional, unrecited elements and/or method steps. The term “consisting essentially of” if used herein in connection with a composition, use or method, denotes that additional elements and/or method steps may be present, but that these additions do not materially affect the manner in which the recited composition, method or use functions. The term “consisting of” if used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the content clearly dictates otherwise. The use of the word “a” or “an” when used herein in conjunction with the term “comprising” may mean “one,” but it is also consistent with the meaning of “one or more,” “at least one” and “one or more than one.”

Unless indicated to be further limited, the term “plurality” as used herein means more than one, for example, two or more, three or more, four or more, and the like.

If used herein, the term “about” refers to an approximately +/−10% variation from a given value. It is to be understood that such a variation is always included in any given value provided herein, whether or not it is specifically referred to.

In this disclosure, the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, protein, chimeric receptor, nucleic acid, at least one nucleic acid, cell, cell, kit, et cetera. None of Sections I, II, III, IV, V, VI and VII should be viewed as independent of the other Sections, but instead should be interpreted as a whole. Unless otherwise indicated, embodiments described in individual sections may further include any combination of features described in the other sections. Definitions presented for terms in any section(s) may be incorporated into other section(s) as a substitute or alternative definition.

As used herein, a “polypeptide” is a chain of amino acid residues, of any size, including without limitation peptides and protein chains. A polypeptide may include amino acid polymers in which one or more of the amino acid residues is an artificial chemical analogue of a corresponding naturally occurring amino acid, or is a completely artificial amino acid with no obvious natural analogue as well as to naturally occurring amino acid polymers.

The term “protein” comprises polypeptides as well as polypeptide complexes, which may or which may not include, without limitation, one or more co-factors, carbohydrate chains, nucleic acids, small molecule or other non-polypeptide moeity, whether covalently or non-covalently bound. Accordingly, a protein may comprise 1, 2, 3, 4, 5, 6, 7, 8, 9, 10 or more than 10 polypeptide chains in covalent and/or non-covalent association. Non-limiting examples of non-covalent interaction include hydrogen bonds, hydrophobic interactions and/or electrostatic interactions. A non-limiting example of a covalent bond between polypeptides is a disulfide bridge.

As used herein, “nucleic acid”, “nucleic acid sequence”, “nucleotide sequence”, “polynucleotide” or similar terms mean oligomers of bases typically linked by a sugar-phosphate backbone, for example but not limited to oligonucleotides, and polynucleotides, or DNA or RNA of genomic or synthetic origin which can be single- or double-stranded, and represent a sense or antisense strand. The terms nucleic acid, polynucleotide, nucleotide and similar terms also specifically include nucleic acids composed of bases other than the five biologically occurring bases (i.e., adenine, guanine, thymine, cytosine and uracil), and also include nucleic acids having non-natural backbone structures. Unless otherwise indicated, a particular nucleic acid sequence of this invention encompasses complementary sequences, in addition to the sequence explicitly indicated.

In this disclosure, “nucleic acid vector”, “vector” and similar terms refer to at least one of a plasmid, bacteriophage, cosmid, artificial chromosome, expression vector, or any other nucleic acid vector. Those skilled in the art, in light of the teachings of this disclosure, will understand that alternative vectors or plasmids may be used, or that the above vectors may be modified in order to combine sequences as desired. For example, vectors or plasmids may be modified by inserting additional origins of replication, or replacing origins of replication, introducing expression cassettes comprising suitable promoter and termination sequences, adding one or more than one DNA binding sequence, DNA recognition site, or adding sequences encoding polypeptides as described herein, other products of interest, polypeptides of interest or proteins of interest, or a combination thereof. In some embodiments adjacent functional components of a vector or plasmid may be joined by linking sequences.

A “coding sequence” or a sequence which is “encoded”, as used herein, includes a nucleotide sequence encoding a product of interest, for example a peptide or polypeptide, or a sequence which encodes RNA that lacks a translation start and/or stop codon or is otherwise unsuitable for translation into a peptide or polypeptide, for example, an RNA precursor of small interfering RNAs (siRNAs) or microRNAs (miRNAs).

A “promoter” is a DNA region, typically but not exclusively 5′ of the site of transcription initiation, sufficient to confer accurate transcription initiation. The promoter nucleic acid typically contains regions of DNA that are involved in recognition and binding of RNA polymerase and other proteins or factors to initiate transcription. In some embodiments, a promoter is constitutively active, while in alternative embodiments, the promoter is conditionally active (e.g., where transcription is initiated only under certain physiological conditions). Conditionally active promoters may thus be “inducible” in the sense that expression of the coding sequence can be controlled by altering the physiological condition.

A “terminator” or “transcription termination site” refers to a 3′ flanking region of a gene or coding sequence that contains nucleotide sequences which regulate transcription termination and typically confer RNA stability.

As used herein, “operably linked”, “operatively linked”, “in operative association” and similar phrases, when/if used in reference to nucleic acids, refer to the linkage of nucleic acid sequences placed in functional relationships with each other. For example, an operatively linked promoter sequence, open reading frame and terminator sequence results in the accurate production of an RNA molecule in a cell environment. In some aspects, operatively linked nucleic acid elements result in the transcription of an open reading frame and ultimately the production of a polypeptide (i.e., expression of the open reading frame). Where transcription of a coding sequence is intended, operable linkage of a coding sequence to a promoter also includes operable linkage of the coding sequence to a terminator, regardless of whether the terminator is explicitly mentioned.

The term “cassette” (e.g. expression cassette or selection cassette) means a configuration of genetic elements including a coding sequence and its regulatory elements (e.g. a promoter, operator(s) and/or a terminator). As used herein, a selection cassette comprises at least a promoter operably linked to a selectable marker gene.

The term “heterologous” generally means that something is non-native to its environment or to another element (e.g. artificially introduced or combined or otherwise derived from a different cell or organism). As used herein, a gene or a protein or a cassette that is “heterologous” to a cell (e.g. a host cell or a biosensor cell) means that the gene or protein or cassette was not found in the native or natural cell, but is an artificial construct or a natural construct obtained from or found in a different cell type or organism. A heterologous sequence or subsequence (or portion or domain of a fusion protein) refers to that sequence/portion/domain being derived from a different gene/protein than another reference sequence/portion/domain, even if the two sequences or domains are from the same source cell or species.

As used herein, the term “fusion protein” means a protein encoded by at least one nucleic acid coding sequence that is comprised of a fusion of two or more coding sequences from separate genes.

The terms “conservative mutant” and “conservatively modified variants” and similar phrases apply to both amino acid and nucleic acid sequences, and have the same meaning as would be understood by a person of skill in the art. With respect to amino acid sequences (or nucleic acids that encode amino acid sequences), one of skill in the art will recognize that individual substitutions, deletions or additions to a nucleic acid, peptide, polypeptide, or protein sequence which alters, adds and/or deletes a single amino acid or a specified percentage of amino acids in the encoded sequence is a “conservative mutant” where the alteration results in substantial maintenance of the structure and function of the peptide, polypeptide or protein. In particular, “conservative mutant” is intended to encompass the substitution of one or more amino acids (e.g. 1% to 50% of amino acids) with chemically similar amino acids. Conservative substitution tables providing functionally similar amino acids are well known in the art. Unless otherwise indicated, a conservatively modified variant do not exclude polymorphic variants, interspecies homologues and alleles. Without limitation, the following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M).

An amino acid sequence which comprises at least 50, 60, 70, 75, 80, 81, 82, 83, 84, 85, 86, 87, 88, 89, 90, 91, 92, 93, 94, 95, 96, 97, 98, 99 or 100% amino acid sequence identity to a specified reference sequence is also a “conservative mutant” so long as it retains a specified activity or fraction of said activity. Sequence identity can be determined using standard sequence alignment software/technologies, e.g. by aligning two sequences using BLAST, ALIGN, or another alignment software or algorithm known in the art using default parameters.

With respect to nucleic acid sequences that encode proteins, a conservative mutant or variant includes without limitation those nucleic acids which encode identical or conservatively substituted amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations”, which are one species of conservative mutants. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

Without limitation, this disclosure presents a chimeric receptor, as well as nucleic acid(s) encoding the chimeric receptor, a vector comprising the nucleic acid(s), a eukaryotic cell comprising the nucleic acid(s), vector and/or chimeric receptor. In certain embodiments, the eukaryotic cell functions as a biosensor and methods/uses related to said function are also presented herein.

Without limitation, this disclosure also relates to libraries of biosensor cells and exemplary methods/uses of said libraries.

### II. Chimeric Receptor, Nucleic Acid(S), Vector & Eukaryotic Cell

Without limitation, this disclosure provides a chimeric receptor comprising a binding portion comprising an extracellular binding site, a transmembrane domain and a signaling portion. The signaling portion comprises a tumor necrosis factor receptor superfamily (TNFRSF) member or a fragment of the TNFRSF member which retains an intracellular signaling domain of the TNFRSF member. Further, the binding site is extracellular and the intracellular signaling domain is intracellular when the chimeric receptor is expressed in a eukaryotic cell. Accordingly, the chimeric receptor retains functional membrane localization and TNFRSF intracellular signaling activity when expressed in a cell. The binding portion of the chimeric receptor comprises an extracellular amino acid sequence that is heterologous (or non-native) to the TNFRSF member.

As used herein, the term “receptor” means a protein that binds a binding substrate (e.g. a small molecule or protein) outside a cell that causes a signal or cellular response inside the cell. As used herein, the term “fusion protein” means a protein encoded by at least one nucleic acid coding sequence that is comprised of a fusion of two or more coding sequences from separate genes.

Unless otherwise indicated, the “chimeric receptor” disclosed herein is not limited to single subunit fusion proteins. In some embodiments, the chimeric receptor may be a single subunit fusion protein, which is encoded by at least one nucleic acid coding sequence that is comprised of a fusion of two or more coding sequences from separate genes. In other embodiments, the chimeric receptor may be assembled from multiple protein subunits that when expressed in the eukaryotic cell associate to form a quaternary structure held together by non-covalent interactions (e.g. electrostatic, Van der Waals and hydrogen bonding) and may further be held together by covalent interactions (e.g. disulfide bridges). For example, but without intending to be limiting, one or both of the binding portion and the signaling portion may comprise multiple subunits. For example, the binding portion may comprise an antibody or antigen binding fragment thereof. The binding portion may be on a separate subunit from the transmembrane domain and signaling portion. The signaling portion may be on a separate subunit from the transmembrane domain and binding portion. For example, but without limitation, the chimeric receptor may be a multi-subunit receptor comprising at least first and second subunits. The first subunit may comprise the binding portion, which may comprise a binding domain fused to a leucine zipper (or other association domain). The second subunit may comprise the transmembrane domain and signaling domain fused to the complementary leucine zipper (or other complementary association domain). As such, the leucine zipper allows for the binding domain to associate via the leucine zipper to the transmembrane domain and signaling domain. In a second non-limiting example, the first subunit may comprise the binding portion, which comprises an extracellular binding domain fused to the transmembrane domain fused to an intracellular leucine zipper (or other association domain). The second subunit may then comprise an intracellular signaling domain fused to the complementary leucine zipper (or other complementary association domain), such that the association of the two subunits is intracellular. In both examples the binding domain and signaling domain are not genetically linked but are functionally linked. Many other association domains besides leucine zippers are known and would be suitable to direct protein-protein interactions in the formation of a multi-subunit chimeric receptor (e.g. comprising 2, 3, 4, 5, 6 or more than 6 subunits).

The TNFRSF is a group of cytokine receptors generally characterized by an ability to bind ligands (such as TNFs) via an extracellular cysteine-rich ligand-binding domain and signal a cellular response when activated by binding. Certain TNFRSF members (e.g. TNFR1, TNFR2, TRAIL and the like) also have a pre-ligand binding assembly domain (PLAD) as part of their extracellular domain that plays a role in pre-assembly of the TNFRSF member in a ligand-unbound state (Chan. Cytokine. 2007; 37(2): 101-107).

In their active (signaling) form, the majority of TNFRSF members form trimeric complexes in the plasma membrane, although some TNFRSF members are soluble or can be cleaved into soluble forms.

While the chimeric receptor requires a transmembrane domain, this transmembrane domain may or may not be part of the signaling portion. In other words, only the intracellular signaling domain of the TNFRSF member is needed when the chimeric receptor further comprises a non-TNFRSF transmembrane domain and/or a non-TNFRSF extracellular domain comprising a non-TNFRSF binding site. The transmembrane domain of the chimeric receptor may or may not be comprised within the TNFRSF member or fragment of the TNFRSF member. The transmembrane domain may be a natural transmembrane domain (e.g. a segment or a plurality of segments from a natural transmembrane protein). The natural transmembrane domain may be from the same TNFRSF member as the signaling portion or from a different TNFRSF member than the signaling portion. The natural transmembrane domain may be a natural transmembrane domain from a heterologous integral membrane protein that is not a TNFRSF member. The transmembrane domain may be an artificial transmembrane domain. The transmembrane domain may be α-helical and have one transmembrane segment (i.e. single-pass) or more than one transmembrane segment (multi-pass). The transmembrane domain may comprise a n-sheet or n-barrel. Prediction of transmembrane domains/segments may be made using publicly available prediction tools (e.g. TMHMM, Krogh et al. Journal of Molecular Biology 2001; 305(3):567-580; OPCONS, Tsirigos et al. 2015 Nucleic Acids Research 43 (Webserver issue), W401-W407; TMpred, Hofmann & Stoffel Biol. Chem. Hoppe-Seyler 1993; 347:166, and the like). The topology of integral membrane proteins is thus predictable, such that it is understood which termini (N- or C-) and loop(s) (if present) are intracellular or extracellular for fusion and/or association with the signaling portion and binding portion of the chimeric receptor. The orientation of the chimeric receptor (an integral membrane protein) in the plasma membrane is determined by the amino acid sequence including the presence/absence of signal peptides, the net electrostatic charge flanking the transmembrane segments, and the length of the transmembrane segments. As a general rule, the flanking segment that carries the highest net positive charge remains on the cytosolic face of the plasma membrane and long hydrophobic segments (>20 residues) tend to adopt an orientation with a cytosolic C-terminus. Certain membrane proteins (e.g. beta-barrels and the like) may use chaperones and other/additional mechanisms for translation and insertion into the plasma membrane.

In some embodiments, the transmembrane domain is a single-pass transmembrane domain, such as but without limitation the transmembrane domain of CD4 or PDGFR. The single-pass transmembrane domain may be a hydrophobic α-helix of about 15 to about 23 amino acids (e.g. 15, 16, 17, 18, 19, 20, 21, 22 or 23 residues), often with positive charges flanking the transmembrane segment.

In some embodiments, the transmembrane domain is a multi-pass transmembrane domain. The multi-pass transmembrane domain may have 2, 3, 4, 5, 6, 7, 8, 9 10 or more than 10 transmembrane segments. In some embodiments, the multi-pass transmembrane domain is a 4-helix transmembrane domain, such as but without limitation the transmembrane domain of CD20. For the transmembrane domain of CD20, both the N-terminus and the C-terminus are intracellular, such that the extracellular domain is within an extracellular loop. In some embodiments, the multi-pass transmembrane domain is a 7-helix transmembrane domain, such as but without limitation the transmembrane domain of glucagon-like peptide 1 receptor (GLP1R) or another G-protein coupled receptor. The N-terminus of the GLP1R transmembrane domain is extracellular and the C-terminus is intracellular.

In some embodiments, the transmembrane domain is selected from the transmembrane domains of integral membrane proteins that are human CD molecules (also known as “clusters of differentiation”, “clusters of designation” or “classification determinants”).

In the chimeric receptor disclosed herein, the binding portion comprises an extracellular binding site that is not native to the TNFRSF member. In other words, the binding portion comprises an amino acid sequence(s) that is non-native (or heterologous) as compared to the TNFRSF member from which the signaling portion is derived, which creates a binding site that is distinct from the ligand binding site of the TNFRSF member. This permits the binding portion to specifically bind a binding substrate that is distinct from the native ligand of the TNFRSF member. Further description of the binding site is provided further below.

In addition to an extracellular ligand-binding domain and a transmembrane domain, TNFRSF members have an intracellular (or cytoplasmic) domain involved in signaling various cellular responses when the TNFRSF member is in a ligand-bound state, not through an intrinsic enzymatic activity of the intracellular domain, but through association of the intracellular domain with adaptor proteins (e.g. TRADD, TRAF, RIP, FADD and the like) which form (or cause the formation of) signaling complexes with accessory proteins having enzymatic activity (e.g. kinase or polyubiquitination activity). TNFRSF members signal a wide range of overlapping cellular responses, including but not limited to proliferation, differentiation, nuclear factor kappa B (NFκB or NF-κB) activation, cell death, and stress-activated protein kinase (SAP kinase). The intracellular domain of TNFRSF members generally lack recognizable common motifs among the members, the exception being a subgroup of TNFRSF members called “death receptors”, which comprise an approximately 80 amino acid long cytoplasmic “death domain”. The death domain binds other death domain-containing proteins.

As used herein in the context of TNFRSF, the term “intracellular domain” (or “ICD”), “cytoplasmic domain”, “signaling domain” or “intracellular signaling domain” all refer to the domain, domains or portions thereof of a TNFRSF member that are required for binding adaptor protein(s). A fragment which retains functional membrane localization and intracellular signaling activity of the TNFRSF member when expressed in a NF-κB competent cell (e.g. a vertebrate cell) may be confirmed using functional assays which assess signaling at any point in the signaling pathway of the TNFRSF member. For example, which is not to be considered limiting, TNFR1 is known to, among other functions, activate NF-κB and cause apoptosis. NF-κB is a highly conserved pathway in eukaryotes (not just vertebrates) and has been characterized in yeast. The yeast retrograde response is a predecessor with many similarities to the central stress-regulator, NF-κB, found in advanced multicellular organisms (Moore et al. Molecular and Cellular Biology 1993; 13:1666-1674). Accordingly, detecting cell death may be used to confirm that intracellular signaling activity is retained in a particular TNFRSF fragment. Alternatively, activated NF-κB can be detected directly or indirectly. Numerous tools/kits are commercially available for detecting activated NF-κB, including enzyme-linked immunosorbent assays (ELISA) and electrophoretic mobility shift assays (EMSAs). Alternatively, since NF-κB is a transcription factor, activated NF-κB may also be detected by linking a screenable marker gene or selectable marker gene to a NF-κB response element.

In certain embodiments, the TNFSRSF member is CD27 (also called TNFRSF7, s152 and Tp55), CD40 (also called TNFRSF5, p50 and Bp50), EDA2R (also called ectodysplasin A2 receptor, XEDAR, EDA-ADA-A2R, TNFRSF27), EDAR (also called ectodysplasin A receptor, ED3, DL, EDS, EDA3, Edar, ED1R, EDA1R), FAS (also called Fas cell surface death receptor, FAS1, APT1, TNFRSF6, CD95, APO-1), LTBR (also called lymphotoxin beta receptor, D125370, TNFCR, TNFR-RP, TNFR2-RP, TNF-R-III, TNFRSF3), NGFR (also called nerve growth factor receptor, TNFRSF16, CD271, p75NTR), RELT (also called RELT tumor necrosis factor receptor, TNFRSF19L, F1114993), TNFR1 (also called TNF receptor 1, TNFRSF1A, TNF-R, TNFAR, TNFR60, TNF-R-I, CD120a, TNF-R55), TNFR2 (also called TNF receptor 2, TNFRSF1B, TNFBR, TNFR80, TNF-R75, TNF-R-II, p75, CD120b), TNFRSF4 (also called TXGP1L, ACT35, OX40, CD134), TNFRSF6B (also called DcR3, DCR3, TR6, M68), TNFRSF8 (also called CD30, D1S166E, KI-1), TNFRSF9 (also called ILA, CD137, 4-1BB), TNFRSF10A (also called DR4, Apo2, TRAILR1, CD261), TNFRSF10B (also called DR5, KILLER, TRICK2A, TRAILR2, TRICKB, CD262), TNFRSF10C (also called DcR1, TRAILR3, LIT, TRID, CD263), TNFRSF10D (also called DcR2, TRUNDD, TRAILR4, CD264), TNFRSF11A (also called PDB2, LOH18CR1, RANK, CD265, FEO), TNFRSF11B (also called OPG, OCIF, TR1), TNFRSF12A (also called FN14, TweakR, CD266), TNFRSF13B (also called TALI, CD267, IGAD2), TNFRSF13C (also called BAFFR, CD268), TNFRSF14 (also called HVEM, ATAR, TR2, LIGHTR, HVEA, CD270), TNFRSF17 (also called BCMA, BCM, CD269, TNFRSF13A), TNFRSF18 (also called AITR, GITR, CD357), TNFRSF19 (also called TAJ-alpha, TROY, TAJ, TRADE), TNFRSF21 (also called DR6, CD358), TNFRSF25 (also called TNFRSF12, DR3, TRAMP, WSL-1, LARD, WSL-LR, DDR3, TR3, APO-3), ora protein having an intracellular signaling domain that has at least 80%, 81%, 82%, 83%, 84%, 85%, 86%, 87%, 88%, 89%, 90%, 91%, 92%, 93%, 94%, 95%, 96%, 97%, 98% or 99% amino acid sequence identity to the intracellular signaling domain of any TNFRSF member listed above and which retains TNFRSF membrane localization and TNFRSF intracellular signaling activity when expressed in a vertebrate cell. In some embodiments, the intracellular signaling domain of the TNFRSF member is a conservative mutant that has at least 80%, 81%, 82%, 83%, 84%, 85%, 86%, 87%, 88%, 89%, 90%, 91%, 92%, 93%, 94%, 95%, 96%, 97%, 98% or 99% amino acid sequence identity to the intracellular signaling domain of any TNFRSF member listed above and which retains sufficient intracellular signaling activity to cause activation of a NF-κB response element when the chimeric receptor is expressed in a eukaryotic cell that is NF-κB competent cell (e.g. a vertebrate cell, a mammalian cell, a human cell or a human cell line). The TNFRSF membrane localization and TNFRSF intracellular signaling activity may be the membrane localization and intracellular signaling activity of CD27, CD40, EDA2R, EDAR, FAS, LTBR, NGFR, RELT, TNFR1, TNFR2, TNFRSF4, TNFRSF6B, TNFRSF8, TNFRSF9, TNFRSF10A, TNFRSF10B, TNFRSF10C, TNFRSF10D, TNFRSF11A, TNFRSF11B, TNFRSF12A, TNFRSF13B, TNFRSF13C, TNFRSF14, TNFRSF17, TNFRSF18, TNFRSF19, TNFRSF21 or TNFRSF25. In embodiments which do not include the extracellular domain and/or transmembrane domain of a TNFRSF member (e.g. as listed above), functional membrane localization only requires that the intracellular signaling domain be intracellular, that the transmembrane domain be localized in the cell membrane, and that the binding site be extracellular. The level of intracellular signaling activity may be the same, higher or lower as compared to CD27, CD40, EDA2R, EDAR, FAS, LTBR, NGFR, RELT, TNFR1, TNFR2, TNFRSF4, TNFRSF6B, TNFRSF8, TNFRSF9, TNFRSF10A, TNFRSF10B, TNFRSF10C, TNFRSF10D, TNFRSF11A, TNFRSF11B, TNFRSF12A, TNFRSF13B, TNFRSF13C, TNFRSF14, TNFRSF17, TNFRSF18, TNFRSF19, TNFRSF21 or TNFRSF25, so long as the signaling portion retains sufficient intracellular signaling activity to cause activation of a NF-κB response element when the chimeric receptor is expressed in a NF-κB competent eukaryotic cell (e.g. without limitation, a vertebrate cell, a mammalian cell, a human cell or a human cell line). The TNFRSF member may be a hybrid of two or more of the abovementioned TNFRSF members, and/or the intracellular domain of the TNFRSF member may be a hybrid of two or more signaling domains from the abovementioned TNFRSF members, so long as the chimeric receptor retains functional transmembrane localization and the intracellular signaling activity of a TNFRSF member.

In certain embodiments, the TNFRSF member is a death receptor. The death receptor may be TNFR1, FAS, TRAILR1, TRAILR2, TRAMP, CD358 or a protein that has at least 80%, 81%, 82%, 83%, 84%, 85%, 86%, 87%, 88%, 89%, 90%, 91%, 92%, 93%, 94%, 95%, 96%, 97%, 98% or 99% amino acid sequence identity to any death receptor listed above and which retains the transmembrane localization and intracellular signaling activity of TNFR1, FAS, TRAILR1, TRAILR2, TRAMP or CD358 when expressed in a vertebrate cell. The level of intracellular signaling activity may be the same, higher or lower as compared to TNFR1, FAS, TRAILR1, TRAILR2, TRAMP or CD358. In some embodiments, the death receptor is TNFR1, FAS, TRAILR1, TRAILR2, TRAMP or CD358. In some embodiments, the death receptor is TNFR1.

In some embodiments, the signaling portion of the chimeric receptor comprises a full-length TNFRSF member, wherein the transmembrane domain of the chimeric receptor is the transmembrane domain from the TNFRSF member. In other embodiments, the signaling portion of the chimeric receptor comprises a fragment of the TNFRSF member which retains transmembrane and intracellular signaling domains of the TNFRSF member when expressed in a NF-κB competent eukaryotic cell (e.g. without limitation, a vertebrate cell, a mammalian cell, a human cell or a human cell line). The fragment may be a deletion construct which omits the ligand-binding domain of the TNFRSF member or a portion of the ligand-binding domain (e.g. omits CRD1, CRD2, CRD3 and/or CRD4 domains or any other sequence(s) within the ligand binding domain), wherein the transmembrane domain of the chimeric receptor is the transmembrane domain from the TNFRSF member. The fragment may be a deletion construct which omits the extracellular domain of the TNFRSF member or a portion of the extracellular domain, wherein the transmembrane domain of the chimeric receptor is the transmembrane domain from the TNFRSF member. The fragment may be a deletion construct which omits the extracellular domain and the transmembrane domain of the TNFRSF member or a portion of the transmembrane domain.

In some embodiments, the signaling portion comprises the amino acid sequence of SEQ ID NO: 63 or 64, or a sequence that has at least 80%, 81%, 82%, 83%, 84%, 85%, 86%, 87%, 88%, 89%, 90%, 91%, 92%, 93%, 94%, 95%, 96%, 97%, 98% or 99% amino acid sequence identity to SEQ ID NO: 63 or 64 and which is capable of activating NF-κB signaling when the chimeric receptor is expressed in a eukaryotic cell (e.g. a vertebrate cell) that is NF-κB competent in response to activation of TNFR1 (for SEQ ID NO: 63) or TRAILR2 (for SEQ ID NO: 64). In some of these embodiments, the sequence differences as compared to SEQ ID NO: 63 or 64 are conservative amino acid substitutions.

Without wishing to be bound by theory, TNFRSF members are thought to be activated through (1) ligand-induced receptor oligomerization, e.g. by receptor cross-linking due to binding to a multivalent ligand such as trimeric TNF, (2) through a change in conformation of a pre-assembled TNFRSF oligomer, e.g. by a change in the interaction of TNFRSF subunits in a trimeric TNFRSF complex, or (3) through a change in oligomerization state, e.g. a change from dimer to trimer (Chan. Cytokine. 2007; 37(2): 101-107). Regardless of the exact mechanism, TNFRSF members can be activated by encouraging the formation of TNFRSF oligomerization, e.g. by ligand-binding or by cross-linking the receptor. Increasing the local concentration of the receptor may also result in non-specific activation by increasing the local concentration of the TNFRSF member. Accordingly, the chimeric receptor can be activated by binding a binding substrate that effectively oligomerizes the signaling portion. For example, if the binding substrate is “multivalent” (i.e. has two binding sites for collectively and simultaneously binding two chimeric receptors), then binding the binding substrate will oligomerize the two chimeric receptors and activate the signaling activity of the signaling portion.

In addition to a signaling domain derived from a TNFRSF member, in some embodiments the chimeric receptor may comprise an additional cytoplasmic domain. This may be a drug selectable marker (e.g. Puro, Hygro or the like) to assist in selection of an inframe chimeric receptor and/or proper orientation in the plasma membrane, a fluorescent protein (e.g. GFP, RFP or the like) to assist in identifying an inframe chimeric receptor and/or proper orientation in the plasma membrane, a transcription factor or non-TNFRSF signaling domain to amplify detection of an inframe chimeric receptor using a reporter linked to a different signaling pathway (e.g. GAL4 or the like), e.g. to boost expression levels of an antibiotic resistance gene (e.g. Puro, Hygro or the like) if inframe expession levels of the resistance gene was too weak, an additional or different TNFRSF signaling domain (e.g. to potentially amplify signaling), a domain that enhances or inhibits TNFRSF signaling (e.g. to optimize the signal to noise ratio). The additional cytoplasmic domain may be directly linked, joined with a linker or joined with a P2A or cleavage sequence.

The extracellular binding site is not native to the TNFRSF member, meaning the amino acid residues that comprise the binding site are not TNFRSF residues and thus the binding substrate is not the natural cognate ligand of the TNFRSF member (e.g. not TNF when the TNFRSF member is TNFR1). In the context of the chimeric receptor, a “binding site” as used herein refers to the amino acids in a protein that are required and responsible for the binding properties of the binding portion. Unless otherwise indicated, the “binding site” of the chimeric receptor is not limited to canonical ligand-binding sites of receptors, substrate-binding sites of enzymes, and antigen-binding sites of antibodies (to name but a few), but instead refers to any amino acid sequence or sequences (including peptides, polypeptides and proteins) longer than 6 residues (e.g. 7 or more amino acids) that is capable of specifically binding, or being specifically bound to or by, the binding substrate (or ligand). In some embodiments, the binding site excludes sequences such as FLAG, V5, Myc, stretches of Histidine sequences or other sequences that are used as “tags” in a fusion protein. The binding substrate (or ligand) may be a peptide, polypeptide, protein, sugar, polysaccharide, DNA, RNA, hapten, small organic molecule or any other molecule. In some embodiments, but without limitation, the binding substrate is a cell surface-anchored or secreted protein, polysaccharide or glycoprotein. The ligand may or may not be known for the binding site (e.g. if the binding site is artificial or derived from an orphan receptor). The binding portion may comprise multiple binding sites. For example, antibodies (such as IgG) contain antigen-binding domains and binding sites in their Fc region.

In some embodiments, the binding site is comprises a peptide of 7 or more randomized amino acids (as have been used in random peptide libraries). Random peptide libraries have been shown to be a powerful tool for studying protein-protein interactions and identifying peptides that can bind target molecules (e.g. phage-displayed peptide libraries were first described in 1985). Peptide libraries have been applied to identify bioactive peptides bound to receptors or proteins, disease-specific antigen mimics, peptides bound to non-protein targets, cell-specific peptides, or organ-specific peptides, and epitope mapping. Peptide libraries have also been utilized in yeast and bacterial systems in a variety of formats and mammalian two-hybrid screening approaches. The current invention allows for another format using biosensors which offers increased sensitivity. In some embodiments, peptides are expressed as the entire binding portion (i.e. as an extracellular binding domain) or as part of the binding portion. For example, the peptide binding site may be expressed as a fusion protein, linked to a transmembrane domain (native or non-native to the TNFRSF member) which is linked to the intracellular signaling domain of the TNFRSF member. In combination with the de novo engineering using V(D)J recombination or viral infection, large libraries of biosensors can be generated that display random peptide libraries.

In some embodiments, the binding portion of the chimeric receptor comprises an antibody or antigen binding fragment thereof. In other embodiments, the binding portion of the chimeric receptor comprises a monobody, an affibody, an anticalin, a DARPin, a Kunitz domain, an avimer or a soluble T-cell receptor, as described in more detail below. In other embodiments, the chimeric receptor comprises, and the binding portion is comprised within, a TCR or an antigen-binding fragment of the TCR.

The antibody may be of any species or may be chimeric or artificial. For example, but without limitation, the antibody may be non-human (e.g.: a camelid, such as dromedary, camel, llama, alpaca, and the like; cartilaginous fish, such as shark and the like; mouse, rat, monkey or other), primatized, humanized or fully human. A chimeric antibody contains amino acid sequences from multiple species, e.g. from human and non-human or from two non-human species. Methods for humanizing (or primatizing) non-human antibodies are well known in the art, e.g. by substituting non-human (or non-primate) constant domains for those of a human antibody (creating a chimeric antibody) or by substituting one or more (e.g. 1, 2, 3, 4, 5 or 6) of the Complementarity Determining Regions (CDRs) of a human (or primate) antibody with a non-human antibody (see, e.g.: Jones et al. Nature 1986; 321:522-525; Riechmann et al. Nature 1988; 332:323-327; Verhoeyen et al. Science 1988; 239:1534-1536; Presta. Curr. Op. Struct. Biol. 1995; 2:593-596; Morrison et al. Proc. Natl. Acad. Sci. USA 1984; 81:6851-6855; Morrison and Oi. Adv. Immunol. 1988; 44:65-92; Padlan. Molec. Immun. 1991; 28:489-498; and Padlan. Molec. Immun. 1994; 31(3):169-217). The antibody may be comprised of two heavy chains and two light chains. The antibody may be a single-chain antibody with the heavy chain and light chain separated by a linker. The antibody may be a heavy chain only antibody (e.g. an dromedary, camel, llama, alpaca or shark antibody which lacks light chains, or a human heavy chain). The antibody may be a single-domain antibody (sdAb).

“Artificial” antibodies include known antibody derivatives, e.g. scFv (i.e. single chain Fv), scFv-Fc, minibodies, nanobodies, diabodies, tri(a)bodies and the like.

As used herein, the term “antigen binding fragment” of an antibody means any antibody fragment which possesses antigen binding activity. In some embodiments, the antigen binding fragment comprises antibody light chain and heavy chain variable domains (i.e. VL and VH domains). In some fragments, the light chain is omitted. Non-limiting examples of antibody fragments include Fab, Fab′ and F(ab′)2.

Non-limiting examples of antibodies and antigen binding fragments include, without limitation: IgA, IgM, IgG, IgE, IgD, sdAb, Fab, Fab′, F(ab′)2, scFv, scFv-Fc, minibodies, nanobodies, diabodies, tri(a)bodies and the like. Other antibodies and fragments are known, a number of non-limiting examples of which are disclosed in Deyev and Lebedenko (2008, BioEssays 30:904-918). In some embodiments, the antibody or antigen binding fragment thereof is a IgA, a IgM, a IgG, a IgE, a IgD, a sdAb, a Fab, a Fab′, a F(ab′)2, a scFv, a scFv-Fc, a minibody, a nanobody, a diabodies or a tri(a)body. In some embodiments, the antibody is a IgG antibody.

In some embodiments, the antibody or antigen binding fragment (e.g. without limitation an IgG antibody or fragment thereof) binds the binding substrate with a dissociation constant (i.e. K_{D}) of less than 500 nM, less than 400 nM, less than 300 nM, less than 200 nM, less than 100 nM or less than 50 nM. In some embodiments, the antibody or antigen binding fragment may bind the binding substrate with a picomolar K_{D}. The affinity and specificity of the antibody or antigen binding fragment may have been engineered, for example, but without limitation, by using in vitro V(D)J recombination, mutagenesis and/or the use of double-stranded breaks together with Tdt such as with restriction enzymes, CRISPR, Zinc Finger or Talon methods or the use of error prone PCR, degenerate oligos or degererate gene synthesis products.

Monobodies (also called Adnectin^{T}) are synthetic binding proteins based on the structure of the tenth extracellular type II domain of human fibronectin. They have exposed loops which resemble the structure, affinity and specificity of antibody CDRs, but are much smaller (approximately 90 amino acids) and lack disculfide bonds, which makes them particularly useful for inclusion in fusion proteins (Lipovsek. Protein Eng Des Sel 2011; 24:3-9).

Affibodies are small proteins (approximately 6 kDa) based on the Z domain of protein A. Compared to antibodies, they are much smaller and lack disulfide bonds, such that they can be readily included into a fusion protein. Affibodies with unique binding properties are generally acquired by modification of 13 amino acids located in two alpha-helices involved in the binding activity, although additional amino acids outside this binding surface may also be modified (see, e.g.: Lofblom, et al. FEBS Lett. 2010; 584:2670-2680; and Nygren, FEBS J. 2008; 275:2668-2676).

Anticalins are artificial proteins derived from human lipocalins. They have a small size of approximately 20 kDa and contain a barrel structure formed by eight antiparallel β-strands pairwise connected by loops and an attached α-helix. Conformational deviations are primarily located in the four loops reaching in the ligand binding site (Gebauer and Skerra. Methods in Enzymology 2012; 503:157-188; Skerra. FEBS J. 2008; 275:2677-2683; and Vogt and Skerra. Chembiochem. 2004; 5:191-199).

DARPins are designed ankyrin repeat proteins. The ankyrin repeat motif consists of approximately 33 amino acids which form a loop, a β-turn, and 2 antiparallel α-helices connected by a tight turn (see, e.g.: Stumpp & Amstutz. Curr. Opin. Drug. Discov. Devel. 2007; 2:153-9; Pluckthun. Annual Review of Pharmacology and Toxicology 2015; 55:489-511; and Martin-Killias, et al. Clin. Cancer Res. 2010; 17:100-110).

Avimers are artificial proteins that comprise two or more A domains of 30 to 35 amino acids each fused together (optionally with linker peptides). The A domains are derived from various membrane receptors and have a rigid structure stabilized by disulfide bonds and calcium. Each A domain can bind to a different epitope of a target protein to increase affinity (i.e. avidity) or can bind epitopes on different target proteins (see, e.g.: Silverman et al. Nat. Biotechnol. 2005; 23(12):1556-61).

Kunitz domains are peptides that form stable structures able to recognize specific targets and have been previously incorporated into fusions proteins (Zhao et al. Int. J. Mol. Med. 2016; 37:1310-1316) and phage display libraries (WO 2004063337).

Soluble TCRs or single-variable domain TCRs have been described, e.g, ImmTAC™ and the like (Oates & Jakobsen. Oncolmmunology 2013; 2:2, e22891) and as described in PCT Patent Publication No. WO/2017/091905. Single-variable domain TCRs are included within the term “a TCR or an antigen-binding fragment of the TCR”, which also includes all other known antigen-binding fragments of TCRs.

Many scaffolds for the binding portion are known which are amendable to engineering to alter the affinity and selectivity of the binding portion. Fusing these scaffolds (optionally with the addition of a linker) allows them to be incorporated into fusion proteins where they retain their binding function. In some embodiments, the binding portion may be fused to the signaling portion by peptide bond, disulfide bond or other covalent bond. For example, but without limitation, a polypeptide chain of the binding portion may be expressed on the same polypeptide chain as a polypeptide chain of the signaling portion, although other polypeptide chains may also be expressed which collectively form the chimeric receptor as a multi-subunit protein complex. As such, the chimeric receptor may be a multi-subunit protein complex or may consist of a single polypeptide chain or single polypeptide chain modified by post-translational modification in vivo.

In some embodiments the binding portion may be fused to the signaling portion using a linker (e.g. a peptide linker), when the signaling portion comprises the transmembrane domain. In embodiments in which the transmembrane domain is not comprised within the TNFRSF member or fragment thereof, a linker (e.g. a peptide linker) may be used at any fusion junction in the chimeric receptor (e.g. between signaling portion and transmembrane domain and/or between binding portion and transmembrane domain).

Fusion protein linkers (including for fusion junctions, monobodies, affibodies, anticalins, avimers, Kunitz domains and others) are known. For example, the linker may be flexible or rigid. Non-limiting examples of rigid and flexible linkers are provided in Chen et al. (Adv Drug Deliv Rev. 2013; 65(10):1357-1369). In some embodiments, the linker is a peptide of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 amino acid residues, wherein each residue in the peptide may independently be Gly, Ser, Glu, Gln, Ala, Leu, Iso, Lys, Arg, Pro, or another amino acid. In some embodiments, the linker is Gly, Ser, Ser-Gly, Gly-Ser, Gly-Gly or Ser-Ser.

In some embodiments, the binding portion comprises the amino acid sequence of SEQ ID NO: 1, 2, 3 or 4 (or any other antibody heavy chain sequence disclosed herein). In some embodiments, the binding portion comprises the amino acid sequence of SEQ ID NO: 27, 29, 31 33, 46 or 47 (or any other antibody light chain sequence disclosed herein). In some embodiments, the signaling portion comprises the amino acid sequence of SEQ ID NO: 6, 7, 8, 9 or 10 (or any other TNFR1 construct sequence disclosed herein). In some embodiments, the chimeric receptor comprises the amino acid sequence of SEQ ID NO: 13, 14, 15, 16, 17, 26, 28, 30, 40, 45, 48 or 49 (or any other chimeric receptor construct sequence disclosed herein).

This disclosure also provides at least one nucleic acid comprising one or more coding sequences which collectively encode the chimeric receptor defined herein. For example, where the chimeric receptor comprises a full length IgG for the binding portion, the light chains of the IgG may be on a separate nucleic acid molecule from the fusion of the TNFRSF member and the heavy chain (e.g. where each is on a separate plasmid or chromosome or one is on a plasmid and the other is chromosomally integrated).

To facilitate expression of the one or more coding sequences which collectively encode the chimeric receptor, in some embodiments the at least one nucleic acid may further comprise at least one promoter operably linked to the one or more coding sequences. The at least one promoter may include weak and/or strong promoter(s).

In some embodiments, the at least one promoter may include a weak promoter. Significant research has been done on the analysis of TATA boxes and other transcription binding sites that modulate transcription activity. These binding sites can be mutated or deleted to compromise the binding to and/or assembly of transcription factors and/or assembly of the RNA polymerase so as to ultimately compromise the rate of transcription. For example, but without limitation, the weak promoter may be a UBC promoter (Ubiquitin C promoter), a PGK promoter (phosphoglycerate kinase 1 promoter), a Thymidine Kinase (TK) promoter or a promoter that has a transcriptional activity that is no more than 100%, 150%, 200%, 250%, 300%, 350%, 400%, 450% or 500% the transcriptional activity of one of the aforementioned weak promoters when transcribing the same reference coding sequence when in operable linkage to said reference coding sequence (e.g. SEQ ID NO: 13, 14, 15, 16, 17, 26, 28 or 30).

The at least one promoter may include regulated or constitutive promoter(s). In some embodiments, the at least one promoter comprises inducible promoter(s). For example, the at least one promoter may comprise binding sites for a repressor, such as the Tet repressor, the Gal4 repressor and the like. In the case of the Tet repressor, operator sequence(s) (e.g. tetO) may be placed upstream of a minimal promoter to permit transcription to be reversibly turned on or off in the presence of tetracycline or one of its derivatives (e.g. doxycycline and the like). Similarly, nucleic acid sequences which bind the Gal4 repressor may be positioned to regulate transcription of genes that are operably linked to a minimal promoter. As used herein, operator sequences and/or other regulator sequences are considered part of the regulated promoter, regardless of their proximity to transcription start site(s) of the coding sequence(s), so long as they are functionally positioned for regulation of transcription. The promoter may be activated upon the binding of a ligand to a receptor.

An advantage of using a weak promoter is a reduction in background signal from intracellular signaling in the absence of bound binding substrate. Without wishing to be bound by theory, it is thought that a weak promoter reduces background signal by lowering expression of the chimeric receptor so as to reduce activation of the signaling portion due to local concentrations of the chimeric receptor exceeding the threshold for activation. In effect, diluting the chimeric receptor on the cell surface reduces self-activation in the absence of binding substrate.

In some embodiments, the one or more coding sequence comprises or is operably linked to one or more genetic elements which, when the chimeric receptor is expressed in an NF-κB competent eukaryotic cell (e.g. without limitation a vertebrate cell), cause expression of the chimeric receptor at a level that is sufficiently low such that signaling caused by binding of the binding substrate to the chimeric receptor is distinguishable over background signaling (e.g. in the absence of the binding substrate). Various such genetic elements are known, which can be used alone or in combination, including for example, but without limitation: a Kozak sequence in the nucleic acid which causes inefficient translation of the chimeric receptor (see, e.g.: Grzegorski, et al. PloS One 2014; 9:e108475; and Kozak, Gene 2005; 361:13-37); codon(s) in the at least one coding sequence which are not optimized for efficient translation in the cell; one or more RNA destabilizing sequences in the nucleic acid which reduces the half-life of an RNA transcribed from the nucleic acid which encodes the chimeric receptor (see e.g.: Dijk et al. RNA 1998; 4:1623-1635; and Day & Tuite. Journal of Endocrinology 1998; 157:361-371); intron and/or exon sequences in the one or more coding sequences which cause inefficient intron splicing (see, e.g.: Fu & Ares Nature Reviews 2014; 15:689-701); and/or ubiquination sequence(s) in the chimeric receptor (e.g. to encourage degradation of the chimeric receptor; see e.g.: Yu et al. J. Biol. Chem. 2016; 291:14526-14539).

In some embodiments, the at least one nucleic acid comprising one or more coding sequences which collectively encode the chimeric receptor is a vector.

This disclosure also provides a eukaryotic cell comprising the at least one nucleic acid defined herein. The eukaryotic cell may or may not be NF-κB competent. In some embodiments, the eukaryotic cell is NF-κB competent (e.g. for use as a biosensor cell). In some embodiments, the eukaryotic cell need not be NF-κB competent (e.g. for storing or reproducing the at least one nucleic acid or vector defined above).

In some embodiments, a promoter that is operably linked to a coding sequence in the at least one nucleic acid comprises an operator sequence and the eukaryotic cell expresses a repressor which binds to the operator sequence. In other words, the repressor binds an operator sequence within a regulated promoter that controls expression of the one or more coding sequence which collectively encode the chimeric receptor described herein. This further reduces the expression of the chimeric receptor which assists achieving low background levels of signaling in the absence of binding substrate. The repressor may be TetR and the operator may be TetO or another nucleotide sequence that binds TetR. The repressor may be Gal4 and the operator may be a nucleotide sequence which binds Ga14.

In some embodiments, the eukaryotic cell further comprises at least one sequence for expressing antisense RNA, miRNA (microRNA) or siRNA (small interfering RNA) configured to reduce expression levels of the chimeric receptor. Nucleic acids comprising such sequences may be separate from or comprise part of the at least one nucleic acid comprising the one or more coding sequence which collectively encode the chimeric receptor. Sequences for expressing antisense RNA, miRNA and siRNA can be readily generated from the sense sequence (i.e. the sequence of the at least one nucleic acid that collectively encodes the chimeric receptor). With respect to antisense RNA, this includes any nucleic acid sequence which when transcribed in the vertebrate cell would bind to the messenger RNA (mRNA) that encodes the chimeric receptor (including without limitation sequences which are 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to the reverse complement of the mRNA or the sequence within the mRNA that encodes the chimeric receptor). Tools for generating antisense RNA, miRNA and siRNA are publicly and commercially available.

As mentioned, activated TNFRSF members in turn activate NF-κB through adaptor proteins and their enzymatic binding partners, either through the canonical and/or noncanonical NF-κB signaling pathways (Wertz and Dixit Cold Spring Harb Perspect Biol 2010; 2(3): a003350). NF-κB is not a single entity, but is a family of dimeric transcription factors consisting of five proteins, p65 (also known as RelA), RelB, c-Rel, p50 and p52 (p105 and p100 are precursor proteins for p50 and p52, respectively). NF-κB proteins associate to form homodimers and heterodimers (e.g. the p65:p50 heterodimer). NF-κB is maintained in an inactive state through association with an IκB (an inhibitor of NF-κB). NF-κB is activated by polyubiquitination of IκB, which targets IκB for proteosomeal degradation and liberates (activated) NF-κB dimers. Ultimately, IκB is ubiquitinated by the activity of the IκK complex, which is activated by signaling complex(es) which ultimately are formed as a result of a signaling cascade initiated by activated TNFRSF members. Accordingly, operably linking a gene of interest (or multiple genes of interest) to a NF-κB response element will enable the transcription of the gene of interest (or the multiple genes of interest) to be controlled by the activation state of the chimeric receptor, which is inactive when unbound by binding substrate and active when bound by binding substrate. Thus, in some embodiments, the eukaryotic cell further comprises a gene of interest (or multiple genes of interest) linked to a second promoter and a NF-κB response element such that expression of the gene of interest (or the multiple genes of interest) is repressed by NF-κB binding to the NF-κB response element and induced in the absence of said NF-κB binding. In these embodiments, the NF-κB response element is configured to be bound by a NF-κB which acts as a transcriptional repressor (e.g. p50 and/or p52). In alternative embodiments, the vertebrate cell further comprises a gene of interest (or multiple genes of interest) linked to a second promoter and a NF-κB response element such that expression of the gene of interest is induced by NF-κB binding to the NF-κB response element and inactive (or repressed) in the absence of said NF-κB binding. In these embodiments, the NF-κB response element is configured to be bound by a NF—κB which acts as a transcriptional activator (e.g. p65:p50 heterodimer or other dimers incorporating p65, RelB and/or c-Rel).

In some embodiments, the multiple genes of interest are part of a polycistronic operon operably linked to the NF-κB response element. For examples, but without limitation the multiple genes may be separated by P2A and/or IRES sequences or other such sequences. In some embodiments, the multiple genes of interest are part of separate operons, each operably linked to a separate NF-κB response element.

In some embodiments, the gene of interest or multiple genes of interest are chromosomally integrated into the eukaryotic cell (e.g. a vertebrate cell). In other embodiments, the gene of interest or multiple genes of interest are stably maintained as a plasmid. For example, but without limitation the stably maintained plasmid may be a yeast artificial chromosome (YAC) and the like, or an OriP containing plasmid where the vertebrate cell expresses EBNA-1 or a similar protein).

In some embodiments, the gene of interest is or causes expression of a marker gene. In some embodiments, the genes of interest comprise or cause expression of a marker gene.

In some embodiments, the marker gene is a screenable marker gene. For example, the screenable marker gene may cause expression of a fluorescent protein (e.g. green fluorescent protein, red fluorescent protein and the like), an enzyme (e.g. β-galactosidase, chloramphenicol acetyltransferase and the like) or a surface antigen (e.g. FLAG epitope, Myc tag, CD19, CD19-PE and the like) when the screenable marker gene is expressed. The screenable marker gene may encode the fluorescent protein, the enzyme or the surface antigen.

In some embodiments, the marker gene is a selectable marker gene. The introduction of a gene(s) into a cell which lacked the gene(s) may be associated with the acquisition of a novel phenotype. This acquired phenotype may then be exploited to select for cells which harbor/express the introduced gene(s). Although selection is often used for tracking the introduction of genetic elements, the biosensor herein may use a selectable marker to select for activated biosensors (e.g. activated due to specific recognition of binding substrate). For example, when starting with a large population of biosensors having a diverse set of binding specificities, the use of a selectable marker may allow for rare populations to be identified that would be a challenge using FACS or magnetic sorting (e.g. when frequencies are well below 1 in a million).

In some embodiments, the marker gene may be a positive selectable marker gene. Positive selection is distinct from a traditional reporter system in that it allows for survival (and growth) and allows for significantly larger numbers of cells to be evaluated than even the highest throughput screening platforms which depend on mechanical detectors to identify activated cells.

Expression of the positive selectable marker gene may encode a protein(s) which confers resistance to a toxic compound. As used herein, the term “toxic compound” includes without limitation any small molecules, peptides, proteins, suicide gene products, and the like, whether natural or artificial, which is poisonous to the eukaryotic cell or causes cell death. In certain embodiments, the positive selectable marker gene may encode an antibiotic resistance protein. For example, genes are known which provide mammalian cells resistance against geneticin, neomycin, Zeocin™, hygromycin B, puromycin, blasticidin and other antibiotics. Alternatively, expression of a MDR (multi-drug resistance) gene(s) may act as a positive selectable marker by providing resistance to a toxic compound(s).

Positive selection may also be accomplished by curing auxotrophy, i.e. the inability of a cell to synthesize a particular compound(s) needed for growth/survival. This selection approach is widely used in yeast selections, but is also used in other eukaryotic cell types, including vertebrate and mammalian cells. Auxotrophy exists for large classes of compounds required for growth including without limitation vitamins, essential nutrients, essential amino acids and essential fatty acids. Certain cells are dependent on specific growth factors for growth and survival. Therefore, acquisition of the gene expressing the growth factor would allow for positive selection. Certain gene products such as hypoxanthine-guanine phosphoribosyltransferase (HPRT) and xanthine phosphoribosyltransferase (GPT) allow for the conversion of compounds to useful metabolites essential for growth. Auxotrophy may also be used with factor dependent cell lines that need certain growth factors or ligands to proliferate (e.g. the TF1 cell line needs erythropoietin or “EPO” supplementation for growth). Accordingly, in certain embodiments, the vertebrate cell is an auxotroph which requires a missing compound for growth or survival and the positive selectable marker gene(s) encodes one or more gene products which permit the eukaryotic cell to synthesize the missing compound.

In certain embodiments, expression of the selectable marker gene permits selection based on chemical detoxification, selection based on exclusion or removal, selection based on increased expression (such as the dihydrofolate reductase or “DHFR” gene, and the like), selection based on pathogen resistance, selection based on heat tolerance, selection based on radiation resistance, selection based on double-strand break sensitivity, selection based on ability to utilize non-metabolized compounds (e.g. HPRT, GPT and the like) and/or selection based on acquisition of a growth factor.

In some embodiments the selectable marker gene may be a negative selectable marker. Negative selection cannot be read by reporter based systems. The selectable marker gene may encode or cause expression of: a toxin or an enzyme (e.g. HPRT, GPT or a suicide gene(s)) which can convert a precursor compound to a toxic compound. A number of suicide gene systems have been described including the herpes simplex virus thymidine kinase gene, the cytosine deaminase gene, the varicella-zoster virus thymidine kinase gene, the nitroreductase gene, and the E. coli Deo gene. The products of these suicide genes metabolize substrates into toxic compounds that are lethal to cells. Accordingly, in some embodiments the negative selectable marker may be a suicide gene. In some embodiments, the negative selectable marker may be HPRT, GPT, herpes simplex virus thymidine kinase gene, cytosine deaminase gene, varicella-zoster virus thymidine kinase gene, nitroreductase gene or E. coli Deo gene. Hormone based dimerization may also be used for negative selection by promoting complementation to assemble or reconstitute a function protein. Two-hybrid approaches may also be deployed to drive the expression of toxic genes either directly or indirectly. Gene modifying approaches that incorporate CRE, FRT, CRISPR or other gene modifying activities may be utilized to induce the expression of a gene of interest. Another non-limiting option for negative selection is induction of apoptosis. Apoptosis or programmed cell death is a conserved process in vertebrates and has also been described in non-vertebrate eukaryotic cells, e.g. yeast (Carmona-Gutierrez et al. Cell Death and Differentiation 2010; 17:763-773). Ycalp is a metacaspase (an ortholog of mammalian caspases) that is required for numerous cell death scenarios. For example, the chimeric receptor may induce apoptosis via death domain-mediated signaling or by causing/increasing expression of a signaling protein that promotes apoptosis.

In some embodiments, the selectable marker gene may encode or cause expression of a chimeric screenable-selectable marker. For example, but without limitation, the marker gene may encode an integral membrane protein that displays an extracellular surface antigen and an intracellular resistance protein. For example, but without limitation, the selectable marker gene may encode, or cause expression of, CD19 fused to puromycin N-acetyl-transferase (Puro), and be configured for intracellular display of Puro and extracellular display of CD19 antigen. In some embodiments, the selectable marker gene comprises or consists of the amino acid sequence of SEQ ID NO:18. Without limitation, SEQ ID NO:19 represents the nucleic acid sequence of a vector for expressing a CD19-Puro fusion having the amino sequence of SEQ ID NO: 18.

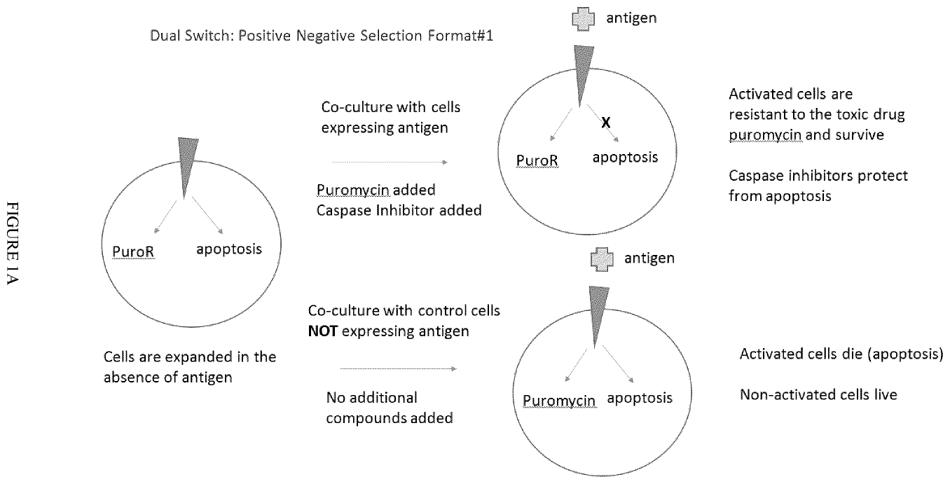

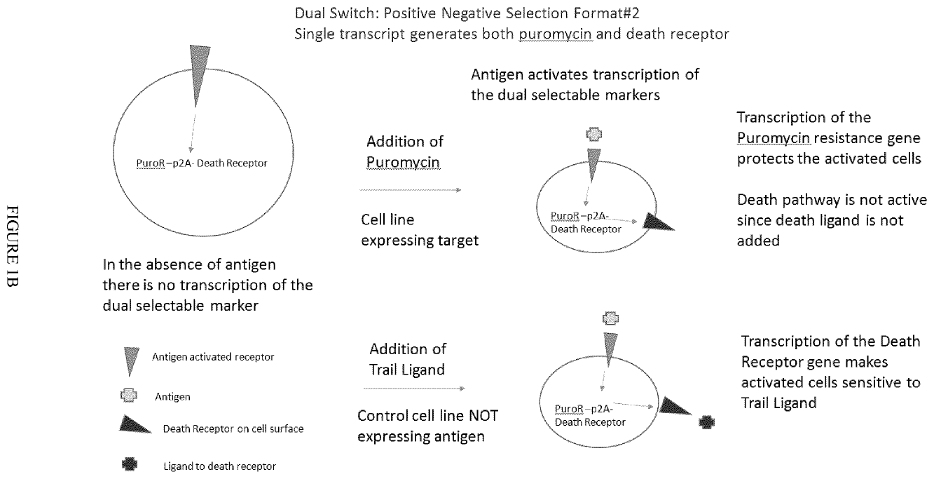

In some embodiments, the eukaryotic cell comprises both a negative selectable marker and a positive selectable marker. For example, when the negative selectable marker and the positive selectable marker are each mediated by a different exogenous mediator, then the biosensor may be used with either positive or negative selection from the activation of a single chimeric receptor. Two representative (but non-limiting) schematics of such a dual selection biosensor are shown in FIGS. 1A and 1B.

In some embodiments, the marker gene is a positive selectable marker gene (under the transcriptional control of NF-κB) and the TNFRSF member is a death receptor. This allows for negative selection in the absence of apoptosis inhibitors (e.g. caspase inhibitors) and positive selection in the presence of apoptosis inhibitors. For example, but without limitation, when the positive selectable marker is Puro expression, then the inclusion of apoptosis inhibitors (e.g. caspase inhibitors) during use allows for positive selection by adding puromycin to the cell media. Any of the aforementioned positive selection markers may likewise be used with a death receptor or death receptor fragment signaling portion to enable negative or positive selection. In certain embodiments, the TNFRSF member need not necessarily be a death receptor as negative selection may be implemented by engineering the eukaryotic cell to express a negative selectable marker which induces apoptosis. This approach may be used for other chimeric or natural receptors which signal through multiple pathways wherein the primary signal may be modified by inhibiting certain pathways while leaving others open.

In some embodiments, the marker gene(s) may be induced in combination with an additional receptor that when bound by a ligand activates NF-κB which would allow for increased sensitivity and longevity of the signal.

The eukaryotic cell may be engineered to inactivate a specific endogenously expressed death receptor in the eukaryotic cell. Inactivation may be accomplished by any known method (e.g. CRISPR/CAS9, zinc fingers, talons or other forms of mutagenesis). As such, the engineered eukaryotic cell may no longer signal apoptosis in response to a particular ligand (called “ligand x” for ease of reference). Then, by engineering the cell to express a death receptor that responds to ligand x when the chimeric receptor is activated, the engineered cell will be enabled for negative selection (i.e. apoptosis) when the chimeric receptor is activated and the cell media contains ligand x. When the engineered cell also expresses a positive selection marker (e.g. an antibiotic and the like), then the biosensor will also be enabled for positive selection in the absence of ligand x. For example, if endogenous DR4 (TRAILR1) and DR5 (TRAILR2) death receptors are both inactivated, then the cell will not die in the presence of the TRAIL ligand. If the cell is then engineered to express DR4 and/or DR5 when the chimeric protein is activated, the cell can be negatively selected in the presence of TRAIL.

In some embodiments, the host cell further comprises an expression cassette for expressing a cell surface protein comprising an extracellular domain for displaying the target binding substrate. This binding substrate may be a multivalent binding substrate (e.g. expressed as a fusion protein with the cell surface protein). The binding substrate may be a univalent binding substrate that forms a multivalent binding substrate through multimerization of the cell surface protein. In certain embodiments, the expression cassette for the cell surface protein may comprise an inducible promoter operably linked to a nucleic acid sequence or sequences which encode(s) the cell surface protein.

In some embodiments, the at least one nucleic acid comprising one or more coding sequences which collectively encode the chimeric receptor is integrated in a chromosome of the eukaryotic cell.

In some embodiments, the eukaryotic cell is a vertebrate cell. In some embodiments, the vertebrate cell is a mammalian cell or a non-mammalian vertebrate cell. The mammalian cell may be a human cell or a non-human cell. In some embodiments, the vertebrate cell is a human cell. In some embodiments, the eukaryotic cell is a human cell-line. In certain embodiments, the eukaryotic cell is a vertebrate cell that is NF-κB competent in response to activation of the TNFRSF member.

Without limitation, this disclosure also provides a method of producing the chimeric receptor defined herein. The method comprises culturing the eukaryotic cell under conditions which express the chimeric receptor. Expression conditions will depend on the particular eukaryotic cell and promoter operably linked to the at least one nucleic acid comprising one or more coding sequence which collectively encode the chimeric receptor.

Without limitation, this disclosure also provides a library (or population or repertoire) of biosensors as described herein, the library of biosensors comprising a plurality of unique biosensors which collectively bind a plurality of uncharacterized epitopes, each biosensor of the plurality of unique biosensors comprising a eukaryotic cell (e.g. a vertebrate cell), which is NF-κB competent in response to the TNFRSF member from which the chimeric receptor is derived, that expresses a unique chimeric receptor as described herein, in which the extracellular binding portion comprises an antibody or antigen binding fragment having unknown binding specificity, the antibody or antigen binding fragment having unknown binding specificity comprising at least one CDR of unique amino acid sequence compared to all other biosensors in the plurality of unique biosensors. The plurality of unique biosensors may comprise any number of biosensors. In some embodiments, the plurality of unique biosensors comprises at least 1000, at least 5000, at least 10,000, at least 50,000, at least 100,000, at least 500,000, at least 1 million, at least 10 million, at least 50 million, at least 100 million, at least 500 million, at least 1 billion, at least 10 billion (or at least any number therebetween the foregoing numbers) unique biosensors. The plurality of unique biosensors may number more than 10 billion. Methods for generating libraries of diverse antibody specificities and affinities are known, including without limitation, using in vitro V(D)J recombination, mutagenesis and/or CRISPR methods, error prone PCR, degenerate oligos or degenerate gene synthesis products.

### III. Methods/Uses of the Biosensor

Without limitation, the chimeric receptor described in Section II may be used for binding a binding substrate that specifically binds the binding portion of the chimeric receptor. This disclosure thus provides a method of binding a binding substrate, comprising contacting the chimeric receptor (any embodiment described in Section II) with a binding substrate that specifically binds the binding site in the binding portion of the chimeric receptor. This disclosure also provides use of the chimeric receptor described in Section II for binding a binding substrate that specifically binds the binding portion of the chimeric receptor. In some embodiments of these methods and uses, the chimeric receptor may be localized in the plasma membrane of a cell.

Without limitation, the chimeric receptor as described in Section II may be used in a biosensor (i.e. a whole cell biosensor) for detecting binding to a multivalent binding substrate. For example, but without limitation, the vertebrate cell described in Section II may be used as a biosensor for detecting binding to a multivalent binding substrate. This disclosure thus provides a method of detecting binding between a biosensor and a multivalent binding substrate. This method comprises contacting the biosensor with the multivalent binding substrate and identifying binding between the biosensor and the multivalent binding substrate based on a level of intracellular signaling activity of the signaling portion of the chimeric receptor compared with a background level (e.g. the level in the absence of the multivalent binding substrate). For example, the biosensor may comprise a first vertebrate cell that expresses a chimeric receptor, in which the chimeric receptor comprises a signaling portion, a transmembrane domain and an binding portion, wherein the signaling portion comprises a TNFRSF member or a fragment of the TNFRSF member which retains an intracellular signaling domain of the TNFRSF member, wherein the binding portion comprises an extracellular binding site which specifically binds the multivalent binding substrate, wherein the extracellular binding site is not native to the TNFRSF member. For clarity, in these embodiments the binding site is extracellular and the intracellular signaling domain is intracellular when the chimeric receptor is expressed in the first vertebrate cell. Binding of the multivalent binding substrate to the extracellular binding site of the binding portion activates the intracellular signaling activity of the signaling portion (e.g. by cross-linking the chimeric receptor). The chimeric receptor may be any described in Section II. The first vertebrate cell may be as described for the vertebrate cell in Section II, namely a vertebrate cell that activates NF-κB signaling (i.e. NF-κB competent) in response to activation of the TNFRSF member.

The binding portion (and the extracellular binding site comprised within) may be any described in Section II, including without limitation, a peptide (e.g. a random peptide of 7 or more amino acids), an antibody or antigen binding fragment thereof, a monobody, an affibody, an anticalin, a DARPin, a Kunitz domain, an avimer or a soluble T-cell receptor. In other embodiments, the chimeric receptor comprises, and the binding portion is comprised within, a TCR or an antigen-binding fragment of the TCR. Each of these is further described in Section II.

In some embodiments, the level of the intracellular signaling activity positively corresponds to a measure of cell death (e.g. but without limitation a rate of cell death). In some embodiments, the level of the intracellular signaling activity positively corresponds to a measure of cell survival (e.g. but without limitation a rate of cell survival).

In some embodiments, the level of the intracellular signaling activity positively corresponds to an expression level of a marker gene(s) (e.g. one or more screenable marker genes, selectable marker genes and/or screenable-selectable marker genes) that is activated or repressed by NF-κB. The marker gene(s) and their regulatory elements may be as described in Section II.

In some embodiments the marker gene(s) may comprise a screenable or screenable-selectable marker gene and said identifying binding may comprise determining an expression level of the screenable marker gene or the screenable-selectable marker gene (including without limitation any screenable marker gene or any screenable-selectable marker gene described in Section II). For example, but without limitation, the marker gene(s) may encode or cause expression of a surface antigen (e.g. CD19 or CD19-Puro and the like) and said identifying binding may comprise determining an expression level of the surface antigen.

In certain embodiments, the selectable marker gene(s) may comprise a positive selectable marker gene (e.g. any described in Section II) and said identifying binding comprises positively selecting based on chemical detoxification, based on exclusion or removal, based on increased expression (such as the dihydrofolate reductase or “DHFR” gene, and the like), based on pathogen resistance, based on heat tolerance, based on radiation resistance, based on double-strand break sensitivity, based on ability to utilize non-metabolized compounds (e.g. HPRT, GPT and the like) and/or based on acquisition of a growth factor. In some embodiments, expression of the marker gene(s) causes resistance to a toxic compound/condition and said identifying binding comprises detecting survival of the first vertebrate cell in the presence of the toxic compound/condition. For example, but without limitation, the marker gene(s) may encode or cause expression of an antibiotic resistance protein (e.g. Puro or CD19-Puro) and identifying binding may comprise contacting the first vertebrate cell with the antibiotic (e.g. puromycin), such as by adding the antibiotic to the cell media.

In some embodiments the selectable marker gene(s) may comprise a negative selectable marker gene (e.g. any described in Section II) and said identifying binding comprises selecting based on cell death. For example, but without limitation, the selectable marker gene may encode or cause expression of a toxin or an enzyme (e.g. HPRT, GPT or a suicide gene(s) such as herpes simplex virus thymidine kinase gene, the cytosine deaminase gene, the varicella-zoster virus thymidine kinase gene, the nitroreductase gene, the E. coli Deo gene and the like) which can convert a precursor compound to a toxic compound.

In some embodiments, the first vertebrate cell may comprise both a negative selectable marker gene and a positive selectable marker gene and identifying binding may comprise selection based on either of cell death and survival, depending on the particular cell conditions or presence/absence of an exogenous mediator and/or an apoptosis inhibitor.

When the TNFRSF member is a death receptor, the method may (in some embodiments) further comprise contacting the biosensor with an apoptosis inhibitor (e.g. a caspase inhibitor such as caspase-8 inhibitor and/or caspase-10 inhibitor or a pan-caspase inhibitor) prior to or during said contacting the biosensor with the multivalent binding substrate. Caspase inhibitors are known (e.g. pan-caspase inhibitor Z-VAD-FMK and the like) and function in vertebrate cells to reduce apoptosis due to activation of death receptors.

In some embodiments, contacting the biosensor with the multivalent binding substrate comprises co-culturing the biosensor with a second cell which comprises the multivalent binding substrate. The multivalent binding substrate may be expressed on the surface of the second cell. The multivalent binding substrate may be secreted from the second cell. The second cell may be a second vertebrate cell or anon-vertebrate cell (e.g. a fungus cell, a bacterial cell, a yeast cell, and the like).

In some embodiments, multivalent binding substrate may be in solution or in a mixture. For example, but without limitation, the multivalent binding substrate may be in a cell lysate, serum sample or other biological sample or analyte.

In some embodiments, the method further comprises preparing the multivalent binding substrate prior to contacting the biosensor with the multivalent binding substrate. For example, but without limitation, the multivalent binding substrate may be prepared by oligomerizing or complexing a binding substrate (e.g. a monovalent binding substrate) and/or by expressing the binding substrate on the surface of a cell such that the multiple units of the binding substrate is displayed on the cell surface in close proximity to each other. Oligomerizing or complexing a protein (such as the binding substrate) may be achieved by various different methods. A common method is to biotinylate the protein and incubate it with avidin which has multiple binding sites for biotin to create a substrate with increased valency. If the protein is biotinylated in multiple positions then the complexes may be larger than mono-biotinylated proteins. The use of cross-linking reagents may also bring multiple proteins/molecules together. Expressing the protein as an Fc-fusion protein creates a dimer of the molecule. The use of a secondary antibody to cross-link the Fc-fusion protein further increases the valency of the substrate. Expression as an IgM or IgA fusion protein may also provide multivalent molecules. Molecules may be linked to beads (e.g. agarose) or ELISA plates to provide for increased surface valency. Molecules expressed on the surface of a cell provides a format that has valency suitable for a substrate to activate the chimeric receptor (e.g. by cross-linking).

In some embodiments, contacting the biosensor with the multivalent binding substrate comprises co-expressing a cell surface protein in the first vertebrate cell with the chimeric protein, the cell surface protein comprising an extracellular domain comprising: the multivalent binding substrate; or a univalent binding substrate that forms the multivalent binding substrate through multimerization of the cell surface protein. In some embodiments, expressing the cell surface protein is inducible and the method further comprises inducing co-expression of the cell surface protein.

Using substrate binding dependent signaling (e.g. antigen dependent signaling) to mediate both positive and negative selection is particularly useful for isolating rare binding specificities from large cell-expressed repertoires. The ability to utilize selection both positive and negative selection is an improvement over only positive or negative selection since it allows even larger repertoires to be interrogated and even rarer events to be isolated. In addition, dual selection allows for the direct elimination of off-target binding events.

Although utilizing a biosensor approach (a cell utilizing a cell surface signal) has the advantage that the target binding substrate does not need to be purified and can be expressed in its native conformation in the plasma membrane of the target cell, applying a large (and diverse) biosensor library has some unique challenges, e.g. when trying to isolate a biosensor that is specific for a particular target binding substrate on a cell surface. Because the target cell has thousands of proteins representing 100s of thousands of binding substrates all potentially activating biosensors, it would be particularly useful to be able to distinguish target-specific activated biosensors from background activated biosensors. Incomplete activation of the biosensor (for example if only 80% of the cells are activated the other 20% will appear as negative but possess the incorrect specificity) and/or incomplete staining generate populations of background cells that represent an undesirable level of background when starting with large library populations (e.g. a billion cells), which may make it difficult or laborious to isolate the rare biosensor with the desired specificity (this is similar to the challenge with phage display where negative panning is inefficient). These limitations may be overcome in some embodiments disclosed herein, where the biosensors are equipped for both functional positive and negative selection.

Biosensor repertoires may be alternatively exposed to cells with and without the target binding to substrate on their cell surface, alternatively being positively and negatively selected to enrich for a biosensor population that is activated only in the presence of a cell expressing the target of interest. A benefit of adding negative selection to positive selection is that it allows for the elimination of cells that are off-target (e.g. cells displaying antigens present on both the target cells and the control cells). An advantage of some such embodiments is that expensive and specialized FACS sorting equipment is not required. Another advantage of some such embodiments it that significantly more cells can be processed to isolate extremely rare binding events. Although there is a limit on how many cells a FACS machine can process in a day, some of these embodiments are not so limited and the size of the biosensor library may be easily scaled up; cultures of 10-100 liters (or more) of cells may be selected with the addition of a drug for selection like puromycin. FACS machines also are not able to routinely isolate rare events at frequencies of less than 1 in 100,000. Accordingly, it would take multiple rounds of FACS sorting to isolate the rare events of interest. Positive selection in some embodiments described herein may be able to detect rare binding events at frequencies of less than 1 in a million or even 1 in 10 million. Negative selection is also possible at the same scale, eliminating biosensors that have been activated in the presence of the control cell line. Therefore, the ability for the same signaling event (i.e. activation of the chimeric receptor) to direct cell survival or cell death allows for alternating selection pressure to isolate rare specificities from extremely large repertoires.

An exemplary (but non-limiting) example of a dual selection method is schematically shown in FIG. 1A. FIG. 1A shows a biosensor cell with a chimeric receptor (shown as a triangle) which, when activated, signals expression of PuroR (puromycin resistance protein) as well as apoptosis. During co-culture with target cells, apoptosis is inhibited by caspase inhibitors. The presence of puromycin positively selects activated biosensors and kills non-activated biosensors and the target cells. After removing puromycin and caspase inhibitors, the positively selected biosensors are co-cultured with control cells, which lack the target binding substrate, in the absence of puromycin and caspase inhibitors. This negatively selects out off-target activated biosensors, leaving only non-activated biosensors (i.e. biosensors previously selected as being target-specifically activated). This method is not limited to puromycin as the specific positive selection mechanism. In alternative embodiments, the target and control co-cultures may be performed in parallel and sequencing used to discriminate the target-specific biosensors from biosensors activated by the control cells.

Another exemplary (but non-limiting) example of a dual selection method is schematically shown in FIG. 1B. FIG. 1B shows a biosensor cell with a chimeric receptor (shown as a grey triangle) which, when activated, signals expression of intracellular PuroR linked to a death receptor which embeds in the plasma membrane. During co-culture with target cells, the presence of puromycin positively selects activated biosensors while killing non-activated biosensors and the target cells. Apoptosis from the death receptor is avoided by the absence of the death receptor ligand. The positively selected biosensors are then co-cultured with control cells, which lack the target binding substrate, in the presence of the death receptor ligand (e.g. TRAIL ligand for DR4 or DR5). This negatively selects out off-target activated biosensors, leaving only non-activated biosensors (i.e. biosensors previously selected as being target-specifically activated). As such, identifying binding may comprise contacting the first vertebrate cell with the ligand of a death receptor which is only expressed in response to activation of the chimeric receptor. This method is not limited to puromycin as the specific positive selection mechanism or to DR4/DR5 as the specific death receptor. This method is also not limited to the positive and negative selection elements being linked as a fusion protein. In alternative embodiments, the target and control co-cultures may be performed in parallel and sequencing used to discriminate the target-specific biosensors from biosensors activated by the control cells.

While traditional library screens can be applied using the described biosensor approach where an exogenous target (or cell line expressing a target of interest) is incubated with the biosensor and activation in trans identifies bisosensors with specificity to the target of interest, the cell based biosensor system also is amendable to configuring the screen in an autocrine manner. In such embodiments the target sequence is expressed in the biosensor cells (along with the biosensor receptor/chimeric receptor) as opposed to being added exogenously. The target of interest can be expressed in an induced manner so that biosensors can be identified that are only activated when the target is expressed. In a non-limiting example, the library of biosensors comprising a plurality of unknown binding specificities is subjected to negative selection. Biosensor cells with extracellular binding sites specific for its own cell surface proteins will be activated in an autocrine fashion to express the negative selectable marker (e.g. death receptor such as DR4, DR5, which can be activated by a death ligand such as TRAIL, or any other negative selectable marker previously described) such that biosensor cells expressing these anti-self binding specificities will be killed and eliminated. Subsequently the expression of the target protein is expressed. Biosensor cells activated following the induced expression of the target will survive positive selection.

### IV. Host Cells & Nucleic Acids

Without limitation, this disclosure provides a host cell comprising a receptor which signals production of a positive selectable marker and/or a negative selectable marker in response to the receptor being bound by a specific binding substrate.

The host cell may be any cell, e.g. a bacterial cell or a eukaryotic cell. In some embodiments, the host cell is a eukaryotic cell. The eukaryotic cell may be any eukaryotic cell. In some embodiments, but without limitation, the eukaryotic cell may be a yeast cell or a vertebrate cell. The yeast cell may be any yeast cell. For example, but without limitation, GPCRs and other vertebrate/mammalian receptors have been expressed in yeast and yeast is known to be capable of reconstituting mammalian growth-signaling pathways (e.g. mediated by EGF-EGFR-Grb2/Shcl-Sos-Ras complex; see Yoshimoto et al., 2014, Sci Rep. 4: 4242). The vertebrate cell may be any vertebrate cell. In some embodiments, but without limitation, the vertebrate cell may be a mammalian cell. In some embodiments, but without limitation, the mammalian cell may be a mammalian cell line, a human cell or a human-derived cell line (e.g. HEK293 or any other human-derived cell line).

As used herein, the term “receptor” means a protein that causes a signal or cellular response inside the cell in response to the protein binding a substrate. Unless otherwise specified, the receptor of the host cell (also called “host cell receptor”) may be intracellular, membrane-associated, or transmembrane. The receptor may be a transmembrane receptor that binds a substrate outside the cell and produces a signal inside the cell. Other receptors may be cytosolic and bind substrate intracellularly and also produce an intracellular signal. The receptor may be a multi-subunit protein or a single subunit protein. The receptor may be artificial or a natural receptor. The receptor may be native to the host cell or heterologous (non-native) to the host cell. In some embodiments, the receptor is a chimeric receptor, e.g. a fusion protein or a fusion protein complex.

The receptor comprises a binding portion and a signaling portion. In some embodiments, the receptor may comprise a binding portion, a transmembrane portion and a signaling portion.

The binding portion of the receptor (or the chimeric receptor) may be any binding moiety. In some embodiments, but without limitation, the binding portion comprises an antibody or antigen binding fragment thereof, which specifically binds the specific binding substrate.

The antibody may be of any species or may be chimeric or artificial. For example, but without limitation, the antibody may be non-human (e.g.: a camelid, such as dromedary, camel, llama, alpaca, and the like; cartilaginous fish, such as shark and the like; mouse, rat, monkey or other), primatized, humanized or fully human. A chimeric antibody contains amino acid sequences from multiple species, e.g. from human and non-human or from two non-human species. Methods for humanizing (or primatizing) non-human antibodies are well known in the art, e.g. by substituting non-human (or non-primate) constant domains for those of a human antibody (creating a chimeric antibody) or by substituting one or more (e.g. 1, 2, 3, 4, 5 or 6) of the Complementarity Determining Regions (CDRs) of a human (or primate) antibody with a non-human antibody (see, e.g.: Jones et al. Nature 1986; 321:522-525; Riechmann et al. Nature 1988; 332:323-327; Verhoeyen et al. Science 1988; 239:1534-1536; Presta. Curr. Op. Struct. Biol. 1995; 2:593-596; Morrison et al. Proc. Natl. Acad. Sci. USA 1984; 81:6851-6855; Morrison and Oi. Adv. Immunol. 1988; 44:65-92; Padlan. Molec. Immun. 1991; 28:489-498; and Padlan. Molec. Immun. 1994; 31(3):169-217). The antibody may be comprised of two heavy chains and two light chains. The antibody may be a single-chain antibody with the heavy chain and light chain separated by a linker. The antibody may be a heavy chain only antibody (e.g. an dromedary, camel, llama, alpaca or shark antibody which lacks light chains, or a human heavy chain). The antibody may be a single-domain antibody (sdAb).

“Artificial” antibodies include known antibody derivatives, e.g. scFv (i.e. single chain Fv), scFv-Fc, minibodies, nanobodies, diabodies, tri(a)bodies and the like.

As used herein, the term “antigen binding fragment” of an antibody means any antibody fragment which possesses antigen binding activity. In some embodiments, the antigen binding fragment comprises antibody light chain and heavy chain variable domains (i.e. VL and VH domains). In some fragments, the light chain is omitted. Non-limiting examples of antibody fragments include Fab, Fab′ and F(ab′)2.

Non-limiting examples of antibodies and antigen binding fragments include, without limitation: IgA, IgM, IgG, IgE, IgD, sdAb, Fab, Fab′, F(ab′)2, scFv, scFv-Fc, minibodies, nanobodies, diabodies, tri(a)bodies and the like. Other antibodies and fragments are known, a number of non-limiting examples of which are disclosed in Deyev and Lebedenko (2008, BioEssays 30:904-918). In some embodiments, the antibody or antigen binding fragment thereof is a IgA, a IgM, a IgG, a IgE, a IgD, a sdAb, a Fab, a Fab′, a F(ab′)2, a scFv, a scFv-Fc, a minibody, a nanobody, a diabodies or a tri(a)body. In some embodiments, the antibody is a IgG antibody.

In some embodiments, the antibody or antigen binding fragment (e.g. without limitation an IgG antibody or fragment thereof) binds the binding substrate with a dissociation constant (i.e. K_{D}) of less than 500 nM, less than 400 nM, less than 300 nM, less than 200 nM, less than 100 nM or less than 50 nM. In some embodiments, the antibody or antigen binding fragment may bind the binding substrate with a picomolar K_{D}. The affinity and specificity of the antibody or antigen binding fragment may have been engineered, for example, but without limitation, by using in vitro V(D)J recombination, mutagenesis and/or the use of double-stranded breaks together with Tdt such as with restriction enzymes, CRISPR, Zinc Finger or Talon methods or the use of error prone PCR, degenerate oligos or degererate gene synthesis products.

In some embodiments, but without limitation, the binding portion of the receptor (or the chimeric receptor) comprises a monobody, an affibody, an anticalin, a DARPin, a Kunitz domain, an avimer or a soluble T-cell receptor, which specifically binds the binding substrate. In other embodiments, the receptor (or the chimeric receptor) comprises, and the binding portion is comprised within, a TCR or an antigen-binding fragment of the TCR.

Monobodies (also called Adnectin^{T}) are synthetic binding proteins based on the structure of the tenth extracellular type II domain of human fibronectin. They have exposed loops which resemble the structure, affinity and specificity of antibody CDRs, but are much smaller (approximately 90 amino acids) and lack disculfide bonds, which makes them particularly useful for inclusion in fusion proteins (Lipovsek. Protein Eng Des Sel 2011; 24:3-9).

Affibodies are small proteins (approximately 6 kDa) based on the Z domain of protein A. Compared to antibodies, they are much smaller and lack disulfide bonds, such that they can be readily included into a fusion protein. Affibodies with unique binding properties are generally acquired by modification of 13 amino acids located in two alpha-helices involved in the binding activity, although additional amino acids outside this binding surface may also be modified (see, e.g.: Lofblom, et al. FEBS Lett. 2010; 584:2670-2680; and Nygren, FEBS J. 2008; 275:2668-2676).

Anticalins are artificial proteins derived from human lipocalins. They have a small size of approximately 20 kDa and contain a barrel structure formed by eight antiparallel β-strands pairwise connected by loops and an attached α-helix. Conformational deviations are primarily located in the four loops reaching in the ligand binding site (Gebauer and Skerra. Methods in Enzymology 2012; 503:157-188; Skerra. FEBS J. 2008; 275:2677-2683; and Vogt and Skerra. Chembiochem. 2004; 5:191-199).

DARPins are designed ankyrin repeat proteins. The ankyrin repeat motif consists of approximately 33 amino acids which form a loop, a β-turn, and 2 antiparallel α-helices connected by a tight turn (see, e.g.: Stumpp & Amstutz. Curr. Opin. Drug. Discov. Devel. 2007; 2:153-9; Pluckthun. Annual Review of Pharmacology and Toxicology 2015; 55:489-511; and Martin-Killias, et al. Clin. Cancer Res. 2010; 17:100-110).

Avimers are artificial proteins that comprise two or more A domains of 30 to 35 amino acids each fused together (optionally with linker peptides). The A domains are derived from various membrane receptors and have a rigid structure stabilized by disulfide bonds and calcium. Each A domain can bind to a different epitope of a target protein to increase affinity (i.e. avidity) or can bind epitopes on different target proteins (see, e.g.: Silverman et al. Nat. Biotechnol. 2005; 23(12):1556-61).

Kunitz domains are peptides that form stable structures able to recognize specific targets and have been previously incorporated into fusions proteins (Zhao et al. Int. J. Mol. Med. 2016; 37:1310-1316) and phage display libraries (WO 2004063337).

Soluble TCRs or single-variable domain TCRs have been described, e.g, ImmTAC™ and the like (Oates & Jakobsen. Oncolmmunology 2013; 2:2, e22891) and as described in PCT Patent Publication No. WO/2017/091905. Single-variable domain TCRs are included within the term “a TCR or an antigen-binding fragment of the TCR”, which also includes all other known antigen-binding fragments of TCRs.

In the context of the receptor herein, a “binding site” refers to the amino acids in a protein that are required and responsible for the binding properties of the binding portion. Unless otherwise indicated, the “binding site” of the chimeric receptor is not limited to canonical ligand-binding sites of receptors, substrate-binding sites of enzymes, and antigen-binding sites of antibodies (to name but a few), but instead refers to any amino acid sequence or sequences (including peptides, polypeptides and proteins) longer than 6 residues (e.g. 7 or more amino acids) that is capable of specifically binding, or being specifically bound to or by, the binding substrate (or ligand). In some embodiments, the binding site excludes sequences such as FLAG, V5, Myc, stretches of Histidine sequences or other sequences that are used as “tags” in a fusion protein. The binding substrate (or ligand) may be a peptide, polypeptide, protein, sugar, polysaccharide, DNA, RNA, hapten, small organic molecule or any other molecule. In some embodiments, but without limitation, the binding substrate is a cell surface-anchored or secreted protein, polysaccharide or glycoprotein. The ligand may or may not be known for the binding site (e.g. if the binding site is artificial or derived from an orphan receptor). The binding portion may comprise multiple binding sites. For example, antibodies (such as IgG) contain antigen-binding domains and binding sites in their Fc region.

Accordingly, in some embodiments, the binding site is comprises a peptide of 7 or more randomized amino acids (as have been used in random peptide libraries). Random peptide libraries have been shown to be a powerful tool for studying protein-protein interactions and identifying peptides that can bind target molecules (e.g. phage-displayed peptide libraries were first described in 1985). Peptide libraries have been applied to identify bioactive peptides bound to receptors or proteins, disease-specific antigen mimics, peptides bound to non-protein targets, cell-specific peptides, or organ-specific peptides, and epitope mapping. Peptide libraries have also been utilized in yeast and bacterial systems in a variety of formats and mammalian two-hybrid screening approaches. The current invention allows for another format using biosensors which offers increased sensitivity. In some embodiments, peptides are expressed as the entire binding portion (i.e. as an extracellular binding domain) or as part of the binding portion. For example, the peptide binding site may be expressed as a fusion protein, linked to a transmembrane domain (native or non-native to the TNFRSF member) which is linked to the intracellular signaling domain of the TNFRSF member. In combination with the de novo engineering using V(D)J recombination or viral infection, large libraries of biosensors can be generated that display random peptide libraries.

When present, the transmembrane portion (or transmembrane domain) of the receptor (or chimeric receptor) may be a natural transmembrane domain (e.g. a segment or segments from a natural transmembrane protein) or an artificial transmembrane domain (e.g. a hydrophobic α-helix of about 20 amino acids, often with positive charges flanking the transmembrane segment). The transmembrane domain may have one transmembrane segment or more than one transmembrane segment. The transmembrane domain may be α-helical and have one transmembrane segment (i.e. single-pass) or more than one transmembrane segment (multi-pass). The transmembrane domain may comprise a n-sheet or n-barrel. Prediction of transmembrane domains/segments may be made using publicly available prediction tools (e.g. TMHMM, Krogh et al. Journal of Molecular Biology 2001; 305(3):567-580; or TMpred, Hofmann & Stoffel Biol. Chem. Hoppe-Seyler 1993; 347:166). The topology of integral membrane proteins is thus predictable, such that it is understood which termini (N- or C-) and loop(s) (if present) are intracellular or extracellular for fusion and/or association with the signaling portion and binding portion of the receptor (or the chimeric receptor). When the receptor (or the chimeric receptor) is an integral membrane protein, its orientation in the plasma membrane is determined by the amino acid sequence including the presence/absence of signal peptides, the net electrostatic charge flanking the transmembrane segments, and the length of the transmembrane segments. As a general rule, the flanking segment that carries the highest net positive charge remains on the cytosolic face of the plasma membrane and long hydrophobic segments (>20 residues) tend to adopt an orientation with a cytosolic C-terminus. Certain membrane proteins (e.g. beta-barrels and the like) may use chaperones and other/additional mechanisms for translation and insertion into the plasma membrane. In some embodiments, the transmembrane domain of the receptor (or the chimeric receptor) is natural and either heterologous or native to the signaling portion.

In some embodiments, the transmembrane domain is a single-pass transmembrane domain, such as but without limitation the transmembrane domain of CD4 or PDGFR. The single-pass transmembrane domain may be a hydrophobic α-helix of about 15 to about 23 amino acids (e.g. 15, 16, 17, 18, 19, 20, 21, 22 or 23 residues), often with positive charges flanking the transmembrane segment.

In some embodiments, the transmembrane domain is a multi-pass transmembrane domain. The multi-pass transmembrane domain may have 2, 3, 4, 5, 6, 7, 8, 9 10 or more than 10 transmembrane segments. In some embodiments, the multi-pass transmembrane domain is a 4-helix transmembrane domain, such as but without limitation the transmembrane domain of CD20. For the transmembrane domain of CD20, both the N-terminus and the C-terminus are intracellular, such that the extracellular domain is within an extracellular loop. In some embodiments, the multi-pass transmembrane domain is a 7-helix transmembrane domain, such as but without limitation the transmembrane domain of glucagon-like peptide 1 receptor (GLP1R) or another G-protein coupled receptor. The N-terminus of the GLP1R transmembrane domain is extracellular and the C-terminus is intracellular.

In some embodiments, the transmembrane domain is selected from the transmembrane domains of integral membrane proteins that are human CD molecules (also known as “clusters of differentiation”, “clusters of designation” or “classification determinants”).

In some embodiments, the signaling portion of the receptor (or the chimeric receptor) may comprise or be obtained from a natural receptor that has intracellular signaling activity when activated by cross-linking or by increasing a local concentration of the natural receptor, or may comprise or be obtained from a fragment of the natural receptor which retains the intracellular signaling activity of the receptor when activated by cross-linking or by increasing local concentration of the receptor.

For example, in some embodiments, the signaling portion comprises or is obtained from a tumor necrosis factor receptor superfamily (TNFRSF) member or a fragment of the TNFRSF member which retains an intracellular signaling domain of the TNFRSF member. Where the receptor is a chimeric transmembrane receptor, the binding site is extracellular and the intracellular signaling domain is intracellular when the chimeric receptor is expressed in the host cell. Accordingly, the chimeric receptor retains functional membrane localization and TNFRSF intracellular signaling activity when expressed in the host cell. In some embodiments, the signaling portion is heterologous to the binding portion.

The TNFRSF is a group of cytokine receptors generally characterized by an ability to bind ligands (such as TNFs) via an extracellular cysteine-rich ligand-binding domain and signal a cellular response when activated by binding. Certain TNFRSF members (e.g. TNFR1, TNFR2, TRAIL and the like) also have a pre-ligand binding assembly domain (PLAD) as part of their extracellular domain that plays a role in pre-assembly of the TNFRSF member in a ligand-unbound state (Chan. Cytokine. 2007; 37(2): 101-107). In their active (signaling) form, the majority of TNFRSF members form trimeric complexes in the plasma membrane, although some TNFRSF members are soluble or can be cleaved into soluble forms.

In addition to an extracellular ligand-binding domain and a transmembrane domain, TNFRSF members have an intracellular (or cytoplasmic) domain involved in signaling various cellular responses when the TNFRSF member is in a ligand-bound state, not through an intrinsic enzymatic activity of the intracellular domain, but through association of the intracellular domain with adaptor proteins (e.g. TRADD, TRAF, RIP, FADD and the like) which form (or cause the formation of) signaling complexes with accessory proteins having enzymatic activity (e.g. kinase or polyubiquitination activity). TNFRSF members signal a wide range of overlapping cellular responses, including but not limited to proliferation, differentiation, nuclear factor kappa B (NF-κB or NF-κB) activation, cell death, and stress-activated protein kinase (SAP kinase). The intracellular domain of TNFRSF members generally lack recognizable common motifs among the members, the exception being a subgroup of TNFRSF members called “death receptors”, which comprise an approximately 80 amino acid long cytoplasmic “death domain”. The death domain binds other death domain-containing proteins. A death receptor ligand may be called a “death ligand”.

As used herein in the context of TNFRSF, the term “intracellular domain”, “cytoplasmic domain” “signaling domain” or “intracellular signaling domain” all refer to the domain, domains or portions thereof of a TNFRSF member that are required for binding adaptor protein(s). A fragment which retains functional membrane localization and intracellular signaling activity of the TNFRSF member when expressed in a NF-κB competent cell (e.g. a vertebrate cell) may be confirmed using functional assays which assess signaling at any point in the signaling pathway of the TNFRSF member. For example, which is not to be considered limiting, TNFR1 is known to, among other functions, activate NF-κB and cause apoptosis. NF-κB is a highly conserved pathway in eukaryotes (not just vertebrates) and has been characterized in yeast. The yeast retrograde response is a predecessor with many similarities to the central stress-regulator, NF-κB found in advanced multicellular organisms (Moore et al. Molecular and Cellular Biology 1993; 13:1666-1674). Accordingly, detecting cell death may be used to confirm that intracellular signaling activity is retained in a particular TNFRSF fragment. Alternatively, activated NF-κB can be detected directly or indirectly. Numerous tools/kits are commercially available for detecting activated NF-κB, including enzyme-linked immunosorbent assays (ELISA) and electrophoretic mobility shift assays (EMSAs). Alternatively, since NF-κB is a transcription factor, activated NF-κB may also be detected by linking a screenable marker gene or selectable marker gene to a NF-κB response element.

In certain embodiments, the TNFSRSF member is CD27 (also called TNFRSF7, s152 and Tp55), CD40 (also called TNFRSF5, p50 and Bp50), EDA2R (also called ectodysplasin A2 receptor, XEDAR, EDA-ADA-A2R, TNFRSF27), EDAR (also called ectodysplasin A receptor, ED3, DL, EDS, EDA3, Edar, ED1R, EDA1R), FAS (also called Fas cell surface death receptor, FAS1, APT1, TNFRSF6, CD95, APO-1), LTBR (also called lymphotoxin beta receptor, D12S370, TNFCR, TNFR-RP, TNFR2-RP, TNF-R-III, TNFRSF3), NGFR (also called nerve growth factor receptor, TNFRSF16, CD271, p75NTR), RELT (also called RELT tumor necrosis factor receptor, TNFRSF19L, F1114993), TNFR1 (also called TNF receptor 1, TNFRSF1A, TNF-R, TNFAR, TNFR60, TNF-R-I, CD120a, TNF-R55), TNFR2 (also called TNF receptor 2, TNFRSF1B, TNFBR, TNFR80, TNF-R75, TNF-R-II, p75, CD120b), TNFRSF4 (also called TXGP1L, ACT35, OX40, CD134), TNFRSF6B (also called DcR3, DCR3, TR6, M68), TNFRSF8 (also called CD30, D1S166E, KI-1), TNFRSF9 (also called ILA, CD137, 4-1BB), TNFRSF10A (also called DR4, Apo2, TRAILR1, CD261), TNFRSF10B (also called DR5, KILLER, TRICK2A, TRAILR2, TRICKB, CD262), TNFRSF10C (also called DcR1, TRAILR3, LIT, TRID, CD263), TNFRSF10D (also called DcR2, TRUNDD, TRAILR4, CD264), TNFRSF11A (also called PDB2, LOH18CR1, RANK, CD265, FEO), TNFRSF11B (also called OPG, OCIF, TR1), TNFRSF12A (also called FN14, TweakR, CD266), TNFRSF13B (also called TACI, CD267, IGAD2), TNFRSF13C (also called BAFFR, CD268), TNFRSF14 (also called HVEM, ATAR, TR2, LIGHTR, HVEA, CD270), TNFRSF17 (also called BCMA, BCM, CD269, TNFRSF13A), TNFRSF18 (also called AITR, GITR, CD357), TNFRSF19 (also called TAJ-alpha, TROY, TAJ, TRADE), TNFRSF21 (also called DR6, CD358), TNFRSF25 (also called TNFRSF12, DR3, TRAMP, WSL-1, LARD, WSL-LR, DDR3, TR3, APO-3), ora protein having an intracellular signaling domain that has at least 80%, 81%, 82%, 83%, 84%, 85%, 86%, 87%, 88%, 89%, 90%, 91%, 92%, 93%, 94%, 95%, 96%, 97%, 98% or 99% amino acid sequence identity to the intracellular signaling domain of any TNFRSF member listed above and which retains TNFRSF membrane localization and TNFRSF intracellular signaling activity when expressed in the host cell. In some embodiments, the intracellular signaling domain of the TNFRSF member is a conservative mutant that has at least 80%, 81%, 82%, 83%, 84%, 85%, 86%, 87%, 88%, 89%, 90%, 91%, 92%, 93%, 94%, 95%, 96%, 97%, 98% or 99% amino acid sequence identity to the intracellular signaling domain of any TNFRSF member listed above and which retains sufficient intracellular signaling activity to cause activation of a NF-κB response element when the chimeric receptor is expressed in a eukaryotic cell that is NF-κB competent cell (e.g. a vertebrate cell, a mammalian cell, a human cell or a human-derived cell line). The TNFRSF membrane localization and TNFRSF intracellular signaling activity may be the membrane localization and intracellular signaling activity of CD27, CD40, EDA2R, EDAR, FAS, LTBR, NGFR, RELT, TNFR1, TNFR2, TNFRSF4, TNFRSF6B, TNFRSF8, TNFRSF9, TNFRSF10A, TNFRSF10B, TNFRSF10C, TNFRSF10D, TNFRSF11A, TNFRSF11B, TNFRSF12A, TNFRSF13B, TNFRSF13C, TNFRSF14, TNFRSF17, TNFRSF18, TNFRSF19, TNFRSF21 or TNFRSF25. In embodiments which do not include the extracellular domain and/or transmembrane domain of a TNFRSF member (e.g. as listed above), functional membrane localization only requires that the intracellular signaling domain be intracellular, that the transmembrane domain be localized in the cell membrane, and that the binding site be extracellular. The level of intracellular signaling activity may be the same, higher or lower as compared to CD27, CD40, EDA2R, EDAR, FAS, LTBR, NGFR, RELT, TNFR1, TNFR2, TNFRSF4, TNFRSF6B, TNFRSF8, TNFRSF9, TNFRSF10A, TNFRSF10B, TNFRSF10C, TNFRSF10D, TNFRSF11A, TNFRSF11B, TNFRSF12A, TNFRSF13B, TNFRSF13C, TNFRSF14, TNFRSF17, TNFRSF18, TNFRSF19, TNFRSF21 or TNFRSF25, so long as the signaling portion retains sufficient intracellular signaling activity to cause activation of a NF-κB response element when the chimeric receptor is expressed in a NF-κB competent eukaryotic cell (e.g. without limitation, a vertebrate cell, a mammalian cell, a human cell or a human-derived cell line). The TNFRSF member may be a hybrid of two or more of the abovementioned TNFRSF members, and/or the intracellular domain of the TNFRSF member may be a hybrid of two or more signaling domains from the abovementioned TNFRSF members, so long as the receptor retains functional transmembrane localization and the intracellular signaling activity of a TNFRSF member.

In certain embodiments, the TNFRSF member is a death receptor. The death receptor may be TNFR1, FAS, TRAILR1, TRAILR2, TRAMP, CD358 or a protein that has at least 80%, 81%, 82%, 83%, 84%, 85%, 86%, 87%, 88%, 89%, 90%, 91%, 92%, 93%, 94%, 95%, 96%, 97%, 98% or 99% amino acid sequence identity to any death receptor listed above and which retains functional localization and intracellular signaling activity of TNFR1, FAS, TRAILR1, TRAILR2, TRAMP or CD358 when expressed in the host cell. The level of intracellular signaling activity may be the same, higher or lower as compared to TNFR1, FAS, TRAILR1, TRAILR2, TRAMP or CD358. In some embodiments, the death receptor is TNFR1, FAS, TRAILR1, TRAILR2, TRAMP or CD358. In some embodiments, the death receptor is TNFR1.

In some embodiments in which the receptor of the host cell is a chimeric receptor, the chimeric receptor (or the signaling portion thereof) comprises a full-length TNFRSF member, wherein the transmembrane portion of the chimeric receptor is the transmembrane domain from the TNFRSF member. In other embodiments, the signaling portion of the chimeric receptor comprises a fragment of the TNFRSF member which retains transmembrane and intracellular signaling domains of the TNFRSF member when expressed in a NF-κB competent eukaryotic cell (e.g. without limitation, a vertebrate cell, a mammalian cell, a human cell or a human-derived cell line). The fragment may be a deletion construct which omits the ligand-binding domain of the TNFRSF member or a portion of the ligand-binding domain (e.g. omits CRD1, CRD2, CRD3 and/or CRD4 domains or any other sequence(s) within the ligand binding domain), wherein the transmembrane domain of the chimeric receptor is the transmembrane domain from the TNFRSF member. The fragment may be a deletion construct which omits the extracellular domain of the TNFRSF member or a portion of the extracellular domain, wherein the transmembrane domain of the chimeric receptor is the transmembrane domain from the TNFRSF member. The fragment may be a deletion construct which omits the extracellular domain and the transmembrane domain of the TNFRSF member or a portion of the transmembrane domain.

In some embodiments in which the receptor of the host cell is a chimeric receptor, the signaling portion comprises the amino acid sequence of SEQ ID NO: 63 or 64, or a sequence that has at least 80%, 81%, 82%, 83%, 84%, 85%, 86%, 87%, 88%, 89%, 90%, 91%, 92%, 93%, 94%, 95%, 96%, 97%, 98% or 99% amino acid sequence identity to SEQ ID NO: 63 or 64 and which is capable of activating NF-κB signaling when the chimeric receptor is expressed in a eukaryotic cell (e.g. a vertebrate cell) that is NF-κB competent in response to activation of TNFR1 (for SEQ ID NO: 63) or TRAILR2 (for SEQ ID NO: 64). In some of these embodiments, the sequence differences as compared to SEQ ID NO: 63 or 64 are conservative amino acid substitutions.

The transmembrane domain of the chimeric receptor may or may not be part of the signaling portion. In other words, only the intracellular signaling domain of the TNFRSF member is needed when the chimeric receptor further comprises a non-TNFRSF transmembrane domain and/or a non-TNFRSF extracellular domain comprising a non-TNFRSF binding site. The transmembrane domain of the chimeric receptor may or may not be comprised within the TNFRSF member or fragment of the TNFRSF member. The transmembrane domain may be a natural transmembrane domain (e.g. a segment or a plurality of segments from a natural transmembrane protein). The natural transmembrane domain may be from the same TNFRSF member as the signaling portion or from a different TNFRSF member than the signaling portion. The natural transmembrane domain may be a natural transmembrane domain from a heterologous integral membrane protein that is not a TNFRSF member.

Without wishing to be bound by theory, TNFRSF members are thought to be activated through (1) ligand-induced receptor oligomerization, e.g. by receptor cross-linking due to binding to a multivalent ligand such as trimeric TNF, (2) through a change in conformation of a pre-assembled TNFRSF oligomer, e.g. by a change in the interaction of TNFRSF subunits in a trimeric TNFRSF complex, or (3) through a change in oligomerization state, e.g. a change from dimer to trimer (Chan. Cytokine. 2007; 37(2): 101-107). Regardless of the exact mechanism, TNFRSF members can be activated by encouraging the formation of TNFRSF oligomerization, e.g. by ligand-binding or by cross-linking the receptor. Increasing the local concentration of the receptor may also result in non-specific activation by increasing the local concentration of the TNFRSF member. Accordingly, when the host cell receptor is a TNFRSF receptor or a chimeric receptor comprising a signaling domain of a TNFRSF member, the host cell receptor can be activated by binding a binding substrate that effectively oligomerizes the signaling portion. For example, if the binding substrate is “multivalent” (i.e. has two binding sites for collectively and simultaneously binding two chimeric receptors), then binding the binding substrate will oligomerize the two chimeric receptors and activate the signaling activity of the signaling portion.

In some embodiments, but without limitation, the signaling portion of the receptor (or the chimeric receptor) may comprise or may be obtained from TPO, TollR4, HER1, HER2 or integrin a5f31 or a fragment of TPO, Tol1R4, HER1, HER2 or integrin a5f31 with signaling activity. As mentioned, TNFRSF members can be activated by binding a substrate that effectively cross-links the receptors (or otherwise brings adjacent signaling domains together). Various other receptors are activiated in analogous ways and, further, chimeric receptors which incorporate the signaling domains of such receptors would also be activated by substrate binding that effectively cross-links the chimeric receptor (or otherwise brings adjacent signaling domains together). Different approaches to direct oligomerization (i.e. cross-linking) upon substrate-binding and prevent oligomerization in the absence of substrate-binding can be used.

In some embodiments, the signaling portion is obtained from a heterodimeric receptor. For example, but without limitation, the binding portion of the chimeric receptor may be an antibody and the signaling portion of the chimeric receptor may be obtained from a naturally heterodimeric receptor. The chimeric receptor may be engineered such that each antibody heavy chain is associated with one half of the heterodimeric receptor. In some embodiments, the heterodimeric signalling molecule is an integrin receptor, which is a heterodimeric receptor with an alpha chain and a beta chain. In order to retain this configuration as an antibody fusion protein while maintaining full length antibody scaffold the antibodies need to be engineered not to homodimerize, i.e. prevent the antibody from bringing two alpha units together. This may be accomplished through the modification of the Fc domain using charged pairs, or knobs and wholes or azymetrics that prevent self-dimerization. In some embodiments, the alpha chain would be directly fused to an IgG-charge pair A and the beta chain would be directly fused to a cognate IgG-chair pair B. The resultant heterodimeric molecule would be a cell surface integrin receptor with the alpha beta pairing being directed by the integrin domains. Aberrant pairing cannot occur because IgG-charge pair A can only pair with IgG-charge pair B. In other words, AA and BB homodimers cannot form.

In another embodiment, in which the extracellular domains (ECDs) of the integrin subunits are removed, the heterodimeric configuration is retained but the extracellular regions of the alpha and beta chains are replaced with antibody sequences. In this configuration, the alpha beta transmembrane and intracellular configuration is still retained.

In another embodiment, this same configuration is retained however the extracellular regions of the alpha and beta chains are replaced with antibody sequences. In this configuration the alpha beta transmembrane is replaced with a non-integrin transmembrane, and intracellular integrin sequences are retained, such that configuration is still retained via the extracellular antibody sequences.

This same approach could be applied to other receptor classes which are active for signalling as heterodimeric molecules, such as cytokine receptors, interleukin receptors, and the like.

In some embodiments, the signaling portion may be obtained from a homodimeric receptor. In this case activation occurs when two monomeric receptors (or signaling portions) are cross-linked. As an antibody is naturally homodimeric, using an antibody or a dimeric antibody fragment as the binding portion is such a chimeric receptor may cause constitutive or aberrant activation as two signalling domains would be brought into proximity for signaling without binding substrate. In order to avoid this, charge pairs may be used as described above to prevent antibody mediated oligomerization. For example, antibody charge pair A may be genetically fused to the receptor and co-expressed with a secreted antibody IgG-charge pair B. The resultant chimeric receptor expressed on the cell surface would have IgG charge pair A bound to secreted IgG charge pair B, i.e. a full IgG expressed on the surface but only a single transmembrane domain. Homodimers in the absence of binding substrate would be specifically avoided as the charge pairs would not allow such an interaction. For example, in some embodiments, IgG charge pair A may be directly fused to a EGFR family member, which is a class of receptors which are known to signal through homodimeric clustering. In certain embodiments, IgG charge pair A may replace the entire ECD of EGFR family member but the transmembrane and intracellular portions would remain the same. In certain embodiments, IgG charge pair A may replace the entire ECD, the transmembrane portion may be from a different protein and the intracellular portion may be from the EGFR family member.

Many scaffolds for the binding portion are known which are amendable to engineering to alter the affinity and selectivity of the binding portion. Fusing these scaffolds (optionally with the addition of a linker) allows them to be incorporated into fusion proteins where they retain their binding function. In some embodiments, the binding portion may be fused to the signaling portion or transmembrane portion by peptide bond, disulfide bond or other covalent bond. For example, but without limitation, a polypeptide chain of the binding portion may be expressed on the same polypeptide chain as a polypeptide chain of the signaling portion, although other polypeptide chains may also be expressed which collectively form the receptor as a multi-subunit protein complex. As such, the receptor may be a multi-subunit protein complex or may consist of a single polypeptide chain or single polypeptide chain modified by post-translational modification in vivo.

In some embodiments the binding portion may be fused to the signaling portion using a linker (e.g. a peptide linker), when the signaling portion comprises the transmembrane domain. In certain embodiments, a linker (e.g. a peptide linker) may be used at any fusion junction in the chimeric receptor (e.g. between signaling portion and transmembrane domain and/or between binding portion and transmembrane domain).

Fusion protein linkers (including for fusion junctions, monobodies, affibodies, anticalins, avimers, Kunitz domains and others) are known. For example, the linker may be flexible or rigid. Non-limiting examples of rigid and flexible linkers are provided in Chen et al. (Adv Drug Deliv Rev. 2013; 65(10):1357-1369). In some embodiments, the linker is a peptide of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 amino acid residues, wherein each residue in the peptide may independently be Gly, Ser, Glu, Gln, Ala, Leu, Iso, Lys, Arg, Pro, or another amino acid. In some embodiments, the linker is Gly, Ser, Ser-Gly, Gly-Ser, Gly-Gly or Ser-Ser.

In addition to the signaling domain, in some embodiments the chimeric receptor may comprise an additional cytoplasmic domain. This may be a drug selectable marker (e.g. Puro, Hygro or the like) to assist in selection of an inframe chimeric receptor and/or proper orientation in the plasma membrane, a fluorescent protein (e.g. GFP, RFP or the like) to assist in identifying an inframe chimeric receptor and/or proper orientation in the plasma membrane, a transcription factor or non-TNFRSF signaling domain to amplify detection of an inframe chimeric receptor using a reporter linked to a different signaling pathway (e.g. GAL4 or the like), e.g. to boost expression levels of an antibiotic resistance gene (e.g. Puro, Hygro or the like) if inframe expession levels of the resistance gene was too weak, an additional or different TNFRSF signaling domain (e.g. to potentially amplify signaling), a domain that enhances or inhibits signaling or TNFRSF signaling (e.g. to optimize the signal to noise ratio). The additional cytoplasmic domain may be directly linked, joined with a linker or joined with a P2A or cleavage sequence.

Unless otherwise indicated, the “receptor” or “chimeric receptor” disclosed herein is not limited to single subunit fusion proteins. In some embodiments, the receptor may be a single subunit fusion protein, which is encoded by at least one nucleic acid coding sequence that is comprised of a fusion of two or more coding sequences from separate genes. In other embodiments, the receptor may be assembled from multiple protein subunits that when expressed in the eukaryotic cell associate to form a quaternary structure held together by non-covalent interactions (e.g. electrostatic, Van der Waals and hydrogen bonding) and may further be held together by covalent interactions (e.g. disulfide bridges). For example, but without intending to be limiting, one or both of the binding portion and the signaling portion may comprise multiple subunits. For example, the binding portion may comprise an antibody or antigen binding fragment thereof. The binding portion may be on a separate subunit from the transmembrane domain and signaling portion. The signaling portion may be on a separate subunit from the transmembrane domain and binding portion. For example, but without limitation, the chimeric receptor may be a multi-subunit receptor comprising at least first and second subunits. The first subunit may comprise the binding portion, which may comprise a binding domain fused to a leucine zipper (or other association domain). The second subunit may comprise the transmembrane domain and signaling domain fused to the complementary leucine zipper (or other complementary association domain). As such, the leucine zipper allows for the binding domain to associate via the leucine zipper to the transmembrane domain and signaling domain. In a second non-limiting example, the first subunit may comprise the binding portion, which comprises an extracellular binding domain fused to the transmemberane domain fused to an intracellular leucine zipper (or other association domain). The second subunit may then comprise an intracellular signaling domain fused to the complementary leucine zipper (or other complementary association domain), such that the association of the two subunits is intracellular. In both examples the binding domain and signaling domain are not genetically linked but are functionally linked. Many other association domains besides leucine zippers are known and would be suitable to direct protein-protein interactions in the formation of a multi-subunit chimeric receptor (e.g. comprising 2, 3, 4, 5, 6 or more than 6 subunits).

In some embodiments, the binding portion comprises the amino acid sequence of SEQ ID NO: 1, 2, 3 or 4 (or any other antibody heavy chain sequence disclosed herein). In some embodiments, the binding portion comprises the amino acid sequence of SEQ ID NO: 27, 29, 31 33, 46 or 47 (or any other antibody light chain sequence disclosed herein). In some embodiments, the signaling portion comprises the amino acid sequence of SEQ ID NO: 6, 7, 8, 9 or 10 (or any other TNFR1 construct sequence disclosed herein). In some embodiments, the chimeric receptor comprises the amino acid sequence of SEQ ID NO: 13, 14, 15, 16, 17, 26, 28, 30, 40, 45, 48 or 49 (or any other chimeric receptor construct sequence disclosed herein).

The receptor may have a known binding specificity or may have unknown binding specificity. In some embodiments, the receptor has unknown binding specificity, meaning that a specific binding substrate for the receptor has not been determined. The receptor may have an unknown amino acid sequence or may be encoded by a polynucleotide (or polynucleotides) of unknown nucleotide sequence. Receptors of unknown binding specificity and/or unknown sequence may be produced in any number of known ways. For example, there are a variety of known methods which use a step that randomly or unpredictably changes the nucleotide sequence of a template gene to insert, delete and/or substitute nucleotides in a desired region (e.g. in the binding site of a receptor or variable region of an antibody or T-cell receptor). Without limitation, such methods include in vitro V(D)J recombination, mutagenesis and/or the use of double-stranded breaks together with Tdt such as with restriction enzymes, CRISPR, Zinc Finger or Talon methods or the use of error prone PCR, degenerate oligos or degererate gene synthesis products.

The receptor or chimeric receptor may be encoded on at least one nucleic acid comprising one or more coding sequences. Accordingly, the host cell may further comprise at least one nucleic acid comprising one or more coding sequences which collectively encode the receptor. For example, where the receptor comprises a full length IgG for the binding portion, the light chains of the IgG may be on a separate nucleic acid molecule from the fusion of the signaling portion, transmembrane domain and the heavy chain (e.g. where each is on a separate plasmid or chromosome or one is on a plasmid and the other is chromosomally integrated).

To facilitate expression of the one or more coding sequences which collectively encode the chimeric receptor, in some embodiments the at least one nucleic acid may further comprise at least one promoter operably linked to the one or more coding sequences. The at least one promoter may include weak and/or strong promoter(s).

In some embodiments, the at least one promoter may include a weak promoter. Significant research has been done on the analysis of TATA boxes and other transcription binding sites that modulate transcription activity. These binding sites can be mutated or deleted to compromise the binding to and/or assembly of transcription factors and/or assembly of the RNA polymerase so as to ultimately compromise the rate of transcription. For example, but without limitation, the weak promoter may be a UBC promoter (Ubiquitin C promoter), a PGK promoter (phosphoglycerate kinase 1 promoter), a Thymidine Kinase (TK) promoter or a promoter that has a transcriptional activity that is no more than 100%, 150%, 200%, 250%, 300%, 350%, 400%, 450% or 500% the transcriptional activity of one of the aforementioned weak promoters when transcribing the same reference coding sequence when in operable linkage to said reference coding sequence (e.g. SEQ ID NO: 13, 14, 15, 16, 17, 26, 28 or 30).

The at least one promoter may include regulated or constitutive promoter(s). In some embodiments, the at least one promoter comprises inducible promoter(s). For example, the at least one promoter may comprise binding sites for a repressor, such as the Tet repressor, the Gal4 repressor and the like. In the case of the Tet repressor, operator sequence(s) (e.g. tetO) may be placed upstream of a minimal promoter to permit transcription to be reversibly turned on or off in the presence of tetracycline or one of its derivatives (e.g. doxycycline and the like). Similarly, nucleic acid sequences which bind the Gal4 repressor may be positioned to regulate transcription of genes that are operably linked to a minimal promoter. As used herein, operator sequences and/or other regulator sequences are considered part of the regulated promoter, regardless of their proximity to transcription start site(s) of the coding sequence(s), so long as they are functionally positioned for regulation of transcription. The promoter may be activated upon the binding of a ligand to a receptor.

An advantage of using a weak promoter in certain embodiments is a reduction in background signal from intracellular signaling in the absence of bound binding substrate. Without wishing to be bound by theory, it is thought that a weak promoter reduces background signal in certain embodiments by lowering expression of the receptor so as to reduce activation of the signaling portion due to local concentrations of the receptor exceeding the threshold for activation. In effect, diluting the receptor on the cell surface reduces self-activation in the absence of binding substrate.

In some embodiments, the one or more coding sequence comprises or is operably linked to one or more genetic elements which, when the receptor is expressed in the host cell (e.g. a vertebrate cell or another NF-κB competent eukaryotic cell), cause expression of the receptor at a level that is sufficiently low such that signaling caused by binding of the binding substrate to the receptor is distinguishable over background signaling (e.g. in the absence of the binding substrate). Various such genetic elements are known, which can be used alone or in combination, including for example, but without limitation: a Kozak sequence in the nucleic acid which causes inefficient translation of the receptor (see, e.g.: Grzegorski, et al. PloS One 2014; 9:e108475; and Kozak, Gene 2005; 361:13-37); codon(s) in the at least one coding sequence which are not optimized for efficient translation in the host cell; one or more RNA destabilizing sequences in the nucleic acid which reduces the half-life of an RNA transcribed from the nucleic acid which encodes the receptor (see e.g.: Dijk et al. RNA 1998; 4:1623-1635; and Day & Tuite. Journal of Endocrinology 1998; 157:361-371); intron and/or exon sequences in the one or more coding sequence which cause inefficient intron splicing (see, e.g.: Fu & Ares Nature Reviews 2014; 15:689-701); and/or ubiquination sequence(s) in the receptor (e.g. to encourage degradation of the receptor; see e.g.: Yu et al. J. Biol. Chem. 2016; 291:14526-14539).

In some embodiments, the at least one nucleic acid comprising one or more coding sequences which collectively encode the receptor is a vector. In some embodiments, the at least one nucleic acid comprising one or more coding sequences which collectively encode the receptor is integrated in a chromosome of the host cell.

In some embodiments, a promoter that is operably linked to a coding sequence in the at least one nucleic acid comprises an operator sequence and the host cell expresses a repressor which binds to the operator sequence. In other words, the repressor binds an operator sequence within a regulated promoter that controls expression of the one or more coding sequence which collectively encode the receptor described herein. This further reduces the expression of the receptor which assists achieving low background levels of signaling in the absence of binding substrate. The repressor may be TetR and the operator may be TetO or another nucleotide sequence that binds TetR. The repressor may be Gal4 and the operator may be a nucleotide sequence which binds Ga14.

In some embodiments, the host cell further comprises at least one sequence for expressing antisense RNA, miRNA (microRNA) or siRNA (small interfering RNA) configured to reduce expression levels of the receptor. Nucleic acids comprising such sequences may be separate from or comprise part of the at least one nucleic acid comprising the one or more coding sequence which collectively encode the receptor. Sequences for expressing antisense RNA, miRNA and siRNA can be readily generated from the sense sequence (i.e. the sequence of the at least one nucleic acid that collectively encodes the receptor). With respect to antisense RNA, this includes any nucleic acid sequence which when transcribed in the vertebrate cell would bind to the messenger RNA (mRNA) that encodes the receptor (including without limitation sequences which are 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to the reverse complement of the mRNA or the sequence within the mRNA that encodes the chimeric receptor). Tools for generating antisense RNA, miRNA and siRNA are publicly and commercially available.

As mentioned, the receptor signals production of a positive selectable marker and/or a negative selectable marker in response to the receptor being bound by a specific binding substrate. In other words, upon activation of the signaling portion of the receptor by substrate binding to the binding portion, the signaling portion mediates a signal or signaling cascade which ultimately causes expression of either or both a positive selectable marker and a negative selectable marker. In some embodiments, the receptor signals production of a positive selectable marker and a negative selectable marker in response to the receptor being bound by a specific binding substrate. In some embodiments, the production of the positive selectable marker and/or the negative selectable marker may be encoded by at least one selection cassette that is heterologous to the host cell.

For example, but without limitation, in embodiments in which the signaling portion of the host cell receptor comprises or is obtained from a TNFRSF member, the activated signaling portion in turn activates NF-κB through adaptor proteins and their enzymatic binding partners, either through the canonical and/or noncanonical NF-κB signaling pathways (Wertz and Dixit Cold Spring Harb Perspect Biol 2010; 2(3): a003350). NF-κB is not a single entity, but is a family of dimeric transcription factors consisting of five proteins, p65 (also known as RelA), RelB, c-Rel, p50 and p52 (p105 and p100 are precursor proteins for p50 and p52, respectively). NF-κB proteins associate to form homodimers and heterodimers (e.g. the p65:p50 heterodimer). NF-κB is maintained in an inactive state through association with an IκB (an inhibitor ofNF-κB). NF-κB is activated by polyubiquitination of IκB, which targets IκB for proteosomeal degradation and liberates (activated) NF-κB dimers. Ultimately, IκB is ubiquitinated by the activity of the IκK complex, which is activated by signaling complex(es) which ultimately are formed as a result of a signaling cascade initiated by activated TNFRSF members. Accordingly, operably linking a gene(s) of interest (such as a selectable marker gene) to a NF-κB response element will enable the transcription of the gene(s) of interest to be controlled by the activation state of the host cell receptor, which is inactive when unbound by binding substrate and active when bound by binding substrate. The gene(s) of interest may be one or both of the positive selectable marker gene and the negative selectable marker gene or the gene(s) of interest may ultimately mediate production of the positive selectable marker gene and the negative selectable marker gene.

Thus, in some embodiments, the at least one selection cassette comprises a positive selectable marker gene and/or a negative selectable marker gene operably linked to a second promoter and a NF-κB response element such that expression of the positive selectable marker gene and/or the negative selectable marker gene is repressed (or otherwise inactivated) by NF-κB binding to the NF-κB response element and induced in the absence of said NF-κB binding. In these embodiments, the NF-κB response element is configured to be bound by NF-κB which acts as a transcriptional repressor (e.g. p50 and/or p52). In alternative embodiments, the at least one selection cassette comprises a positive selectable marker gene and/or a negative selectable marker gene operably linked to a second promoter and a NF-κB response element such that expression of the positive selectable marker gene and the negative selectable marker gene is induced by NF-κB binding to the NF-κB response element and inactive or repressed in the absence of said NF-κB binding. In these embodiments, the NF-κB response element is configured to be bound by a NF-κB which acts as a transcriptional activator (e.g. p65:p50 heterodimer or other dimers incorporating p65, RelB and/or c-Rel).

In some embodiments, the positive selectable marker gene and the negative selectable marker gene are part of a polycistronic operon operably linked to the NF-κB response element. For examples, but without limitation, the positive selectable marker gene and the negative selectable marker gene may be separated by P2A and/or IRES sequences or other such sequences.

In some embodiments, the at least one selection cassette comprises two selection cassettes: a positive selection cassette comprising a third promoter operably linked to the positive selectable marker gene and a negative selection cassette comprising a fourth promoter operably linked to the negative selectable marker gene, wherein the third promoter and the fourth promoter are operably linked to a separate NF-κB response element.

In some embodiments, the gene(s) of interest, selectable markers and/or the selection cassette(s) are chromosomally integrated into the host cell. In other embodiments, the the gene(s) of interest, selectable markers and/or the selection cassette(s) are stably maintained as a plasmid. For example, but without limitation the stably maintained plasmid may be a yeast artificial chromosome (YAC) and the like, or an OriP containing plasmid where the host cell expresses EBNA-1 or a similar protein).

In some embodiments, the gene(s) of interest is or causes expression of the positive selectable marker and the negative selectable marker. As used herein, the expression “selectable marker” means “selection” in the sense of providing a selection advantage for survival or growth/reproduction and excludes purely screenable markers (such as GFP or detectable surface antigens). Selection as used herein includes but is not limited to selection by survival or by cell death. More generally, the introduction of a gene(s) into a cell which lacked said gene(s) may be associated with the acquisition of a novel phenotype. This acquired phenotype may then be exploited to select for cells which harbor/express the introduced gene(s). Although selection is often used for tracking the introduction of genetic elements, the host cell herein uses selectable marker(s) to select for activated receptors (e.g. activated due to specific recognition of binding substrate). For example, when starting with a large population of biosensor host cells having a diverse set of binding specificities, the use of a selectable marker may allow for rare populations to be identified that would be a challenge using FACS or magnetic sorting (e.g. when frequencies are well below 1 in a million). In some embodiments, the positive selectable marker mediates survival of the host cell and/or the negative selectable marker mediates death of the host cell.

Positive selection is distinct from a traditional reporter system in that it allows for survival (and growth) and allows for significantly larger numbers of cells to be evaluated than even the highest throughput screening platforms which depend on mechanical detectors to identify activated cells.

The positive selectable marker gene may encode a protein(s) which confers resistance to a toxic compound. As used herein, the term “toxic compound” includes without limitation any small molecules, peptides, proteins, suicide gene products, and the like, whether natural or artificial, which is poisonous to the eukaryotic cell (e.g. vertebrate cell) or causes cell death. In certain embodiments, the positive selectable marker gene may encode an antibiotic resistance protein. For example, genes are known which provide mammalian cells resistance against geneticin, neomycin, Zeocin™, hygromycin B, puromycin, blasticidin and other antibiotics. Alternatively, expression of a MDR (multi-drug resistance) gene(s) may act as a positive selectable marker by providing resistance to a toxic compound(s).

Positive selection may also be accomplished by curing auxotrophy, i.e. the inability of a cell to synthesize a particular compound(s) needed for growth/survival. This selection approach is widely used in yeast selections, but is also used in other eukaryotic cell types, including mammalian cells. Auxotrophy exists for large classes of compounds required for growth including without limitation vitamins, essential nutrients, essential amino acids and essential fatty acids. Certain cells are dependent on specific growth factors for growth and survival. Therefore, acquisition of the gene expressing the growth factor would allow for positive selection. Certain gene products such as hypoxanthine-guanine phosphoribosyltransferase (HPRT) and xanthine phosphoribosyltransferase (GPT) allow for the conversion of compounds to useful metabolites essential for growth. Auxotrophy may also be used with factor dependent cell lines that need certain growth factors or ligands to proliferate (e.g. the TF1 cell line needs erythropoietin or “EPO” supplementation for growth). Accordingly, in certain embodiments, the host cell is an auxotroph which requires a missing compound for growth or survival and the positive selectable marker gene(s) encodes one or more gene products which permit the host cell to synthesize the missing compound.

In certain embodiments, expression of the positive selectable marker gene permits selection based on chemical detoxification, selection based on exclusion or removal, selection based on increased expression (such as the dihydrofolate reductase or “DHFR” gene, and the like), selection based on pathogen resistance, selection based on heat tolerance, selection based on radiation resistance, selection based on double-strand break sensitivity, selection based on ability to utilize non-metabolized compounds (e.g. HPRT, GPT and the like) and/or selection based on acquisition of a growth factor.

Negative selection cannot be read by reporter based systems. The negative selectable marker gene may encode or cause expression of: a toxin or an enzyme (e.g. HPRT, GPT or a suicide gene(s)) which can convert a precursor compound to a toxic compound. A number of suicide gene systems have been described including the herpes simplex virus thymidine kinase gene, the cytosine deaminase gene, the varicella-zoster virus thymidine kinase gene, the nitroreductase gene, and the E. coli Deo gene. The products of these suicide genes metabolize substrates into toxic compounds that are lethal to cells. Accordingly, in some embodiments the negative selectable marker gene(s) may be a suicide gene(s). In some embodiments, the negative selectable marker gene may be HPRT, GPT, herpes simplex virus thymidine kinase gene, cytosine deaminase gene, varicella-zoster virus thymidine kinase gene, nitroreductase gene or E. coli Deo gene. Hormone based dimerization may also be used for negative selection by promoting complementation to assemble or reconstitute a function protein. Two-hybrid approaches may also be deployed to drive the expression of toxic genes either directly or indirectly. Gene modifying approaches that incorporate CRE, FRT, CRISPR or other gene modifying activities may be utilized to induce the expression of a gene of interest. Another non-limiting option for negative selection is induction of apoptosis. Apoptosis or programmed cell death is a conserved process in vertebrates and many non-vertebrate eukaryotic cells, e.g. yeast (Carmona-Gutierrez et al. Cell Death and Differentiation 2010; 17:763-773). Ycalp is a metacaspase (an ortholog of mammalian caspases) that is required for numerous cell death scenarios. For example, the receptor may induce apoptosis via death domain-mediated signaling or by causing/increasing expression of a signaling protein that promotes apoptosis. In some embodiments, the negative selectable marker is a death receptor that activates apoptosis of the host cell in response to a death receptor ligand.

In some embodiments, the positive selectable marker gene and/or the negative selectable marker gene may encode or cause expression of a chimeric screenable-selectable marker. For example, but without limitation, the marker gene may encode an integral membrane protein that displays an extracellular surface antigen and an intracellular resistance protein. For example, but without limitation, the positive selectable marker gene may encode or cause expression of CD19 fused to puromycin N-acetyl-transferase (Puro), and be configured for intracellular display of Puro and extracellular display of CD19 antigen. In some embodiments, the positive selectable marker gene comprises or consists of the amino acid sequence of SEQ ID NO:18. Without limitation, SEQ ID NO:19 represents the nucleic acid sequence of a vector for expressing a CD19-Puro fusion having the amino sequence of SEQ ID NO: 18.

The negative selectable marker and the positive selectable marker may each be mediated by a different exogenous mediator, such that only positive selection or negative selection is effected from the activation of a single chimeric receptor, depending on the presence of the corresponding exogenous mediator. Two representative (but non-limiting) schematics of such a dual selection biosensor are shown in FIGS. 1A and 1B.

In some embodiments, the positive selectable marker gene is under the transcriptional control of NF-κB and the TNFRSF member is a death receptor. This allows for negative selection in the absence of apoptosis inhibitors (e.g. caspase inhibitors) and positive selection in the presence of apoptosis inhibitors. For example, but without limitation, when the positive selectable marker is Puro expression, then the inclusion of apoptosis inhibitors (e.g. caspase inhibitors) during use allows for positive selection by adding puromycin to the cell media. Any of the aforementioned positive selection markers may likewise be used with a death receptor or death receptor fragment signaling portion to enable negative or positive selection. In certain embodiments, the TNFRSF member need not necessarily be a death receptor as negative selection may be implemented by engineering the eukatyotic cell (e.g. vertebrate cell) to express a negative selectable marker which induces apoptosis. This approach may be used for other chimeric or natural receptors which signal through multiple pathways wherein the primary signal may be modified by inhibiting certain pathways while leaving others open.

In some embodiments, the positive and negative selectable marker genes may be induced in combination with an additional receptor that when bound by a ligand activates NF-κB which would allow for increased sensitivity and longevity of the signal.

The host cell may be engineered to inactivate a specific endogenously expressed death receptor in the host cell. Inactivation may be accomplished by any known method (e.g. CRISPR/CAS9, zinc fingers, talons or other forms of mutagenesis). As such, the engineered host cell may no longer signal apoptosis in response to a particular ligand (called “ligand x” for ease of reference). Then, by engineering the cell to express a death receptor that responds to ligand x when the chimeric receptor is activated, the engineered cell will be enabled for negative selection (i.e. apoptosis) when the receptor is activated and the cell media contains ligand x. When the engineered cell also expresses a positive selection marker (e.g. an antibiotic and the like), then the biosensor will also be enabled for positive selection in the absence of ligand x. For example, if endogenous DR4 (TRAILR1) and DR5 (TRAILR2) death receptors are both inactivated, then the cell will not die in the presence of the TRAIL ligand. If the host cell is then engineered to express DR4 and/or DR5 when the receptor is activated, the cell can be negatively selected in the presence of TRAIL.

In some embodiments, the host cell further comprises an expression cassette for expressing a cell surface protein comprising an extracellular domain for displaying the target binding substrate. This binding substrate may be a multivalent binding substrate (e.g. expressed as a fusion protein with the cell surface protein). The binding substrate may be a univalent binding substrate that forms a multivalent binding substrate through multimerization of the cell surface protein. In certain embodiments, the expression cassette for the cell surface protein may comprise an inducible promoter operably linked to a nucleic acid sequence or sequences which encode(s) the cell surface protein.

### V. Biosensor Libraries & Exemplary Methods/Uses Thereof

This disclosure also presents a library of biosensor cells comprising a plurality of unique biosensor cells which collectively bind a plurality of unknown binding substrates. The unique biosensor cells may be any host cell described in Section IV. In some embodiments, the biosensor cell comprises a receptor with unknown binding specificity or unknown sequence, the receptor being natural or artificial, which signals production of a positive selectable marker and a negative selectable marker in response to the receptor being bound by a specific binding substrate, wherein the production of the positive selectable marker and the negative selectable marker is encoded by at least one selection cassette that is heterologous to the host cell.

As mentioned in Section IV, a population of cells that express receptors with unknown binding specificities or sequences and which collectively bind a diverse plurality of binding substrates may be made by various known methods. In some embodiments, the plurality of unique biosensor cells comprises at least 1000, at least 10,000, at least 100,000, at least 1 million, at least 10 million, at least 100, million, at least 1 billion, or at least 10 billion unique biosensor cells (or any number of cells therebetween). In some embodiments, the plurality of unique biosensor cells comprises more than 10 billion biosensor cells.

Without limitation, the library of biosensor cells may be used for specifically binding a binding substrate (e.g. an unknown substrate, a substrate that is not known to be specifically bound by a binding moiety, or a substrate in a heterogeneous mixture). Accordingly, this disclosure also provides an in vitro method of identifying a biosensor cell from the library of biosensor cells defined herein that is specifically activated by a target substrate. Depending on the binding portion of the receptor in the host cell, the target substrate may be any molecule or molecular complex. For example, but without limitation, the binding substrate may be a small molecule, a peptide, protein, a nucleic acid, a polynucleotide, an oligosaccharide, a glycoprotein, or a fusion or complex of any of the preceding. The binding substrate may be an antigen. The in vitro method comprises: (a) contacting the library with the target substrate under positive selection conditions; (b) contacting the library with a control substrate under negative selection conditions; and (c) identifying biosensor cells which survive (a) and (b) as biosensor cells which are specifically activated by the target substrate.

In some embodiments, step (a) precedes step (b). In some embodiments, step (b) precedes step (a). In some embodiments, steps (a) and (b) are iterative.

The positive selectable marker gene and the negative selectable marker gene may be any described in Section IV. In this method, positive selection conditions are conditions which selectively kill those cells which do not express the positive selectable marker. Similarly, negative selection conditions are those which selectively kill those cells which express the negative selectable marker. In some embodiments, the method further comprises performing (a) and/or (b) in the presence of an exogenous mediator. For example, (a) may be carried out in the presence of an apoptosis inhibitor or another compound which blocks negative selection. When the negative selectable marker mediates caspase-dependent apoptosis, then in some embodiments (a) may be carried out in the presence of a caspase inhibitor, such as caspase-8 inhibitor and/or caspase-10 inhibitor or a pan-caspase inhibitor. Various caspase inhibitors are known and commercially available (e.g. pan-caspase inhibitor Z-VAD-FMK and the like).

In some embodiments, contacting in steps (a) and/or (b) comprises co-culturing the plurality of unique biosensor cells with a target cell(s) which comprises the target substrate. The target substrate may be expressed on the surface of the target cell. The target substrate may be secreted from the target cell. The target cell may be any cell type (e.g. a fungus cell, a bacterial cell, a yeast cell, a vertebrate cell, a mammalian cell, a human cell, a cancer cell, and the like).

In some embodiments, target substrate may be in solution or in a mixture. For example, but without limitation, the target substrate may be in a cell lysate, serum sample or other biological sample or analyte.

In some embodiments, the method further comprises preparing the target substrate prior to contacting steps (a) and/or (b). For example, but without limitation, the multivalent binding substrate may be prepared by oligomerizing or complexing a binding substrate (e.g. a monovalent binding substrate) and/or by expressing the binding substrate on the surface of a cell such that the multiple units of the binding substrate is displayed on the cell surface in close proximity to each other. Oligomerizing or complexing a protein (such as the binding substrate) may be achieved by various different methods. A common method is to biotinylate the protein and incubate it with avidin which has multiple binding sites for biotin to create a substrate with increased valency. If the protein is biotinylated in multiple positions then the complexes may be larger than mono-biotinylated proteins. The use of cross-linking reagents may also bring multiple proteins/molecules together. Expressing the protein as an Fc-fusion protein creates a dimer of the molecule. The use of a secondary antibody to cross-link the Fc-fusion protein further increases the valency of the substrate. Expression as an IgM or IgA fusion protein may also provide multivalent molecules. Molecules may be linked to beads (e.g. agarose) or ELISA plates to provide for increased surface valency. Molecules expressed on the surface of a cell provides a format that has valency suitable for a substrate to activate the chimeric receptor (e.g. by cross-linking).

In some embodiments, contacting the biosensor with the multivalent binding substrate comprises co-expressing a cell surface protein in the first vertebrate cell with the chimeric protein, the cell surface protein comprising an extracellular domain comprising: the multivalent binding substrate; or a univalent binding substrate that forms the multivalent binding substrate through multimerization of the cell surface protein. In some embodiments, expressing the cell surface protein is inducible and the method further comprises inducing expression of the cell surface protein.

Using substrate binding dependent signaling (e.g. antigen dependent signaling) to mediate both positive and negative selection is particularly useful for isolating rare binding specificities from large cell-expressed repertoires. The ability to utilize selection both positive and negative selection is an improvement over only positive or negative selection since it allows even larger repertoires to be interrogated and even rarer events to be isolated. In addition, dual selection allows for the direct elimination of off-target binding events.

Although utilizing a biosensor approach (a cell utilizing a cell surface signal) has the advantage that the target binding substrate does not need to be purified and can be expressed in its native conformation in the plasma membrane of the target cell, applying a large (and diverse) biosensor library has some unique challenges, e.g. when trying to isolate a biosensor that is specific for a particular target binding substrate on a cell surface. Because the target cell has thousands of proteins representing 100s of thousands of binding substrates all potentially activating biosensors, it would be particularly useful to be able to distinguish target-specific activated biosensors from background activated biosensors. Incomplete activation of the biosensor (for example if only 80% of the cells are activated the other 20% will appear as negative but possess the incorrect specificity) and/or incomplete staining generate populations of background cells that represent an undesirable level of background when starting with large library populations (e.g. a billion cells), which may make it difficult or laborious to isolate the rare biosensor with the desired specificity (this is similar to the challenge with phage display where negative panning is inefficient). These limitations may be overcome in some embodiments disclosed herein, where the biosensors are equipped for both functional positive and negative selection.

Biosensor repertoires may be alternatively exposed to cells with and without the target binding substrate on their cell surface, alternatively being positively and negatively selected to enrich for a biosensor population that is activated only in the presence of a cell expressing the target of interest. A benefit of adding negative selection to positive selection is that it allows for the elimination of cells that are off-target (e.g. cells displaying antigens present on both the target cells and the control cells). An advantage of some such embodiments is that expensive and specialized FACS sorting equipment is not required. Another advantage of some such embodiments it that significantly more cells can be processed to isolate extremely rare binding events. Although there is a limit on how many cells a FACS machine can process in a day, some of these embodiments are not so limited and the size of the biosensor library may be easily scaled up; cultures of 10-100 liters (or more) of cells may be selected with the addition of a drug for selection like puromycin. FACS machines also are not able to routinely isolate rare events at frequencies of less than 1 in 100,000. Accordingly, it would take multiple rounds of FACS sorting to isolate the rare events of interest. Positive selection in some embodiments described herein may be able to detect rare binding events at frequencies of less than 1 in a million or even 1 in 10 million. Negative selection is also possible at the same scale, eliminating biosensors that have been activated in the presence of the control cell line. Therefore, the ability for the same signaling event (i.e. activation of the chimeric receptor) to direct cell survival or cell death allows for alternating selection pressure to isolate rare specificities from extremely large repertoires.

An exemplary (but non-limiting) example of a dual selection method is schematically shown in FIG. 1A. FIG. 1A shows a biosensor cell with a chimeric receptor (shown as a triangle) which, when activated, signals expression of PuroR (puromycin resistance protein) as well as apoptosis. During co-culture with target cells, apoptosis is inhibited by caspase inhibitors. The presence of puromycin positively selects activated biosensors and kills non-activated biosensors and the target cells. After removing puromycin and caspase inhibitors, the positively selected biosensors are co-cultured with control cells, which lack the target binding substrate, in the absence of puromycin and caspase inhibitors. This negatively selects out off-target activated biosensors, leaving only non-activated biosensors (i.e. biosensors previously selected as being target-specifically activated). This method is not limited to puromycin as the specific positive selection mechanism. In alternative embodiments, the target and control co-cultures may be performed in parallel and sequencing used to discriminate the target-specific biosensors from biosensors activated by the control cells.

Another exemplary (but non-limiting) example of a dual selection method is schematically shown in FIG. 1B. FIG. 1B shows a biosensor cell with a chimeric receptor (shown as a grey triangle) which, when activated, signals expression of intracellular PuroR linked to a death receptor which embeds in the plasma membrane. During co-culture with target cells, the presence of puromycin positively selects activated biosensors while killing non-activated biosensors and the target cells. Apoptosis from the death receptor is avoided by the absence of the death receptor ligand. The positively selected biosensors are then co-cultured with control cells, which lack the target binding substrate, in the presence of the death receptor ligand (e.g. TRAIL ligand for DR4 or DR5). This negatively selects out off-target activated biosensors, leaving only non-activated biosensors (i.e. biosensors previously selected as being target-specifically activated). As such, identifying binding may comprise contacting the first vertebrate cell with the ligand of a death receptor which is only expressed in response to activation of the chimeric receptor. This method is not limited to puromycin as the specific positive selection mechanism or to DR4/DR5 as the specific death receptor. This method is also not limited to the positive and negative selection elements being linked as a fusion protein. In alternative embodiments, the target and control co-cultures may be performed in parallel and sequencing used to discriminate the target-specific biosensors from biosensors activated by the control cells.

While traditional library screens can be applied using the described biosensor approach where an exogenous target (or cell line expressing a target of interest) is incubated with the biosensor and activation in trans identifies bisosensors with specificity to the target of interest, the cell based biosensor system also is amendable to configuring the screen in an autocrine manner. In such embodiments the target sequence is expressed in the biosensor cells (along with the biosensor receptor/chimeric receptor) as opposed to being added exogenously. The target of interest can be expressed in an induced manner so that biosensors can be identified that are only activated when the target is expressed. In a non-limiting example, the library of biosensors comprising a plurality of unknown binding specificities is subjected to negative selection. Biosensor cells with extracellular binding sites specific for its own cell surface proteins will be activated in an autocrine fashion to express the negative selectable marker (e.g. death receptor such as DR4, DR5, which can be activated by a death ligand such as TRAIL, or any other negative selectable marker previously described) such that biosensor cells expressing these anti-self binding specificities will be killed and eliminated. Subsequently the expression of the target protein is expressed. Biosensor cells activated following the induced expression of the target will survive positive selection.

This disclosure also provides a product or method substantially as hereinbefore described (e.g. in Sections I, II, III, IV and/or V) with reference to any one of the Examples below or to any one of the accompanying drawings.

### VI. Sequences

Table 1 describes various sequences referenced herein.

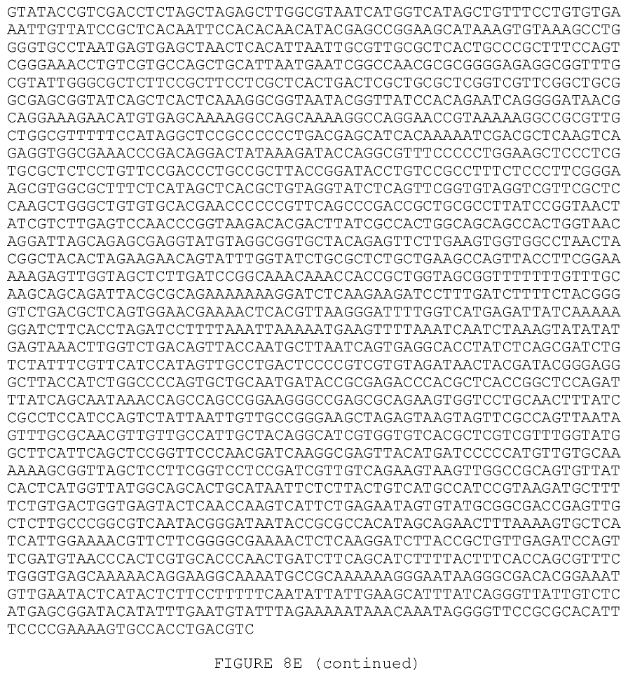

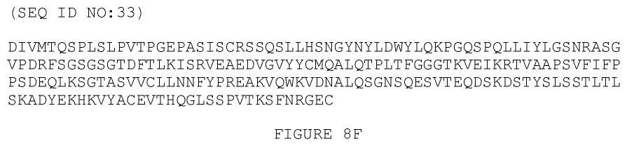

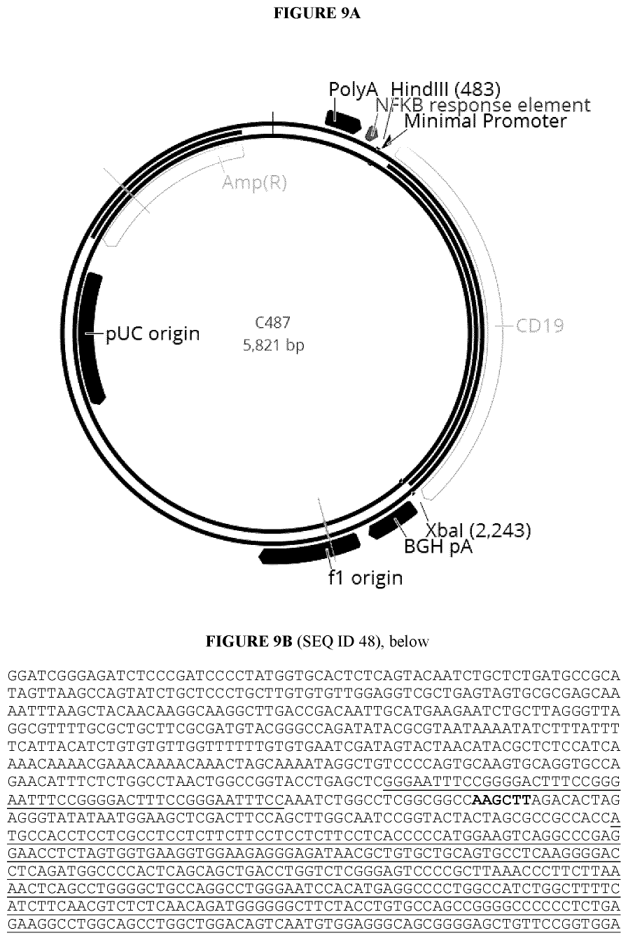

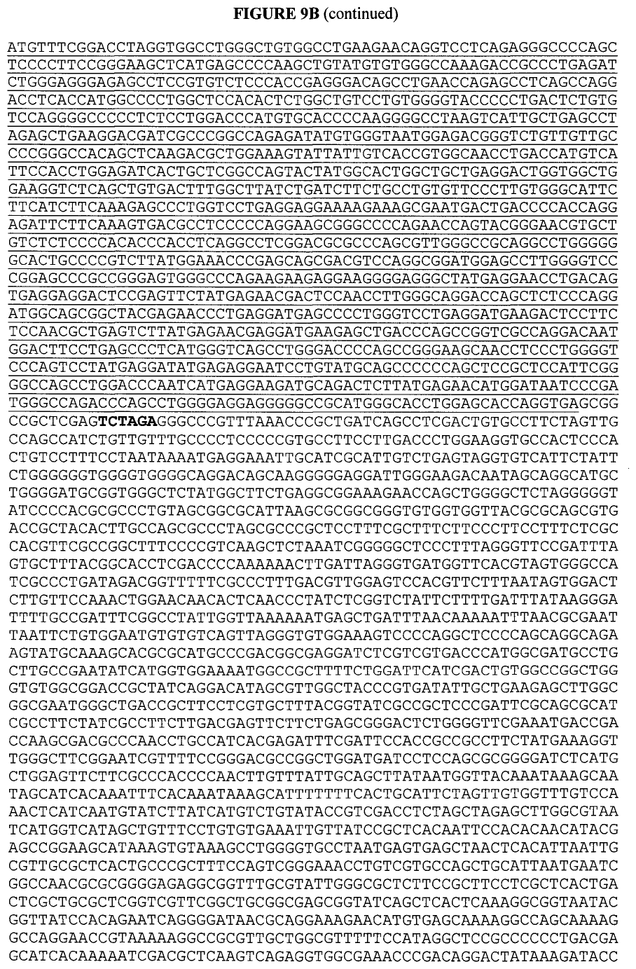

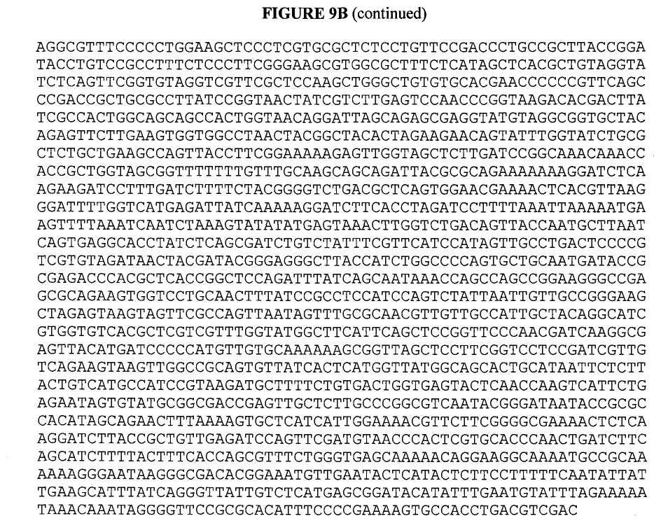

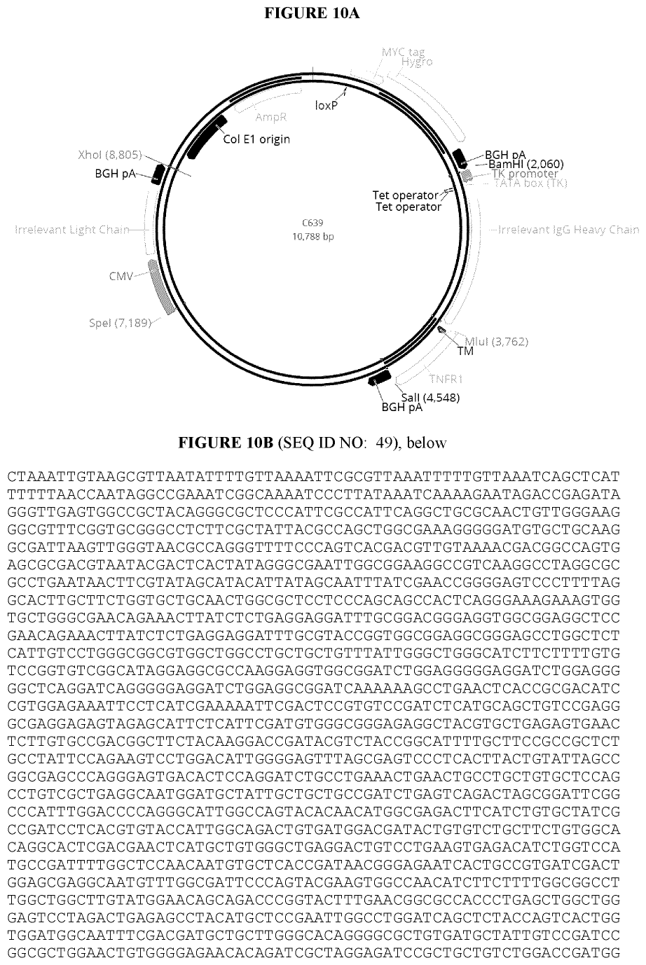

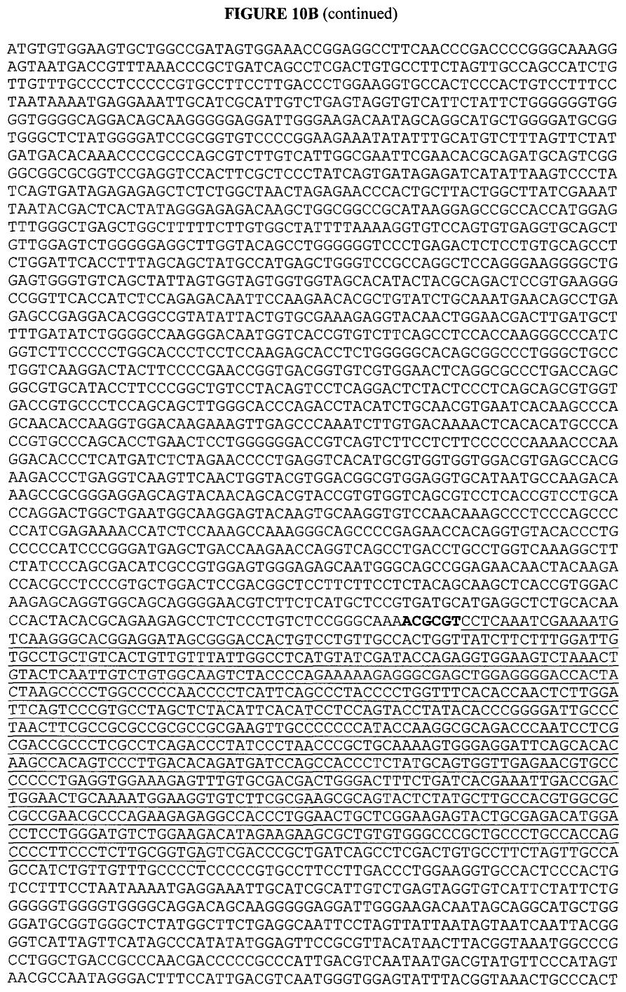

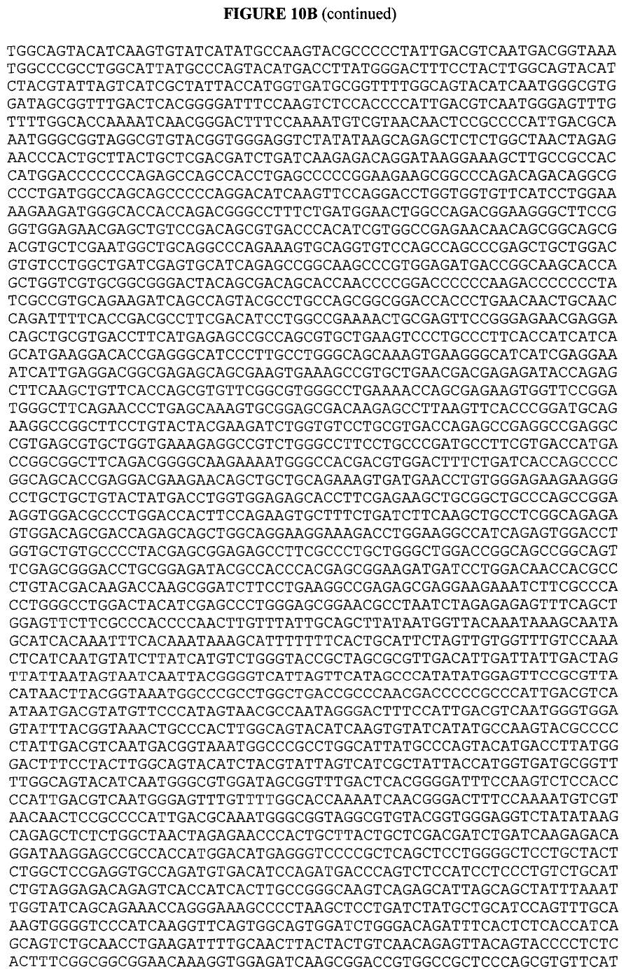

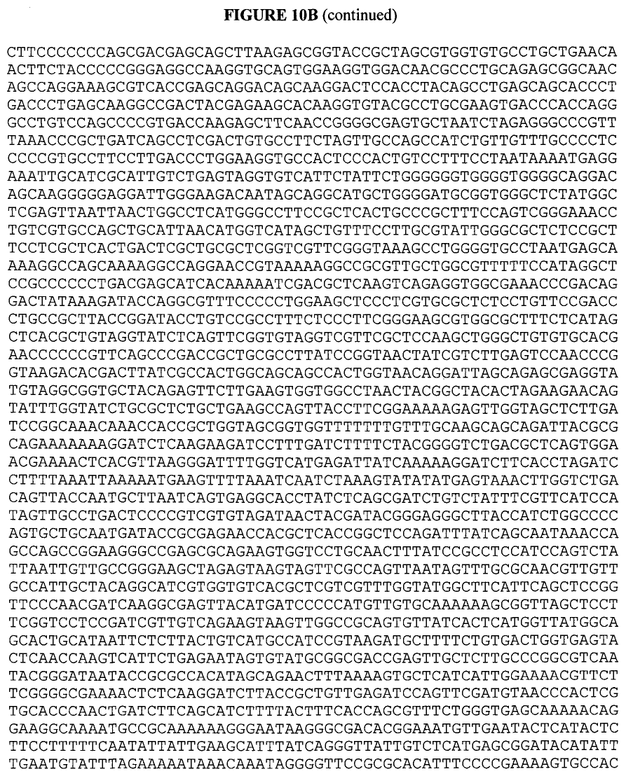

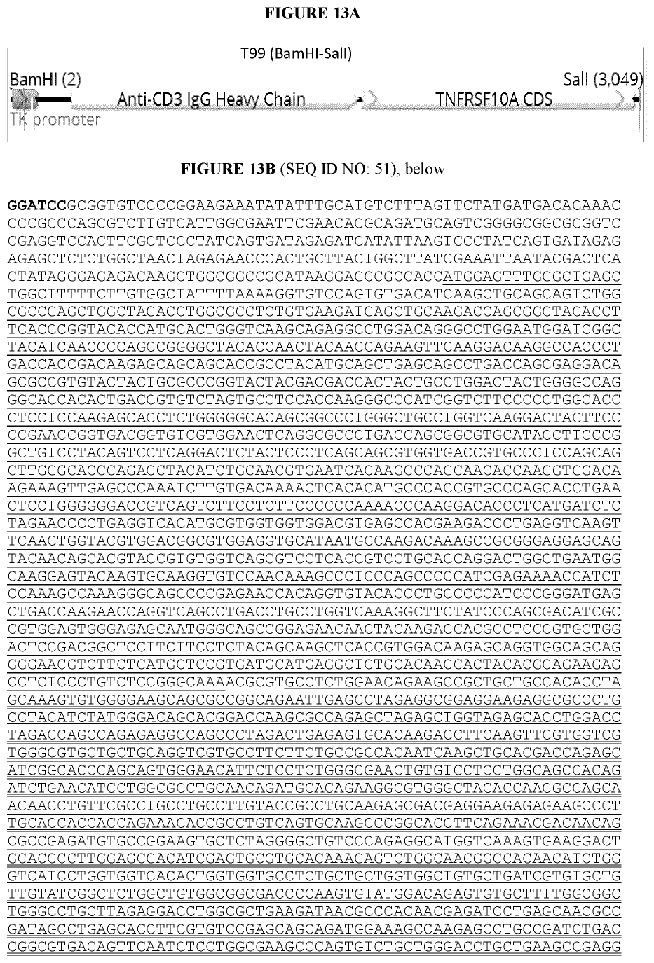

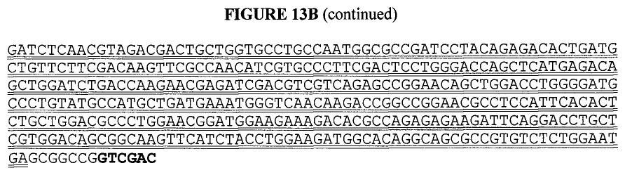

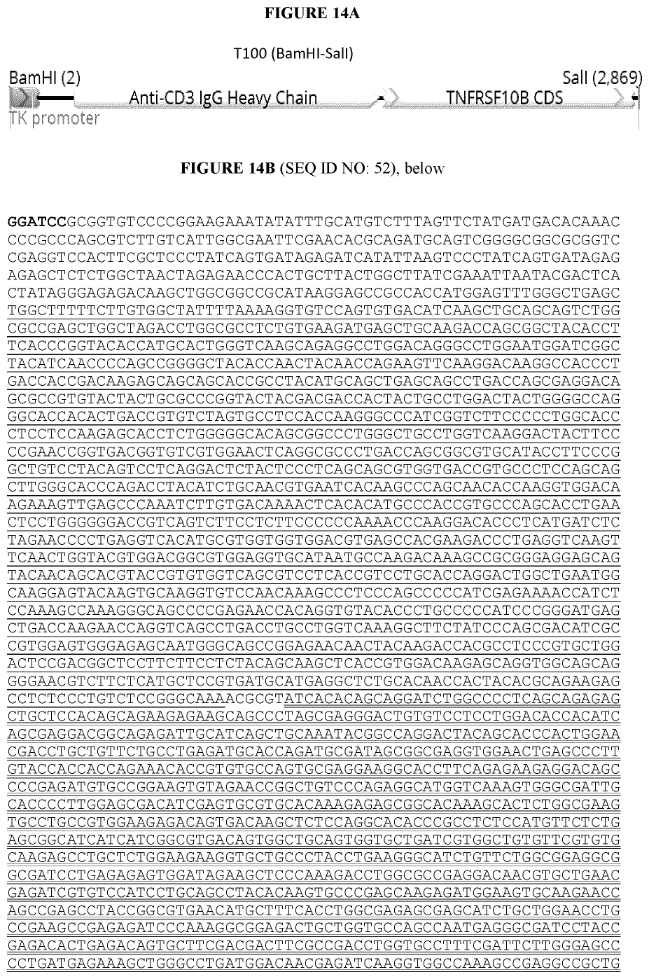

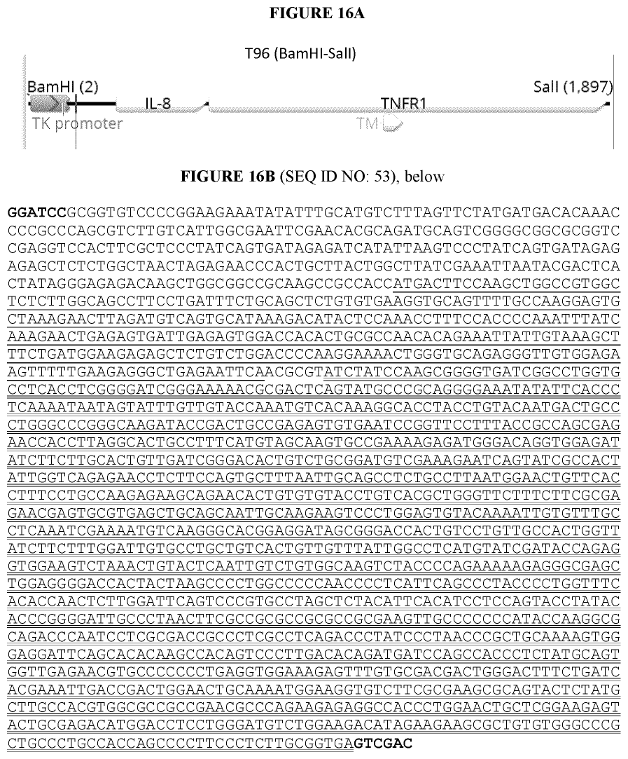

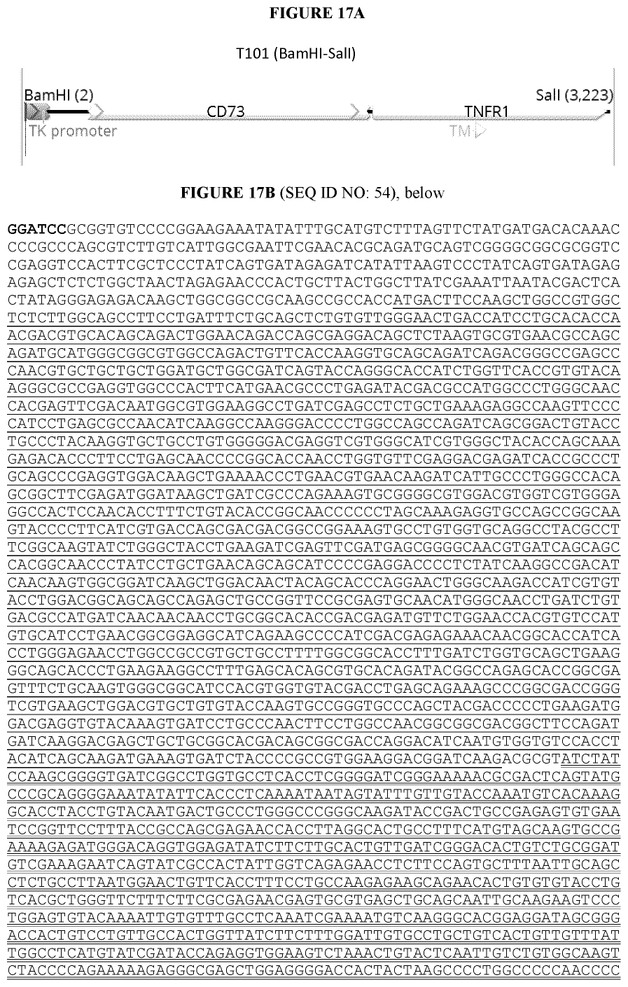

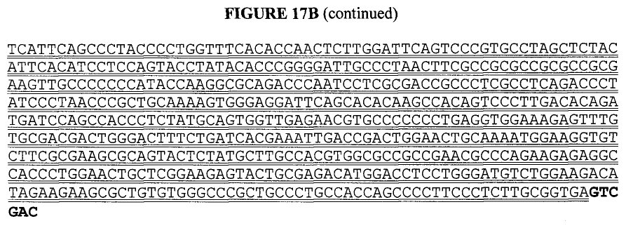

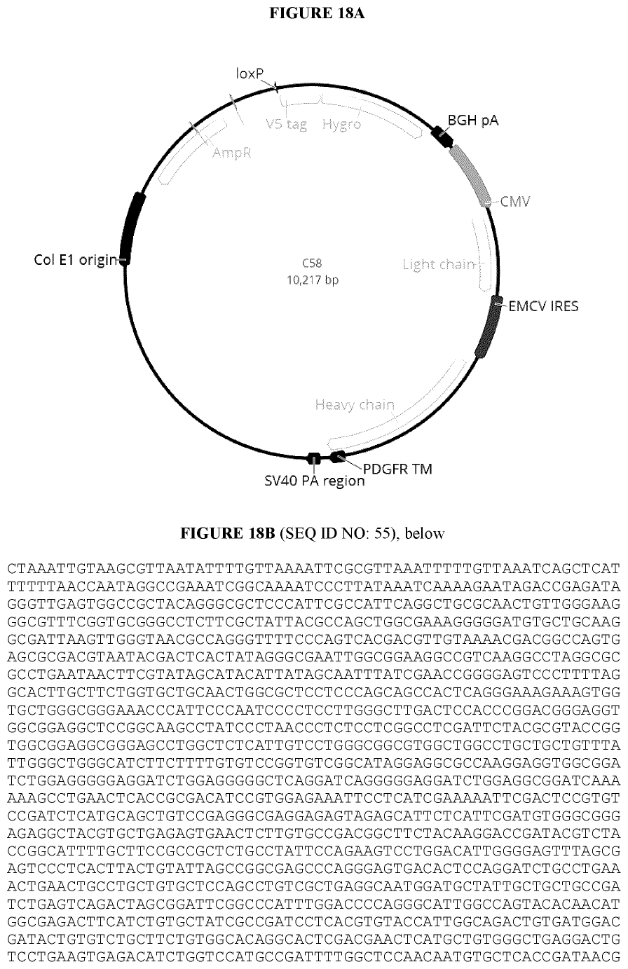

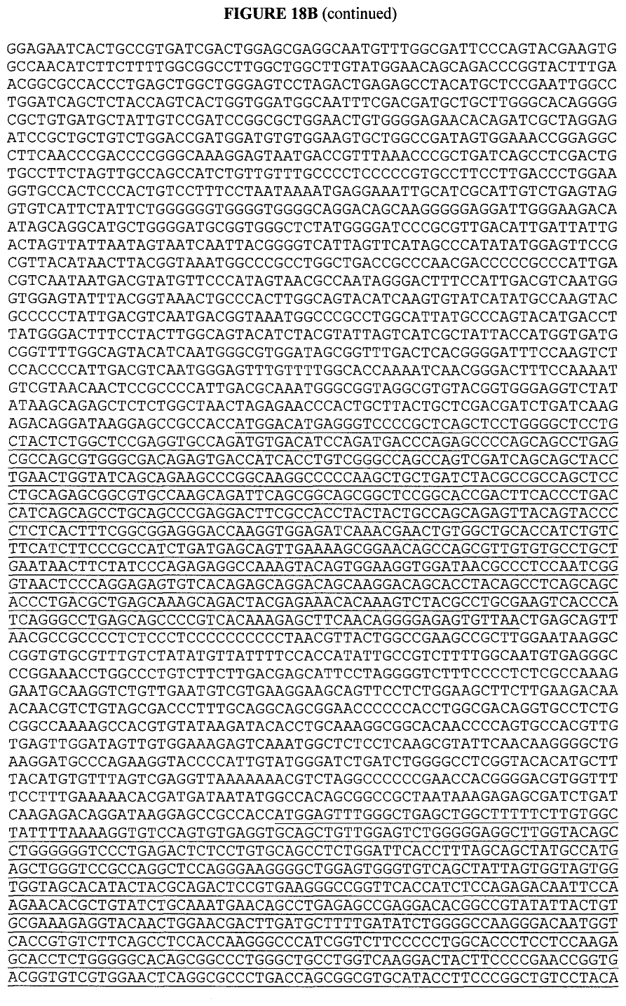

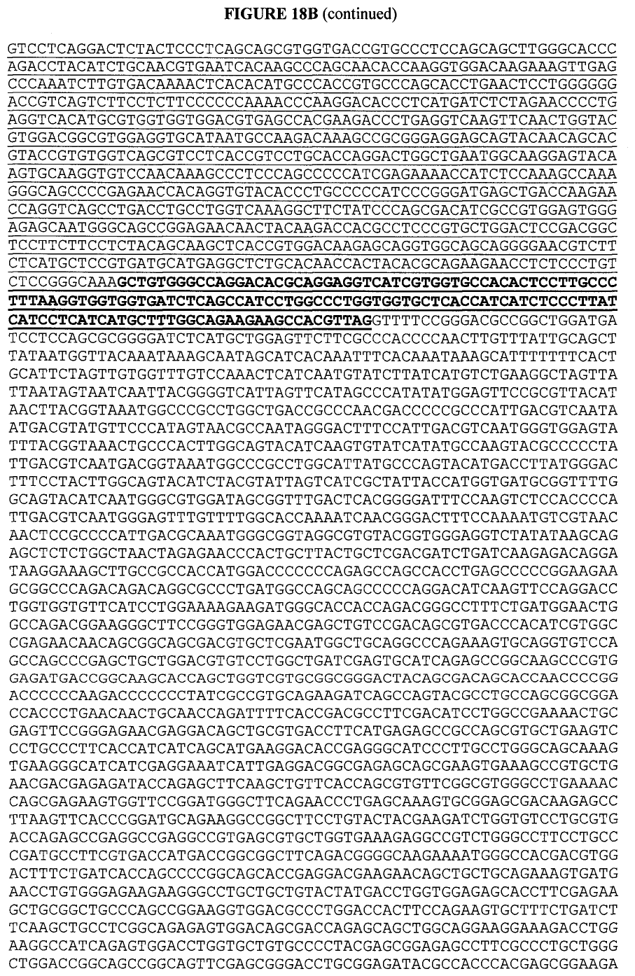

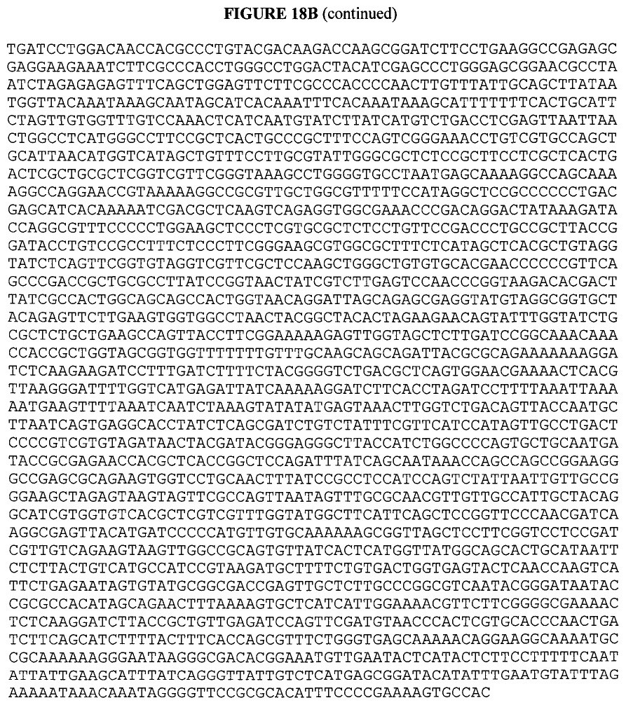

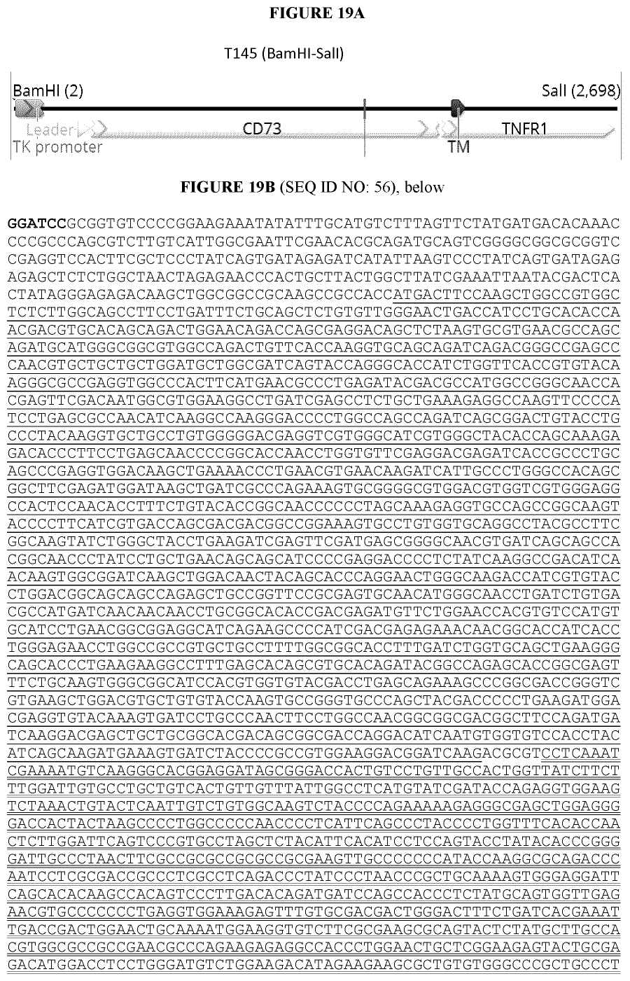

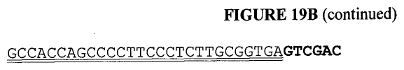

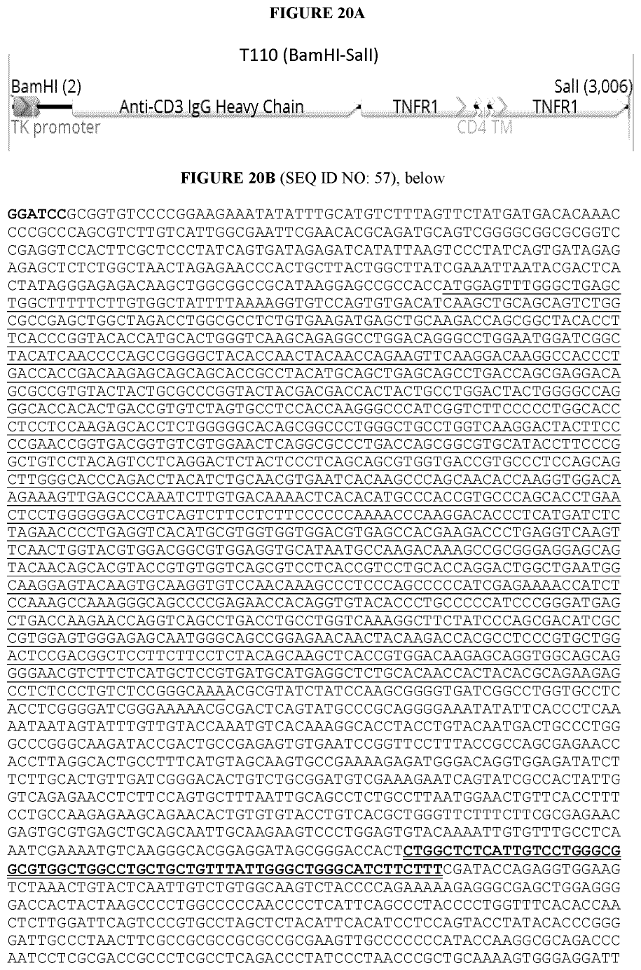

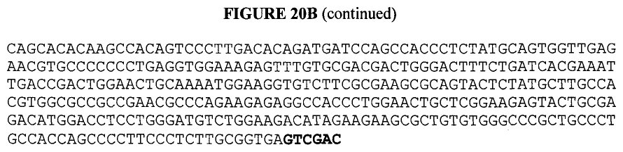

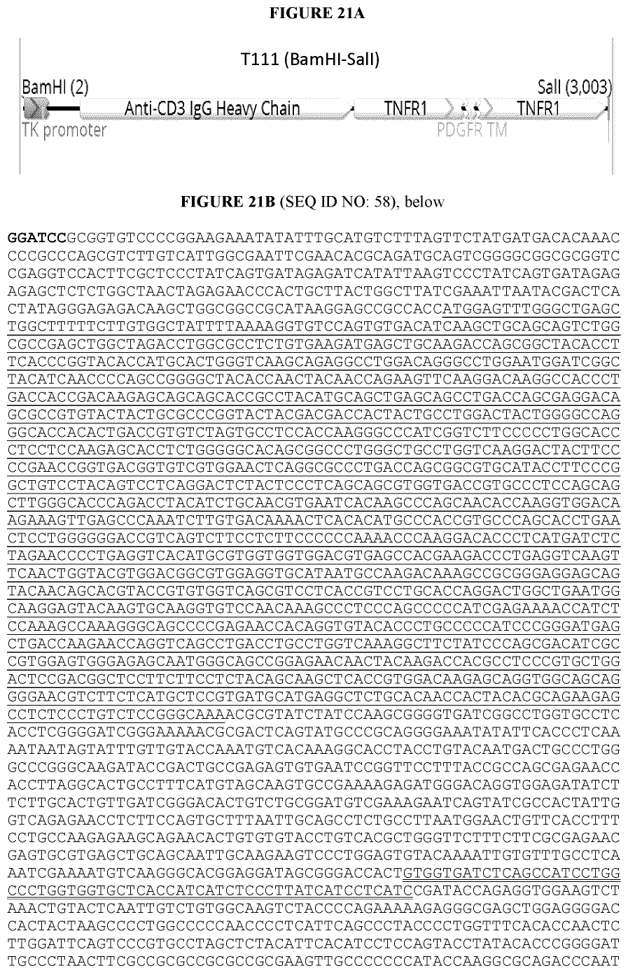

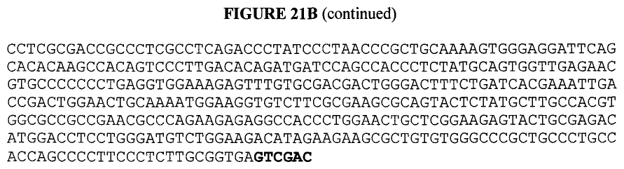

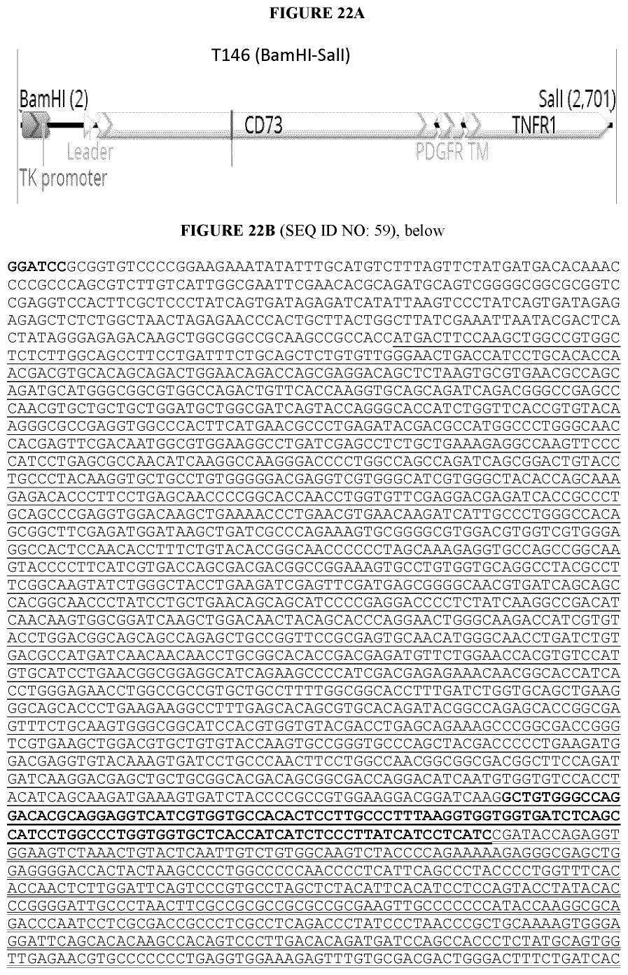

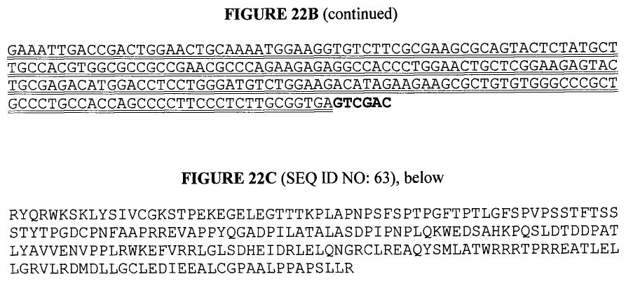

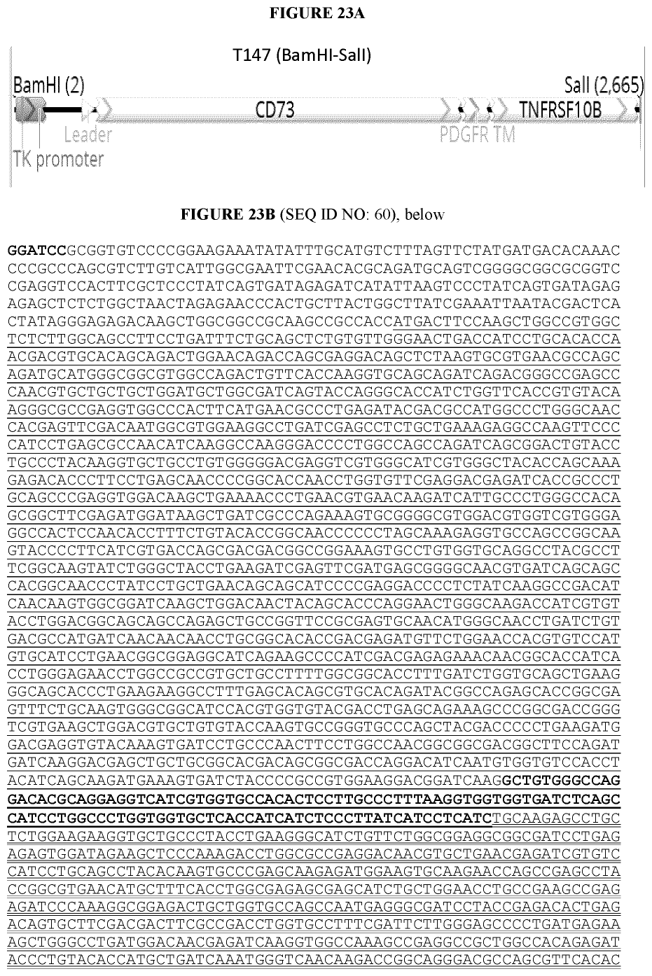

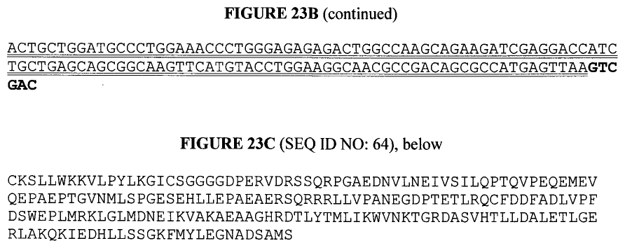

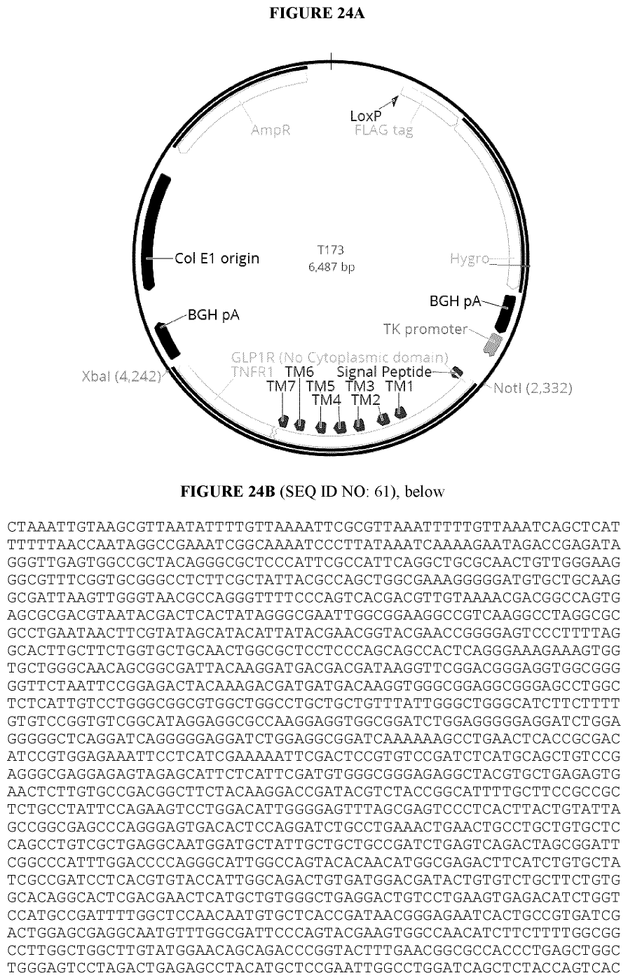

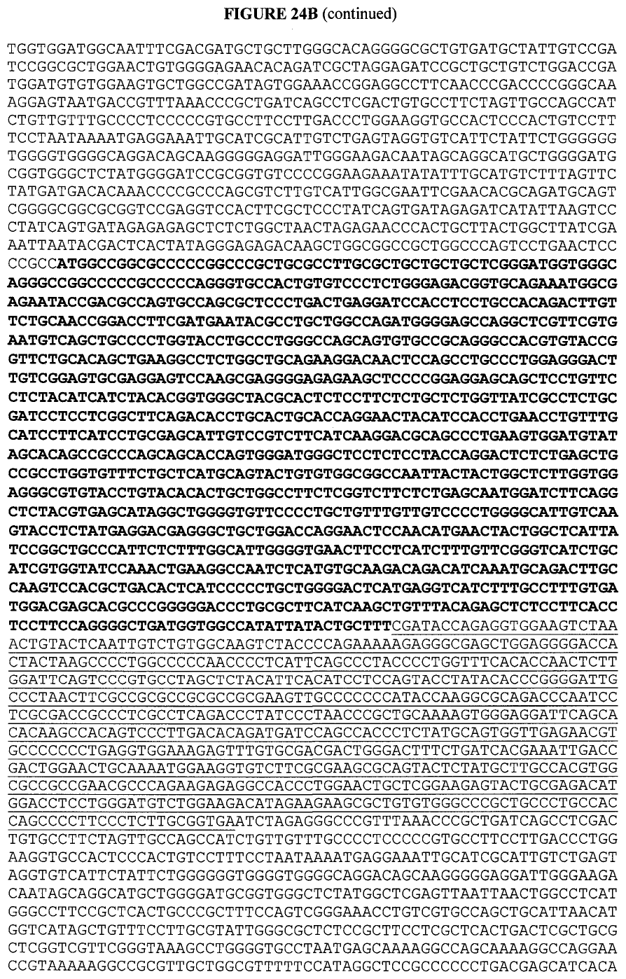

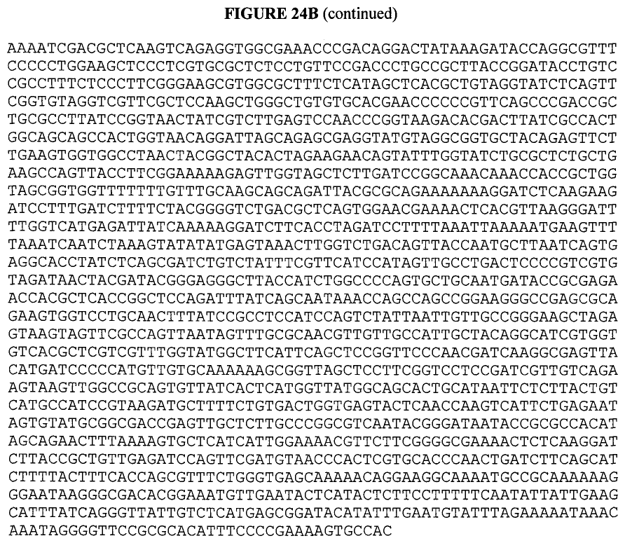

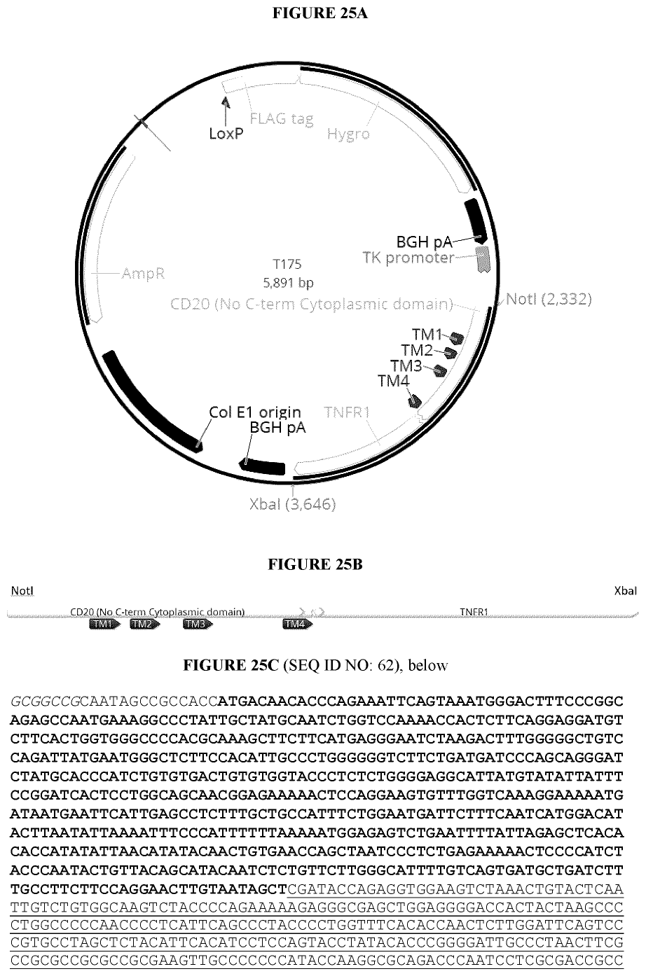

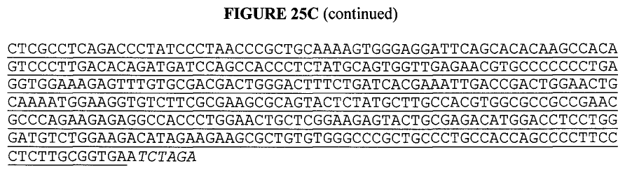

TABLE 1
List of sequences:

| SEQ ID | |
|---|---|
| NO: | Description |
| 1 | Amino acid sequence of heavy chain of antibody-based binding portion of IgG- |
| | TNFR1 chimeric receptor which specifically binds human CD3, encoded by |
| | plasmid C601. |
| 2 | Amino acid sequence of heavy chain of antibody-based binding portion of control |
| | IgG-TNFR1 chimeric receptor which has uncharacterized binding specificity, |
| | encoded by plasmid C638. |
| 3 | Amino acid sequence of heavy chain of antibody-based binding portion of IgG- |
| | TNFR1 chimeric receptor encoded by ITS017-V030/V032/V033/V034/V035 |
| | plasmids. |
| 4 | Amino acid sequence of heavy chain of antibody-based binding portion of IgG- |
| | TNFR1 chimeric receptor encoded by C644 or C645 plasmid. |
| 5 | Purposely left blank |
| 6 | Amino acid sequence of full-length TNFR1 in IgG-TNFR1 chimeric receptor |
| | ITS017-V030. |
| 7 | Amino acid sequence of TNFR1 deletion construct in IgG-TNFR1 chimeric |
| | receptor ITS017-V032. |
| 8 | Amino acid sequence of TNFR1 deletion construct in IgG-TNFR1 chimeric |
| | receptor ITS017-V033. |
| 9 | Amino acid sequence of TNFR1 deletion construct in IgG-TNFR1 chimeric |
| | receptor ITS017-V034. |
| 10 | Amino acid sequence of TNFR1 deletion construct in IgG-TNFR1 chimeric |
| | receptor ITS017-V035. |
| 11 | Purposefully left blank. |
| 12 | Purposefully left blank. |
| 13 | Amino acid sequence of IgG-TNFR1 chimeric receptor ITS017-V030. |
| 14 | Amino acid sequence of IgG-TNFR1 chimeric receptor ITS017-V032. |
| 15 | Amino acid sequence of IgG-TNFR1 chimeric receptor ITS017-V033. |
| 16 | Amino acid sequence of IgG-TNFR1 chimeric receptor ITS017-V034. |
| 17 | Amino acid sequence of IgG-TNFR1 chimeric receptor ITS017-V035. |
| 18 | Amino acid sequence of CD19-Puro fusion protein. |
| 19 | Expression vector C659 encoding CD19-Puro fusion protein. |
| 20 | Expression vector encoding chimeric receptor ITS017-V030. |
| 21 | Nucleic acid sequence of HER2ECD-PDFR. |
| 22 | Amino acid sequence of HER2ECD-PDFR. |
| 23 | Nucleic acid sequence of plasmid C601. |
| 24 | Nucleic acid sequence of plasmid C638. |
| 25 | Nucleic acid sequence of plasmid C645. |
| 26 | Amino acid sequence of IgG(heavy chain)-TNFR1(full length) with leader |
| | sequence for surface expression, encoded by plasmid C601. |
| 27 | Amino acid sequence of IgG(light chain) (no leader sequence), encoded by plasmid C601. |
| 28 | Amino acid sequence of IgG(heavy chain)-TNFR1(full length) with leader |
| | sequence for surface expression, encoded by plasmid C638. |
| 29 | Amino acid sequence of IgG(light chain) (with leader sequence), encoded by |
| | plasmid C638 or C644. |
| 30 | Amino acid sequence of the IgG(heavy chain)-TNFR1 encoded by plasmid C644 or |
| | C645, with the leader sequence for surface expression. |
| 31 | Amino acid sequence of the IgG(light chain) encoded by plasmid C645. |
| 32 | Nucleic acid sequence of plasmid V707. |
| 33 | Amino acid sequence of the light chain encoded on plasmid V707 (no leader). |
| 34 | Nucleic acid sequence of plasmid C112. |
| 35 | tracrRNA |
| 36 | CPcrRNA9 |
| 37 | CPcrRNA10 |
| 38 | CPcrRNA11 |
| 39 | CPcrRNA12 |
| 40 | SEQ ID NO: 13 minus leader |
| 41-44 | Purposely left blank |
| 45 | SEQ ID NO: 30 minus leader |
| 46 | SEQ ID NO: 31 minus leader |
| 47 | SEQ ID NO: 29 minus leader |
| 48 | Nucleic acid sequence of plasmid C487 (FIG. 9B) |
| 49 | Nucleic acid sequence of plasmid C639 (FIG. 10B) |
| 50 | Nucleic acid sequence of plasmid C884 (FIG. 11B) |
| 51 | Nucleic acid sequence of plasmid T99 (FIG. 13B) |
| 52 | Nucleic acid sequence of plasmid T100 (FIG. 14B) |
| 53 | Nucleic acid sequence of plasmid T96 (FIG. 16B) |
| 54 | Nucleic acid sequence of plasmid T101 (FIG. 17B) |
| 55 | Nucleic acid sequence of plasmid C58 (FIG. 18B) |
| 56 | Nucleic acid sequence of plasmid T145 (FIG. 19B) |
| 57 | Nucleic acid sequence of plasmid T110 (FIG. 20B) |
| 58 | Nucleic acid sequence of plasmid T111 (FIG. 21B) |
| 59 | Nucleic acid sequence of plasmid T146 (FIG. 22B) |
| 60 | Nucleic acid sequence of plasmid T147 (FIG. 23B) |
| 61 | Nucleic acid sequence of plasmid T173 (FIG. 24B) |
| 62 | Nucleic acid sequence of plasmid T175 (FIG. 25B) |
| 63 | Amino acid sequence containing the ICD of TNFR1 (FIG. 22C) |
| 64 | Amino acid sequence containing the ICD of TRAILR2 (FIG. 23C) |

### VII. Examples

The present invention will be further illustrated in the following examples.

### Example 1: Generation of an NE-κB Reporter Cell Line (L1087.4H) by Random Integration of C659, a Plasmid Encoding CD19-Puro, a Screenable-Selectable Marker for NE-κB Activation

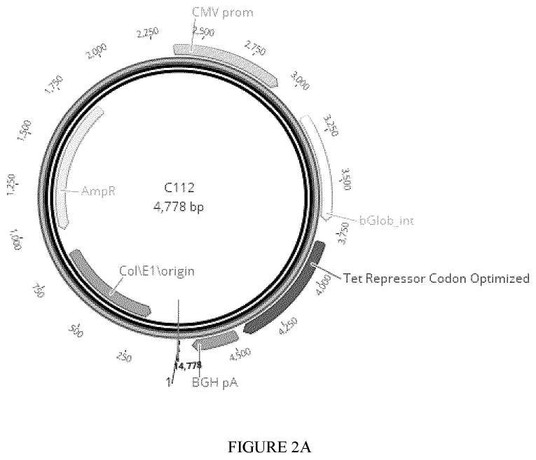

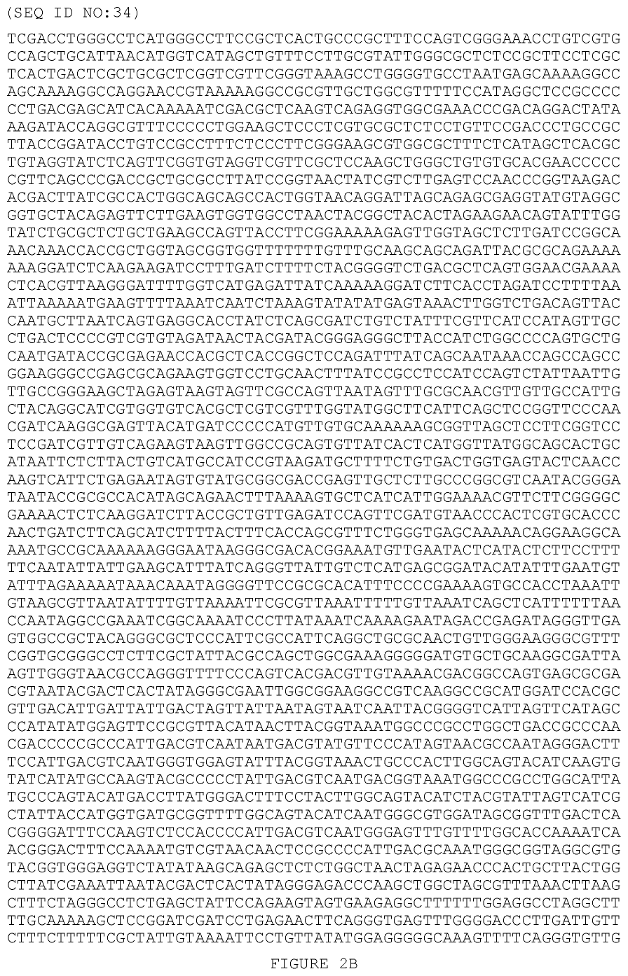

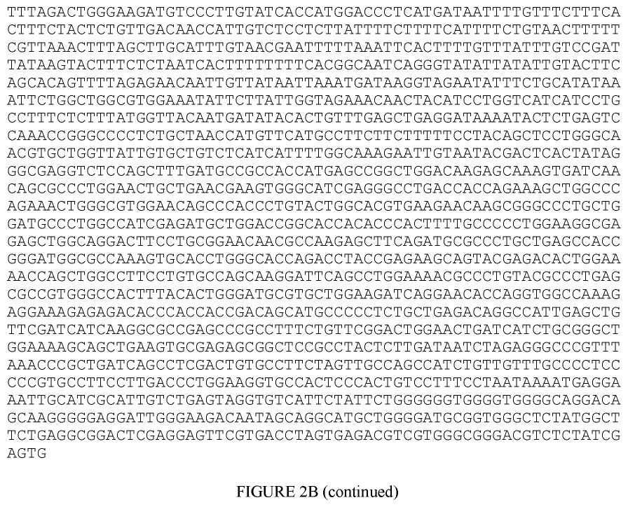

Cell line L707.3 was made from random integration of plasmid C112 (FIG. 2; SEQ ID NO:34) into HEK293 cells, resulting in cells that overexpress Tet repressor (TetR) from a codon-optimized tetR gene. The expression of TetR allows for regulation of expression levels from promoters engineered with repressor binding sites. In the absence of tetracycline, TetR binds to specific DNA sequences that are positioned flanking the TATA box and disrupts the engagement of RNA II polymerase to disrupt transcription initiation. The L707.3 cell line also had an integrated loxP site. The loxP site allows for different expression vectors to be integrated as a single copy and to be evaluated from the same chromosomal location so that expression levels are normalized.

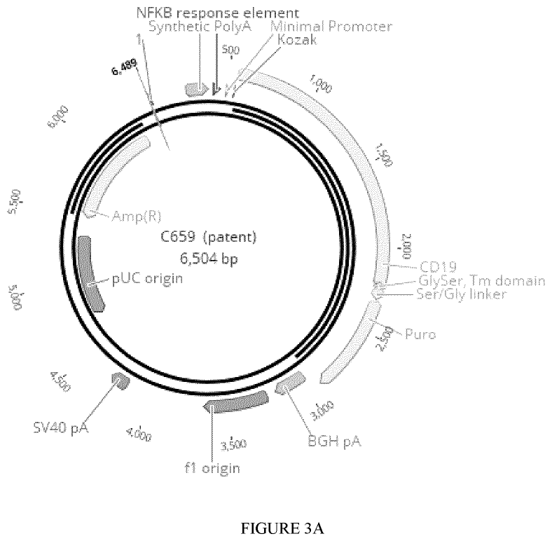

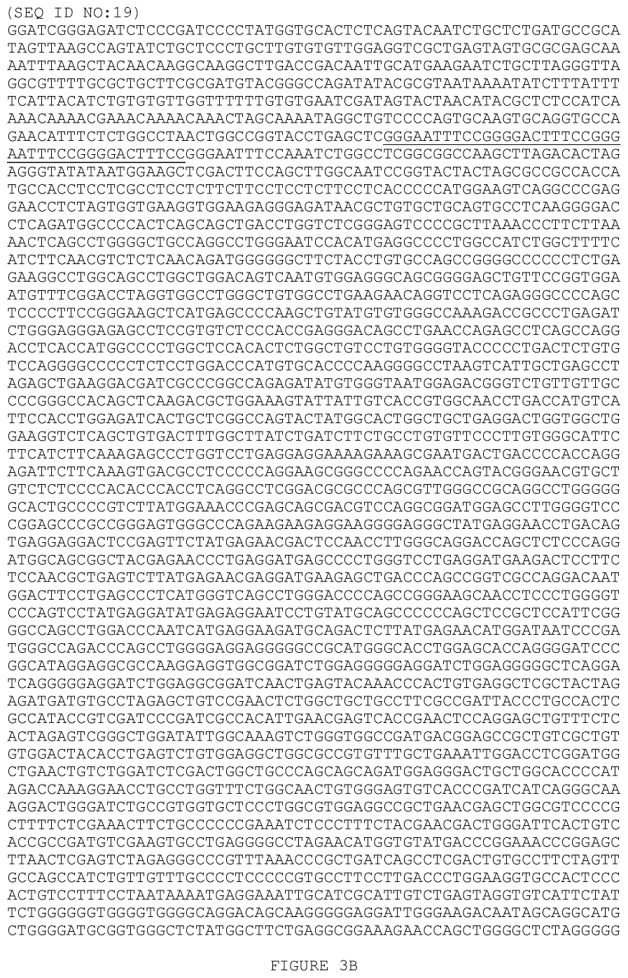

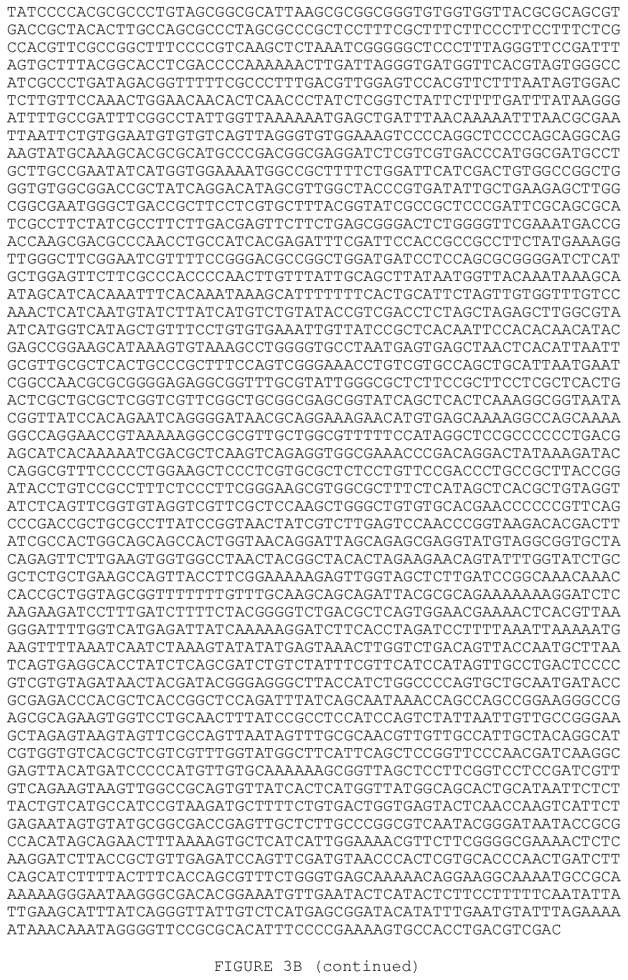

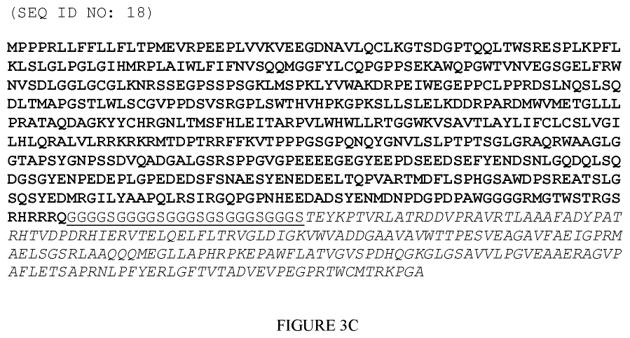

L707.3 cells were used to seed a 10-cm tissue culture treated dish. Approximately 10 million cells were seeded in DMEM (Dulbecco's Modified Eagle's Medium) supplemented with non-essential amino acids, L-glutamine, penicillin/streptomycin and 10% (v/v) fetal calf serum. The next day, 36 μg of polyethyleneimine (PEI) was diluted in Pro293™s media to a final volume of 750 μl following by a 5-minute incubation. In addition, 12 μg of plasmid C659 (FIG. 3; SEQ ID NO: 19) was diluted in Pro293™s to a final volume of 750 μl. Among other things, C659 encodes the chimeric screenable-selectable marker CD19-Puro (SEQ ID NO: 18) under the transcriptional control of activator NF-κB. The PEI and C659 samples were then mixed followed by a 20-minute incubation at room temperature. The mixed sample was then added to the L707.3 cells. Transfected cells were subsequently expanded and maintained in culture for about 1 week. Transfected cells then were treated with 5 ng/ml TNFα to induce TNFR1-mediated signaling through NF-κB. The next day, cells were incubated with an anti-CD19 antibody phycoerythrin (PE) conjugate (anti-CD19 PE; commercially available), for an hour with rotation, washed once with FACS buffer (PBS containing 1% FBS) and then sorted for PE positive cells using flow cytometry. Sorted cells were expanded. The day prior to FACS sorting to isolate single clones, cells were then treated with 2 μM Z-VAD-FMK (a pan-specific caspase inhibitor) and 5 ng/ml TNFα. The next day, cells were incubated with anti-CD19 PE and then single-cell sorted for PE positive cells into 96-well plates. Clones derived from single cells were expanded and then tested for CD19 expression and resistance to puromycin in the presence of 2 μM Z-VAD-FMK, either untreated or treated with 5 ng/ml TNFα. Clone L1087.4H (and others) were found to express low levels of CD19-Puro in the absence of TNFα treatment and high levels of CD19-Puro in the presence of TNFα treatment. Clone L1087.4H was also found to be sensitive to puromycin in the absence of TNFα and resistant to puromycin in the presence of TNFα. Such clones were called “NF-κB reporter cell lines”.

### Example 2: Generation of IgG-TNFR1 Expression Lines from NE-κB Reporter Cell Line

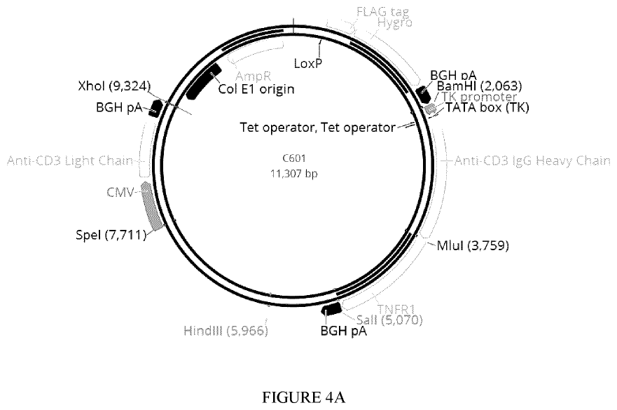

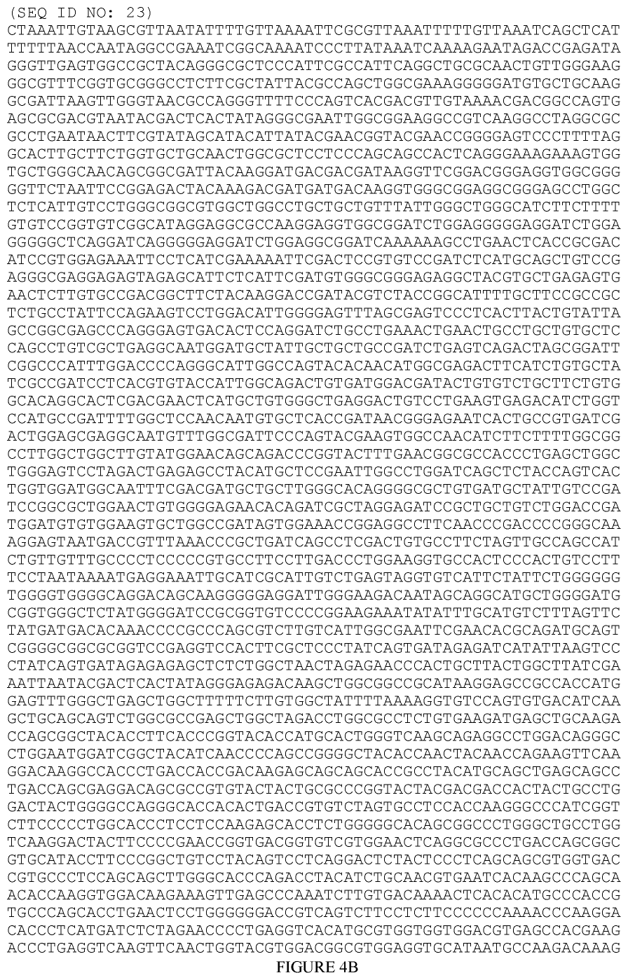

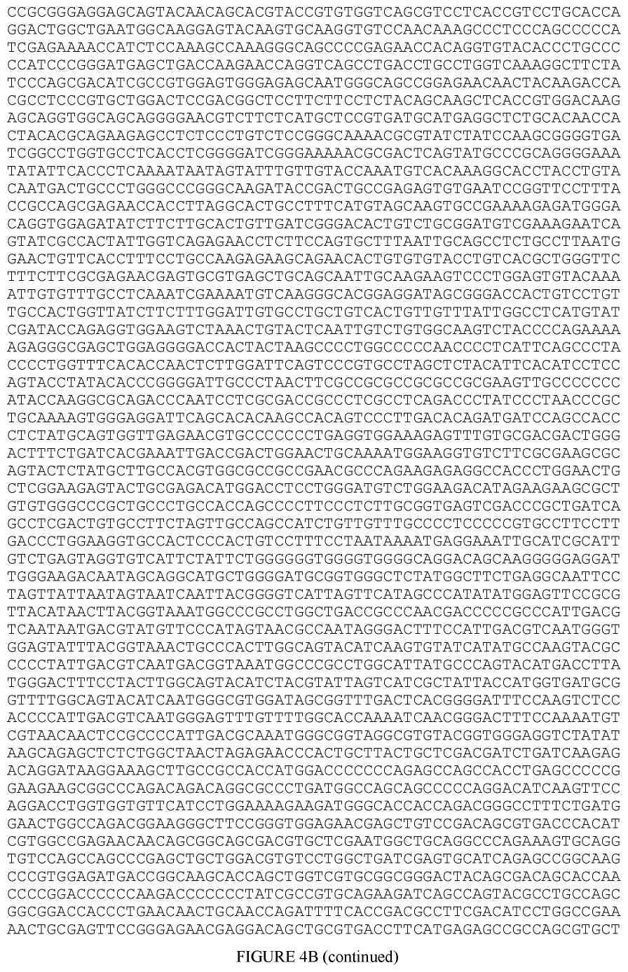

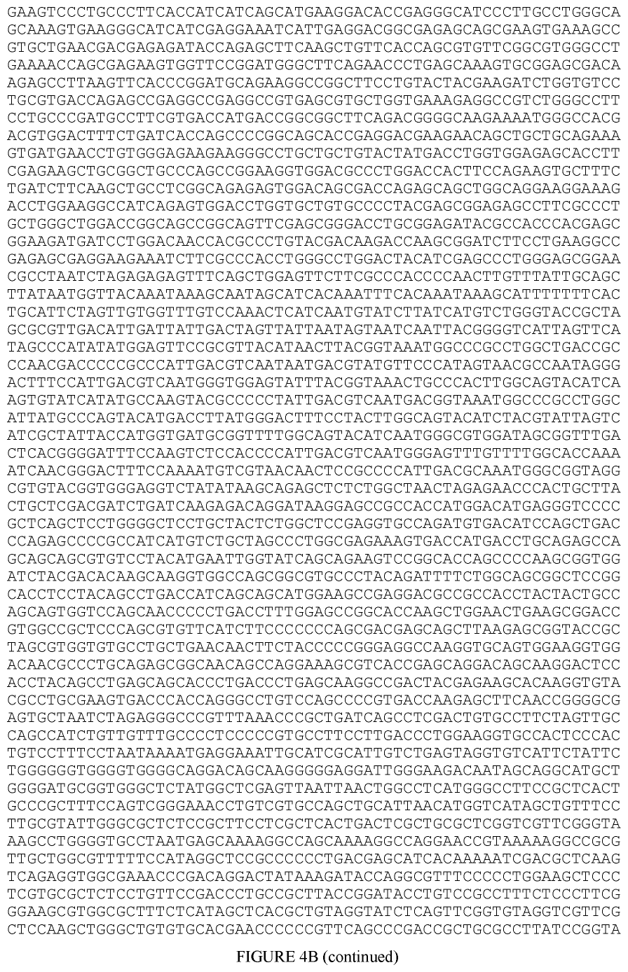

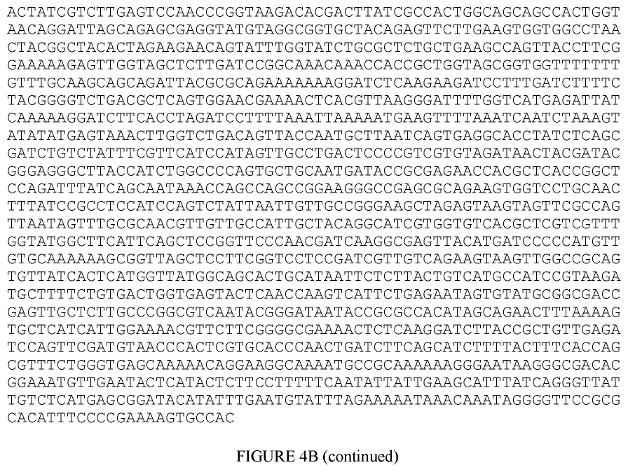

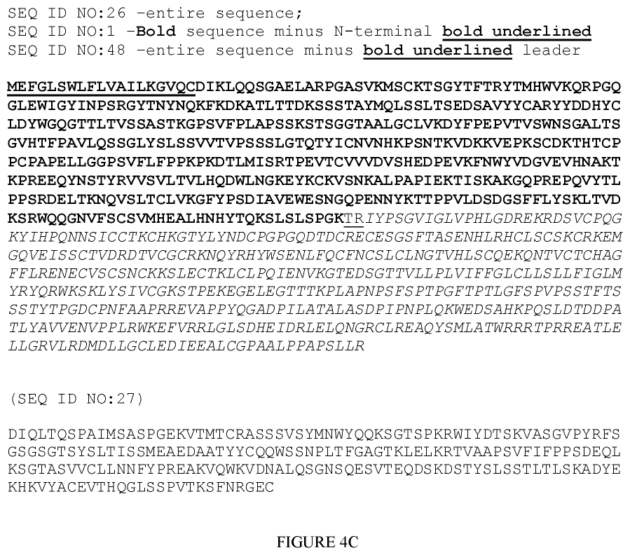

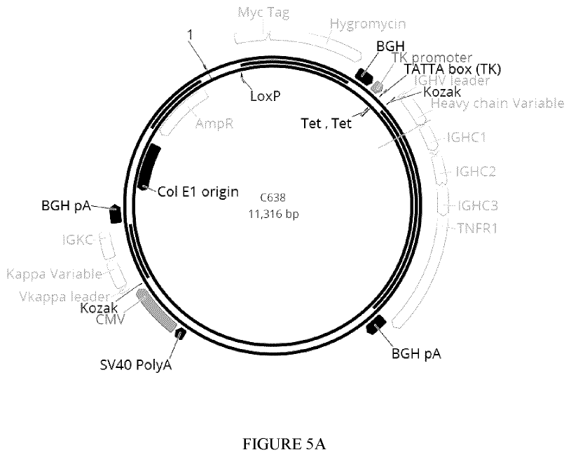

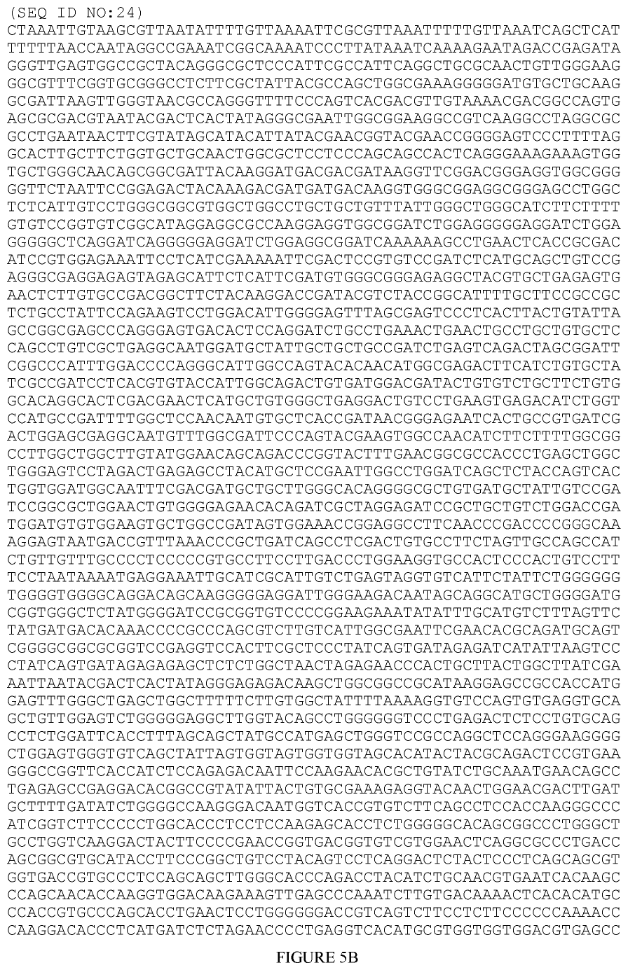

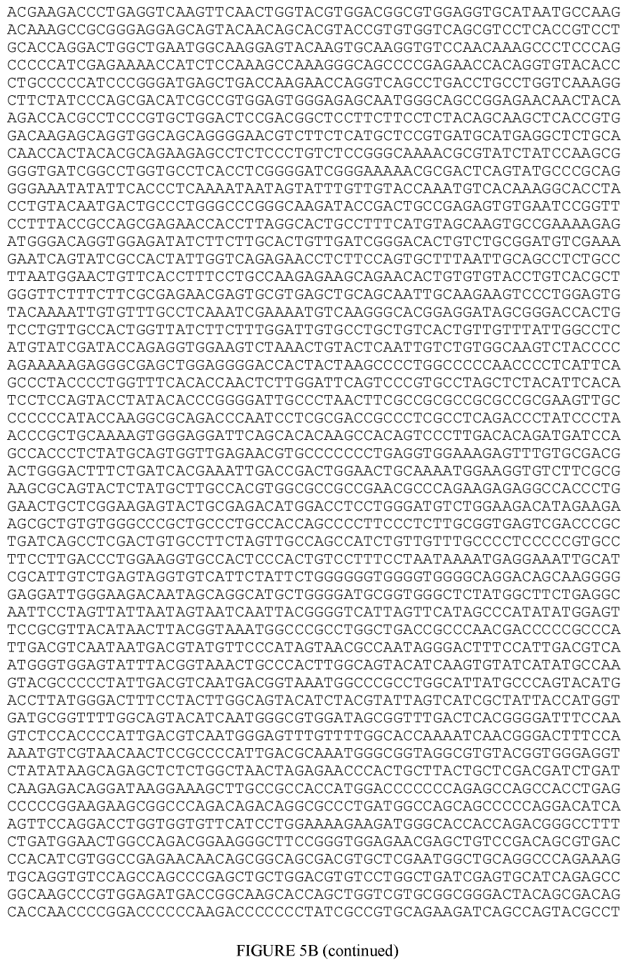

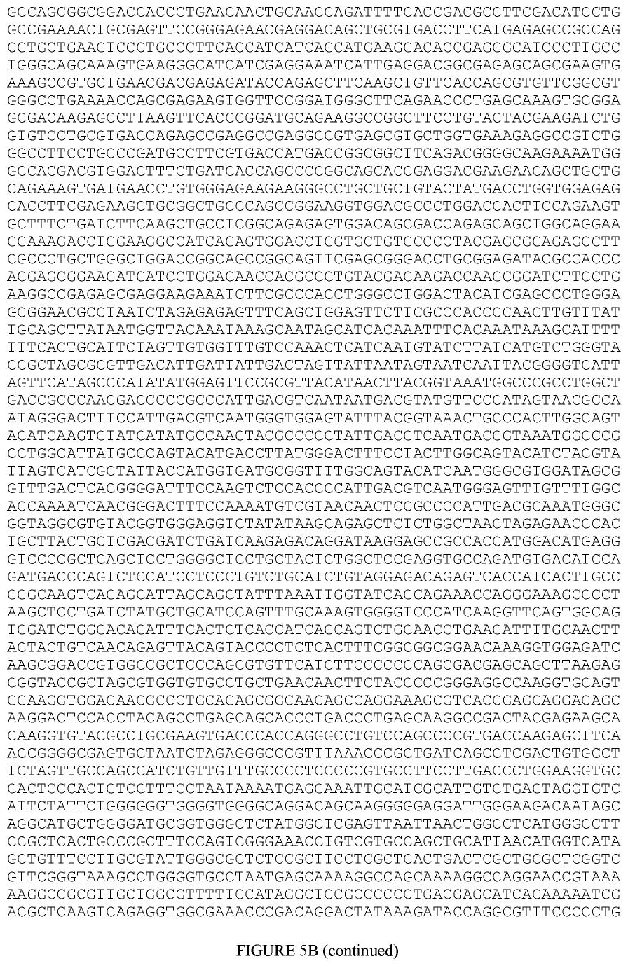

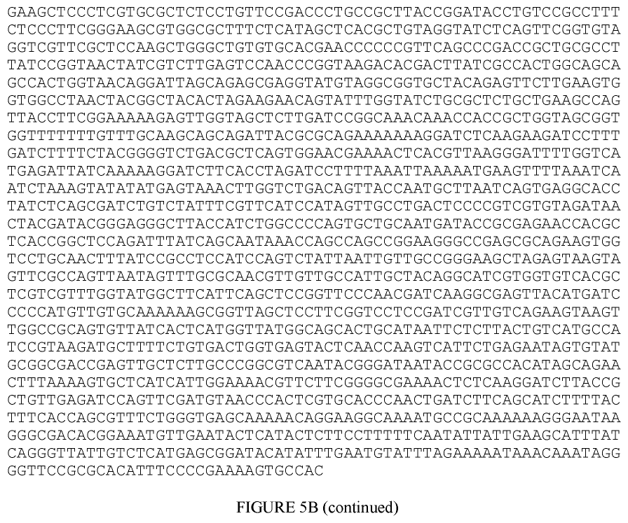

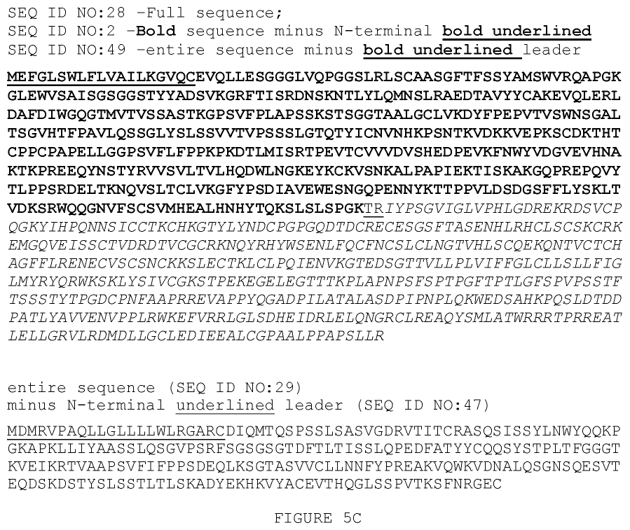

L1087.4H cells (from EXAMPLE 1) have an engineered chromosomal loxP site that permits Cre-mediated integration of plasmids that also encode a loxP site. Plasmids C601 and C638 (FIGS. 4 and 5, respectively; SEQ ID NOs: 23 and 24, respectively) encode marker genes that only express when integration at the chromosomal loxP site has occurred. Cre-mediated integration of C601 results in the expression of a fusion protein comprised of an extracellular FLAG tag, a transmembrane domain and an intracellular hygromycin resistance marker. Similarly, Cre-mediated integration of C638 results in the expression of a fusion protein comprised of an extracellular Myc tag, a transmembrane domain and an intracellular hygromycin resistance marker.

Cre-mediated recombination allows for the stable chromosomal integration of a LoxP-containing plasmid. The generation of stable cell lines containing plasmids C601 and C638 were generated as follows. L1087.4H cells were used to seed a tissue culture treated 6-well plate at approximately 1.6 million cells per well in DMEM supplemented with non-essential amino acids, L-glutamine, penicillin/streptomyin and 10% (v/v) fetal calf serum. The next day, for each transfection 2 μg PEI was diluted in Pro293™s to 125 μl followed by a 5 minute incubation. Next, 1.8 μg of C601 with 0.2 μg V503 (a Cre recombinase expression vector based on the sequences in pBS185 CMV-Cre; commercially available from Addgene, Cambridge Mass. USA; Sauer & Henderson. New Biol 1990; 2(5): 441-9) or 1.8 μg C638 with 0.2 μg V503 was diluted in Pro293™s to 125 each sample in triplicate. Diluted stocks of PEI and C601 or PEI and C638 were mixed followed by 20 minute incubations at room temperature. Samples were then added to wells of the 6-well plate seeded previously with L1087.4H cells. Cells were subsequently expanded and then stained with anti-FLAG or anti-Myc mouse IgG antibodies followed by PE-conjugated anti-Mouse IgG to detect marker gene expression. Cells were then enriched for marker gene expression using magnetic beads. The cell populations underwent further enrichment by treating with 100 μg/ml hygromycin B. The resulting lines were given the names L1122 (C601) and L1123 (C638).

### Example 3: Activation Studies with L1122 and L1123

L1122 and L1123 cell lines were tested to see if they would upregulate expression of CD19-Puro when treated with an antibody that binds human IgG Fc antibody (anti-human IgG Fc). Increased CD19-Puro expression would show that anti-human IgG Fc is acting as a multivalent binding substrate for IgG-TNFR1 by cross-linking the Fc domain in the binding portion of IgG-TNFR1. To test this, each cell line was used to seed a 24-well plate at 500,000 cells per well in the presence of 2 μM Z-VAD-FMK. Cells were either untreated or treated with 1 μg/ml polyclonal goat anti-human IgG Fc followed by overnight incubation. The next day, CD19-Puro gene expression was assessed by staining with an anti-CD19 PE antibody followed by analysis by flow cytometry. As shown in Table 2, treatment with anti-human IgG Fc showed strong upregulation of CD19-Puro gene expression in both cells lines.

L1122 and L1123 cells express IgG-TNFR1 fusion proteins with different antibody variable regions. The IgG-TNFR1 chimeric receptor expressed by L1123 has antibody variable regions of unknown specificity for use as a negative control (amino acid sequence of L1123 IgG-TNFR1 is shown in FIG. 5C and SEQ ID NOs: 28 and 29). The IgG-TNFR1 chimeric receptor expressed by L1122 has antibody variable regions derived from OKT3, an antibody that binds to human CD3 (amino acid sequence of L1122 IgG-TNFR1 is shown in FIG. 4C and SEQ ID NOs: 26 and 27). CD3 antigen is part of the TCR complex which comprises CD3γ, CD3δ and two CD3ε chains as well as the TCR alpha and beta chains. OKT3 (an anti-CD3 antibody) has been shown to at least bind CD3ε (Kjer-Nielsen et al. PNAS 2004; 101:7675-7680), such that CD3 is considered a multivalent binding substrate. Furthermore, cell surface expression of CD3 should also provide a multivalent CD3 binding substrate by displaying the CD3 molecules in close proximity to each other.

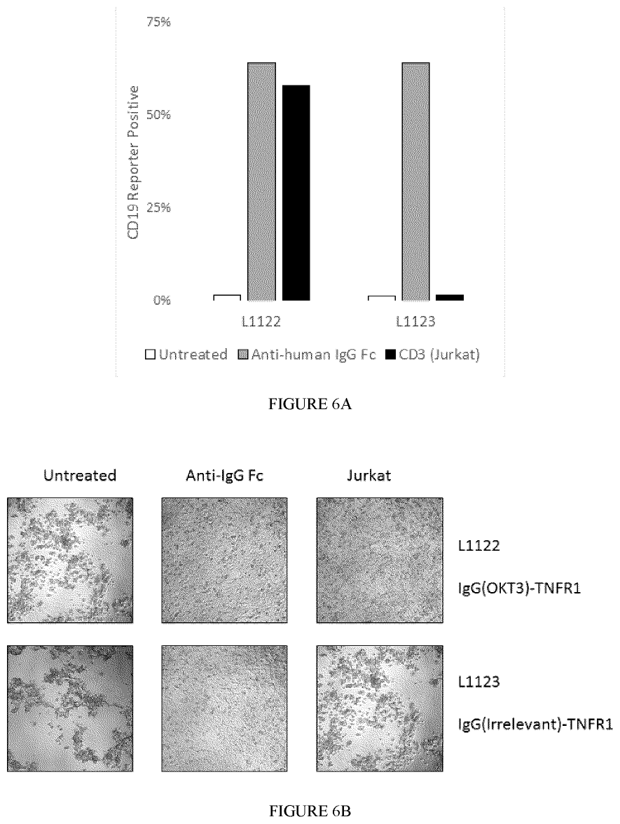

L1122 and L1123 cell lines were tested for upregulation of CD19-Puro when co-cultured with Jurkat cells, a line derived from human T cells that expresses CD3. Each cell line was used to seed a 24-well plate at 500,000 cells per well in the presence of 2 μM Z-VAD-FMK. Cells were either untreated or co-cultured with 500,000 Jurkat cells followed by overnight incubation. The next day, CD19-Puro gene expression was assessed by staining samples with an anti-CD19 PE antibody followed by analysis by flow cytometry. As shown in FIG. 6A and Table 2, co-culture with Jurkat cells showed strong upregulation of CD19-Puro gene expression in L1122 cells, but not L1123 cells, showing antigen-specific activation of CD19-Puro gene expression. Lines L1120 (anti-CD3) and L1121 (control antibody) were generated in a different NF-κB reporter cell line clone that contained the CD19-Puro reporter (i.e. not clone Li 087.4H). Although activation was observed in this cell line as well it was not as robust (64.3% versus 29.8% and 58.2% versus 30.8%). In addition, background activity was observed to be higher in the control antibody condition (4.4% versus 1.3% for L1121). The low level of background and the observed response to a multivalent binding substrate (anti-IgG Fc or CD3) shows that the bivalent structure of the antibody binding portion alone (i.e. unbound) is not responsible for non-specifically activating NF-κB reporter activity.

TABLE 2
Expression of reporter in cell lines expressing chimeric
receptors when cross-linked with anti-IgG Fc antibody
or CD3 antigen (expressed in Jurkat cells)

| Reporter | Integration | Cell Line | | anti-IgG | CD3 |
|---|---|---|---|---|---|
| cell line | Plasmid | (Ab) | Untreated | Fc | (Jurkat) |
| clone 1 | C638 | L1120 | 4.4% | 30% | 31% |
| | | (anti-CD3) | | | |
| clone 1 | C638 | L1121 | 4.4% | 38% | 4.6% |
| | | (control) | | | |
| clone 2 | C601 | L1122 | 1.6% | 64% | 58% |
| (L1087.4H) | | (anti-CD3) | | | |
| clone 2 | C601 | L1123 | 1.3% | 64% | 1.7% |
| (L1087.4H) | | (control) | | | |

L1122 and L1123 cells were also tested for their ability to gain resistance to puromycin when IgG-TNFR1 signaling is activated. Each cell line was used to seed a 24-well plate at 500,000 cells/well in the presence of 2 μM Z-VAD-FMK (caspase inhibitor). Cells were either untreated, treated with 1 μg/ml anti-IgG Fc or treated with 500,000 Jurkat cells. The next day 1.5 μg/ml puromycin was added to each well. The following day, the wells were observed for cytotoxicity. As shown in FIG. 6B, puromycin was toxic to both L1122 and L1123 when untreated. Pretreatment of each line with anti-IgG Fc resulted in resistance to puromycin whereas only L1122 was resistant to puromycin when co-cultured Jurkat cells.

### Example 4: Expression Level of IgG-TNFR1 and Background Signaling

Tetracycline was not present during the above experiments with L1120, L1121, L1122 and L1123. The observed CD19-Puro expression levels therefore correspond to repressed expression of IgG-TNFR1. It was observed that the chimeric receptors were exquisitely sensitive to binding substrate and high levels of chimeric receptor expression were correlated with increased background activity (signaling in the absence of a cross-linker/binding substrate). The optimal NF-κB reporter cell lines that were identified had levels of the chimeric receptor which were extremely low, near the levels of detection and barely detectable using FACS. The example cell lines described above utilized a weak promoter and the tetracycline repressor system to reduce the levels of transcription to optimize levels of expression for improved use as a reporter/biosensor (although other strategies for optimizing expression levels of chimeric receptors may be used, including those described herein).

To optimize the responsiveness of the biosensor, the expression level of the chimeric receptor (e.g. IgG-TNFR1) may be adjusted for optimal binding substrate-dependent expression of the marker gene (screenable, selectable or screenable-selectable). If the levels are too low, upregulation of marker gene expression is poorly observed. If the levels are too high, the marker gene expression is poorly distinguished over background. This is demonstrated in this Example. L1123 cells were used to seed a 24-well plate at 300,000 cells per well. Levels of IgG-TNFR1 were varied by adding different concentrations of tetracycline, which derepresses the TK-tet promoter controlling IgG-TNFR1 expression. The next day, CD19-Puro gene expression was assessed by staining samples with anti-CD19 PE antibody followed by analysis by flow cytometry. As the concentration of tetracycline was increased, upregulation of CD19-Puro was confirmed from the observation of CD19 on the cell surface, even in the absence of treatment with binding substrate (data not shown).

### Example 5: Antibody Affinities in IgG-TNFR1 Chimeric Receptors

This example demonstrates that antibodies with K_{D} values of 100 nM or less when converted to chimeric receptors (e.g. IgG-TNFR1) can be activated by antigen (i.e. a binding substrate). This example also demonstrates that biosensors described herein may be activated by antigens expressed on target cells. Furthermore, this example demonstrates that biosensors described herein may be used to discriminate affinity of antibodies. This example also provides additional evidence that co-culture is a viable approach to presenting antigen and that native antigen expressed on the cell surface broadens the applications beyond soluble antigens.

The antigen in this example was the HER2 protein expressed on the surface of HEK293 cells. HER2 is a transmembrane glycoprotein consisting of an extracellular domain having four subdomains, a transmembrane Domain™, and an intracellular domain (ICD). HER2 is an orphan receptor (i.e. it has no known ligand) but is known to form monomers, homodimers, heterodimers (with other erB family members) and oligomers when expressed on the cell surface, depending on its activation state (Brennan et al., Oncogene 2000; 19: 6093-6101). The extracellular domain of HER2 in particular is thought to mediate dimerization/oligomerization (Brennan et al., Oncogene 2000; 19: 6093-6101). In addition to its intrinsic ability to form dimers alone, cell surface expression of HER2 would also be expected provide a multivalent HER2 binding substrate by displaying the HER2 in clusters or cross-linked in the cell membrane.

Antibodies having different affinities to HER2 were made into IgG-TNFR1 chimeric receptors. As shown in Table 3, one antibody had affinity K_{D} value of 107 nM as measured by Biacore™ and the second had significantly higher K_{D} (weaker affinity), estimated to be several hundred nM.

TABLE 3
IgG-TNFR1 constructs with differing affinity for HER2

| Plasmid ID | Construct Description |
|---|---|
| C644 | IgG(HER2, ITS001-V928-based, K_{D} higher than 200 nM)- |
| | TNFR1 fusion |
| C645 | IgG(HER2, ITS001-V737-based, K_{D} 107 nM)-TNFR1 |
| | fusion |

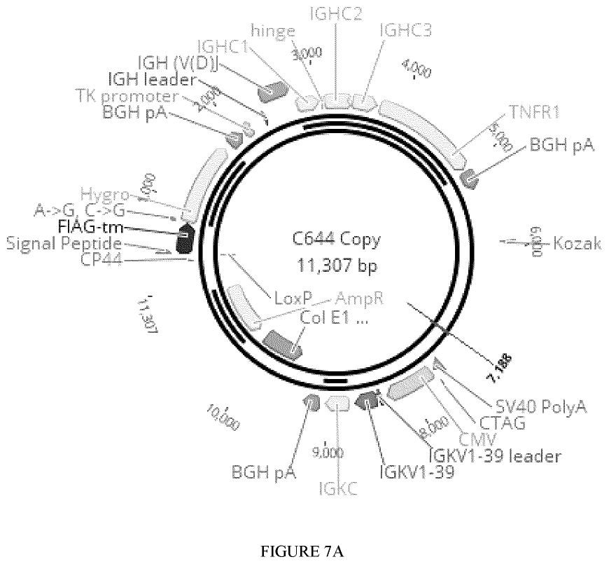

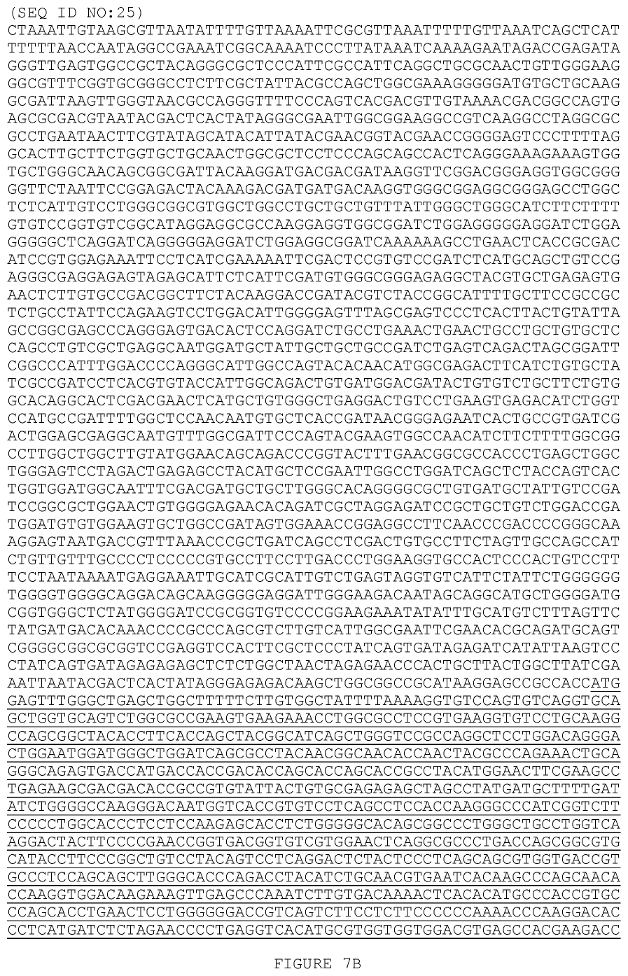

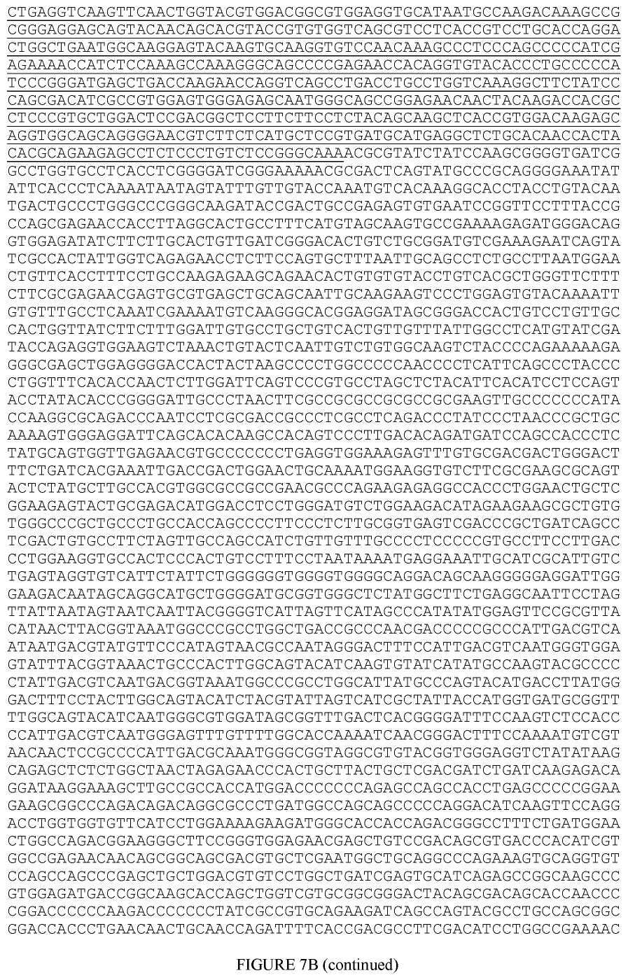

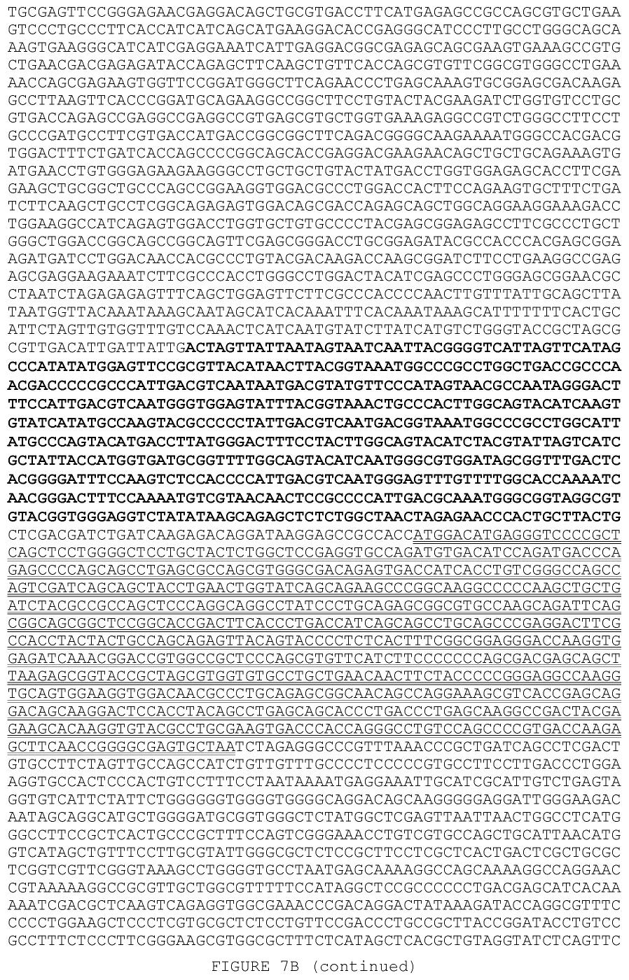

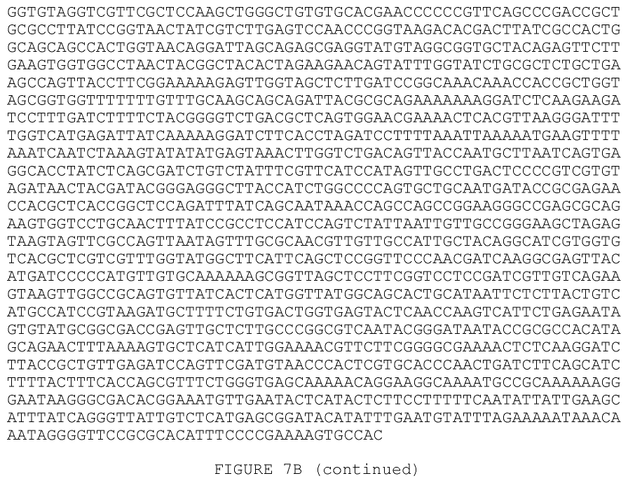

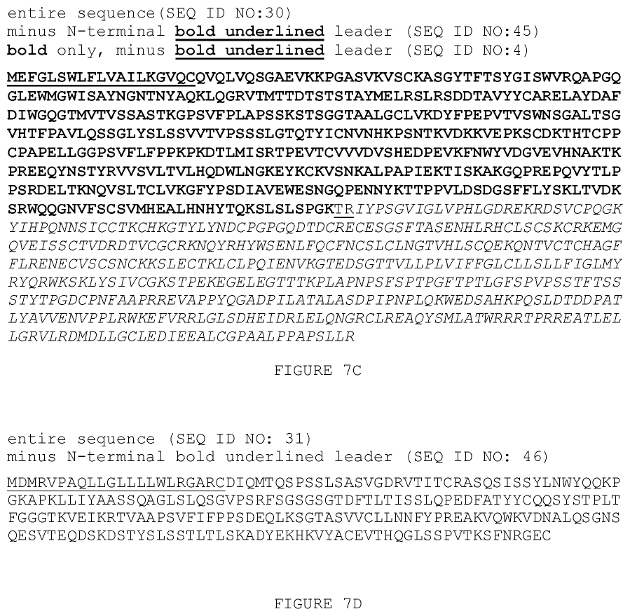

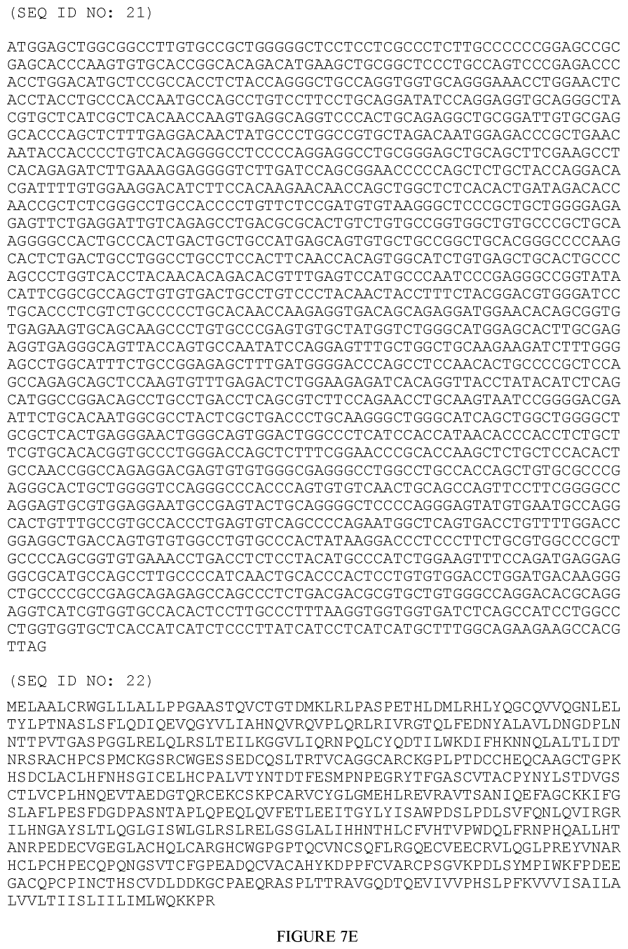

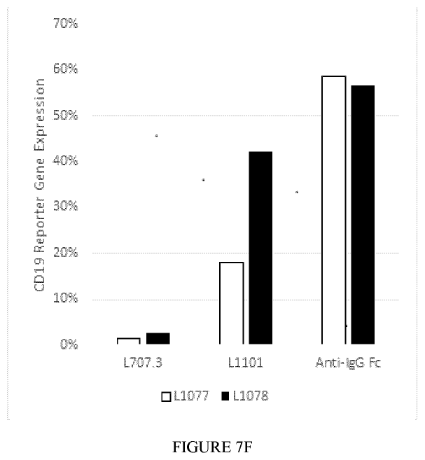

The plasmid schematic of C644 is shown in FIG. 7A and the nucleotide sequence of C645 is shown in FIG. 7B (SEQ ID NO:25). Plasmid C645 is identical to plasmid C644 except the antibody in IgG-TNFR1 encoded by C645 has a 5 amino acid insertion (SQAGL) in the light chain which decreases the K_{D} from 100s of nM to 107 nM. The amino acid sequence of the IgG(heavy chain)-TNFR1 encoded by both C644 and C645, with the leader sequence for surface expression, is shown in FIG. 7C (SEQ ID NO:30). The amino acid sequence of the IgG(light chain) encoded by C644 is shown in FIG. 5C (SEQ ID NO:29) and the amino acid sequence of IgG(light chain) encoded by C645 is shown in FIG. 7D (SEQ ID NO:31).

Plasmids C644 and C645 were introduced into an NF-κB reporter line (expressing CD19-Puro) by Cre-mediated integration to generate lines L1077 and L1078, respectively. Each line was tested for its ability to undergo upregulation of CD19-Puro gene expression when co-cultured with L1101 cells, which overexpresses HER2 extracellular domain (HER2ECD) as a HER2ECD-PDGFR fusion (also called V964; FIG. 7E and SEQ ID NOs: 21 and 22 for nucleic acid and amino acid sequences respectively) using a CMV promoter (“PDGFR” is platelet derived growth factor receptor which functions to anchor the HER2ECD to the cell membrane) or with L707.3 control cells (low HER2 expression). L1077 and L1078 cells were seeded at 300,000 cells per well in a 24-well plate with 300,000 L707.3 cells, 300,000 L1101 cells or 1 μg/ml anti-IgG Fc. The next day, cells were stained for expression of CD19-Puro using anti-CD19 PE and then analysed by flow cytometry. Low levels of CD19-Puro gene expression were observed when L1077 or L1078 were co-cultured with L707.3 cells (these cells have low levels of approximately 10,000 copies of the HER2 antigen). CD19-Puro gene expression increased when cells were co-cultured with L1101 cells that have high levels of HER2 and the magnitude of increase correlated well with antibody affinity for HER2. Each cell line had similarly high levels of CD19-Puro gene expression when incubated with anti-IgG Fc (not specific for the IgG variable region), as expected. This data is presented in FIG. 7F.

### Example 6: IgG-TNFR1 Extracellular Domain (ECD) Deletion Constructs

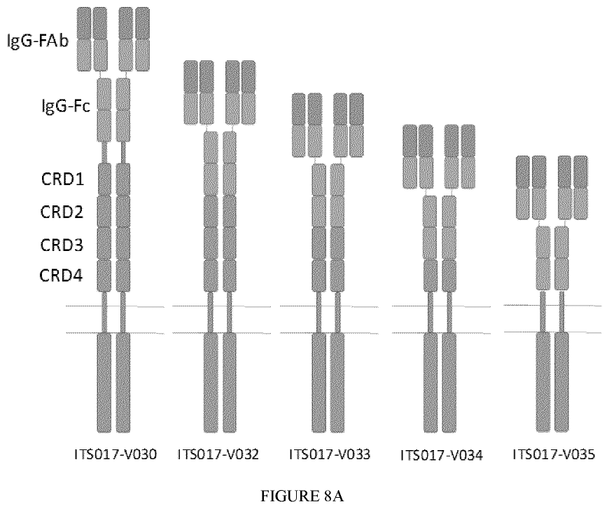

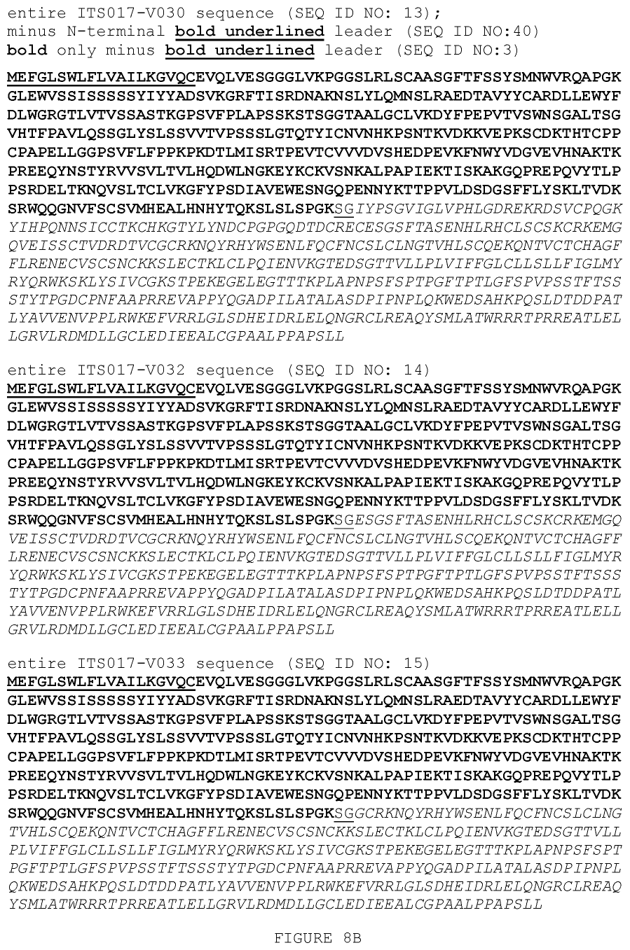

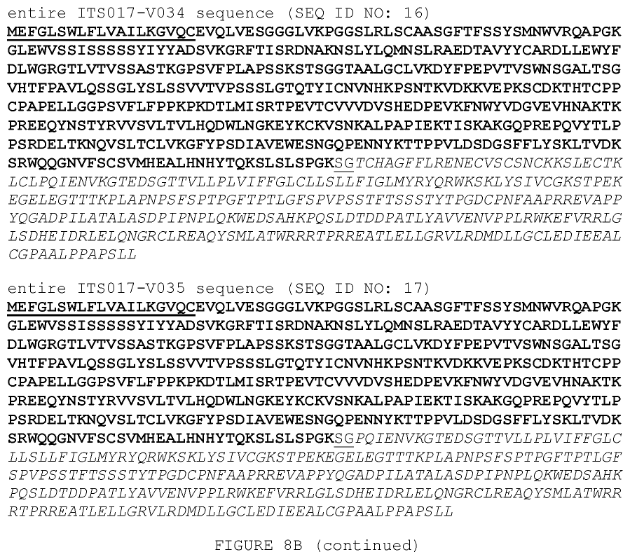

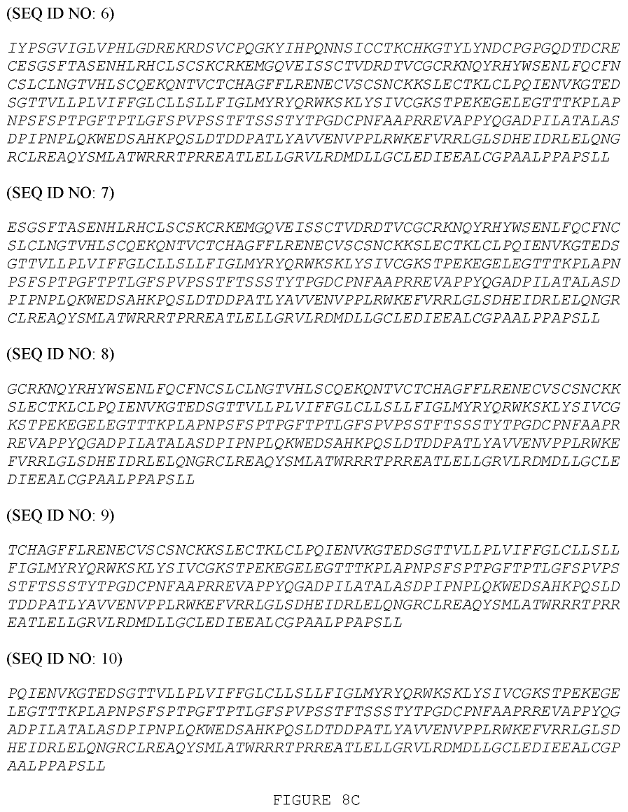

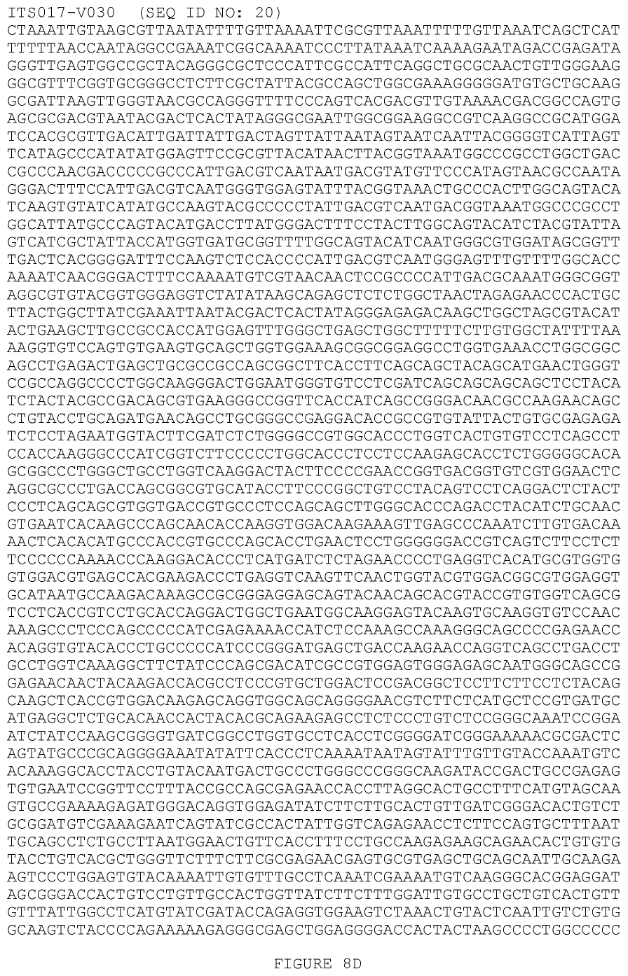

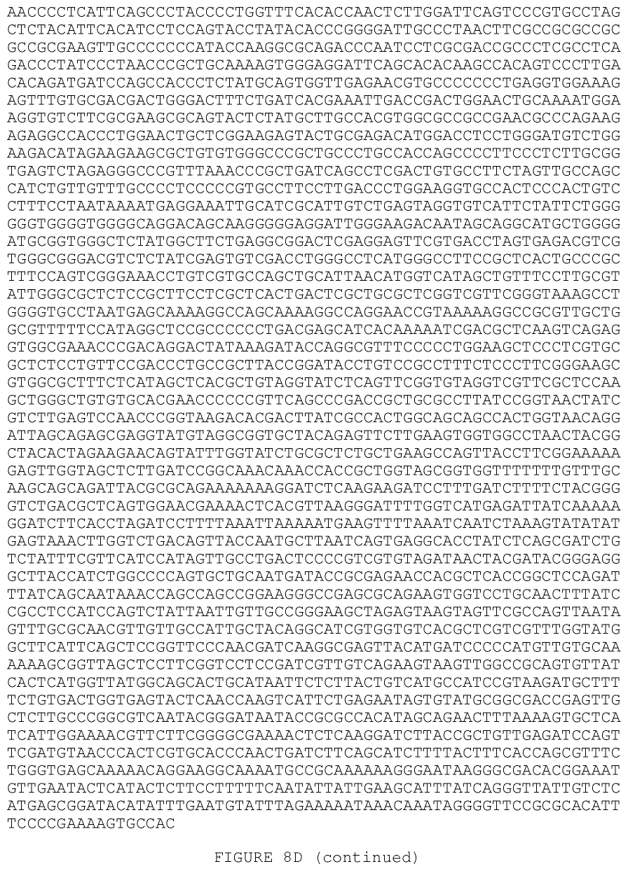

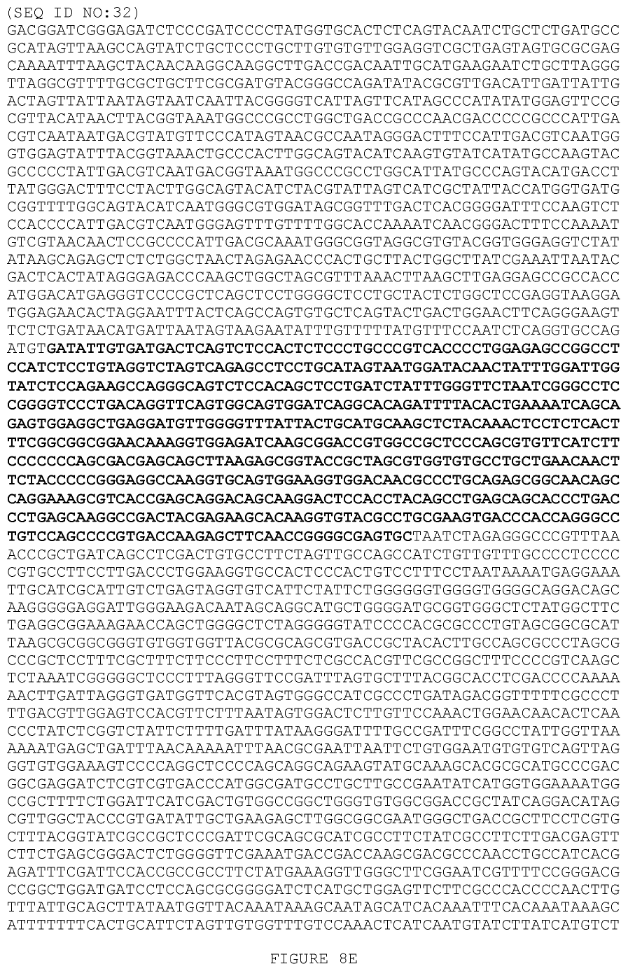

The above examples used IgG-TNFR1 constructs in which the full-length TNFR1 was fused to the antibody heavy chain. In this example, deletion constructs (schematically shown in FIG. 8A; IgG(heavy chain)-TNFR1 construct amino acid sequences shown in FIG. 8B and SEQ ID NOs: 13-17; and amino acid sequences of TNFR1 portions thereof shown in FIG. 8C and SEQ ID NOs: 6-10) in which portions of the TNFR1 ECD were deleted were tested for IgG-TNFR1-mediated NF-κB reporter activity by transiently transfecting a NF-κB reporter cell line with expression vectors for each construct. The nucleotide sequence of the vector encoding IgG(heavy chain)-TNFR1(full-length) is shown in FIG. 8D (SEQ ID NO:20). The light chain was encoded on a separate expression plasmid, V707. The nucleic acid sequence of plasmid V707 (SEQ ID NO:32) is shown in FIG. 8E. The amino acid sequence of the light chain encoded on plasmid V707 (SEQ ID NO:33) is shown in FIG. 8F. All of these constructs, including those with full-length TNFR1 (ITS017-V030; SEQ ID NO: 13 for heavy chain) and truncated TNFR1 (ITS017-V032N033N034N035; SEQ ID NOs: 14-17 for heavy chain), were found to induce NF-κB reporter activity (i.e. expression of CD19-Puro; data not shown). The high level of expression from transient expression results in receptor activation without requiring the presence of a multivalent binding substrate for receptor cross-linking. This example shows that the native TNFR1 ECD (or any TNFRSF member ECD) is nonessential for the chimeric receptor to work in a biosensor cell and further confirms that the full length IgG does not sterically inhibit the chimeric receptor from being able to be activated.

Plasmid constructs and cell lines referenced below are summarized in Table 4, with reference to FIGS. 9A, 9B, 10A and 10B. An IgG-truncated TNFR1 chimeric receptor was shown to be functional when expressed by a stable cell line. An expression vector encoding IgG(unknown specificity)-TNFR1(no ECD) (construct C639) was introduced into L998.1 cells using Cre-mediated integration (as described above). The resulting cell line was assigned the name L1076. L998.1 is an NF-κB responsive reporter line derived from L707.3 and C487 using a method similar to that described in EXAMPLE 1, but without screening for puromycin resistance. Among other things, C487 encodes CD19 under the transcriptional control of activator NF-κB.

TABLE 4
Summary of plasmids & cell lines

| Plasmid/ | |
|---|---|
| Cell Line | Description |
| C487 | NF-κB responsive CD19 reporter plasmid (FIGS. 9A and 9B) |
| C639 | Expression vector encoding an IgG(unknown specificity)- |
| | TNFR1(lacking the extracellular domain) chimeric receptor |
| | driven by TK-tet promoter (FIGS. 10A and 10B) |
| L998.1 | NF-κB responsive CD19 reporter cell line |
| L1076 | NF-κB responsive CD19 reporter cell line with an |
| | IgG(unknown specificity)-truncated TNFR1 chimeric receptor |
| | derived from cell line L998.1 and Cre-integrated C639 |

To test signaling, L1076 was used to seed a 24-well tissue culture treated plate with approximately 400,000 cells per well. Wells were either left untreated or treated with 1 μg/ml polyclonal anti-human IgG Fc. The next day, cells were stained for CD19 expression and then analyzed by flow cytometry (as described above). As shown in Table 5, L1076 unregulated CD19 expression when treated with anti-human IgG Fc relative the untreated control, providing addition evidence that chimeric receptors that use TNFR1 to do not require the extracellular domain to be functional.

TABLE 5
Expression of CD19 reporter in cell line
L1076 when treated with anti-human IgG Fc

| Cell | Chimeric | Binding Portion | | Anti-human |
|---|---|---|---|---|
| line | Receptor | Specificity | Untreated | IgG Fc |
| L1076 | IgG-TNFR1(no | Unknown | 3.0% | 39.5% |
| | ECD) | | | |

### Example 7: Generation of Biosensor Cell Capable of Both Positive and Negative Selection from Activation of a Single Chimeric Receptor

Additional plasmid constructs and cell lines referenced below are summarized in Table 6.

TABLE 6
Summary of plasmids & cell lines

| Plasmid/ | |
|---|---|
| Cell Line | Description |
| C884 | NF-κB responsive TRAILR1 reporter plasmid constructed by cloning into |
| | HindIII/Xbal sites of parent plasmid C487 (FIGS. 11A and 11B) |
| L1181 | Derivate of L1087.4H (NF-κB responsive CD19/Puro) with disrupted endogenous |
| | death receptor expression |
| L1231 | NF-κB responsive CD19/Puro/TRAILR1 reporter line derived from L1181 and |
| | randomly integrated plasmid C884 |
| L1240 | NF-κB responsive CD19/Puro/TRAILR1 reporter line with an IgG(anti-CD3)-TNFR1 |
| | chimeric receptor derived from cell line L1231 and Cre-integrated plasmid C601 |
| L1262 | NF-κB responsive CD19/Puro/TRAILR1 reporter line derived from L1181 and |
| | randomly integrated plasmid C884 |
| L1280 | NF-κB responsive CD19/Puro/TRAILR1 reporter line with an IgG(unknown |
| | specificity)-TNFR1 chimeric receptor derived from cell line L1262 and Cre- |
| | integrated plasmid C638 |

This example describes a dual selection biosensor in which antigen-dependent signaling results in the expression of a death receptor (TRAILR1 or TRAILR2) and the expression of a positive selection gene (PuroR). This makes it possible to control when the signal will or will not result in the death of the cell by controlling the addition of a ligand (in this case TRAIL). The cell line can be easily cultured without any selection. If following antigen induced signaling, the ligand TRAIL is not present then positive selection can proceed (in the presence of caspase inhibitor) by adding the drug puromycin. If on the other hand negative selection of cells with signaling chimeric receptors is desired, then ligand for the death receptor is added to the culture. In this manner the fate of a biosensor cell with a single signaling chimeric receptor is dependent on the ligand or toxic compound (e.g. antibiotic) that is added to the culture.

The first step in generating a cell line that can be negatively selected in a ligand dependent manner, for example, by TRAIL-mediated apoptosis in response to antigen-mediated signaling was to disrupt endogenous genes that would otherwise make the cell constitutively sensitive to TRAIL-mediated apoptosis. TRAILR1 (TNFRSF10A) and TRAILR2 (TNFRSF10B) are known TRAIL receptors and were targeted for disruption using the CRISPR-Cas9 genome editing technology. Disruption was carried out using the Alt-R™ CRISPR-Cas9 System from Integrated DNA Technologies (IDT).

First, L1087.4H cells were used to seed wells of a 6-well tissue culture treated plate at about 1.6 million cells/well in 2 ml DMEM supplemented with 10% (v/v) fetal calf serum (FCS), 1× non-essential amino acids (NEAA), 1× L-glutamine and 1× penicillin/streptomycin. The next day, cells were transfected with Alt-R™ S.p. Cas9 Expression Plasmid (purchased from IDT). For each well, 7.5 μl TransIT-X2™ (Mirus Bio LLC) was mixed with 117.5 μl OptiMEM™ in a final volume of 125 μl followed by a 5-minute incubation. Meanwhile, 2 μg Alt-R™ S.p. Cas9 Expression Plasmid DNA was mixed with OptiMEM™ in a final volume of 125 μl. Next, diluted TransIT-X2™ was added to the diluted DNA followed by mixing and then a 20-minute incubation. Mixes of DNA/TransIT-X2™ were then added to wells of the 6-well plate, 250 μl per well. Cells were then incubated overnight in a humidified tissue culture incubator at 37° C. in the presence of about 5% carbon dioxide.

The next day, culture supernatants were removed and the cells washed with 1 ml phosphate buffered saline (PBS). Next, 200 μl trypsin was added followed by a brief incubation in the tissue culture incubator. Cells were then resuspended in 1 ml supplemented DMEM followed by centrifugation and removal of the supernatant. Pellets were then resuspended in supplemented DMEM and ¾ of the cells were used to seed a 6-well tissue culture treated plate in 2 ml.

Stocks of tracrRNA and crRNA were provided by IDT as shown in Table 7.

TABLE 7
tracrRNA and crRNA

| Sequence ID | Sequence | Description | SEQ ID NO: |
|---|---|---|---|
| tracrRNA | AGCAUAGCAAGUUAAAAUAAGGCUAGUCCGUUAUCAACUUGAA | tracrRNA | 35 |
| | AAAGUGGCACCGAGUCGGUGCUUU | | |
| CPcrRNA9 | GAAGUCCCUGCACCACGACCGUUUUAGAGCUAUGCU | crRNA targeting DR4 | 36 |
| | | (TNFRSF10A, TRAILR1) | |
| CPcrRNA10 | ACAGCAUGUCAGUGCAAACCGUUUUAGAGCUAUGCU | crRNA targeting DR4 | 37 |
| | | (TNFRSF10A, TRAILR1) | |
| CPcrRNA11 | AUAGUCCUGUCCAUAUUUGCGUUUUAGAGCUAUGCU | crRNA targeting DR5 | 38 |
| | | (TNFRSF108, TRAILR2) | |
| CPcrRNA12 | AGGUCGGUGAUUGUACACCCGUUUUAGAGCUAUGCU | crRNA targeting DR5 | 39 |
| | | (TNFRSF108, TRAILR2 | |

The stocks were resususpend in Nuclease Free Duplex Buffer (IDT) to a final concentration of 100 μM. Next, tracrRNA/crRNA mixes were prepared by combining 3 μl 100 μM tracrRNA with 3 μl 100 μM CPcrRNA9, CPcrRNA10, CPcrRNA11 or CPcrRNA12 and 94 μM Nuclease Free Duplex Buffer (IDT). Next, four transfection samples were prepared. First, 12 μl each of 3 μM CPcrRNA9/tracrRNA and CPcrRNA11/tracrRNA, CPcrRNA9/tracrRNA and CPcrRNA12/tracrRNA, CPcrRNA10/tracrRNA and CPcrRNA11/tracrRNA or CPcrRNA10/tracrRNA and CPcrRNA12/tracrRNA were mixed followed by the addition of 12 μl Lipofectamine™ RNAiMAX Transfection Reagent and OptiMEM™ to a final volume of 800 μl. Samples were incubated 20 minutes and then added to the L1087.4H cells previously transfected with the Alt-R™ S.p. Cas9 Expression Plasmid. Two days later, each transfected sample was expanded to a 10-dish in 10 ml supplemented DMEM. About 12.5 million cells from each sample were subsequently used to seed T175 flasks in 35 ml supplemented DMEM. Next, each line was maintained in 20 ng/ml TRAIL (R&D Systems) to enrich for cells with disrupted death receptor expression. Any cells in which TRAILR1 and TRAILR2 were not disrupted will die in the presence of TRAIL. A monoclonal anti-TRAILR2 antibody conjugated to Alexa Fluor™ 647 confirmed initial disruption of TRAILR2 expression in about 50% of cells in each sample. In contrast, expression of TRAILR1 could not be detected with an anti-TRAILR1 monoclonal antibody conjugated to PE, even in the parent L1087.4H line. All four disrupted lines were pooled, stained for TRAILR2 expression using the anti-TRAILR2 Alexa Fluor™ 647 conjugate and then sorted by flow cytometry for TRAILR2 negative cells. The final population of TRAILR2 negative cells was shown to be resistant to TRAIL-mediated apoptosis (data not shown). This cell line was assigned the name L1181.

With a modified biosensor cell line resistant to TRAIL-mediated apoptosis, it is possible to introduce both positive and negative selection in response to antigen dependent signaling. For example, a plasmid with a minimal promoter controlled by an NF-κB response element can be subsequently linked to an open reading frame encoding a fusion of the transmembrane protein CD19 and PuroR linked to a death receptor (TRAILR1 or TRAILR2) by an IRES or a P2A ribosomal skipping sequence (i.e. for co-translation of CD19-Puro and TRAILR1/R2 in response to NF-κB signaling due to chimeric receptor binding of substrate/antigen). This plasmid could then be introduced into cells by random (or specific) integration and those with the desired properties—CD19 expression, resistance to puromycin and sensitivity to TRAIL only in response to antigen dependent signaling—could be selected. Alternatively, a two plasmid system could be used where one plasmid has a minimal promoter controlled by an NF-κB response element linked to a CD19-PuroR gene and the other plasmid has a minimal promoter/NF-κB response element linked to the death receptor gene (TRAILR1 or TRAILR2) or to a different cell surface marker such as CD4 linked by IRES or P2A sequences to the death receptor gene. These plasmid cassettes can be randomly (or specifically) integrated in a sequential fashion to ensure that each cassette is expressed at its optimal level. The CD19-puroR cassette should have low levels of the puroR gene such that (1) in the absence of chimeric receptor signal activation, levels of CD19-PuroR expression are insufficient to protect the cell from killing when puromycin is added to the culture, but (2) do provide for cell survival when the chimeric receptor is activated by substrate binding. Similarly, the CD4-TRAILR1 or CD4-TRAILR2 cassette should have low enough levels of expression in the absence of chimeric receptor signal activation such that the cell will not die in the presence of the TRAIL ligand unless the chimeric receptor is activated by binding of its substrate/antigen. The use of the CD19 and CD4 markers (as an example) would allow tracking of the response of each of the NF-κB cassettes in the absence of selection.

As an example, a dual selection reporter line was made as follows. L1181 cells were used to seed wells of a 6-well tissue culture treated plate. Approximately 1.6 million cells were seeded in triplicate in DMEM (Dulbecco's Modified Eagle's Medium) supplemented with non-essential amino acids, L-glutamine, penicillin/streptomycin and 10% (v/v) fetal calf serum. The next day, for each well 8 μg of polyethyleneimine (PEI) was diluted in Pro293S medium to a final volume of 125 μl followed by a 5-minute incubation. In addition, 2 μg of plasmid C884 (see Table 6, above) was diluted in Pro293S to a final volume of 125 μl. Among other things, C884 encodes TRAILR1 under the transcriptional control of activator NF-κB. The PEI and C884 samples were then mixed followed by a 20-minute incubation at room temperature. The mixed sample was then added to the L1181 cells. Transfected cells were subsequently expanded and maintained in culture for about 1 week. Transfected cells then were treated with 5 ng/ml TNFα to induce TNFR1-mediated signaling through NF-κB. The next day, cells were stained for TRAILR1 expression using an anti-TRAILR1 monoclonal antibody conjugated to PE. TRAILR1 positive cells were enriched using flow cytometry. The enriched population was treated a second time with TNFα and the next day, TRAILR1 positive cells were single-cell sorted by flow cytometry. The resulting cell line clones were expanded and then screened for those that underwent apoptosis in the presence of a combination of 5 μg/ml TNFα and 20 μg/ml TRAIL. A clone with the desired properties was identified and assigned the name L1231 (see Table 6, above).

A chimeric receptor was introduced into L1231 to demonstrate dual selection following activation of NF-κB signaling. Expression plasmid C601 (see FIGS. 4A and 4B), a construct encoding IgG(anti-CD3)-TNFR1 driven by a TK-tet promoter, was introduced into L1231 using Cre-mediated integration. The resulting stable cell line was assigned the name L1240 (see Table 6, above).

L1240 was used to seed a 24-well tissue culture treated plate with approximately 400,000 cells per well. Wells were either left untreated or treated with 1 μg/ml polyclonal anti-human IgG Fc, 20 ng/ml TRAIL or 2 μM Z-VAD-FMK. Treatments were carried out either alone or in combination as indicated in Table 8. The next day, some wells were treated with 1.5 μg/ml puromycin. The following day, cytotoxicity was assessed by estimating the fraction of the well surface occupied by cells. The results are summarized in Table 8. As shown, NF-κB signaling resulting from activation of the chimeric receptor (due to substrate binding) sensitized cells to TRAIL-mediated apoptosis (sample 4). The effect was blocked when cells were treated with Z-VAD-FMK (sample 5), a pan caspase inhibitor that protects cells from apoptosis. NF-κB signaling resulting from activation of the chimeric receptor (due to substrate binding) also protected cells from puromycin-mediated cytotoxicity (compare samples 7 and 9). This demonstrates the successful creation of a dual selection reporter cell line that can positively or negatively select for cells in which the chimeric receptor binds substrate depending on the choice of treatments (e.g. treatment with puromycin or TRAIL).

TABLE 8
L1240 cells are protected from puromycin-mediated cytotoxicity
or sensitized to TRAIL-mediated apoptosis following activation
of NF-κB signaling by chimeric receptor cross-linking.

| | | | | | Estimated per- |
|---|---|---|---|---|---|
| | | Polyclonal | | | centage of the |
| Sample | Z-VAD- | anti-human | | Puro- | well surface oc- |
| No. | FMK | IgG Fc | TRAIL | mycin | cupied by cells |
| 1 | — | — | — | — | 95% |
| 2 | — | Yes | — | — | 90% |
| 3 | — | — | Yes | — | 95% |
| 4 | — | Yes | Yes | — | 50% |
| 5 | Yes | Yes | Yes | — | 95% |
| 6 | Yes | — | — | — | 95% |
| 7 | Yes | — | — | Yes | 0% |
| 8 | Yes | Yes | — | — | 95% |
| 9 | Yes | Yes | — | Yes | 95% |

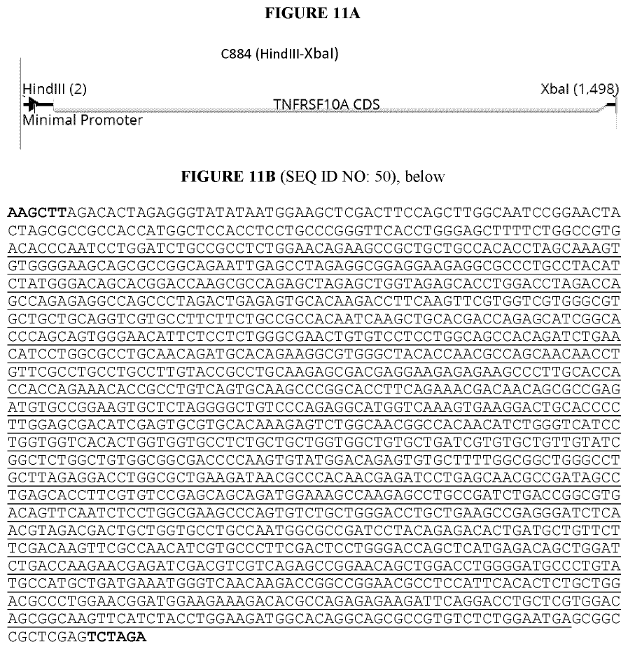

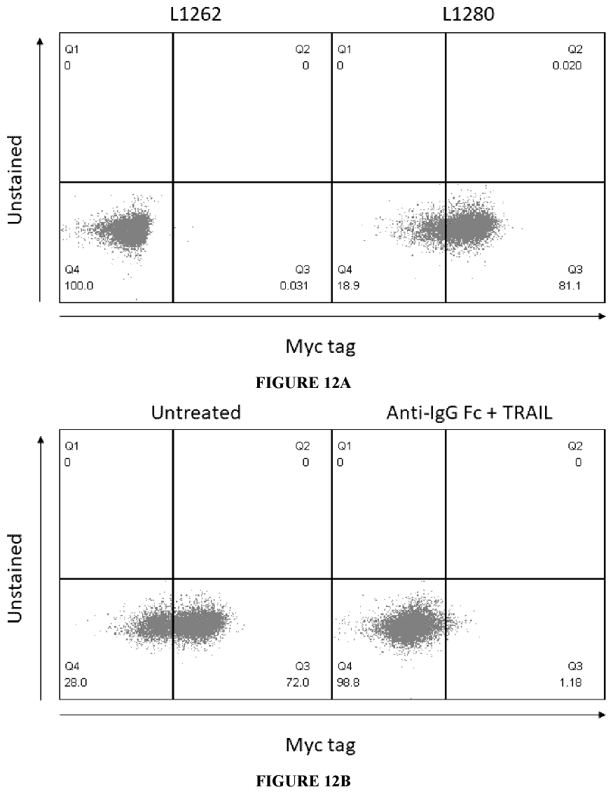

In another example of chimeric receptor mediated negative selection, an enrichment experiment was carried out. L1262 is a cell line clone derived from the same pool as L1231 and has similar positive and negative selection properties. A plasmid expression construct encoding an IgG(unknown specificity)-TNFR1 chimeric receptor (construct C638; see FIGS. 5A and 5B) was introduced into L1262 cells by Cre-mediated integration (as previously described) to generate line L1280 (see Table 6, above). This line expresses a Myc tag on the cell surface and thus can be easily distinguished from the parent line by staining for Myc expression with a monoclonal anti-Myc tag antibody linked to Alexa 647 (see FIG. 12A).

To demonstrate enrichment of a non-signaling cell line over a signaling cell line using chimeric receptor mediated negative selection, an excess of L1280 cells was mixed with L1262 cells. The mix was either untreated or treated with 1 μg/ml anti-human IgG Fc (to activate chimeric receptor signaling) and 20 ng/ml TRAIL (to activate TRAILR1-mediated apoptosis). Since L1280 cells express an IgG-TNFR1 chimeric receptor, treatment with anti-human IgG Fc should upregulate TRAILR1 expression and make the cells sensitive to TRAIL-mediated apoptosis. L1262 cells do not express an IgG-TNFR1 chimeric receptor and thus should not upregulate TRAILR1 in response to anti-human IgG Fc treatment or become sensitive to TRAIL-mediated apoptosis. Following 8 days in culture, the treated and untreated cell line mixes were stained for Myc tag expression. As shown in FIG. 12B, the L1262/L1280 mix treated with anti-IgG and TRAIL (FIG. 12B, right pane) was substantially depleted of Myc positive cells relative to the untreated mix (FIG. 12B, left pane). These results indicate that L1280 cells undergoing chimeric receptor signaling can be effectively depleted from a culture mixed with non-signaling cells using TRAIL/TRAILR1-mediated negative selection.

### Example 8: Generation of Chimeric Receptors with Signaling Portions from Death Receptors Other than TNFR1 (TRAILR1 & TRAILR2)

In addition to TNFR1, other death receptors in the TNFR superfamily can serve as the signaling portion of a chimeric receptor. In this example, chimeric receptors are made in which TRAILR1 and TRAILR2 are substituted for TNFR1 used in previous examples.

Additional plasmid constructs and cell lines referenced below are summarized in Table 9.

TABLE 9
Summary of plasmids & cell lines

| Plasmid/ | |
|---|---|
| Cell Line | Description |
| T99 | Expression vector encoding an IgG(anti-CD3)-TRAILR1 |
| | chimeric receptor with a TK-tet promoter (FIGS. 13A |
| | and 13B) |
| T100 | Expression vector encoding an IgG(anti-CD3)-TRAILR2 |
| | chimeric receptor with a TK-tet promoter (FIGS. 14A |
| | and 14B) |
| L1294 | NF-κB responsive CD19/Puro/TRAILR1 reporter line |
| | with an IgG(anti-CD3)-TRAILR1 chimeric receptor |
| | derived from cell line L1231 and Cre-integrated T99 |
| L1295 | NF-κB responsive CD19/Puro/TRAILR2 reporter line |
| | with an IgG(anti-CD3)-TRAILR2 chimeric receptor |
| | derived from cell line L1231 and Cre-integrated T100 |

Expression vectors encoding IgG(anti-CD3)-TRAILR1 (assigned the name T99) or IgG(anti-CD3)-TRAILR2 (assigned the name T100) were constructed and introduced into the NF-κB reporter line L1231 using Cre-mediated integration. The resulting stable cell lines were assigned the names L1294 (IgG(anti-CD3)-TRAILR1) and L1295 (IgG(anti-CD3)-TRAILR2).

To test signaling, each stable line was used to seed a 24-well tissue culture treated plate with approximately 400,000 cells in the presence of 2 μM Z-VAD-FMK (pan caspase inhibitor). Wells were either left untreated or treated with 1 μg/ml anti-human IgG Fc or approximately 300,000 Jurkat cells (CD3 positive). The next day, cells were stained for CD19 expression and FLAG expression (biosensor lines are FLAG tag positive, Jurkat cells are FLAG tag negative) and then analyzed by flow cytometry. The fraction of FLAG positive cells expressing CD19 was determined for each condition. As shown in Table 10, both of L1294 and L1295 lines upregulated CD19 expression when treated with anti-human IgG Fc or Jurkat cells indicating that chimeric receptors with signaling portions from TRAILR1 or TRAILR2 are functional at signaling NF-κB in response to substrate binding (receptor crosslinking).

TABLE 10
Expression of CD19 reporter in cell lines L1294 and
L1295 when cross-linked with anti-human IgG Fc antibody
or CD3 antigen (expressed by Jurkat cells)

| Cell | Chimeric | Binding Portion | Un- | Anti- | CD3 |
|---|---|---|---|---|---|
| line | Receptor | Specificity | treated | IgG Fc | (Jurkat) |
| L1294 | IgG-TRAILR1 | CD3 | 7.6% | 36% | 32% |
| L1295 | IgG-TRAILR2 | CD3 | 6.8% | 74% | 62% |

### Example 9: A Chimeric Receptor with a Signaling Portion from a Non-Death Receptor TNFR Superfamily Member (CD27)

To show that signaling portions from non-death receptor TNFRSF members may be used in chimeric receptors, biosensors and methods disclosed herein, an IgG-CD27 chimeric receptor was tested. CD27 is a member of the TNFR superfamily, but unlike TNFR1, TRAILR1 and TRAILR2, it is not a death receptor and does not have an intracellular death domain.

Additional plasmid constructs and cell lines referenced below are summarized in Table 11.

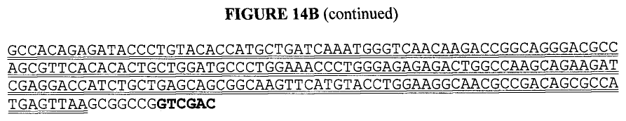

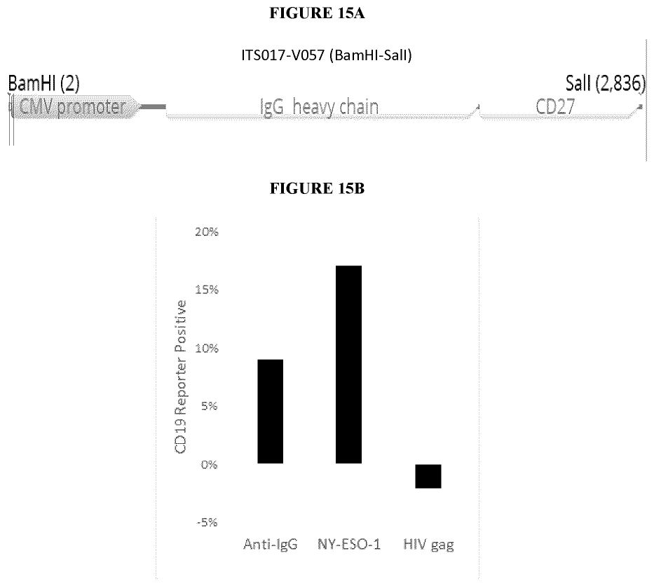

TABLE 11
Summary of plasmids & cell lines

| Plasmid/ | |
|---|---|
| Cell Line | Description |
| ITS017-V057 | Expression vector encoding an |
| | IgG(anti-HLA-A*02: 01-restricted |
| | NY-ESO-1 (SLLMWITQC) antigenic |
| | peptide)-CD27 chimeric receptor with a |
| | CMV promoter (FIG. 15A) |
| ITS017-L021 | NF-κB responsive CD19 reporter line |
| | with an IgG(anti-HLA-A*02: 01- |
| | restriced NY-ESO-1 (SLLMWITQC) anti- |
| | genic peptide)-CD27 chimeric receptor |
| | derived from cell line L998. 1 and |
| | Cre-integrated ITS017-V057 |

An expression vector encoding an IgG-CD27 chimeric receptor is shown in FIG. 15A. The antibody variable region binds to HLA-A*02:01-restricted NY-ESO-1 (SLLMWITQC) antigenic peptide but not to HLA-A*02:01-restricted HIV gag (SLYNTVATL) antigenic peptide (both purchased from Proimmune Inc. (Sarasota Fla., USA)). Expression of the chimeric receptor is driven by a CMV promoter. The construct was introduced into the NF-κB reporter line L998.1 using Cre-mediated integration (as previously described) and the resulting stable cell line was assigned the name ITS017-L021.

ITS017-L021 cells were seeded at approximated 300,000 cells per well in a 24-well tissue culture treated plate. Wells were either left untreated or treated with 1 μg/ml anti-human IgG Fc, 0.5 μg/ml biotin-labeled ProS MHC Class 1 Pentamers (HLA-A*02:01, SLLMWITQC (NY-ESO-1)) with 1 μg/ml streptavidin or 0.5 μg/ml biotin-labeled ProS MHC Class 1 Pentamers (HLA-A*02:01, SLYNTVATL (HIV gag)) with 1 μg/ml streptavidin. Streptavidin, a tetramer, bind to biotin and was added to generate large multivalent binding substrates by complexing with the biotin-labeled ProS MHC Class 1 Pentamers. The next day, cells were stained for CD19 expression and then analyzed by flow cytometry (as previously described). As shown in FIG. 15B and Table 12, treatment with anti-human IgG Fc or the biotin-labeled NY-ESO-1 MHC/Streptavidin complex upregulated CD19 expression relative to the untreated sample. In contrast, no upregulation was observed with the biotin labeled HIV gag MHC/Streptavidin complex which served as a negative control. These results indicate that a signaling portion from a non-death receptor members of the TNFRSF (such as CD27) is functional in chimeric receptors at signaling NF-κB in response to substrate binding (receptor crosslinking).

TABLE 12
Expression of reporter in cell line ITS017-L021 when treated with
anti-human IgG Fc, biotin-labeled NY-ESO-1 MHC/Steptavidin complex
or biotin-labeled HIV gag MHC/Streptavidin complex

| | | Binding | | Anti- | NY-ESO-1 | HIV gag MHC/ |
|---|---|---|---|---|---|---|
| Cell | Chimeric | Portion | | human | Steptavidin | Streptavidin |
| line | Receptor | Specificity | Untreated | IgG Fc | Complex | Complex |
| ITS017- | IgG- | HLA-A*02: 01 | 29% | 38% | 46% | 27% |
| L021 | CD27 | restricted NY- | | | | |
| | | ESO-1 | | | | |
| | | (SLLMWITQC) | | | | |
| | | antigenic | | | | |
| | | peptide | | | | |

### Example 10: Chimeric Receptors with Binding Portions Derived from Non-IgG Proteins

In the examples above, chimeric receptors comprising an IgG binding portion and one of several signaling portions from the TNFR superfamily were shown to function as biosensors in combination with an NF-κB responsive reporter line. To show that binding portions from proteins other than IgG may be used in chimeric receptors, biosensors and methods disclosed herein, chimeric receptors comprising IL-8 and CD73 were tested in this example.

Additional plasmid constructs and cell lines referenced below are summarized in Table 13.

TABLE 13
Summary of plasmids & cell lines

| Plasmid/ | |
|---|---|
| Cell Line | Description |
| C58 | Expression vector encoding the heavy chain and light chain sequence of a membrane |
| | anchored antibody of unknown specificity (FIGS. 18A and 18B) |
| C962 | Expression vector encoding the heavy chain and light chain sequence of a membrane |
| | anchored antibody specific for CD73 (light chain and heavy chain sequences derived from |
| | patent Pub. WO 2016/081748 A2, specifically SEQ ID NOs: 12 and 133, |
| | respectively) |
| T96 | Expression vector encoding an IL8-TNFR1 chimeric receptor with a TK-tet promoter |
| | (FIGS. 16A and 16B) |
| T101 | Expression vector encoding a CD73-TNFR1 chimeric receptor with a TK-tet promoter |
| | (FIG. 17A and 17B) |
| T117 | Expression vector encoding the heavy chain for a membrane anchored antibody specific |
| | for IL-8 (derived from K4.3 in U.S. patent Pub. 2008/0098490 A1) |
| V27 | Expression vector encoding the light chain sequence for a membrane anchored antibody |
| | specific for IL-8 (derived from K4.3 in U.S. patent Pub. 2008/0098490 A1) |
| L1288 | NF-κB responsive CD19/Puro/TRAILR1 reporter line with a CD73-TNFR1 chimeric |
| | receptor derived from cell line L1231 and Cre-integrated T101 |
| L1291 | NF-κB responsive CD19/Puro/TRAILR1 reporter line with an IL8-TNFR1 chimeric receptor |
| | derived from cell line L1231 and Cre-integrated T96 |

IL-8 is a CXC chemokine secreted by macrophages. CD73 is an enzyme that converts adenosine monophosphate to adenosine and is linked to the outer surface of the plasma membrane by a glycosyl phosphatidyl inositol anchor. Plasmid constructs encoding IL8-TNFR1 (assigned the name T96) or CD73(no anchor residues)-TNFR1 (assigned the name T101) were constructed and introduced into the NF-κB responsive reporter line L1231 by Cre-mediated integration (as previously described). The resulting cell lines were assigned the names L1288 (CD73-TNFR1) and L1291 (IL8-TNFR1).

L1288 and L1291 cells were seeded in a 24-well tissue culture treated plate at approximately 400,000 cells per well. The next day, wells were transfected with plasmids that express membrane anchored antibodies of (a) unknown specificity (construct C58), (b) IL-8 specificity (co-transfection of T117 and V27) or (c) CD73 specificity (C962). For each condition, 1.6 μg of polyethyleneimine (PEI) was diluted in Pro293S medium to a final volume of 25 μl followed by a 5-minute incubation. In addition, 400 ng of plasmid DNA (or 200 ng plasmid DNA per construct for co-transfections) was diluted in Pro293S to a final volume of 25 ill. The PEI and plasmid DNA samples were then mixed followed by a 20-minute incubation at room temperature. The mixed sample was then added to the L1188 and L1291 cells. The next day, cells were stained for CD19 expression and analyzed by flow cytometry.

As shown in Table 14, for the CD73-TNFR1 line, upregulation of CD19 expression was observed when cells were transfected with plasmid DNA encoding the CD73-specific antibody but not when transfected with plasmid DNA encoding the IL-8-specific antibody or the unknown specificity antibody. In contrast, for the IL8-TNFR1 line, upregulation of CD19 expression was observed when cells were transfected with plasmid DNA encoding the IL-8 specific antibody but not when transfected with plasmid DNA encoding the other antibodies. These results show that both IL8-TNFR1 and CD73-TNFR1 are functional in chimeric receptors and indicate that binding portions derived from a broad range of proteins are functional in chimeric receptors (or other biosensor receptors) at signaling NF-κB in response to substrate binding (receptor crosslinking).

TABLE 14
Expression of CD19 reporter in cell lines L1288 and L1291 when transfected
with various membrane anchored antibody expression constructs

| | | Transfection with | Transfection with | Transfection with |
|---|---|---|---|---|
| | | plasmid encoding an | plasmid encoding | plasmids encoding |
| | | antibody of unknown | an antibody | an antibody |
| Cell | Chimeric | specificity | specific for CD73 | specific for IL-8 |
| line | Receptor | (C58) | (C962) | (T117 with V27) |
| L1288 | CD73-TNFR1 | 1.5% | 28% | 1.6% |
| L1291 | IL8-TNFR1 | 1.0% | 1.8% | 13.4% |

### Example 11: Chimeric Receptor with a Binding Portion Derived from a Non-IgG Protein Linked to a TNFR1 Deletion Construct Lacking the Extracellular Domain

In Example 6, it was shown that a binding portion derived from IgG and a signaling portion derived from TNFR1 lacking the extracellular domain (ECD) was functional as a chimeric receptor. Chimeric receptors with binding portions derived from non-IgG proteins fused to TNFR1 lacking the extracellular domain are also functional.

Additional plasmid constructs and cell lines referenced below are summarized in Table 15.

TABLE 15
Summary of plasmids & cell lines

| Plasmid/ | |
|---|---|
| Cell Line | Description |
| T145 | Expression vector encoding a CD73(no anchor)-TNFR1(no |
| | ECD) chimeric receptor with a TK-tet promoter (FIGS. |
| | 19A and 19B) |
| L1326 | NF-κB responsive CD19/Puro/TRAILR1 reporter line |
| | with a CD73(no anchor)-TNFR1 (no ECD) chimeric receptor |
| | derived from cell line L1231 and Cre-integrated T145 |

An expression vector encoding a CD73(no anchor)-TNFR1(no ECD) chimeric receptor was constructed (assigned the name T145; Table 15) and introduced into the NF-κB responsive reporter line L1231 by Cre-mediated integration (as previously described). The resulting cell line was assigned the name L1326.

L1326 cells were seeded in a 24-well tissue culture treated plate at approximately 400,000 cells per well. The next day wells were either untreated or transfected with membrane anchored antibody expression constructs of either unknown specificity (construct C58) or CD73 specificity (C962). The next day, cells were stained for CD19 expression and analyzed by flow cytometry. As shown in Table 16, relative to the untreated control, strong upregulation of CD19 expression was observed in the sample transfected with the plasmid DNA encoding the CD73-specific antibody but not the sample transfected with plasmid DNA encoding the antibody of unknown specificity.

TABLE 16
Expression of CD19 reporter in cell line L1326 when transfected
with various membrane anchored antibody expression constructs

| | | | Transfection with | Transfection with |
|---|---|---|---|---|
| | | | plasmid encoding | plasmid encoding |
| | | | antibody of un- | antibody specific |
| Cell | Chimeric | Un- | known specificity | for CD73 |
| line | Receptor | treated | (C58) | (C962) |
| L1326 | CD73-trun- | 0.91% | 0.48% | 39% |
| | cated | | | |
| | TNFR1 (no | | | |
| | extracellular | | | |
| | domain) | | | |

In addition, a cell line was tested that expressed a chimeric receptor comprising Her2 ECD linked to TNFR1 lacking its extracellular domain. As observed with L1326, expression of reporter was observed in cells transfected with plasmid DNA encoding a HER2 ECD-specific antibody but not in cells transfected with plasmid DNA encoding an antibody of unknown specificity (data not shown). As such, this result demonstrates chimeric receptors with binding portions derived from HER2 are also functional.

These results, in combination with those presented in Example 6, provide examples of functional chimeric receptors with diverse binding portions that do not require the TNFR1 extracellular domain to form functional chimeric receptors (or other biosensor receptors) which will signal NF-κB in response to substrate binding (receptor crosslinking). That chimeric receptors with binding portions derived from a diverse set of proteins (IgG, CD73, IL8 and HER2) are all functional suggests that other binding portions, such as those derived from peptides or peptide/MHC complexes would also be functional.

### Example 12: Chimeric Receptors with a Transmembrane Domain not Derived from TNFR Superfamily Members

In the examples described above, all chimeric receptors utilize a transmembrane domain and a signaling portion derived from a TNFR superfamily member. To show that receptors with a transmembrane domain that is derived from proteins outside the TNFR superfamily are also functional, a construct was tested that substituted the transmembrane domain of TNFR1 with a CD4 or PDGFR transmembrane domain. A heterologous transmembrane domain was therefore placed between the TNFR1 extracellular domain (ECD) and intracellular domain (ICD).

Additional plasmid constructs and cell lines referenced below are summarized in Table 17.

TABLE 17
Summary of plasmids & cell lines

| Plasmid/ | |
|---|---|
| Cell Line | Description |
| T110 | Expression vector encoding an IgG(anti-CD3)-TNFR1 chimeric receptor with a CD4 |
| | transmembrane domain, driven by a TK-tet promoter (FIGS. 20A and 20B) |
| T111 | Expression vector encoding an IgG(anti-CD3)-TNFR1 chimeric receptor with a PDGFR |
| | transmembrane domain, driven by a TK-tet promoter (FIGS. 21A and 21B) |
| L1298 | NF-κB responsive CD19/Puro/TRAILR1 reporter line with an IgG(anti-CD3)-TNFR1 |
| | chimeric receptor with a CD4 transmembrane domain derived from cell line L1231 and |
| | Cre-integrated T110 |
| L1299 | NF-κB responsive CD19/Puro/TRAILR1 reporter line with an IgG(anti-CD3)-TNFR1 |
| | chimeric receptor with a PDGFR transmembrane domain derived from cell line L1231 and |
| | Cre-integrated T111 |

Expression vectors encoding an IgG(anti-CD3)-TNFR1 chimeric receptor with a transmembrane domain derived from CD4 (assigned the name T110; Table 17) or from PDGFR (assigned the name T111; Table 17) were constructed and introduced into the NF-κB responsive reporter line L1231 by Cre-mediated integration (as previously described). The resulting cell lines were assigned the names L1298 (CD4 transmembrane domain; Table 17) or L1299 (PDGFR transmembrane domain; Table 17).

To test signaling, each stable line was used to seed a 24-well tissue culture treated plate with approximately 400,000 cells in the presence of 2 μM Z-VAD-FMK (pan caspase inhibitor). Wells were either left untreated or treated with 1 μg/ml anti-human IgG Fc or approximately 300,000 Jurkat cells (CD3 positive). The next day, cells were stained for CD19 expression and FLAG expression (biosensor lines are FLAG tag positive, Jurkat cells are FLAG tag negative) and then analyzed by flow cytometry. The fraction of FLAG positive cells expressing CD19 was determined for each condition. As shown in Table 18, both L1298 and L1299 upregulated CD19 expression when treated with anti-human IgG Fc or Jurkat cells indicating that chimeric receptors with transmembrane domains derived from proteins outside the TNFR superfamily form functional chimeric receptors which will signal NF-κB in response to substrate binding (receptor crosslinking).

TABLE 18
Expression of CD19 reporter in cell lines L1298 and L1299 when
treated with anti-human IgG Fc or CD3 positive Jurkat cells

| | | Binding | | Anti- | |
|---|---|---|---|---|---|
| Cell | Chimeric | Portion | Un- | human | CD3 |
| line | Receptor | Specificity | treated | IgG Fc | (Jurkat) |
| L1298 | IgG-TNFR1 with a | CD3 | 5.2% | 82% | 70% |
| | transmembrane | | | | |
| | domain derived | | | | |
| | from CD4 | | | | |
| L1299 | IgG-TNFR1 with a | CD3 | 2.0% | 73% | 63% |
| | transmembrane | | | | |
| | domain derived | | | | |
| | from PDGFR | | | | |

### Example 13: Chimeric Receptors Lacking Extracellular and Transmembrane Domains of a TNFR Superfamily Member

In the examples presented above, chimeric receptors have included the cytoplasmic domain (intracellular domain or ICD) of a TNFR superfamily member in combination with either a transmembrane Domain™, an extracellular domain (ECD) or both a TM and an ECD derived from the same TNFR superfamily member. To show that receptors with an ECD and TM derived from proteins outside the TNFR superfamily are also functional, this example describes constructs that substitute both the ECD and TM of TNFR1 or TRAILR2 with heterologous domains

Additional plasmid constructs and cell lines referenced below are summarized in Table 19.

TABLE 19
Summary of plasmids & cell lines

| Plasmid/ | |
|---|---|
| Cell Line | Description |
| T146 | Expression vector encoding a CD73(no anchor)-PDGFR(TM)-TNFR1(ICD) chimeric |
| | receptor with a TK-tet promoter (FIGS. 22A and 22B) |
| T147 | Expression vector encoding a CD73(no anchor)-PDGFR(TM)-TRAILR2(ICD) chimeric |
| | receptor with a TK-tet promoter (FIGS. 23A and 23B) |
| T173 | Expression vector encoding a GLP1R(no ICD)-TNFR1(ICD) chimeric receptor with a TK- |
| | tet promoter (FIGS. 24A and 24B) |
| T175 | Expression vector encoding a CD20(no C-terminal ICD)-TNFR1(ICD) chimeric receptor |
| | with a TK-tet promoter (FIGS. 25A, 25B and 25C) |
| E485 | Expression vector encoding the heavy chain and light chain sequence of a membrane |
| | anchored antibody specific for CD20 (derived from antibody 1.5.3, 1.5.3, ATCC No. PTA- |
| | 7330, see patent U.S. 2007/0014720 A1) |
| ITS007- | Expression vector encoding the heavy chain sequence for a membrane anchored |
| V024 | antibody specific for GLP1R (described in Table 5 of U.S. patent Pub. No. |
| | 2015/0240243 A1, specifically anti-GLP1R clone 9, SEQ ID NO: 80 therein) |
| L1330 | NF-κB responsive CD19/Puro/TRAILR1 reporter line with a CD73(no anchor)- |
| | PDGFR(TM)-TNFR1(ICD) chimeric receptor derived from cell line L1231 and Cre- |
| | integrated T146 |
| L1332 | NF-κB responsive CD19/Puro/TRAILR1 reporter line with a CD73(no anchor)- |
| | PDGFR(TM)-TRAILR2(ICD) chimeric receptor derived from cell line L1231 and Cre- |
| | integrated T147 |
| L1348 | NF-κB responsive CD19/Puro/TRAILR1 reporter line with a GLP1R(no ICD)-TNFR1(ICD) |
| | chimeric receptor derived from cell line L1231 and Cre-integrated T173 |
| L1350 | NF-κB responsive CD19/Puro/TRAILR1 reporter line with a CD20(no C-terminal ICD)- |
| | TNFR1(ICD) chimeric receptor derived from cell line L1231 and Cre-integrated T175 |

Expression vectors encoding CD73(no anchor)-PDGFR(TM)-TNFR1(ICD) (assigned the name T146; Table 19) and CD73(no anchor)-PDGFR(TM)-TRAILR2(ICD), respectively (assigned the name T147; Table 19) were constructed and introduced into the NF-κB responsive reporter line L1231 by Cre-mediated integration (as previously described). The resulting cell lines were assigned the names L1330 and L1332 for T146 and T147, respectively (Table 19). Likewise, expression vectors encoding GLPR1 truncated after the final TM helix and fused to the ICD of TNFR1 (assigned the name T173; Table 19) and CD20 truncated after the final TM helix and fused to the ICD of TNFR1 (assigned the name T175; Table 19) are constructed and introduced into the NF-κB responsive reporter line L1231 by Cre-mediated integration (as previously described). The resulting cell lines are assigned the names L1348 and L1350 for T173 (GLPR1(no ICD)-TNFR1(ICD)) and T175 (CD20(no C-terminal ICD)-TNFR1(ICD)), respectively (Table 19).

L1330 and L1332 cells were seeded in a 24-well tissue culture treated plate at approximately 400,000 cells per well. The next day wells were either untreated or transfected with membrane anchored antibody expression constructs of either unknown specificity (C58; previously described) or CD73 specificity (C962; previously described). The next day, cells were stained for CD19 expression and analyzed by flow cytometry (as previously described). As shown in Table 20, relative to the untreated controls, both cell lines showed strong upregulation of CD19 expression in samples transfected with plasmid DNA encoding the CD73-specific antibody, but not in samples transfected with plasmid DNA encoding the antibody of unknown specificity. This indicates that the part of a TNFR superfamily member required to generate a functional chimeric receptor (or other biosensor receptor) is the signaling portion.

Likewise, L1348 and L1350 cells are seeded in a 24-well tissue culture treated plate at approximately 400,000 cells per well. The next day wells are either untreated or transfected with plasmid DNA encoding membrane anchored antibodies of unknown specificity (C58, previously described), GLP1R specificity (co-transfection with ITS007-V024, Table 19, and an expression vector encoding the same light chain as in plasmid C639) or CD20 specificity (E485; Table 19). The next day, cells are stained for CD19 expression and analyzed by flow cytometry (as previously described). Relative to the untreated control, upregulation of CD19 expression is observed when the GLP1R(no ICD)-TNFR1(ICD) line (i.e. L1348) is transfected with plasmid DNA encoding the GLP1R-specific antibody, but not when transfected with plasmid DNA encoding the CD20-specific antibody or the antibody of unknown specificity. Upregulation of CD19 expression is also observed when the CD20(no C-terminal ICD)-TNFR1(ICD) line (i.e. L1350) is transfected with plasmid DNA encoding the CD20-specific antibody, but not when transfected with plasmid DNA encoding the GLP1R-specific antibody or the antibody of unknown specificity.

TABLE 20
Expression of CD19 reporter in cell lines L1330
and L1332 when transfected with various membrane
anchored antibody expression constructs

| | | | Transfection with | Transfection |
|---|---|---|---|---|
| | | | plasmid encoding | with plasmid |
| | | | an antibody | encoding an |
| | | | of unknown | antibody spe- |
| Cell | Chimeric | Un- | specificity | cific for CD73 |
| line | Receptor | treated | (C58) | (C962) |
| L1330 | CD73(no | 1.4% | 2.7% | 46.4% |
| | anchor)- | | | |
| | PDGFR(TM)- | | | |
| | TNFR1(ICD) | | | |
| L1332 | CD73(no | 0.7% | 1.6% | 41.4% |
| | anchor)- | | | |
| | PDGFR(TM)- | | | |
| | TRAILR2(ICD) | | | |

All citations are hereby incorporated by reference, along with all citations cited in these references.

The scope of the invention as defined by the attached claims should not be limited by the specific embodiments set forth in the examples, but should be given the broadest interpretation consistent with the specification as a whole.

1. A library of biosensor cells comprising a plurality of at least 1000 unique biosensor cells which collectively bind a plurality of unknown binding substrates, each unique biosensor cell being a eukaryotic host cell comprising a chimeric receptor;
wherein the chimeric receptor comprises:
a signaling portion comprising a tumor necrosis factor receptor superfamily (TNFRSF) member or a fragment of the TNFRSF member which retains an intracellular signaling domain of the TNFRSF member;
a transmembrane domain; and
a binding portion comprising an extracellular binding site which has unique binding specificity compared to other chimeric receptors in the plurality of unique biosensor cells, wherein the binding portion comprises a monobody, an affibody, an anticalin, a DARPin, a Kunitz domain, an avimer, a soluble T-cell receptor (TCR), an antibody or an antigen-binding fragment of the antibody, or wherein the chimeric receptor comprises, and the binding portion is comprised within, a TCR or an antigen-binding fragment of the TCR;
wherein the eukaryotic host cell comprises at least one nucleic acid comprising one or more coding sequences which collectively encode the chimeric receptor, the one or more coding sequences operably linked to at least one promoter; and

2. (canceled)

3. (canceled)

4. (canceled)

5. (canceled)

6. (canceled)

7. (canceled)

8. The library of biosensor cells according to claim 1, wherein the TNFRSF member has at least 80% sequence identity to TNFR1, FAS, TRAILR1, TRAILR2, TRAMP, CD358 or CD27 and retains functional membrane localization and TNFRSF intracellular signaling activity when expressed in the eukaryotic cell.

9. (canceled)

10. The library of biosensor cells according to claim 1, wherein the binding portion comprises an antibody or an antigen-binding fragment of the antibody.

11. (canceled)

12. (canceled)

13. (canceled)

14. The library of biosensor cells according to claim 1, wherein the one or more coding sequences comprise or are operably linked to one or more genetic elements which cause expression of the chimeric receptor at a level that is sufficiently low such that signaling caused by specific binding of a binding substrate to the binding portion of the chimeric receptor is distinguishable over background signaling in the absence of the binding substrate.

15. The library of biosensor cells according to claim 14, wherein the one or more genetic elements comprise:
a Kozak sequence in the nucleic acid which causes inefficient translation of the chimeric receptor;
codons in the at least one coding sequence which are not optimized for efficient translation in the eukaryotic host cell;
one or more RNA destabilizing sequences in the nucleic acid for reducing the half-life of an RNA transcribed from the nucleic acid which encodes the chimeric receptor;
intron and/or exon sequences in the one or more coding sequences which cause inefficient intron splicing;
the chimeric receptor encoded by the at least one nucleic acid further comprises one or more ubiquination sequences; or
a combination thereof.

16. The library of biosensor cells according to claim 1, wherein the at least one promoter comprises one or both of a weak promoter and an inducible promoter.

17. The library of biosensor cells according to claim 16, wherein the inducible promoter is a tetracycline-regulated promoter.

18. (canceled)

19. The library of biosensor cells according to claim 1, wherein the at least one promoter comprises an inducible promoter, and wherein the eukaryotic host cell expresses a repressor which binds an operator of the inducible promoter.

20. The library of biosensor cells according to claim 1, wherein the at least one nucleic acid further comprises at least one nucleic acid sequence encoding antisense RNA or RNAi configured to reduce expression levels of the chimeric receptor.

21. (canceled)

22. (canceled)

23. (canceled)

24. (canceled)

25. (canceled)

26. (canceled)

27. The library of biosensor cells according to claim 1, wherein the marker gene is operably linked to a second promoter and a NF-κB response element such that expression of the marker gene is activated by NF-κB binding the NF-κB response element and inactive or repressed in the absence of said NF-κB binding.

28. The library of biosensor cells according to claim 1, wherein the one or more genes of interest comprises two or more genes of interest, and wherein:
at least two of the two or more genes of interest are in a polycistronic operon that is operably linked to a second promoter and a NF-κB response element; and/or
at least two of the two or more genes of interest are in separate operons that are operably linked to two or more additional promoters and NF-κB response elements;
such that expression of the two or more genes of interest is activated by NF-κB binding the NF-κB response element and inactive or repressed in the absence of said NF-κB binding.

29. The library of biosensor cells according to claim 1, further comprising an expression cassette configured to express a cell surface protein which comprises an extracellular domain that comprises: a multivalent binding substrate; or a univalent binding substrate that forms the multivalent binding substrate through multimerization of the cell surface protein.

30. The library of biosensor cells according to claim 29, wherein the expression cassette configured to express the cell surface protein comprises an inducible promoter operably linked to a nucleic acid sequence or sequences encoding the cell surface protein.

31. (canceled)

32. (canceled)

33. (canceled)

34. (canceled)

35. (canceled)

36. A method of detecting binding between a biosensor and a multivalent binding substrate, the method comprising:
contacting the library of biosensor cells according to claim 1 with the multivalent binding substrate,
wherein binding of the multivalent binding substrate to the extracellular binding site of a chimeric receptor of a biosensor cell in the library activates intracellular signaling activity of the signaling portion of the chimeric receptor; and
identifying binding between the biosensor and the multivalent binding substrate based on a level of the intracellular signaling activity compared with a background level, wherein the level of the intracellular signaling activity positively corresponds to an expression level of a marker that is expressed from, or caused to be expressed as a result of expression of, the one or more genes of interest, the marker being one or more of a screenable marker, a selectable marker or a screenable-selectable marker.

37. The method of claim 36, wherein the marker is the selectable marker or the screenable-selectable marker and the level of the intracellular signaling activity positively corresponds to a measure of cell death of the biosensor or positively corresponds to a measure of cell survival of the biosensor.

38. (canceled)

39. The method of claim 37, wherein the marker gene is a death receptor that is activated by a ligand that does not activate other death receptors expressed by the biosensor cell if the other death receptors are present, and wherein the method further comprises contacting the library with the ligand.

40. The method of claim 36, wherein the TNFRSF member is a death receptor, and wherein the method further comprises contacting the biosensor with a caspase inhibitor prior to or during said contacting the library with the multivalent binding substrate.

41. (canceled)

42. The method of claim 36, further comprising preparing the multivalent binding substrate prior to said contacting the biosensor with the multivalent binding substrate by oligomerizing a binding substrate.

43. A method of detecting binding between a biosensor and a multivalent binding substrate, the method comprising:
contacting the biosensor with the multivalent binding substrate, the biosensor comprising a first vertebrate cell that expresses a chimeric protein, wherein the chimeric protein comprises:
a signaling portion comprising a transmembrane tumor necrosis factor receptor superfamily (TNFRSF) member or a fragment of the TNFRSF member which retains an intracellular signaling domain of the TNFRSF member;
a transmembrane domain; and
a binding portion comprising an extracellular binding site which specifically binds a binding substrate, wherein the extracellular binding site is not native to the TNFRSF member;
wherein said contacting the biosensor with the multivalent binding substrate comprises co-expressing a cell surface protein in the first vertebrate cell with the chimeric protein, the cell surface protein comprising an extracellular domain comprising: the multivalent binding substrate; or a univalent binding substrate that forms the multivalent binding substrate through multimerization of the cell surface protein; and
wherein binding of the multivalent binding substrate to the extracellular binding site activates intracellular signaling activity of the signaling portion; and
identifying binding between the biosensor and the multivalent binding substrate based on a level of the intracellular signaling activity compared with a background level.

44. The method of claim 43, wherein the level of the intracellular signaling activity positively corresponds to a measure of cell death of the biosensor or positively corresponds to a measure of cell survival of the biosensor.

45. (canceled)

46. The method of claim 43, wherein the level of the intracellular signaling activity positively corresponds to an expression level of a marker gene that is activated by NF-κB, the marker gene being a one or more of a screenable marker gene, a selectable marker gene or a screenable-selectable marker gene.

47. The method of claim 46, wherein the marker gene is a death receptor that is activated by a ligand that does not activate other death receptors expressed by the first vertebrate cell if the other death receptors are present, and wherein the method further comprises contacting the biosensor with the ligand.

48. The method of claim 43, wherein the TNFRSF member is a death receptor, and
wherein the method further comprises contacting the biosensor with a caspase inhibitor prior to or during said contacting the biosensor with the multivalent binding substrate.

49. (canceled)

50. (canceled)

51. (canceled)

52. A library of biosensor cells comprising a plurality of unique biosensor cells which collectively bind a plurality of unknown binding substrates, each unique biosensor cell being a host cell comprising:
receptor comprising a binding site having unique binding specificity compared to other receptors in the plurality of unique biosensor cells, wherein the receptor is artificial, wherein the receptor signals production of a positive selectable marker and a negative selectable marker in response to the binding site being bound by a specific binding substrate, and wherein the production of the positive selectable marker and/or the negative selectable marker is encoded by at least one selection cassette that is heterologous to the host cell;
wherein the plurality of unique biosensor cells comprises at least 1000, at least 10,000, at least 100,000, at least 1 million, at least 10 million, at least 100, million, at least 1 billion, or at least 10 billion unique biosensor cells.

53. (canceled)

54. (canceled)

55. (canceled)

56. (canceled)

57. The library of claim 52, wherein the positive selectable marker mediates survival of the host cell and/or the negative selectable marker mediates death of the host cell.

58. (canceled)

59. (canceled)

60. (canceled)

61. An in vitro method of identifying a biosensor cell from the library defined in claim 52 that is specifically activated by a target substrate or target substrates, wherein the receptor of each unique biosensor cell signals production of both a positive selectable marker and a negative selectable marker in response to the binding site being bound by the specific binding substrate for that unique biosensor cell, the method comprising:
(a) contacting the library with the target substrate or the target substrates under positive selection conditions;
(b) contacting the library with a control substrate or control substrates under negative selection conditions; and
(c) identifying biosensor cells which survive (a) and (b) as biosensor cells which are specifically activated by the target substrate or the target substrates.

62-63. (canceled)


SEQUENCE LISTING (64 sequences)

SEQ ID NO: 1 - protein, 449 aa, artificial sequence
  Amino acid sequence of heavy chain of antibody- based binding portion of IgG-TNFR1 chimeric receptor which specifically binds human CD3, encoded by plasmid C601
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 451 aa, artificial sequence
  Amino acid sequence of heavy chain of antibody- based binding portion of control IgG-TNFR1 chimeric receptor which has uncharacterized binding specificity, encoded by plasmid C638
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEVQLERLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 448 aa, artificial sequence
  Amino acid sequence of heavy chain of antibody- based binding portion of IgG-TNFR1 chimeric receptor encoded by ITS017-V030/V032/V033/V034/V035 plasmids
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDLLEWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 448 aa, artificial sequence
  Amino acid sequence of heavy chain of antibody- based binding portion of IgG-TNFR1 chimeric receptor encoded by C644 or C645 plasmid
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARELAYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - skipped

SEQ ID NO: 6 - protein, 433 aa, artificial sequence
  Amino acid sequence of full-length TNFR1 in IgG-TNFR1 chimeric receptor ITS017-V030
IYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 7 - protein, 370 aa, artificial sequence
  Amino acid sequence of TNFR1 deletion construct in IgG-TNFR1 chimeric receptor ITS017-V032
ESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 8 - protein, 329 aa, artificial sequence
  Amino acid sequence of TNFR1 deletion construct in IgG-TNFR1 chimeric receptor ITS017-V033
GCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 9 - protein, 288 aa, artificial sequence
  Amino acid sequence of TNFR1 deletion construct in IgG-TNFR1 chimeric receptor ITS017-V034
TCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 10 - protein, 258 aa, artificial sequence
  Amino acid sequence of TNFR1 deletion construct in IgG-TNFR1 chimeric receptor ITS017-V035
PQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 11-12 - skipped (2 entries)

SEQ ID NO: 13 - protein, 902 aa, artificial sequence
  Amino acid sequence of IgG-TNFR1 chimeric receptor ITS017-V030
MEFGLSWLFLVAILKGVQCEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDLLEWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 14 - protein, 839 aa, artificial sequence
  Amino acid sequence of IgG-TNFR1 chimeric receptor ITS017-V032
MEFGLSWLFLVAILKGVQCEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDLLEWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 15 - protein, 798 aa, artificial sequence
  Amino acid sequence of IgG-TNFR1 chimeric receptor ITS017-V033
MEFGLSWLFLVAILKGVQCEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDLLEWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 16 - protein, 757 aa, artificial sequence
  Amino acid sequence of IgG-TNFR1 chimeric receptor ITS017-V034
MEFGLSWLFLVAILKGVQCEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDLLEWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 17 - protein, 727 aa, artificial sequence
  Amino acid sequence of IgG-TNFR1 chimeric receptor ITS017-V035
MEFGLSWLFLVAILKGVQCEVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDLLEWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 18 - protein, 786 aa, artificial sequence
  Amino acid sequence of CD19-Puro fusion protein
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTRGSRHRRRQGGGGSGGGGSGGGSGSGGGSGGGSTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 19 - DNA, 6504 bp, artificial sequence
  Expression vector C659 encoding CD19-Puro fusion protein
ggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgggaatttccggggactttccgggaatttccggggactttccgggaatttccaaatctggcctcggcggccaagcttagacactagagggtatataatggaagctcgacttccagcttggcaatccggtactactagcgccgccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggggatcccggcataggaggcgccaaggaggtggcggatctggagggggaggatctggagggggctcaggatcagggggaggatctggaggcggatcaactgagtacaaacccactgtgaggctcgctactagagatgatgtgcctagagctgtccgaactctggctgctgccttcgccgattaccctgccactcgccataccgtcgatcccgatcgccacattgaacgagtcaccgaactccaggagctgtttctcactagagtcgggctggatattggcaaagtctgggtggccgatgacggagccgctgtcgctgtgtggactacacctgagtctgtggaggctggcgccgtgtttgctgaaattggacctcggatggctgaactgtctggatctcgactggctgcccagcagcagatggagggactgctggcaccccatagaccaaaggaacctgcctggtttctggcaactgtgggagtgtcacccgatcatcagggcaaaggactgggatctgccgtggtgctccctggcgtggaggccgctgaacgagctggcgtccccgcttttctcgaaacttctgccccccgaaatctccctttctacgaacgactgggattcactgtcaccgccgatgtcgaagtgcctgaggggcctagaacatggtgtatgacccggaaacccggagcttaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcacgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgac

SEQ ID NO: 20 - DNA, 6093 bp, artificial sequence
  Expression vector encoding chimeric receptor ITS017-V030
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatccacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagagacaagctggctagcgtacatactgaagcttgccgccaccatggagtttgggctgagctggctttttcttgtggctattttaaaaggtgtccagtgtgaagtgcagctggtggaaagcggcggaggcctggtgaaacctggcggcagcctgagactgagctgcgccgccagcggcttcaccttcagcagctacagcatgaactgggtccgccaggcccctggcaagggactggaatgggtgtcctcgatcagcagcagcagctcctacatctactacgccgacagcgtgaagggccggttcaccatcagccgggacaacgccaagaacagcctgtacctgcagatgaacagcctgcgggccgaggacaccgccgtgtattactgtgcgagagatctcctagaatggtacttcgatctctggggccgtggcaccctggtcactgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaatccggaatctatccaagcggggtgatcggcctggtgcctcacctcggggatcgggaaaaacgcgactcagtatgcccgcaggggaaatatattcaccctcaaaataatagtatttgttgtaccaaatgtcacaaaggcacctacctgtacaatgactgccctgggcccgggcaagataccgactgccgagagtgtgaatccggttcctttaccgccagcgagaaccaccttaggcactgcctttcatgtagcaagtgccgaaaagagatgggacaggtggagatatcttcttgcactgttgatcgggacactgtctgcggatgtcgaaagaatcagtatcgccactattggtcagagaacctcttccagtgctttaattgcagcctctgccttaatggaactgttcacctttcctgccaagagaagcagaacactgtgtgtacctgtcacgctgggttctttcttcgcgagaacgagtgcgtgagctgcagcaattgcaagaagtccctggagtgtacaaaattgtgtttgcctcaaatcgaaaatgtcaagggcacggaggatagcgggaccactgtcctgttgccactggttatcttctttggattgtgcctgctgtcactgttgtttattggcctcatgtatcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggactcgaggagttcgtgacctagtgagacgtcgtgggcgggacgtctctatcgagtgtcgacctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 21 - DNA, 2112 bp, artificial sequence
  Nucleic acid sequence of HER2ECD-PDFR
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgacgcgtgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttag

SEQ ID NO: 22 - protein, 703 aa, artificial sequence
  Amino acid sequence of HER2ECD-PDFR
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTTRAVGQDTQEVIVVPHSLPFKVVVISAILALVVLTIISLIILIMLWQKKPR

SEQ ID NO: 23 - DNA, 11307 bp, artificial sequence
  Nucleic acid sequence of plasmid C601
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatacgaacggtacgaaccggggagtcccttttaggcacttgcttctggtgctgcaactggcgctcctcccagcagccactcagggaaagaaagtggtgctgggcaacagcggcgattacaaggatgacgacgataaggttcggacgggaggtggcgggggttctaattccggagactacaaagacgatgatgacaaggtgggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcataaggagccgccaccatggagtttgggctgagctggctttttcttgtggctattttaaaaggtgtccagtgtgacatcaagctgcagcagtctggcgccgagctggctagacctggcgcctctgtgaagatgagctgcaagaccagcggctacaccttcacccggtacaccatgcactgggtcaagcagaggcctggacagggcctggaatggatcggctacatcaaccccagccggggctacaccaactacaaccagaagttcaaggacaaggccaccctgaccaccgacaagagcagcagcaccgcctacatgcagctgagcagcctgaccagcgaggacagcgccgtgtactactgcgcccggtactacgacgaccactactgcctggactactggggccagggcaccacactgaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaacgcgtatctatccaagcggggtgatcggcctggtgcctcacctcggggatcgggaaaaacgcgactcagtatgcccgcaggggaaatatattcaccctcaaaataatagtatttgttgtaccaaatgtcacaaaggcacctacctgtacaatgactgccctgggcccgggcaagataccgactgccgagagtgtgaatccggttcctttaccgccagcgagaaccaccttaggcactgcctttcatgtagcaagtgccgaaaagagatgggacaggtggagatatcttcttgcactgttgatcgggacactgtctgcggatgtcgaaagaatcagtatcgccactattggtcagagaacctcttccagtgctttaattgcagcctctgccttaatggaactgttcacctttcctgccaagagaagcagaacactgtgtgtacctgtcacgctgggttctttcttcgcgagaacgagtgcgtgagctgcagcaattgcaagaagtccctggagtgtacaaaattgtgtttgcctcaaatcgaaaatgtcaagggcacggaggatagcgggaccactgtcctgttgccactggttatcttctttggattgtgcctgctgtcactgttgtttattggcctcatgtatcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcaattcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggaaagcttgccgccaccatggacccccccagagccagccacctgagcccccggaagaagcggcccagacagacaggcgccctgatggccagcagcccccaggacatcaagttccaggacctggtggtgttcatcctggaaaagaagatgggcaccaccagacgggcctttctgatggaactggccagacggaagggcttccgggtggagaacgagctgtccgacagcgtgacccacatcgtggccgagaacaacagcggcagcgacgtgctcgaatggctgcaggcccagaaagtgcaggtgtccagccagcccgagctgctggacgtgtcctggctgatcgagtgcatcagagccggcaagcccgtggagatgaccggcaagcaccagctggtcgtgcggcgggactacagcgacagcaccaaccccggaccccccaagaccccccctatcgccgtgcagaagatcagccagtacgcctgccagcggcggaccaccctgaacaactgcaaccagattttcaccgacgccttcgacatcctggccgaaaactgcgagttccgggagaacgaggacagctgcgtgaccttcatgagagccgccagcgtgctgaagtccctgcccttcaccatcatcagcatgaaggacaccgagggcatcccttgcctgggcagcaaagtgaagggcatcatcgaggaaatcattgaggacggcgagagcagcgaagtgaaagccgtgctgaacgacgagagataccagagcttcaagctgttcaccagcgtgttcggcgtgggcctgaaaaccagcgagaagtggttccggatgggcttcagaaccctgagcaaagtgcggagcgacaagagccttaagttcacccggatgcagaaggccggcttcctgtactacgaagatctggtgtcctgcgtgaccagagccgaggccgaggccgtgagcgtgctggtgaaagaggccgtctgggccttcctgcccgatgccttcgtgaccatgaccggcggcttcagacggggcaagaaaatgggccacgacgtggactttctgatcaccagccccggcagcaccgaggacgaagaacagctgctgcagaaagtgatgaacctgtgggagaagaagggcctgctgctgtactatgacctggtggagagcaccttcgagaagctgcggctgcccagccggaaggtggacgccctggaccacttccagaagtgctttctgatcttcaagctgcctcggcagagagtggacagcgaccagagcagctggcaggaaggaaagacctggaaggccatcagagtggacctggtgctgtgcccctacgagcggagagccttcgccctgctgggctggaccggcagccggcagttcgagcgggacctgcggagatacgccacccacgagcggaagatgatcctggacaaccacgccctgtacgacaagaccaagcggatcttcctgaaggccgagagcgaggaagaaatcttcgcccacctgggcctggactacatcgagccctgggagcggaacgcctaatctagagagagtttcagctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgggtaccgctagcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggacatgagggtccccgctcagctcctggggctcctgctactctggctccgaggtgccagatgtgacatccagctgacccagagccccgccatcatgtctgctagccctggcgagaaagtgaccatgacctgcagagccagcagcagcgtgtcctacatgaattggtatcagcagaagtccggcaccagccccaagcggtggatctacgacacaagcaaggtggccagcggcgtgccctacagattttctggcagcggctccggcacctcctacagcctgaccatcagcagcatggaagccgaggacgccgccacctactactgccagcagtggtccagcaaccccctgacctttggagccggcaccaagctggaactgaagcggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 24 - DNA, 11316 bp, artificial sequence
  Nucleic acid sequence of plasmid C638
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatacgaacggtacgaaccggggagtcccttttaggcacttgcttctggtgctgcaactggcgctcctcccagcagccactcagggaaagaaagtggtgctgggcaacagcggcgattacaaggatgacgacgataaggttcggacgggaggtggcgggggttctaattccggagactacaaagacgatgatgacaaggtgggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcataaggagccgccaccatggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtgtcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaggtacaactggaacgacttgatgcttttgatatctggggccaagggacaatggtcaccgtgtcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaacgcgtatctatccaagcggggtgatcggcctggtgcctcacctcggggatcgggaaaaacgcgactcagtatgcccgcaggggaaatatattcaccctcaaaataatagtatttgttgtaccaaatgtcacaaaggcacctacctgtacaatgactgccctgggcccgggcaagataccgactgccgagagtgtgaatccggttcctttaccgccagcgagaaccaccttaggcactgcctttcatgtagcaagtgccgaaaagagatgggacaggtggagatatcttcttgcactgttgatcgggacactgtctgcggatgtcgaaagaatcagtatcgccactattggtcagagaacctcttccagtgctttaattgcagcctctgccttaatggaactgttcacctttcctgccaagagaagcagaacactgtgtgtacctgtcacgctgggttctttcttcgcgagaacgagtgcgtgagctgcagcaattgcaagaagtccctggagtgtacaaaattgtgtttgcctcaaatcgaaaatgtcaagggcacggaggatagcgggaccactgtcctgttgccactggttatcttctttggattgtgcctgctgtcactgttgtttattggcctcatgtatcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcaattcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggaaagcttgccgccaccatggacccccccagagccagccacctgagcccccggaagaagcggcccagacagacaggcgccctgatggccagcagcccccaggacatcaagttccaggacctggtggtgttcatcctggaaaagaagatgggcaccaccagacgggcctttctgatggaactggccagacggaagggcttccgggtggagaacgagctgtccgacagcgtgacccacatcgtggccgagaacaacagcggcagcgacgtgctcgaatggctgcaggcccagaaagtgcaggtgtccagccagcccgagctgctggacgtgtcctggctgatcgagtgcatcagagccggcaagcccgtggagatgaccggcaagcaccagctggtcgtgcggcgggactacagcgacagcaccaaccccggaccccccaagaccccccctatcgccgtgcagaagatcagccagtacgcctgccagcggcggaccaccctgaacaactgcaaccagattttcaccgacgccttcgacatcctggccgaaaactgcgagttccgggagaacgaggacagctgcgtgaccttcatgagagccgccagcgtgctgaagtccctgcccttcaccatcatcagcatgaaggacaccgagggcatcccttgcctgggcagcaaagtgaagggcatcatcgaggaaatcattgaggacggcgagagcagcgaagtgaaagccgtgctgaacgacgagagataccagagcttcaagctgttcaccagcgtgttcggcgtgggcctgaaaaccagcgagaagtggttccggatgggcttcagaaccctgagcaaagtgcggagcgacaagagccttaagttcacccggatgcagaaggccggcttcctgtactacgaagatctggtgtcctgcgtgaccagagccgaggccgaggccgtgagcgtgctggtgaaagaggccgtctgggccttcctgcccgatgccttcgtgaccatgaccggcggcttcagacggggcaagaaaatgggccacgacgtggactttctgatcaccagccccggcagcaccgaggacgaagaacagctgctgcagaaagtgatgaacctgtgggagaagaagggcctgctgctgtactatgacctggtggagagcaccttcgagaagctgcggctgcccagccggaaggtggacgccctggaccacttccagaagtgctttctgatcttcaagctgcctcggcagagagtggacagcgaccagagcagctggcaggaaggaaagacctggaaggccatcagagtggacctggtgctgtgcccctacgagcggagagccttcgccctgctgggctggaccggcagccggcagttcgagcgggacctgcggagatacgccacccacgagcggaagatgatcctggacaaccacgccctgtacgacaagaccaagcggatcttcctgaaggccgagagcgaggaagaaatcttcgcccacctgggcctggactacatcgagccctgggagcggaacgcctaatctagagagagtttcagctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgggtaccgctagcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagcggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 25 - DNA, 11322 bp, artificial sequence
  Nucleic acid sequence of plasmid C645
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatacgaacggtacgaaccggggagtcccttttaggcacttgcttctggtgctgcaactggcgctcctcccagcagccactcagggaaagaaagtggtgctgggcaacagcggcgattacaaggatgacgacgataaggttcggacgggaggtggcgggggttctaattccggagactacaaagacgatgatgacaaggtgggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcataaggagccgccaccatggagtttgggctgagctggctttttcttgtggctattttaaaaggtgtccagtgtcaggtgcagctggtgcagtctggcgccgaagtgaagaaacctggcgcctccgtgaaggtgtcctgcaaggccagcggctacaccttcaccagctacggcatcagctgggtccgccaggctcctggacagggactggaatggatgggctggatcagcgcctacaacggcaacaccaactacgcccagaaactgcagggcagagtgaccatgaccaccgacaccagcaccagcaccgcctacatggaacttcgaagcctgagaagcgacgacaccgccgtgtattactgtgcgagagagctagcctatgatgcttttgatatctggggccaagggacaatggtcaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaacgcgtatctatccaagcggggtgatcggcctggtgcctcacctcggggatcgggaaaaacgcgactcagtatgcccgcaggggaaatatattcaccctcaaaataatagtatttgttgtaccaaatgtcacaaaggcacctacctgtacaatgactgccctgggcccgggcaagataccgactgccgagagtgtgaatccggttcctttaccgccagcgagaaccaccttaggcactgcctttcatgtagcaagtgccgaaaagagatgggacaggtggagatatcttcttgcactgttgatcgggacactgtctgcggatgtcgaaagaatcagtatcgccactattggtcagagaacctcttccagtgctttaattgcagcctctgccttaatggaactgttcacctttcctgccaagagaagcagaacactgtgtgtacctgtcacgctgggttctttcttcgcgagaacgagtgcgtgagctgcagcaattgcaagaagtccctggagtgtacaaaattgtgtttgcctcaaatcgaaaatgtcaagggcacggaggatagcgggaccactgtcctgttgccactggttatcttctttggattgtgcctgctgtcactgttgtttattggcctcatgtatcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcaattcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggaaagcttgccgccaccatggacccccccagagccagccacctgagcccccggaagaagcggcccagacagacaggcgccctgatggccagcagcccccaggacatcaagttccaggacctggtggtgttcatcctggaaaagaagatgggcaccaccagacgggcctttctgatggaactggccagacggaagggcttccgggtggagaacgagctgtccgacagcgtgacccacatcgtggccgagaacaacagcggcagcgacgtgctcgaatggctgcaggcccagaaagtgcaggtgtccagccagcccgagctgctggacgtgtcctggctgatcgagtgcatcagagccggcaagcccgtggagatgaccggcaagcaccagctggtcgtgcggcgggactacagcgacagcaccaaccccggaccccccaagaccccccctatcgccgtgcagaagatcagccagtacgcctgccagcggcggaccaccctgaacaactgcaaccagattttcaccgacgccttcgacatcctggccgaaaactgcgagttccgggagaacgaggacagctgcgtgaccttcatgagagccgccagcgtgctgaagtccctgcccttcaccatcatcagcatgaaggacaccgagggcatcccttgcctgggcagcaaagtgaagggcatcatcgaggaaatcattgaggacggcgagagcagcgaagtgaaagccgtgctgaacgacgagagataccagagcttcaagctgttcaccagcgtgttcggcgtgggcctgaaaaccagcgagaagtggttccggatgggcttcagaaccctgagcaaagtgcggagcgacaagagccttaagttcacccggatgcagaaggccggcttcctgtactacgaagatctggtgtcctgcgtgaccagagccgaggccgaggccgtgagcgtgctggtgaaagaggccgtctgggccttcctgcccgatgccttcgtgaccatgaccggcggcttcagacggggcaagaaaatgggccacgacgtggactttctgatcaccagccccggcagcaccgaggacgaagaacagctgctgcagaaagtgatgaacctgtgggagaagaagggcctgctgctgtactatgacctggtggagagcaccttcgagaagctgcggctgcccagccggaaggtggacgccctggaccacttccagaagtgctttctgatcttcaagctgcctcggcagagagtggacagcgaccagagcagctggcaggaaggaaagacctggaaggccatcagagtggacctggtgctgtgcccctacgagcggagagccttcgccctgctgggctggaccggcagccggcagttcgagcgggacctgcggagatacgccacccacgagcggaagatgatcctggacaaccacgccctgtacgacaagaccaagcggatcttcctgaaggccgagagcgaggaagaaatcttcgcccacctgggcctggactacatcgagccctgggagcggaacgcctaatctagagagagtttcagctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgggtaccgctagcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggacatgagggtccccgctcagctcctggggctcctgctactctggctccgaggtgccagatgtgacatccagatgacccagagccccagcagcctgagcgccagcgtgggcgacagagtgaccatcacctgtcgggccagccagtcgatcagcagctacctgaactggtatcagcagaagcccggcaaggcccccaagctgctgatctacgccgccagctcccaggcaggcctatccctgcagagcggcgtgccaagcagattcagcggcagcggctccggcaccgacttcaccctgaccatcagcagcctgcagcccgaggacttcgccacctactactgccagcagagttacagtacccctctcactttcggcggagggaccaaggtggagatcaaacggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 26 - protein, 904 aa, artificial sequence
  Amino acid sequence of IgG(heavy chain)-TNFR1 (full length) with leader sequence for surface expression, encoded by plasmid C601
MEFGLSWLFLVAILKGVQCDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTRIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 27 - protein, 213 aa, artificial sequence
  Amino acid sequence of IgG(light chain) (no leader sequence), encoded by plasmid C601
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 906 aa, artificial sequence
  Amino acid sequence of IgG(heavy chain)-TNFR1 (full length) with leader sequence for surface expression, encoded by plasmid C638
MEFGLSWLFLVAILKGVQCEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEVQLERLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTRIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 29 - protein, 236 aa, artificial sequence
  Amino acid sequence of IgG(light chain) (with leader sequence), encoded by plasmid C638 or C644
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 903 aa, artificial sequence
  Amino acid sequence of the IgG(heavy chain)- TNFR1 encoded by plasmid C644 or C645, with the leader sequence for surface expression
MEFGLSWLFLVAILKGVQCQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARELAYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTRIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 31 - protein, 241 aa, artificial sequence
  Amino acid sequence of the IgG(light chain) encoded by plasmid C645
MDMRVPAQLLGLLLLWLRGARCDIQMTQSPSSLSASVGDRVTITCRASQSISSYLNWYQQKPGKAPKLLIYAASSQAGLSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 5357 bp, artificial sequence
  Nucleic acid sequence of plasmid V707
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgaggagccgccaccatggacatgagggtccccgctcagctcctggggctcctgctactctggctccgaggtaaggatggagaacactaggaatttactcagccagtgtgctcagtactgactggaacttcagggaagttctctgataacatgattaatagtaagaatatttgtttttatgtttccaatctcaggtgccagatgtgatattgtgatgactcagtctccactctccctgcccgtcacccctggagagccggcctccatctcctgtaggtctagtcagagcctcctgcatagtaatggatacaactatttggattggtatctccagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggcggaacaaaggtggagatcaagcggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcacgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 33 - protein, 219 aa, artificial sequence
  Amino acid sequence of the light chain encoded on plasmid V707 (no leader)
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - DNA, 4778 bp, artificial sequence
  Nucleic acid sequence of plasmid C112
tcgacctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggatccacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctttctagggcctctgagctattccagaagtagtgaagaggcttttttggaggcctaggcttttgcaaaaagctccggatcgatcctgagaacttcagggtgagtttggggacccttgattgttctttctttttcgctattgtaaaattcctgttatatggagggggcaaagttttcagggtgttgtttagactgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtccgattataagtactttctctaatcacttttttttcacggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagctgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaggtctccagctttgatgccgccaccatgagccggctggacaagagcaaagtgatcaacagcgccctggaactgctgaacgaagtgggcatcgagggcctgaccaccagaaagctggcccagaaactgggcgtggaacagcccaccctgtactggcacgtgaagaacaagcgggccctgctggatgccctggccatcgagatgctggaccggcaccacacccacttttgccccctggaaggcgagagctggcaggacttcctgcggaacaacgccaagagcttcagatgcgccctgctgagccaccgggatggcgccaaagtgcacctgggcaccagacctaccgagaagcagtacgagacactggaaaaccagctggccttcctgtgccagcaaggattcagcctggaaaacgccctgtacgccctgagcgccgtgggccactttacactgggatgcgtgctggaagatcaggaacaccaggtggccaaagaggaaagagagacacccaccaccgacagcatgccccctctgctgagacaggccattgagctgttcgatcatcaaggcgccgagcccgcctttctgttcggactggaactgatcatctgcgggctggaaaagcagctgaagtgcgagagcggctccgcctactcttgataatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggactcgaggagttcgtgacctagtgagacgtcgtgggcgggacgtctctatcgagtg

SEQ ID NO: 35 - RNA, 67 nt, artificial sequence
  TracrRNA
agcauagcaaguuaaaauaaggcuaguccguuaucaacuugaaaaaguggcaccgagucggugcuuu

SEQ ID NO: 36 - RNA, 36 nt, artificial sequence
  CPcrRNA9
gaagucccugcaccacgaccguuuuagagcuaugcu

SEQ ID NO: 37 - RNA, 36 nt, artificial sequence
  CPcrRNA10
acagcaugucagugcaaaccguuuuagagcuaugcu

SEQ ID NO: 38 - RNA, 36 nt, artificial sequence
  CPcrRNA11
auaguccuguccauauuugcguuuuagagcuaugcu

SEQ ID NO: 39 - RNA, 36 nt, artificial sequence
  CPcrRNA12
aggucggugauuguacacccguuuuagagcuaugcu

SEQ ID NO: 40 - protein, 883 aa, artificial sequence
  SEQ ID NO:13 minus leader
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDLLEWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLL

SEQ ID NO: 41-44 - skipped (4 entries)

SEQ ID NO: 45 - protein, 884 aa, artificial sequence
  SEQ ID NO:30 minus leader
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARELAYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTRIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 46 - protein, 219 aa, artificial sequence
  SEQ ID NO:31 minus leader
DIQMTQSPSSLSASVGDRVTITCRASQSISSYLNWYQQKPGKAPKLLIYAASSQAGLSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 214 aa, artificial sequence
  SEQ ID NO:29 minus leader
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 5821 bp, artificial sequence
  Nucleic acid sequence of plasmid C487
ggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgggaatttccggggactttccgggaatttccggggactttccgggaatttccaaatctggcctcggcggccaagcttagacactagagggtatataatggaagctcgacttccagcttggcaatccggtactactagcgccgccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcacgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgac

SEQ ID NO: 49 - DNA, 10788 bp, artificial sequence
  Nucleic acid sequence of plasmid C639
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaccggggagtcccttttaggcacttgcttctggtgctgcaactggcgctcctcccagcagccactcagggaaagaaagtggtgctgggcgaacagaaacttatctctgaggaggatttgcggacgggaggtggcggaggctccgaacagaaacttatctctgaggaggatttgcgtaccggtggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcataaggagccgccaccatggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtgtcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaggtacaactggaacgacttgatgcttttgatatctggggccaagggacaatggtcaccgtgtcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaacgcgtcctcaaatcgaaaatgtcaagggcacggaggatagcgggaccactgtcctgttgccactggttatcttctttggattgtgcctgctgtcactgttgtttattggcctcatgtatcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcaattcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggaaagcttgccgccaccatggacccccccagagccagccacctgagcccccggaagaagcggcccagacagacaggcgccctgatggccagcagcccccaggacatcaagttccaggacctggtggtgttcatcctggaaaagaagatgggcaccaccagacgggcctttctgatggaactggccagacggaagggcttccgggtggagaacgagctgtccgacagcgtgacccacatcgtggccgagaacaacagcggcagcgacgtgctcgaatggctgcaggcccagaaagtgcaggtgtccagccagcccgagctgctggacgtgtcctggctgatcgagtgcatcagagccggcaagcccgtggagatgaccggcaagcaccagctggtcgtgcggcgggactacagcgacagcaccaaccccggaccccccaagaccccccctatcgccgtgcagaagatcagccagtacgcctgccagcggcggaccaccctgaacaactgcaaccagattttcaccgacgccttcgacatcctggccgaaaactgcgagttccgggagaacgaggacagctgcgtgaccttcatgagagccgccagcgtgctgaagtccctgcccttcaccatcatcagcatgaaggacaccgagggcatcccttgcctgggcagcaaagtgaagggcatcatcgaggaaatcattgaggacggcgagagcagcgaagtgaaagccgtgctgaacgacgagagataccagagcttcaagctgttcaccagcgtgttcggcgtgggcctgaaaaccagcgagaagtggttccggatgggcttcagaaccctgagcaaagtgcggagcgacaagagccttaagttcacccggatgcagaaggccggcttcctgtactacgaagatctggtgtcctgcgtgaccagagccgaggccgaggccgtgagcgtgctggtgaaagaggccgtctgggccttcctgcccgatgccttcgtgaccatgaccggcggcttcagacggggcaagaaaatgggccacgacgtggactttctgatcaccagccccggcagcaccgaggacgaagaacagctgctgcagaaagtgatgaacctgtgggagaagaagggcctgctgctgtactatgacctggtggagagcaccttcgagaagctgcggctgcccagccggaaggtggacgccctggaccacttccagaagtgctttctgatcttcaagctgcctcggcagagagtggacagcgaccagagcagctggcaggaaggaaagacctggaaggccatcagagtggacctggtgctgtgcccctacgagcggagagccttcgccctgctgggctggaccggcagccggcagttcgagcgggacctgcggagatacgccacccacgagcggaagatgatcctggacaaccacgccctgtacgacaagaccaagcggatcttcctgaaggccgagagcgaggaagaaatcttcgcccacctgggcctggactacatcgagccctgggagcggaacgcctaatctagagagagtttcagctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgggtaccgctagcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagcggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 50 - DNA, 1502 bp, artificial sequence
  Nucleic acid sequence of plasmid C884
aagcttagacactagagggtatataatggaagctcgacttccagcttggcaatccggaactactagcgccgccaccatggctccacctcctgcccgggttcacctgggagcttttctggccgtgacacccaatcctggatctgccgcctctggaacagaagccgctgctgccacacctagcaaagtgtggggaagcagcgccggcagaattgagcctagaggcggaggaagaggcgccctgcctacatctatgggacagcacggaccaagcgccagagctagagctggtagagcacctggacctagaccagccagagaggccagccctagactgagagtgcacaagaccttcaagttcgtggtcgtgggcgtgctgctgcaggtcgtgccttcttctgccgccacaatcaagctgcacgaccagagcatcggcacccagcagtgggaacattctcctctgggcgaactgtgtcctcctggcagccacagatctgaacatcctggcgcctgcaacagatgcacagaaggcgtgggctacaccaacgccagcaacaacctgttcgcctgcctgccttgtaccgcctgcaagagcgacgaggaagagagaagcccttgcaccaccaccagaaacaccgcctgtcagtgcaagcccggcaccttcagaaacgacaacagcgccgagatgtgccggaagtgctctaggggctgtcccagaggcatggtcaaagtgaaggactgcaccccttggagcgacatcgagtgcgtgcacaaagagtctggcaacggccacaacatctgggtcatcctggtggtcacactggtggtgcctctgctgctggtggctgtgctgatcgtgtgctgttgtatcggctctggctgtggcggcgaccccaagtgtatggacagagtgtgcttttggcggctgggcctgcttagaggacctggcgctgaagataacgcccacaacgagatcctgagcaacgccgatagcctgagcaccttcgtgtccgagcagcagatggaaagccaagagcctgccgatctgaccggcgtgacagttcaatctcctggcgaagcccagtgtctgctgggacctgctgaagccgagggatctcaacgtagacgactgctggtgcctgccaatggcgccgatcctacagagacactgatgctgttcttcgacaagttcgccaacatcgtgcccttcgactcctgggaccagctcatgagacagctggatctgaccaagaacgagatcgacgtcgtcagagccggaacagctggacctggggatgccctgtatgccatgctgatgaaatgggtcaacaagaccggccggaacgcctccattcacactctgctggacgccctggaacggatggaagaaagacacgccagagagaagattcaggacctgctcgtggacagcggcaagttcatctacctggaagatggcacaggcagcgccgtgtctctggaatgagcggccgctcgagtctaga

SEQ ID NO: 51 - DNA, 3053 bp, artificial sequence
  Nucleic acid sequence of plasmid T99
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcataaggagccgccaccatggagtttgggctgagctggctttttcttgtggctattttaaaaggtgtccagtgtgacatcaagctgcagcagtctggcgccgagctggctagacctggcgcctctgtgaagatgagctgcaagaccagcggctacaccttcacccggtacaccatgcactgggtcaagcagaggcctggacagggcctggaatggatcggctacatcaaccccagccggggctacaccaactacaaccagaagttcaaggacaaggccaccctgaccaccgacaagagcagcagcaccgcctacatgcagctgagcagcctgaccagcgaggacagcgccgtgtactactgcgcccggtactacgacgaccactactgcctggactactggggccagggcaccacactgaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaacgcgtgcctctggaacagaagccgctgctgccacacctagcaaagtgtggggaagcagcgccggcagaattgagcctagaggcggaggaagaggcgccctgcctacatctatgggacagcacggaccaagcgccagagctagagctggtagagcacctggacctagaccagccagagaggccagccctagactgagagtgcacaagaccttcaagttcgtggtcgtgggcgtgctgctgcaggtcgtgccttcttctgccgccacaatcaagctgcacgaccagagcatcggcacccagcagtgggaacattctcctctgggcgaactgtgtcctcctggcagccacagatctgaacatcctggcgcctgcaacagatgcacagaaggcgtgggctacaccaacgccagcaacaacctgttcgcctgcctgccttgtaccgcctgcaagagcgacgaggaagagagaagcccttgcaccaccaccagaaacaccgcctgtcagtgcaagcccggcaccttcagaaacgacaacagcgccgagatgtgccggaagtgctctaggggctgtcccagaggcatggtcaaagtgaaggactgcaccccttggagcgacatcgagtgcgtgcacaaagagtctggcaacggccacaacatctgggtcatcctggtggtcacactggtggtgcctctgctgctggtggctgtgctgatcgtgtgctgttgtatcggctctggctgtggcggcgaccccaagtgtatggacagagtgtgcttttggcggctgggcctgcttagaggacctggcgctgaagataacgcccacaacgagatcctgagcaacgccgatagcctgagcaccttcgtgtccgagcagcagatggaaagccaagagcctgccgatctgaccggcgtgacagttcaatctcctggcgaagcccagtgtctgctgggacctgctgaagccgagggatctcaacgtagacgactgctggtgcctgccaatggcgccgatcctacagagacactgatgctgttcttcgacaagttcgccaacatcgtgcccttcgactcctgggaccagctcatgagacagctggatctgaccaagaacgagatcgacgtcgtcagagccggaacagctggacctggggatgccctgtatgccatgctgatgaaatgggtcaacaagaccggccggaacgcctccattcacactctgctggacgccctggaacggatggaagaaagacacgccagagagaagattcaggacctgctcgtggacagcggcaagttcatctacctggaagatggcacaggcagcgccgtgtctctggaatgagcggccggtcgac

SEQ ID NO: 52 - DNA, 2873 bp, artificial sequence
  Nucleic acid sequence of plasmid T100
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcataaggagccgccaccatggagtttgggctgagctggctttttcttgtggctattttaaaaggtgtccagtgtgacatcaagctgcagcagtctggcgccgagctggctagacctggcgcctctgtgaagatgagctgcaagaccagcggctacaccttcacccggtacaccatgcactgggtcaagcagaggcctggacagggcctggaatggatcggctacatcaaccccagccggggctacaccaactacaaccagaagttcaaggacaaggccaccctgaccaccgacaagagcagcagcaccgcctacatgcagctgagcagcctgaccagcgaggacagcgccgtgtactactgcgcccggtactacgacgaccactactgcctggactactggggccagggcaccacactgaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaacgcgtatcacacagcaggatctggcccctcagcagagagctgctccacagcagaagagaagcagccctagcgagggactgtgtcctcctggacaccacatcagcgaggacggcagagattgcatcagctgcaaatacggccaggactacagcacccactggaacgacctgctgttctgcctgagatgcaccagatgcgatagcggcgaggtggaactgagcccttgtaccaccaccagaaacaccgtgtgccagtgcgaggaaggcaccttcagagaagaggacagccccgagatgtgccggaagtgtagaaccggctgtcccagaggcatggtcaaagtgggcgattgcaccccttggagcgacatcgagtgcgtgcacaaagagagcggcacaaagcactctggcgaagtgcctgccgtggaagagacagtgacaagctctccaggcacacccgcctctccatgttctctgagcggcatcatcatcggcgtgacagtggctgcagtggtgctgatcgtggctgtgttcgtgtgcaagagcctgctctggaagaaggtgctgccctacctgaagggcatctgttctggcggaggcggcgatcctgagagagtggatagaagctcccaaagacctggcgccgaggacaacgtgctgaacgagatcgtgtccatcctgcagcctacacaagtgcccgagcaagagatggaagtgcaagaaccagccgagcctaccggcgtgaacatgctttcacctggcgagagcgagcatctgctggaacctgccgaagccgagagatcccaaaggcggagactgctggtgccagccaatgagggcgatcctaccgagacactgagacagtgcttcgacgacttcgccgacctggtgcctttcgattcttgggagcccctgatgagaaagctgggcctgatggacaacgagatcaaggtggccaaagccgaggccgctggccacagagataccctgtacaccatgctgatcaaatgggtcaacaagaccggcagggacgccagcgttcacacactgctggatgccctggaaaccctgggagagagactggccaagcagaagatcgaggaccatctgctgagcagcggcaagttcatgtacctggaaggcaacgccgacagcgccatgagttaagcggccggtcgac

SEQ ID NO: 53 - DNA, 1901 bp, artificial sequence
  Nucleic acid sequence of plasmid T96
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcaagccgccaccatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcaacgcgtatctatccaagcggggtgatcggcctggtgcctcacctcggggatcgggaaaaacgcgactcagtatgcccgcaggggaaatatattcaccctcaaaataatagtatttgttgtaccaaatgtcacaaaggcacctacctgtacaatgactgccctgggcccgggcaagataccgactgccgagagtgtgaatccggttcctttaccgccagcgagaaccaccttaggcactgcctttcatgtagcaagtgccgaaaagagatgggacaggtggagatatcttcttgcactgttgatcgggacactgtctgcggatgtcgaaagaatcagtatcgccactattggtcagagaacctcttccagtgctttaattgcagcctctgccttaatggaactgttcacctttcctgccaagagaagcagaacactgtgtgtacctgtcacgctgggttctttcttcgcgagaacgagtgcgtgagctgcagcaattgcaagaagtccctggagtgtacaaaattgtgtttgcctcaaatcgaaaatgtcaagggcacggaggatagcgggaccactgtcctgttgccactggttatcttctttggattgtgcctgctgtcactgttgtttattggcctcatgtatcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgac

SEQ ID NO: 54 - DNA, 3227 bp, artificial sequence
  Nucleic acid sequence of plasmid T101
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcaagccgccaccatgacttccaagctggccgtggctctcttggcagccttcctgatttctgcagctctgtgttgggaactgaccatcctgcacaccaacgacgtgcacagcagactggaacagaccagcgaggacagctctaagtgcgtgaacgccagcagatgcatgggcggcgtggccagactgttcaccaaggtgcagcagatcagacgggccgagcccaacgtgctgctgctggatgctggcgatcagtaccagggcaccatctggttcaccgtgtacaagggcgccgaggtggcccacttcatgaacgccctgagatacgacgccatggccctgggcaaccacgagttcgacaatggcgtggaaggcctgatcgagcctctgctgaaagaggccaagttccccatcctgagcgccaacatcaaggccaagggacccctggccagccagatcagcggactgtacctgccctacaaggtgctgcctgtgggggacgaggtcgtgggcatcgtgggctacaccagcaaagagacacccttcctgagcaaccccggcaccaacctggtgttcgaggacgagatcaccgccctgcagcccgaggtggacaagctgaaaaccctgaacgtgaacaagatcattgccctgggccacagcggcttcgagatggataagctgatcgcccagaaagtgcggggcgtggacgtggtcgtgggaggccactccaacacctttctgtacaccggcaacccccctagcaaagaggtgccagccggcaagtaccccttcatcgtgaccagcgacgacggccggaaagtgcctgtggtgcaggcctacgccttcggcaagtatctgggctacctgaagatcgagttcgatgagcggggcaacgtgatcagcagccacggcaaccctatcctgctgaacagcagcatccccgaggacccctctatcaaggccgacatcaacaagtggcggatcaagctggacaactacagcacccaggaactgggcaagaccatcgtgtacctggacggcagcagccagagctgccggttccgcgagtgcaacatgggcaacctgatctgtgacgccatgatcaacaacaacctgcggcacaccgacgagatgttctggaaccacgtgtccatgtgcatcctgaacggcggaggcatcagaagccccatcgacgagagaaacaacggcaccatcacctgggagaacctggccgccgtgctgccttttggcggcacctttgatctggtgcagctgaagggcagcaccctgaagaaggcctttgagcacagcgtgcacagatacggccagagcaccggcgagtttctgcaagtgggcggcatccacgtggtgtacgacctgagcagaaagcccggcgaccgggtcgtgaagctggacgtgctgtgtaccaagtgccgggtgcccagctacgaccccctgaagatggacgaggtgtacaaagtgatcctgcccaacttcctggccaacggcggcgacggcttccagatgatcaaggacgagctgctgcggcacgacagcggcgaccaggacatcaatgtggtgtccacctacatcagcaagatgaaagtgatctaccccgccgtggaaggacggatcaagacgcgtatctatccaagcggggtgatcggcctggtgcctcacctcggggatcgggaaaaacgcgactcagtatgcccgcaggggaaatatattcaccctcaaaataatagtatttgttgtaccaaatgtcacaaaggcacctacctgtacaatgactgccctgggcccgggcaagataccgactgccgagagtgtgaatccggttcctttaccgccagcgagaaccaccttaggcactgcctttcatgtagcaagtgccgaaaagagatgggacaggtggagatatcttcttgcactgttgatcgggacactgtctgcggatgtcgaaagaatcagtatcgccactattggtcagagaacctcttccagtgctttaattgcagcctctgccttaatggaactgttcacctttcctgccaagagaagcagaacactgtgtgtacctgtcacgctgggttctttcttcgcgagaacgagtgcgtgagctgcagcaattgcaagaagtccctggagtgtacaaaattgtgtttgcctcaaatcgaaaatgtcaagggcacggaggatagcgggaccactgtcctgttgccactggttatcttctttggattgtgcctgctgtcactgttgtttattggcctcatgtatcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgac

SEQ ID NO: 55 - DNA, 10217 bp, artificial sequence
  Nucleic acid sequence of plasmid C58
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaccggggagtcccttttaggcacttgcttctggtgctgcaactggcgctcctcccagcagccactcagggaaagaaagtggtgctgggcgggaaacccattcccaatcccctccttgggcttgactccacccggacgggaggtggcggaggctccggcaagcctatccctaaccctctcctcggcctcgattctacgcgtaccggtggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggacatgagggtccccgctcagctcctggggctcctgctactctggctccgaggtgccagatgtgacatccagatgacccagagccccagcagcctgagcgccagcgtgggcgacagagtgaccatcacctgtcgggccagccagtcgatcagcagctacctgaactggtatcagcagaagcccggcaaggcccccaagctgctgatctacgccgccagctccctgcagagcggcgtgccaagcagattcagcggcagcggctccggcaccgacttcaccctgaccatcagcagcctgcagcccgaggacttcgccacctactactgccagcagagttacagtacccctctcactttcggcggagggaccaaggtggagatcaaacgaactgtggctgcaccatctgtcttcatcttcccgccatctgatgagcagttgaaaagcggaacagccagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcagccccgtcacaaagagcttcaacaggggagagtgttaactgagcagttaacgccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacagcggccgctaataaagagagcgatctgatcaagagacaggataaggagccgccaccatggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtgtcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaggtacaactggaacgacttgatgcttttgatatctggggccaagggacaatggtcaccgtgtcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagaacctctccctgtctccgggcaaagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaaggctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggaaagcttgccgccaccatggacccccccagagccagccacctgagcccccggaagaagcggcccagacagacaggcgccctgatggccagcagcccccaggacatcaagttccaggacctggtggtgttcatcctggaaaagaagatgggcaccaccagacgggcctttctgatggaactggccagacggaagggcttccgggtggagaacgagctgtccgacagcgtgacccacatcgtggccgagaacaacagcggcagcgacgtgctcgaatggctgcaggcccagaaagtgcaggtgtccagccagcccgagctgctggacgtgtcctggctgatcgagtgcatcagagccggcaagcccgtggagatgaccggcaagcaccagctggtcgtgcggcgggactacagcgacagcaccaaccccggaccccccaagaccccccctatcgccgtgcagaagatcagccagtacgcctgccagcggcggaccaccctgaacaactgcaaccagattttcaccgacgccttcgacatcctggccgaaaactgcgagttccgggagaacgaggacagctgcgtgaccttcatgagagccgccagcgtgctgaagtccctgcccttcaccatcatcagcatgaaggacaccgagggcatcccttgcctgggcagcaaagtgaagggcatcatcgaggaaatcattgaggacggcgagagcagcgaagtgaaagccgtgctgaacgacgagagataccagagcttcaagctgttcaccagcgtgttcggcgtgggcctgaaaaccagcgagaagtggttccggatgggcttcagaaccctgagcaaagtgcggagcgacaagagccttaagttcacccggatgcagaaggccggcttcctgtactacgaagatctggtgtcctgcgtgaccagagccgaggccgaggccgtgagcgtgctggtgaaagaggccgtctgggccttcctgcccgatgccttcgtgaccatgaccggcggcttcagacggggcaagaaaatgggccacgacgtggactttctgatcaccagccccggcagcaccgaggacgaagaacagctgctgcagaaagtgatgaacctgtgggagaagaagggcctgctgctgtactatgacctggtggagagcaccttcgagaagctgcggctgcccagccggaaggtggacgccctggaccacttccagaagtgctttctgatcttcaagctgcctcggcagagagtggacagcgaccagagcagctggcaggaaggaaagacctggaaggccatcagagtggacctggtgctgtgcccctacgagcggagagccttcgccctgctgggctggaccggcagccggcagttcgagcgggacctgcggagatacgccacccacgagcggaagatgatcctggacaaccacgccctgtacgacaagaccaagcggatcttcctgaaggccgagagcgaggaagaaatcttcgcccacctgggcctggactacatcgagccctgggagcggaacgcctaatctagagagagtttcagctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgacctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 56 - DNA, 2700 bp, artificial sequence
  Nucleic acid sequence of plasmid T145
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcaagccgccaccatgacttccaagctggccgtggctctcttggcagccttcctgatttctgcagctctgtgttgggaactgaccatcctgcacaccaacgacgtgcacagcagactggaacagaccagcgaggacagctctaagtgcgtgaacgccagcagatgcatgggcggcgtggccagactgttcaccaaggtgcagcagatcagacgggccgagcccaacgtgctgctgctggatgctggcgatcagtaccagggcaccatctggttcaccgtgtacaagggcgccgaggtggcccacttcatgaacgccctgagatacgacgccatggccgggcaaccacgagttcgacaatggcgtggaaggcctgatcgagcctctgctgaaagaggccaagttccccatcctgagcgccaacatcaaggccaagggacccctggccagccagatcagcggactgtacctgccctacaaggtgctgcctgtgggggacgaggtcgtgggcatcgtgggctacaccagcaaagagacacccttcctgagcaaccccggcaccaacctggtgttcgaggacgagatcaccgccctgcagcccgaggtggacaagctgaaaaccctgaacgtgaacaagatcattgccctgggccacagcggcttcgagatggataagctgatcgcccagaaagtgcggggcgtggacgtggtcgtgggaggccactccaacacctttctgtacaccggcaacccccctagcaaagaggtgccagccggcaagtaccccttcatcgtgaccagcgacgacggccggaaagtgcctgtggtgcaggcctacgccttcggcaagtatctgggctacctgaagatcgagttcgatgagcggggcaacgtgatcagcagccacggcaaccctatcctgctgaacagcagcatccccgaggacccctctatcaaggccgacatcaacaagtggcggatcaagctggacaactacagcacccaggaactgggcaagaccatcgtgtacctggacggcagcagccagagctgccggttccgcgagtgcaacatgggcaacctgatctgtgacgccatgatcaacaacaacctgcggcacaccgacgagatgttctggaaccacgtgtccatgtgcatcctgaacggcggaggcatcagaagccccatcgacgagagaaacaacggcaccatcacctgggagaacctggccgccgtgctgccttttggcggcacctttgatctggtgcagctgaagggcagcaccctgaagaaggcctttgagcacagcgtgcacagatacggccagagcaccggcgagtttctgcaagtgggcggcatccacgtggtgtacgacctgagcagaaagcccggcgaccgggtcgtgaagctggacgtgctgtgtaccaagtgccgggtgcccagctacgaccccctgaagatggacgaggtgtacaaagtgatcctgcccaacttcctggccaacggcggcgacggcttccagatgatcaaggacgagctgctgcggcacgacagcggcgaccaggacatcaatgtggtgtccacctacatcagcaagatgaaagtgatctaccccgccgtggaaggacggatcaagacgcgtcctcaaatcgaaaatgtcaagggcacggaggatagcgggaccactgtcctgttgccactggttatcttctttggattgtgcctgctgtcactgttgtttattggcctcatgtatcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgac

SEQ ID NO: 57 - DNA, 3010 bp, artificial sequence
  Nucleic acid sequence of plasmid T110
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcataaggagccgccaccatggagtttgggctgagctggctttttcttgtggctattttaaaaggtgtccagtgtgacatcaagctgcagcagtctggcgccgagctggctagacctggcgcctctgtgaagatgagctgcaagaccagcggctacaccttcacccggtacaccatgcactgggtcaagcagaggcctggacagggcctggaatggatcggctacatcaaccccagccggggctacaccaactacaaccagaagttcaaggacaaggccaccctgaccaccgacaagagcagcagcaccgcctacatgcagctgagcagcctgaccagcgaggacagcgccgtgtactactgcgcccggtactacgacgaccactactgcctggactactggggccagggcaccacactgaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaacgcgtatctatccaagcggggtgatcggcctggtgcctcacctcggggatcgggaaaaacgcgactcagtatgcccgcaggggaaatatattcaccctcaaaataatagtatttgttgtaccaaatgtcacaaaggcacctacctgtacaatgactgccctgggcccgggcaagataccgactgccgagagtgtgaatccggttcctttaccgccagcgagaaccaccttaggcactgcctttcatgtagcaagtgccgaaaagagatgggacaggtggagatatcttcttgcactgttgatcgggacactgtctgcggatgtcgaaagaatcagtatcgccactattggtcagagaacctcttccagtgctttaattgcagcctctgccttaatggaactgttcacctttcctgccaagagaagcagaacactgtgtgtacctgtcacgctgggttctttcttcgcgagaacgagtgcgtgagctgcagcaattgcaagaagtccctggagtgtacaaaattgtgtttgcctcaaatcgaaaatgtcaagggcacggaggatagcgggaccactctggctctcattgtcctgggcggcgtggctggcctgctgctgtttattgggctgggcatcttctttcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgac

SEQ ID NO: 58 - DNA, 3007 bp, artificial sequence
  Nucleic acid sequence of plasmid T111
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcataaggagccgccaccatggagtttgggctgagctggctttttcttgtggctattttaaaaggtgtccagtgtgacatcaagctgcagcagtctggcgccgagctggctagacctggcgcctctgtgaagatgagctgcaagaccagcggctacaccttcacccggtacaccatgcactgggtcaagcagaggcctggacagggcctggaatggatcggctacatcaaccccagccggggctacaccaactacaaccagaagttcaaggacaaggccaccctgaccaccgacaagagcagcagcaccgcctacatgcagctgagcagcctgaccagcgaggacagcgccgtgtactactgcgcccggtactacgacgaccactactgcctggactactggggccagggcaccacactgaccgtgtctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaacgcgtatctatccaagcggggtgatcggcctggtgcctcacctcggggatcgggaaaaacgcgactcagtatgcccgcaggggaaatatattcaccctcaaaataatagtatttgttgtaccaaatgtcacaaaggcacctacctgtacaatgactgccctgggcccgggcaagataccgactgccgagagtgtgaatccggttcctttaccgccagcgagaaccaccttaggcactgcctttcatgtagcaagtgccgaaaagagatgggacaggtggagatatcttcttgcactgttgatcgggacactgtctgcggatgtcgaaagaatcagtatcgccactattggtcagagaacctcttccagtgctttaattgcagcctctgccttaatggaactgttcacctttcctgccaagagaagcagaacactgtgtgtacctgtcacgctgggttctttcttcgcgagaacgagtgcgtgagctgcagcaattgcaagaagtccctggagtgtacaaaattgtgtttgcctcaaatcgaaaatgtcaagggcacggaggatagcgggaccactgtggtgatctcagccatcctggccctggtggtgctcaccatcatctcccttatcatcctcatccgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgac

SEQ ID NO: 59 - DNA, 2705 bp, artificial sequence
  Nucleic acid sequence of plasmid T146
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcaagccgccaccatgacttccaagctggccgtggctctcttggcagccttcctgatttctgcagctctgtgttgggaactgaccatcctgcacaccaacgacgtgcacagcagactggaacagaccagcgaggacagctctaagtgcgtgaacgccagcagatgcatgggcggcgtggccagactgttcaccaaggtgcagcagatcagacgggccgagcccaacgtgctgctgctggatgctggcgatcagtaccagggcaccatctggttcaccgtgtacaagggcgccgaggtggcccacttcatgaacgccctgagatacgacgccatggccctgggcaaccacgagttcgacaatggcgtggaaggcctgatcgagcctctgctgaaagaggccaagttccccatcctgagcgccaacatcaaggccaagggacccctggccagccagatcagcggactgtacctgccctacaaggtgctgcctgtgggggacgaggtcgtgggcatcgtgggctacaccagcaaagagacacccttcctgagcaaccccggcaccaacctggtgttcgaggacgagatcaccgccctgcagcccgaggtggacaagctgaaaaccctgaacgtgaacaagatcattgccctgggccacagcggcttcgagatggataagctgatcgcccagaaagtgcggggcgtggacgtggtcgtgggaggccactccaacacctttctgtacaccggcaacccccctagcaaagaggtgccagccggcaagtaccccttcatcgtgaccagcgacgacggccggaaagtgcctgtggtgcaggcctacgccttcggcaagtatctgggctacctgaagatcgagttcgatgagcggggcaacgtgatcagcagccacggcaaccctatcctgctgaacagcagcatccccgaggacccctctatcaaggccgacatcaacaagtggcggatcaagctggacaactacagcacccaggaactgggcaagaccatcgtgtacctggacggcagcagccagagctgccggttccgcgagtgcaacatgggcaacctgatctgtgacgccatgatcaacaacaacctgcggcacaccgacgagatgttctggaaccacgtgtccatgtgcatcctgaacggcggaggcatcagaagccccatcgacgagagaaacaacggcaccatcacctgggagaacctggccgccgtgctgccttttggcggcacctttgatctggtgcagctgaagggcagcaccctgaagaaggcctttgagcacagcgtgcacagatacggccagagcaccggcgagtttctgcaagtgggcggcatccacgtggtgtacgacctgagcagaaagcccggcgaccgggtcgtgaagctggacgtgctgtgtaccaagtgccgggtgcccagctacgaccccctgaagatggacgaggtgtacaaagtgatcctgcccaacttcctggccaacggcggcgacggcttccagatgatcaaggacgagctgctgcggcacgacagcggcgaccaggacatcaatgtggtgtccacctacatcagcaagatgaaagtgatctaccccgccgtggaaggacggatcaaggctgtgggccaggacacgcaggaggtcatcgtggtgccacactccttgccctttaaggtggtggtgatctcagccatcctggccctggtggtgctcaccatcatctcccttatcatcctcatccgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgagtcgac

SEQ ID NO: 60 - DNA, 2669 bp, artificial sequence
  Nucleic acid sequence of plasmid T147
ggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgcaagccgccaccatgacttccaagctggccgtggctctcttggcagccttcctgatttctgcagctctgtgttgggaactgaccatcctgcacaccaacgacgtgcacagcagactggaacagaccagcgaggacagctctaagtgcgtgaacgccagcagatgcatgggcggcgtggccagactgttcaccaaggtgcagcagatcagacgggccgagcccaacgtgctgctgctggatgctggcgatcagtaccagggcaccatctggttcaccgtgtacaagggcgccgaggtggcccacttcatgaacgccctgagatacgacgccatggccctgggcaaccacgagttcgacaatggcgtggaaggcctgatcgagcctctgctgaaagaggccaagttccccatcctgagcgccaacatcaaggccaagggacccctggccagccagatcagcggactgtacctgccctacaaggtgctgcctgtgggggacgaggtcgtgggcatcgtgggctacaccagcaaagagacacccttcctgagcaaccccggcaccaacctggtgttcgaggacgagatcaccgccctgcagcccgaggtggacaagctgaaaaccctgaacgtgaacaagatcattgccctgggccacagcggcttcgagatggataagctgatcgcccagaaagtgcggggcgtggacgtggtcgtgggaggccactccaacacctttctgtacaccggcaacccccctagcaaagaggtgccagccggcaagtaccccttcatcgtgaccagcgacgacggccggaaagtgcctgtggtgcaggcctacgccttcggcaagtatctgggctacctgaagatcgagttcgatgagcggggcaacgtgatcagcagccacggcaaccctatcctgctgaacagcagcatccccgaggacccctctatcaaggccgacatcaacaagtggcggatcaagctggacaactacagcacccaggaactgggcaagaccatcgtgtacctggacggcagcagccagagctgccggttccgcgagtgcaacatgggcaacctgatctgtgacgccatgatcaacaacaacctgcggcacaccgacgagatgttctggaaccacgtgtccatgtgcatcctgaacggcggaggcatcagaagccccatcgacgagagaaacaacggcaccatcacctgggagaacctggccgccgtgctgccttttggcggcacctttgatctggtgcagctgaagggcagcaccctgaagaaggcctttgagcacagcgtgcacagatacggccagagcaccggcgagtttctgcaagtgggcggcatccacgtggtgtacgacctgagcagaaagcccggcgaccgggtcgtgaagctggacgtgctgtgtaccaagtgccgggtgcccagctacgaccccctgaagatggacgaggtgtacaaagtgatcctgcccaacttcctggccaacggcggcgacggcttccagatgatcaaggacgagctgctgcggcacgacagcggcgaccaggacatcaatgtggtgtccacctacatcagcaagatgaaagtgatctaccccgccgtggaaggacggatcaaggctgtgggccaggacacgcaggaggtcatcgtggtgccacactccttgccctttaaggtggtggtgatctcagccatcctggccctggtggtgctcaccatcatctcccttatcatcctcatctgcaagagcctgctctggaagaaggtgctgccctacctgaagggcatctgttctggcggaggcggcgatcctgagagagtggatagaagctcccaaagacctggcgccgaggacaacgtgctgaacgagatcgtgtccatcctgcagcctacacaagtgcccgagcaagagatggaagtgcaagaaccagccgagcctaccggcgtgaacatgctttcacctggcgagagcgagcatctgctggaacctgccgaagccgagagatcccaaaggcggagactgctggtgccagccaatgagggcgatcctaccgagacactgagacagtgcttcgacgacttcgccgacctggtgcctttcgattcttgggagcccctgatgagaaagctgggcctgatggacaacgagatcaaggtggccaaagccgaggccgctggccacagagataccctgtacaccatgctgatcaaatgggtcaacaagaccggcagggacgccagcgttcacacactgctggatgccctggaaaccctgggagagagactggccaagcagaagatcgaggaccatctgctgagcagcggcaagttcatgtacctggaaggcaacgccgacagcgccatgagttaagtcgac

SEQ ID NO: 61 - DNA, 6487 bp, artificial sequence
  Nucleic acid sequence of plasmid T173
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatacgaacggtacgaaccggggagtcccttttaggcacttgcttctggtgctgcaactggcgctcctcccagcagccactcagggaaagaaagtggtgctgggcaacagcggcgattacaaggatgacgacgataaggttcggacgggaggtggcgggggttctaattccggagactacaaagacgatgatgacaaggtgggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccgcggtgtccccggaagaaatatatttgcatgtctttagttctatgatgacacaaaccccgcccagcgtcttgtcattggcgaattcgaacacgcagatgcagtcggggcggcgcggtccgaggtccacttcgctccctatcagtgatagagatcatattaagtccctatcagtgatagagagagctctctggctaactagagaacccactgcttactggcttatcgaaattaatacgactcactatagggagagacaagctggcggccgctggcccagtcctgaactccccgccatggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttctctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttggcattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 62 - DNA, 1321 bp, artificial sequence
  Nucleic acid sequence of plasmid T175
gcggccgcaatagccgccaccatgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctcgataccagaggtggaagtctaaactgtactcaattgtctgtggcaagtctaccccagaaaaagagggcgagctggaggggaccactactaagcccctggcccccaacccctcattcagccctacccctggtttcacaccaactcttggattcagtcccgtgcctagctctacattcacatcctccagtacctatacacccggggattgccctaacttcgccgcgccgcgccgcgaagttgcccccccataccaaggcgcagacccaatcctcgcgaccgccctcgcctcagaccctatccctaacccgctgcaaaagtgggaggattcagcacacaagccacagtcccttgacacagatgatccagccaccctctatgcagtggttgagaacgtgccccccctgaggtggaaagagtttgtgcgacgactgggactttctgatcacgaaattgaccgactggaactgcaaaatggaaggtgtcttcgcgaagcgcagtactctatgcttgccacgtggcgccgccgaacgcccagaagagaggccaccctggaactgctcggaagagtactgcgagacatggacctcctgggatgtctggaagacatagaagaagcgctgtgtgggcccgctgccctgccaccagccccttccctcttgcggtgaatctaga

SEQ ID NO: 63 - protein, 221 aa, artificial sequence
  Amino acid sequence containing the ICD of TNFR1
RYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 64 - protein, 209 aa, artificial sequence
  Amino acid sequence containing the ICD of TRAILR2
CKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS